SEQUENCE LISTING (186 sequences)

SEQ ID NO: 1 - RNA, 20 nt, synthetic construct
  source 1..20: mol_type = other RNA
  ncRNA 1..20: ncRNA_class = guide_RNA
tctctccatgtgcagtagga

SEQ ID NO: 2 - RNA, 20 nt, synthetic construct
  source 1..20: mol_type = other RNA
  ncRNA 1..20: ncRNA_class = guide_RNA
ctggatgtcggaggagtacg

SEQ ID NO: 3 - RNA, 20 nt, synthetic construct
  source 1..20: mol_type = other RNA
  ncRNA 1..20: ncRNA_class = guide_RNA
gtctccggaaactcgaggtg

SEQ ID NO: 4 - RNA, 20 nt, synthetic construct
  source 1..20: mol_type = other RNA
  ncRNA 1..20: ncRNA_class = guide_RNA
acagtgtagacttgattgac

SEQ ID NO: 5 - RNA, 20 nt, synthetic construct
  source 1..20: mol_type = other RNA
  ncRNA 1..20: ncRNA_class = guide_RNA
cgtgagtaaacctgaatctt

SEQ ID NO: 6 - protein, 21 aa, synthetic construct
MALPVTALLLPLALLLHAARP

SEQ ID NO: 7 - protein, 20 aa, synthetic construct
METDTLLLWVLLLWVPGSTG

SEQ ID NO: 8 - protein, 22 aa, synthetic construct
MLLLVTSLLLCELPHPAFLLIP

SEQ ID NO: 9 - protein, 45 aa, synthetic construct
TTTPAPRPPTPAPTIASQPLSLRPEACRPAAGGAVHTRGLDFACD

SEQ ID NO: 10 - protein, 39 aa, synthetic construct
IEVMYPPPYLDNEKSNGTIIHVKGKHLCPSPLFPGPSKP

SEQ ID NO: 11 - protein, 12 aa, synthetic construct
ESKYGPPCPPCP

SEQ ID NO: 12 - protein, 12 aa, synthetic construct
ESKYGPPCPSCP

SEQ ID NO: 13 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 14 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 15 - protein, 27 aa, synthetic construct
FWVLVVVGGVLACYSLLVTVAFIIFWV

SEQ ID NO: 16 - protein, 42 aa, synthetic construct
KRGRKKLLYIFKQPFMRPVQTTQEEDGCSCRFPEEEEGGCEL

SEQ ID NO: 17 - protein, 41 aa, synthetic construct
RSKRSRLLHSDYMNMTPRRPGPTRKHYQPYAPPRDFAAYRS

SEQ ID NO: 18 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 21 - protein, 6 aa, synthetic construct
QDISKY

SEQ ID NO: 22 - skipped

SEQ ID NO: 23 - protein, 9 aa, synthetic construct
QQGNTLPYT

SEQ ID NO: 24 - protein, 18 aa, synthetic construct
GSTSGSGKPGSGEGSTKG

SEQ ID NO: 25 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 8 aa, synthetic construct
GVSLPDYG

SEQ ID NO: 27 - protein, 7 aa, synthetic construct
IWGSETT

SEQ ID NO: 28 - protein, 14 aa, synthetic construct
AKHYYYGGSYAMDY

SEQ ID NO: 29 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 30 - protein, 15 aa, synthetic construct
GGGGSGGGGSGGGGS

SEQ ID NO: 31 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 34 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 36 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 38 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 39 - protein, 10 aa, synthetic construct
RASSSVNYMD

SEQ ID NO: 40 - protein, 7 aa, synthetic construct
ATSNLAS

SEQ ID NO: 41 - protein, 9 aa, synthetic construct
QQWSFNPPT

SEQ ID NO: 42 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 43 - protein, 5 aa, synthetic construct
SYNMH

SEQ ID NO: 44 - protein, 17 aa, synthetic construct
AIYPGNGDTSYNQKFKG

SEQ ID NO: 45 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 46 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 47 - protein, 10 aa, synthetic construct
GDSVSSNSAA

SEQ ID NO: 48 - protein, 9 aa, synthetic construct
TYYRSKWYN

SEQ ID NO: 49 - protein, 14 aa, synthetic construct
AREVTGDLEDAFDI

SEQ ID NO: 50 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 6 aa, synthetic construct
QTIWSY

SEQ ID NO: 52 - skipped

SEQ ID NO: 53 - protein, 9 aa, synthetic construct
QQSYSIPQT

SEQ ID NO: 54 - protein, 246 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 55 - protein, 124 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 56 - protein, 10 aa, synthetic construct
GDSVSSNSVA

SEQ ID NO: 57 - protein, 9 aa, synthetic construct
TYYRSTWYN

SEQ ID NO: 58 - protein, 14 aa, synthetic construct
AREVTGDLEDAFDI

SEQ ID NO: 59 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 60 - protein, 6 aa, synthetic construct
QTIWSY

SEQ ID NO: 61 - skipped

SEQ ID NO: 62 - protein, 9 aa, synthetic construct
QQSYSIPQT

SEQ ID NO: 63 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 64 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 65 - protein, 15 aa, synthetic construct
RASESVSVIGAHLIH

SEQ ID NO: 66 - protein, 7 aa, synthetic construct
LASNLET

SEQ ID NO: 67 - protein, 9 aa, synthetic construct
LQSRIFPRT

SEQ ID NO: 68 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - protein, 5 aa, synthetic construct
DYSIN

SEQ ID NO: 70 - protein, 17 aa, synthetic construct
WINTETREPAYAYDFRG

SEQ ID NO: 71 - protein, 8 aa, synthetic construct
DYSYAMDY

SEQ ID NO: 72 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 73 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 74 - protein, 15 aa, synthetic construct
RASESVTILGSHLIY

SEQ ID NO: 75 - protein, 7 aa, synthetic construct
LASNVQT

SEQ ID NO: 76 - protein, 9 aa, synthetic construct
LQSRTIPRT

SEQ ID NO: 77 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 78 - protein, 5 aa, synthetic construct
HYSMN

SEQ ID NO: 79 - protein, 17 aa, synthetic construct
RINTESGVPIYADDFKG

SEQ ID NO: 80 - protein, 8 aa, synthetic construct
DYLYSLDF

SEQ ID NO: 81 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 82 - protein, 8 aa, synthetic construct
GFTFSSYA

SEQ ID NO: 83 - protein, 8 aa, synthetic construct
ISGSGDYI

SEQ ID NO: 84 - protein, 14 aa, synthetic construct
AKEGTGANSSLADY

SEQ ID NO: 85-112 - skipped (28 entries)

SEQ ID NO: 113 - protein, 42 aa, synthetic construct
AAAIEVMYPPPYLDNEKSNGTIIHVKGKHLCPSPLFPGPSKP

SEQ ID NO: 114 - protein, 28 aa, synthetic construct
MFWVLVVVGGVLACYSLLVTVAFIIFWV

SEQ ID NO: 115 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1467 bp, synthetic construct
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 117 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 119 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 120 - protein, 6 aa, synthetic construct
QSISSY

SEQ ID NO: 121 - skipped

SEQ ID NO: 122 - protein, 8 aa, synthetic construct
QQKYDLLT

SEQ ID NO: 123 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 124 - protein, 10 aa, synthetic construct
GGSISSSSYY

SEQ ID NO: 125 - protein, 7 aa, synthetic construct
ISYSGST

SEQ ID NO: 126 - protein, 11 aa, synthetic construct
ARDRGDTILDV

SEQ ID NO: 127 - DNA, 1503 bp, synthetic construct
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 128 - protein, 501 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 119 aa, synthetic construct
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 130 - protein, 107 aa, synthetic construct
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 131 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 132 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 133 - protein, 667 aa, synthetic construct
QVQLQQSGAELARPGASVKMSCKASGYTFTTYTIHVWRRRPGHDLEWIGYINPSSGCSDYNQNFKGKTTLTADKSSNTAYMQLNSLTSEDSAVYYCARRADYGNYEYTWFAYVVGQGTTVTVSSSGGGSGGGGSGGGGSVIELTQSPKFMSTSVGDRVNVTYKASQNVGTNVAWFQQKPGQSPKVLIYSASYRYSGVPDRFTGSGSGTDFTLTISNVQSEDLAEYFCQQYHTYPLTFGGGTKLEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFVWLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 536 aa, synthetic construct
MEFGLSWLFLVAILKGVQCSRDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKGGGSGGGGQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAGSELPTQGTFSNVSTNVSPAPRPPTPAPTIASQPLSLRPEACRPAAGGAVHTRGLDFACDIYIWAPLAGTCGVLLLSLVITLYCNHRNEFRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 219 aa, synthetic construct
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCARDPYGYIFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 136 - protein, 215 aa, synthetic construct
DIVMTQSQKFMSTSVGDRVSITCKASQNVHTAVAEWYQQKAGQSPKALIYLASNRHTGVPDRFTGSGSGTDFTLTISNVQSEDLADYFCLQHWNNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 137 - protein, 115 aa, synthetic construct
EVQLLESGGGLVQPGGSLRLSCAASGFTFSGYAMSWVRQAPGKGLEWVSEIAPPGLNTRYADSVKGRFTISRDNSKNTLYLQMNSLRAEDTAVYYCAKSDTAFDYWGQGTLVTVS

SEQ ID NO: 138 - protein, 111 aa, synthetic construct
STDIQMTQSPSSLSASVGDRVTITCRASQSISSYLNWYQQKPGKAPKLLIYLASNLQSGVPSRFSGSGSGTDFTLTISSLQPEDFATYYCQQAEYMPLTFGQGTKVEIKRA

SEQ ID NO: 139 - protein, 241 aa, synthetic construct
EVQLLESGGGLVQPGGSLRLSCAASGFTFSGYAMSWVRQAPGKGLEWVSEIAPPGLNTRYADSVKGRFTISRDNSKNTLYLQMNSLRAEDTAVYYCAKSDTAFDYWGQGTLVTVSSGGGGSGGGGSGGGGSTDIQMTQSPSSLSASVGDRVTITCRASQSISSYLNWYQQKPGKAPKLLIYLASNLQSGVPSRFSGSGSGTDFTLTISSLQPEDFATYYCQQAEYMPLTFGQGTKVEIKRA

SEQ ID NO: 140 - protein, 237 aa, synthetic construct
QVQLVQSGSELKKPGSSVKVSCKASGGTFSSYAISWVRQAPGQGLEWMGGIIPIFGTANYAQKFQGRVTITADKSTSTAYMELSSLRSEDTAVYYCARGRDGMDVWGQGTLVTVSSGGGGSGGGGSGGGGSQPGLTQPPSVSVSPGQTASITCSGDELGNRYAYWYQQKPGQSPVLVIYQDRKRPSGIPERFSGSNSGNTATLTISGTQAMDEADYYCQAWASGTGVFGTGTKVTVL

SEQ ID NO: 141 - protein, 234 aa, synthetic construct
VQLVQSGAEVKKPGSSVKVSCKASGVTFSSYGINWVRQAPGQGLEWMGGIIPIFGTGNYAQKFQGRLTISADESTSTAYMELNSLRSEDTAVYYCARGNPFGQTWGQGTLVTVSSGGGGSGGGGSGGGGSQPGLTQPPSVSPGQTASITCSGDKLGDKYASWYQLKPGQSPLLVIYQDVKRPSGIPERFSGSNSGNTATLTISGTQAMDEADYYCQAWDSGTAVFGGGTKLTVL

SEQ ID NO: 142 - protein, 238 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPYLGWGQGTMVTVSSGGGGSGGGGSGGGGSNFMLTQPHSVSESPGKTVNISCTRSSGSIASHYVQWFQQRPGSAPATVIYEDKQRPSGVPDRFSGSIDSSSNSASLTISGLRTEDEADYYCQSYDTGTWVFGGGTKLTVL

SEQ ID NO: 143 - protein, 132 aa, synthetic construct
METLLGLLILWLQLQWVSSKQEVTQIPAALSVPEGENVLVLNCSFTDSAIYNLQWFRQDPGKGLTSLLLIQSSQREQTSGRLNASLDKSSGRSTLYIAASQPGDSATYLCAVLMDSNYQLIWGAGTKLIIKP

SEQ ID NO: 144 - protein, 132 aa, synthetic construct
MATRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSEDGMNTEAFFGQGTRLTVV

SEQ ID NO: 145 - protein, 129 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGSGAGSYQLTFGKGTKLSVIP

SEQ ID NO: 146 - protein, 134 aa, synthetic construct
MASRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLEGQASSYEQYFGPGTRLTVT

SEQ ID NO: 147 - protein, 131 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAATLWVFGNEKLTFGTGTRLTIIP

SEQ ID NO: 148 - protein, 132 aa, synthetic construct
MALLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDVRGTDVYEQYFGPGTRLTVT

SEQ ID NO: 149 - protein, 205 aa, synthetic construct
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSAEAHQPYRLNFGKGTSLLVTPHIQNPDPAWQLRDSKSSDKSVCLFTDFDSQTNVSQSKDSDWITDKCVLDMRSMDFKSNSAVAWSNKSDFACANAFNNSIIPEDTFFPSPESS

SEQ ID NO: 150 - protein, 207 aa, synthetic construct
QKEVEQDPGPLSVPEGAIVSLNCTYSNSAFQYFMWYRQYSRKGPELLMYLPGGGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 151 - protein, 207 aa, synthetic construct
QKEVEQDPGPLSVPEGAIVSLNCTYSNSAFQYFMWYRQYSRKGPELLMYQFQDGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 152 - protein, 207 aa, synthetic construct
QKEVEQDPGPLSVPEGAIVSLNCTYSNPHMAYFMWYRQYSRKGPELLMYTYSSGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 153 - protein, 207 aa, synthetic construct
QKEVEQDPGPLSVPEGAIVSLNCTYSNPHMAYFMWYRQYSRKGPELLMYTYSSGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 154 - protein, 207 aa, synthetic construct
QKEVEQDPGPLSVPEGAIVSLNCTYSNPHMAYFMWYRQYSRKGPELLMYQFQDGNKEDGRFTAQVDKSSKYISLFIRDSQPSDSATYLCAMSADAYGHYALNFGKGTSLLVTPHIQNPDPAVYQLRDSKSSDKSVCLFTDFDSQTNVSQSKDSDVYITDKFVLDMRSMDFKSNSAVAWSNKSDFACANAFNNSIIPEDTFFPSPESS

SEQ ID NO: 155 - protein, 207 aa, synthetic construct
QKEVEQDPGPLSVPEGAIVSLNCTYSNPHMAYFMWYRQYSRKGPELLMYQFQDGNKEDGRFTAQVDKSSKYISLFIRDSQPSDSATYLCAMSAEAHQPYRLNFGKGTSLLVTPHIQNPDPAVYQLRDSKSSDKSVCLFTDFDSQTNVSQSKDSDVYITDKFVLDMRSMDFKSNSAVAWSNKSDFACANAFNNSIIPEDTFFPSPESS

SEQ ID NO: 156 - protein, 242 aa, synthetic construct
EAGVAQSPRYKIIEKRQSVAFWCNPISGHATLYWYQQILGQGPKLLIQFVVAASVDDSQLPKDRFSAERLKGVDSTLKIQPAKLEDSAVYLCASSLGGYEQYFGPGTRLTVTEDLKNVFPPEVAVFEPSEAEISHTQKATLVCLATGFYPDHVELSVVWVNGKEVHSGVTDPQPLKEQPALNDSRYALSSRLRVSATFWQDPRNHFRCQVQFYGLSENDEWTQDRAKPVTQIVSAEAWGRAD

SEQ ID NO: 157 - protein, 121 aa, synthetic construct
QVQLKESGPGLVQPSQTLSLTCTVSGLSLTSNGVSWIRQPPGKGLVHEWLGVIWSNGGTDYNSAIKSRLSFSRDTSKSQVFLKMNSLQTEDTAMYFCARPRYNSGYFFDYWGQGVMVTVSS

SEQ ID NO: 158 - protein, 107 aa, synthetic construct
DTVLTQSPALAVSPGERVTISCKASESVSTRMHWYRQKPGQQPKLLIYKTSNLASGVPARFSGSGSGTDFTLTLDPVEADDTATYFCQQSWNGPLTFGSGTKLEIKR

SEQ ID NO: 159 - protein, 124 aa, synthetic construct
QVQLKESGPGLVQPSQTLSLTCTVSGFSLTSYHVHWVRQPPGKGLEWMGVMWSDGDTLYNSALKSRLSISRDTSKSQVLLQMDSLQSEDTATYYCARLQVFGYPGIRDYVMDAWGQGASVTVSS

SEQ ID NO: 160 - protein, 114 aa, synthetic construct
DLVMTQSPFSLAVSEGEMVTIKCKSSQSLLSSRHQKNFLAWYRQKPGQSPKLLIYHASTRQSGVPDRFIGSGSGTDFTLTISDVQAEDLADYYCLQHYTSPYTFGAGTKLELKR

SEQ ID NO: 161 - protein, 119 aa, synthetic construct
EVQLVESGGGLVQPGGSLRLSCAASGSSFTDYTMNWMRQSPGKGLEWIGLINPYNGGTRYADSVKGRATLSLDKSKNTAYLQMNSLRAEDTAIYYCAGGLRRVNWFAYWGQGTLVTVSS

SEQ ID NO: 162 - protein, 111 aa, synthetic construct
DAQLTQSPILLSASVGDRVTITCRSSTGAVTTSNYANWVQERTNGSFRGLIGGTNNRVPGVPSRFSGSLSGDDATLTISSLQPEDEADYFCVLWHSNHWVFGAGTKVEIKR

SEQ ID NO: 163 - protein, 110 aa, synthetic construct
SVVLTQPPSVSVAPGKTARITCGGNNIGSKSVHWYQQKPGQAPVLVIYYDSDRPSGIPERFSGSNSGNTATLTISRVEAGDEADVYCQVWDSSSDHPLYVFGTGTKVTVL

SEQ ID NO: 164 - protein, 123 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYVCARALEMGYRSGFPFDYWGQGTLVTVSS

SEQ ID NO: 165 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYVCQQANSFPLTFGGGTKVEIK

SEQ ID NO: 166 - protein, 130 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYVCARDRVLGAHGGNPLNGHVYGMDVWGQGTTVTVSS

SEQ ID NO: 167 - protein, 106 aa, synthetic construct
SVELTQPPSVSVSPGQTASITCSGDKLGDKYACWYQQKPGQSPVLVIYQDSKRPSGIPERFSGSNSGNTATLTISGTQAMDEADYVCQAWDSSTVVFGGGTKLTVL

SEQ ID NO: 168 - protein, 123 aa, synthetic construct
QVTLKESGPTLVKPTQTLTLTCTFSGFSLSTSGVGVGWIRQPPGKALEWLALIYWDDDKRYGPSLKSRLTITKDTSKNQVVLTMTNMDPVDTATYYCAHRDKLYSGYVKFDYWGQGTLVTVSS

SEQ ID NO: 169 - protein, 114 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYVCMQGTHWPPMYTFGQGTKLEIK

SEQ ID NO: 170 - protein, 126 aa, synthetic construct
QVQLVQSGAEVKKPGSSVKVSCKASGGTFSSYAISWVRQAPGQGLEWMGGIIPLLGIANYAQKFQGRVTITADKSTSTAYMELSSLRSEDTAVYYCARDVPGQCTRTSCYNFSSQWGQGTLVTVSS

SEQ ID NO: 171 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPRTFGQGTKVEIK

SEQ ID NO: 172 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGAGDYLGWFDPWGQGTLVTVSS

SEQ ID NO: 173 - protein, 6 aa, synthetic construct
  REGION >6: note = The entire sequence of amino acids 1-6 can be repeated one to ten times
SGGGGS

SEQ ID NO: 174 - protein, 5 aa, synthetic construct
  REGION >5: note = The entire sequence of amino acids 1-5 can be repeated one to ten times
GGGGS

SEQ ID NO: 175 - protein, 7 aa, synthetic construct
  REGION >7: note = The entire sequence of amino acids 1-7 can be repeated one to ten times
GGGGSSS

SEQ ID NO: 176 - protein, 16 aa, synthetic construct
SGGGGSGGGGSGGGGS

SEQ ID NO: 177 - protein, 21 aa, synthetic construct
SGGGGSGGGGSGGGGSGGGGS

SEQ ID NO: 178 - protein, 4 aa, synthetic construct
  REGION >4: note = The entire sequence of amino acids 1-4 can be repeated one to six times
GGGS

SEQ ID NO: 179 - protein, 5 aa, synthetic construct
  REGION >5: note = The entire sequence of amino acids 1-5 can be repeated one to six times
GGGGS

SEQ ID NO: 180 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 181 - protein, 24 aa, synthetic construct
IYIWAPLAGTCGVLLLSLVITLYC

SEQ ID NO: 182 - RNA, 20 nt, synthetic construct
  source 1..20: mol_type = other RNA
  ncRNA 1..20: ncRNA_class = guide_RNA
gatattggcataagcctccc

SEQ ID NO: 183 - RNA, 20 nt, synthetic construct
  source 1..20: mol_type = other RNA
  ncRNA 1..20: ncRNA_class = guide_RNA
agagtctctcagctggtaca

SEQ ID NO: 184 - RNA, 12 nt, synthetic construct
  source 1..12: mol_type = other RNA
  ncRNA 1..12: ncRNA_class = guide_RNA
gttttagagcta

SEQ ID NO: 185 - RNA, 63 nt, synthetic construct
  source 1..63: mol_type = other RNA
  ncRNA 1..63: ncRNA_class = guide_RNA
tagcaagttaaaataaggctagtccgttatcaacttgaaaaagtggcaccgagtcggtgcttt

SEQ ID NO: 186 - RNA, 99 nt, synthetic construct
  source 1..99: mol_type = other RNA
  ncRNA 1..99: ncRNA_class = guide_RNA
nnnnnnnnnnnnnnnnnnnngttttagagctagaaatagcaagttaaaataaggctagtccgttatcaacttgaaaaagtggcaccgagtcggtgcttt

(19) United States
(12) Patent Application Publication
(10) Pub. No.: US 2025/0302876 A1
(43) Pub. Date: Oct. 2, 2025

(54) CHIMERIC ANTIGEN RECEPTOR (CAR) T CELLS FOR TREATING AUTOIMMUNE DISEASE AND ASSOCIATED METHODS

(71) Applicant: Sana Biotechnology, Inc., Seattle, WA (US)

(72) Inventors: Sunil Agarwal, Seattle, WA (US); Sonja Schrepfer, San Mateo, CA (US); Terry James Fry, Denver, CO (US); Paul Brunetta, San Francisco, CA (US); Constantine Stephen Djedjos, Belmont, CA (US); Steve Harr, Seattle, WA (US)

(21) Appl. No.: 18/722,924

(22) PCT Filed: Dec. 23, 2022
(86) PCT No.: PCT/US2022/053971

§ 371 Date: Jun. 21, 2024

Related U.S. Application Data
(60) Provisional application No. 63/293,637, filed on Dec. 23, 2021.
(60) Provisional application No. 63/320,672, filed on Mar. 16, 2022.

(51) Int. Cl. A61K 35/17; A61K 40/11; A61K 40/20; A61K 40/31; A61K 40/42; C07K 14/705; C07K 14/725; C07K 16/28

(52) U.S. Cl. CPC A61K 35/17; A61K 40/11; A61K 40/20; A61K 40/31; A61K 40/4211; A61K 40/4212; C07K 14/7051; C07K 14/70517; C07K 14/70521; C07K 16/2803; A61K 2239/00; A61K 2239/13; A61K 2239/17; A61K 2239/21; A61K 2239/31

(57) ABSTRACT

Disclosed herein are engineered cells and/or hypoimmunogenic cells including engineered and/or hypoimmunogenic stem cells, engineered and/or hypoimmunogenic cells differentiated therefrom, engineered and/or hypoimmunogenic CAR-T cells (primary or differentiated from engineered and/or hypoimmunogenic stem cells) and related methods of their use and generation for use in the treatment of autoimmune diseases/disorders and/or inflammatory diseases/disorders. Provided herein are engineered and/or hypoimmunogenic cells exhibiting reduced expression of MHC class I and/or MHC class II human leukocyte antigens and T-cell receptors for use in the treatment of autoimmune diseases/disorders and/or inflammatory diseases/disorders. In some embodiments, such cells also exogenously express one or more tolerogenic factors such as CD47 and one or more chimeric antigen receptors (CARS).

### CROSS REFERENCE TO RELATED APPLICATIONS

This application claims priority to and the benefit of U.S. Provisional Application No. 63/293,637, filed Dec. 23, 2021 and U.S. Provisional Application No. 63/320,672, filed Mar. 16, 2022, the contents of which are incorporated herein by reference in their entirety.

### SEQUENCE LISTING

The instant application contains a Sequence Listing which has been submitted in XML format and is hereby incorporated by reference in its entirety. Said XML copy, created on Jul. 15, 2024, is named 2017428-0022.xml and is 168,727 bytes in size.

### SUMMARY

Off-the-shelf CAR-T cells and other therapeutic cells can offer advantages over autologous cell-based strategies, including ease of manufacturing, quality control and avoidance of malignant contamination and T cell dysfunction. However, the vigorous host-versus-graft immune response against histoincompatible T cells prevents expansion and persistence of allogeneic CAR-T cells and mitigates the efficacy of this approach.

There is substantial evidence in both animal models and human patients that hypoimmunogenic cell transplantation is a scientifically feasible and clinically promising approach to the treatment of numerous disorders, conditions, and diseases, in particular for the treatment of autoimmune diseases/disorders and/or inflammatory diseases/disorders.

There remains a need for novel approaches, compositions and methods for producing cell-based therapies that avoid detection by the recipient's immune system.

In some embodiments, provided herein is an engineered cell comprising reduced expression of HLA-A, HLA-B, HLA-C, HLA-DP, HLA-DQ, HLA-DR, B2M, CIITA, TCR-alpha, and/or TCR-beta relative to a wild-type cell or a control cell, the engineered cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell.

In some embodiments, the specific locus is selected from the group consisting of a safe harbor or target locus, a B2M locus, a CIITA locus, a TRAC locus and a TRB locus. In many embodiments, the first exogenous polynucleotide encoding CD47 is inserted into the specific locus selected from the group consisting of a safe harbor or target locus, a B2M locus, a CIITA locus, a TRAC locus and a TRB locus. In some embodiments, the second exogenous polynucleotide encoding the CAR is inserted into the specific locus selected from the group consisting of a safe harbor or target locus, a B2M locus, a CIITA locus, a TRAC locus and a TRB locus.

In some embodiments, the first exogenous polynucleotide encoding CD47 and the second exogenous polynucleotide encoding the CAR are inserted into different loci. In many embodiments, the first exogenous polynucleotide encoding CD47 and the second exogenous polynucleotide encoding the CAR are inserted into the same locus. In several embodiments, the first exogenous polynucleotide encoding CD47 and the second exogenous polynucleotide encoding the CAR are inserted into the B2M locus. In some embodiments, the first exogenous polynucleotide encoding CD47 and the second exogenous polynucleotide encoding the CAR are inserted into the CIITA locus. In many embodiments, the first exogenous polynucleotide encoding CD47 and the second exogenous polynucleotide encoding the CAR are inserted into the TRAC locus. In some embodiments, the first exogenous polynucleotide encoding CD47 and the second exogenous polynucleotide encoding the CAR are inserted into the TRB locus. In some embodiments, the first exogenous polynucleotide encoding CD47 and the second exogenous polynucleotide encoding the CAR are inserted into the safe harbor or target locus. In some embodiments, the safe harbor or target locus is selected from the group consisting of a CCR5 gene locus, a CXCR4 gene locus, a PPP1R12C (also known as AAVS1) gene locus, an albumin gene locus, a SHS231 gene locus, a CLYBL gene locus, a Rosa gene locus (e.g., ROSA26 gene locus), an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. In various embodiments, the safe harbor or target locus is selected from the group consisting of the AAVS1 locus, the CCR5 locus, and the ROSA26 locus.

In some embodiments, the CAR is selected from the group consisting of a CD19-specific CAR, a CD22-specific CAR, and a CD20-specific CAR. In some embodiments, the CAR is a bispecific CAR. In some embodiments, the CAR is a CD19-specific CAR. In some embodiments, the CAR is a CD22-specific CAR. In some embodiments, the CAR is a CD20-specific CAR. In some embodiments, the CAR is a bispecific CAR. In some embodiments, the CAR is a CD19/CD20-bispecific CAR. In some embodiments, the CAR is a CD19/CD22-bispecific CAR.

In many embodiments, the engineered cell does not express HLA-A, HLA-B, and/or HLA-C antigens. In some embodiments, the engineered cell does not express B2M. In other embodiments, the engineered cell does not express HLA-DP, HLA-DQ, and/or HLA-DR antigens. In some embodiments, the engineered cell does not express CIITA. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell does not express TCR-alpha and/or TCR-beta.

In many embodiments, the engineered cell is a pluripotent stem cell. In some embodiments, the engineered cell is an induced pluripotent stem cell.

In some embodiments, the engineered cell is a differentiated cell derived from an induced pluripotent stem cell. In various embodiments, the differentiated cell is selected from the group consisting of an NK cell and a T cell.

In some embodiments, the engineered cell is a cell derived from a primary T cell. In many embodiments, the cell derived from the primary T cell is derived from a pool of T cells comprising primary T cells from one or more donor subjects who are different from a recipient subject.

In some embodiments, the engineered cell is a cell derived from a primary NK cell. In many embodiments, the cell derived from the primary NK cell is derived from a pool of NK cells comprising primary NK cells from one or more donor subjects who are different from a recipient subject.

In some embodiments, the engineered cell retains pluripotency and/or retains differentiation potential.

In many embodiments, following transfer into a first subject, the engineered cell exhibits one or more responses selected from the group consisting of (a) a T cell response, (b) an NK cell response, and (c) a macrophage response, that are reduced as compared to a wild-type cell following transfer into a second subject. In some instances, the first subject and the second subject are different subjects. In some instances, the macrophage response is engulfment. In various embodiments, following transfer into a subject the engineered cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject. In many embodiments, following transfer into a subject the engineered cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising first exogenous polynucleotide encoding CD47 and/or the second exogenous polynucleotide encoding CAR inserted into the TRAC locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising the first exogenous polynucleotide encoding CD47 and the second exogenous polynucleotide encoding CAR inserted into the TRAC locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel} CIITA^{indel/indel}, and/or TRAC^{indel/indel} cell comprising first exogenous polynucleotide encoding CD47 and/or the second exogenous polynucleotide encoding CAR inserted into the TRB locus. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising the first exogenous polynucleotide encoding CD47 and the second exogenous polynucleotide encoding CAR inserted into the TRB locus. In numerous embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising first exogenous polynucleotide encoding CD47 and/or the second exogenous polynucleotide encoding CAR inserted into the B2M locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising the first exogenous polynucleotide encoding CD47 and the second exogenous polynucleotide encoding CAR inserted into a B2M locus. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising first exogenous polynucleotide encoding CD47 and/or the second exogenous polynucleotide encoding CAR inserted into the CIITA locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRAC^{indel/indel} cell comprising the first exogenous polynucleotide encoding CD47 and the second exogenous polynucleotide encoding CAR inserted into a CIITA locus.

In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising first exogenous polynucleotide encoding CD47 and/or the second exogenous polynucleotide encoding CAR inserted into the TRAC locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising the first exogenous polynucleotide encoding CD47 and the second exogenous polynucleotide encoding CAR inserted into the TRAC locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising first exogenous polynucleotide encoding CD47 and/or the second exogenous polynucleotide encoding CAR inserted into the TRB locus. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising the first exogenous polynucleotide encoding CD47 and the second exogenous polynucleotide encoding CAR inserted into the TRB locus. In numerous embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising first exogenous polynucleotide encoding CD47 and/or the second exogenous polynucleotide encoding CAR inserted into the B2M locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising the first exogenous polynucleotide encoding CD47 and the second exogenous polynucleotide encoding CAR inserted into a B2M locus. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising first exogenous polynucleotide encoding CD47 and/or the second exogenous polynucleotide encoding CAR inserted into the CIITA locus. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, and/or TRB^{indel/indel} cell comprising the first exogenous polynucleotide encoding CD47 and the second exogenous polynucleotide encoding CAR inserted into a CIITA locus.

In some embodiments, the engineered cell is a B2M^{indel/indel}, CIITA^{indel/indel} TRAC^{indel/indel}, and/or TRB^{indel/indel} cell comprising first exogenous polynucleotide encoding CD47 and/or the second exogenous polynucleotide encoding CAR inserted into the TRAC locus. In many embodiments, the engineered cell is a B2M^{indel/indel}, CIITA^{indel/indel}, TRAC^{indel/indel}, and/or TRB^{indel/indel} cell comprising the first exogenous polynucleotide encoding CD47 and the second exogenous polynucleotide encoding CAR inserted into the TRAC locus. In many embodiments, the engineered cell is a B2M^{indel/indel}, CIITA^{indel/indel}, TRAC^{indel/indel}, and/or TRB^{indel/indel} cell comprising first exogenous polynucleotide encoding CD47 and/or the second exogenous polynucleotide encoding CAR inserted into the TRB locus. In some embodiments, the engineered cell is a B2M^{indel/indel}, CIITA^{indel/indel}, TRAC^{indel/indel}, and/or TRB^{indel/indel} cell comprising the first exogenous polynucleotide encoding CD47 and the second exogenous polynucleotide encoding CAR inserted into the TRB locus. In numerous embodiments, the engineered cell is a B2M^{indel/indel}, CIITA^{indel/indel}, TRAC^{indel/indel}, and/or TRB^{indel/indel} cell comprising first exogenous polynucleotide encoding CD47 and/or the second exogenous polynucleotide encoding CAR inserted into the B2M locus. In many embodiments, the engineered cell is a B2M^{indel/indel} CIITA^{indel/indel}, TRAC^{indel/indel}, and/or TRB^{indel/indel} cell comprising the first exogenous polynucleotide encoding CD47 and the second exogenous polynucleotide encoding CAR inserted into a B2M locus. In some embodiments, the engineered cell is a B2M^{indel/indel}, CIITA^{indel/indel}, TRAC^{indel/indel} and/or TRB^{indel/indel} cell comprising first exogenous polynucleotide encoding CD47 and/or the second exogenous polynucleotide encoding CAR inserted into the CIITA locus. In many embodiments, the engineered cell is a B2M^{indel/indel}, CIITA^{indel/indel}, TRAC^{indel/indel}, and/or TRB^{indel/indel} cell comprising the first exogenous polynucleotide encoding CD47 and the second exogenous polynucleotide encoding CAR inserted into a CIITA locus.

In some embodiments, provided is an engineered cell comprising reduced expression of HLA-A, HLA-B, HLA-C, HLA-DP, HLA-DQ, HLA-DR, B2M, CIITA, TCR-alpha, and/or TCR-beta relative to a wild-type cell or a control cell.

In some embodiments, the engineered cell does not express HLA-A, HLA-B and/or HLA-C antigens. In many embodiments, the engineered cell does not express CIITA.

In many embodiments, the engineered cell does not express HLA-DP, HLA-DQ, and/or HLA-DR antigens. In some embodiments, the engineered cell does not express B2M.

In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell does not express TCR-alpha. In many embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell does not express TCR-beta.

In various embodiments, the engineered cell overexpresses CD47 relative to a wild-type cell or a control cell.

In some embodiments, the engineered cell is a pluripotent stem cell. In many embodiments, the engineered cell is an induced pluripotent stem cell.

In many embodiments, the engineered cell is a differentiated cell derived from an induced pluripotent stem cell. In some embodiments, the differentiated cell is selected from the group consisting of an NK cell and a T cell.

In many embodiments, the engineered cell is a cell derived from a primary T cell. In several embodiments, the cell derived from the primary T cell is derived from a pool of T cells comprising primary T cells from one or more donor subjects who are different from a recipient subject.

In various embodiments, the engineered cell retains pluripotency and/or retains differentiation potential.

In some embodiments, following transfer into a subject the engineered cell elicits one or more response selected from the group consisting of (a) a T cell response, (b) an NK cell response, and (c) a macrophage response, that are reduced as compared to a wild-type cell following transfer into a second subject. In some instances, the first subject and the second subject are different subjects. In some instances, the macrophage response is engulfment.

In various embodiments, following transfer into a subject the engineered cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject. In many embodiments, following transfer into a subject the engineered cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the engineered cell is a B2M^{indel/indel}, CIITA^{indel/indel} TRAC^{indel/indel}, and/or TRB^{indel/indel} cell. In some instances, the engineered cell is a B2M^{indel/indel}, CIITA^{indel/indel}, TRAC^{indel/indel}, and/or TRB^{indel/indel} primary T cell. In some instances, the engineered cell is a B2M^{indel/indel}, CIITA^{indel/indel}, TRAC^{indel/indel}, and/or TRB^{indel/indel} T cell differentiated from a hypoimmunogenic induced pluripotent stem cell. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, TRAC^{indel/indel} cell. In some instances, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, TRAC^{indel/indel} primary T cell. In some embodiments, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, TRB^{indel/indel} cell. In some instances, the engineered cell is selected from the group consisting of a pluripotent stem cell, an induced pluripotent stem cell, a T cell differentiated from an induced pluripotent stem cell, a primary T cell, and a cell derived from a primary T cell, and the engineered cell is a B2M^{indel/indel}, CIITA^{indel/indel}, TRB^{indel/indel} primary T cell. In some instances, the engineered cell is a B2M^{indel/indel}, CIITA^{indel/indel}, and/or TRAC^{indel/indel} T cell differentiated from a hypoimmunogenic induced pluripotent stem cell. In some embodiments, the engineered cell is a B2M^{indel/indel}, CIITA^{indel/indel}, and/or TRB^{indel/indel} cell. In some instances, the engineered cell is a B2M^{indel/indel}, CIITA^{indel/indel}, and/or TRB^{indel/indel} primary T cell. In some instances, the engineered cell is a B2M^{indel/indel}, CIITA^{indel/indel}, and/or TRB^{indel/indel} T cell differentiated from a hypoimmunogenic induced pluripotent stem cell.

In some embodiments, the engineered cell is a hypoimmunogenic cell.

In some embodiments, provided is a pharmaceutical composition comprising a population of any of the engineered cells described herein and a pharmaceutically acceptable additive, carrier, diluent or excipient.

In some embodiments, the pharmaceutically acceptable additive, carrier, diluent or excipient comprises one or more selected from the group consisting of Plasma-Lyte A®, dextrose, dextran, sodium chloride, human serum albumin (HSA), dimethylsulfoxide (DMSO), and a combination thereof. In some embodiments, the pharmaceutical composition further comprises a pharmaceutically acceptable buffer. In some embodiments, the pharmaceutically acceptable buffer is neutral buffer saline or phosphate buffered saline.

In some embodiments, provided is a pharmaceutical composition comprising a population of any of the engineered cells described herein, a base solution of CryoStor® CSB at a concentration of about 70-80% w/w, and one or more of about 20-30% w/w PlasmaLyte-A™, about 0.3-5.3% w/v human serum albumin (HSA), about 0-20% v/v dimethylsulfoxide (DMSO), and about 100-400 mM trehalose.

In some embodiments, provided is a pharmaceutical composition comprising a population of any of the engineered cells described herein, a base solution of PlasmaLyte-A™ at a concentration of about 20-30% w/w, and one or more of about 70-80% w/w CryoStor® CSB, about 0.3-5.3% w/v human serum albumin (HSA), about 0-20% v/v dimethylsulfoxide (DMSO), and about 100-400 mM trehalose.

In some embodiments, provided is a pharmaceutical composition comprising a population of any of the engineered cells described herein, about 0.3-5.3% w/v human serum albumin (HSA), and one or more of about 70-80% w/w CryoStor® CSB, about 20-30% w/w PlasmaLyte-A™, about 0-20% v/v dimethylsulfoxide (DMSO), and about 100-400 mM trehalose.

In some embodiments, provided is a pharmaceutical composition comprising a population of any of the engineered cells described herein, about 0-20% v/v dimethylsulfoxide (DMSO), and one or more of about 70-80% w/w CryoStor® CSB, about 20-30% w/w PlasmaLyte-A™, about 0.3-5.3% w/v human serum albumin (HSA), and about 100-400 mM trehalose.

In some embodiments, provided is a pharmaceutical composition comprising a population of any of the engineered cells described herein, about 100-400 mM trehalose, and one or more of about 70-80% w/w CryoStor® CSB, about 20-30% w/w PlasmaLyte-A™, about 0.3-5.3% w/v human serum albumin (HSA), and about 0-20% v/v dimethylsulfoxide (DMSO).

In some embodiments, the pharmaceutical composition comprises about 75% w/w of CryoStor® CSB. In some embodiments, the pharmaceutical composition comprises about 25% w/w of PlasmaLyte-A™. In some embodiments, the pharmaceutical composition comprises about 0.3% w/v of HSA. In some embodiments, the pharmaceutical composition comprises about 7.5% v/v of DMSO.

In some embodiments, provided is a pharmaceutical composition comprising a population of any of the engineered cells described herein, a base solution of CryoStor® CSB at a concentration of about 75% w/w, about 25% w/w PlasmaLyte-A™, about 0.3% w/v human serum albumin (HSA), and about 7.5% v/v dimethylsulfoxide (DMSO).

In some embodiments, the population of the engineered cells is up to about 8.0×10⁸ cells. In many embodiments, the population of the engineered cells is up to about 6.0×10⁸ cells. In other embodiments, the population of the engineered cells is from about 1.0×10⁶ to about 2.5×10⁸ cells. In some embodiments, the population of the engineered cells is from about 2.0×10⁶ to about 2.0×10⁸ cells.

In various embodiments, the population of the engineered cells ranges from about 5 ml to about 80 ml. In many embodiments, the population of the engineered cells ranges from about 10 ml to about 70 ml. In some embodiments, the population of the engineered cells ranges from about 10 ml to about 50 ml.

In some embodiments, the composition is formulated for administration in a single dose. In many embodiments, the composition is formulated for administration in up to three doses.

In some embodiments, the composition is formulated for administration of a single dose to a subject takes a duration of time of about 60 minutes or less. In many embodiments, the composition is formulated for administration of a single dose to a subject takes a duration of time of about 30 minutes or less.

In some embodiments, the population of engineered cells of the pharmaceutical composition or progeny thereof exhibit at least 40% survival in a subject after 10 days following administration. In various embodiments, the population of engineered cells of the pharmaceutical composition or progeny thereof exhibit at least 80% survival in a subject after about 2 weeks following administration. In several embodiments, the population of engineered cells of the pharmaceutical composition or progeny thereof exhibit at least 100% survival in a subject after about 3 weeks following administration. In many embodiments, the population of engineered cells of the pharmaceutical composition or progeny thereof exhibit at least 150% survival in a subject after about 4 weeks following administration.

In another embodiment, provided is a dosage regimen for treating a disease or disorder in a subject comprising administration of a pharmaceutical composition comprising a population of any of the engineered cells described herein and a pharmaceutically acceptable additive, carrier, diluent or excipient, wherein the pharmaceutical composition is administered in about 1-3 doses.

In some embodiments, the pharmaceutical composition administered is up to about 6.0×10⁸ cells in about 1-3 doses. In some embodiments, the pharmaceutical composition administered is from about 0.6×10⁶ to about 6.0×10⁸ cells in about 1-3 doses. In some embodiments, the pharmaceutical composition administered is from about 0.2×10⁶ to about 5.0×10⁶ cells per kg of the subject's body weight in about 1-3 doses, if the subject has a body weight of 50 kg or less. In some embodiments, the pharmaceutical composition administered is from about 0.1×10⁸ to about 2.5×10⁸ cells in about 1-3 doses, if the subject has a body weight greater than 50 kg. In some embodiments, the pharmaceutical composition administered is from about 2.0×10⁶ cells per kg of the subject's body weight and up to about 2×10⁸ cells in about 1-3 doses.

In some embodiments, the administration of a single dose to the subject takes a duration of time of about 60 minutes or less. In some embodiments, the administration of a single dose to the subject takes a duration of time of about 30 minutes or less.

In some embodiments, the pharmaceutically acceptable additive, carrier, diluent or excipient comprises one or more selected from the group consisting of Plasma-Lyte A, dextrose, dextran, sodium chloride, human serum albumin (HSA), dimethylsulfoxide (DMSO), and a combination thereof.

In some embodiments, the pharmaceutical composition further comprises a pharmaceutically acceptable buffer. In some embodiments, the pharmaceutically acceptable buffer is neutral buffer saline or phosphate buffered saline.

In some embodiments, after the administration of the pharmaceutical composition, the population of cells or progeny thereof are present in the subject up to 9 months. In some embodiments, after the administration of the pharmaceutical composition, the population of cells or progeny thereof are present in the subject at least 2 years or more.

In some embodiments, after the administration of the pharmaceutical composition, the population of engineered cells or progeny thereof exhibit at least 40% survival in a subject after about 10 days following administration. In some embodiments, after the administration of the pharmaceutical composition, the population of engineered cells or progeny thereof exhibit at least 80% survival in a subject after about 2 weeks following administration. In some embodiments, after the administration of the pharmaceutical composition, the population of engineered cells or progeny thereof exhibit at least 100% survival in a subject after about 3 weeks following administration. In some embodiments, after the administration of the pharmaceutical composition, the population of engineered cells or progeny thereof exhibit at least 150% survival in a subject after about 4 weeks following administration.

In some embodiments, the administration of 2-3 doses to the subject occurs such that each dose is administered ranging from 1 to 24 hours apart. In some embodiments, the administration of 2-3 doses to the subject occurs such that each dose is administered ranging from 1 to 28 days apart. In some embodiments, the administration of 2-3 doses to the subject occurs such that each dose is administered ranging from 1 to 6 weeks apart. In some embodiments, the administration of 2-3 doses to the subject occurs such that each dose is administered ranging from 1 to 12 months or more apart.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta, the engineered cell further comprising a set of exogenous polynucleotides encoding CD47 and a chimeric antigen receptor (CAR). In some embodiments, the set of exogenous polynucleotides are inserted into at least one allele of the T cell using viral transduction, for example, with a vector. In some embodiments, the vector is a pseudotyped, self-inactivating lentiviral vector that carries the set of exogenous polynucleotides. In some embodiments, the vector is a self-inactivating lentiviral vector pseudotyped with a vesicular stomatitis virus glycoprotein (VSV-G) envelope, and which carries the set of exogenous polynucleotides. In some embodiments, set of exogenous polynucleotides are inserted into at least one allele of the T cell using a lentivirus based viral vector. In some embodiments, the set of exogenous polynucleotides are inserted into a safe harbor or target locus of at least one allele of the cell; and (ii) a pharmaceutically acceptable additive, carrier, diluent or excipient, wherein the pharmaceutical composition comprises up to about 6.0×10⁸ cells.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta, the engineered cell further comprising a set of exogenous polynucleotides encoding CD47 and a chimeric antigen receptor (CAR). In some embodiments, the set of exogenous polynucleotides are inserted into at least one allele of the T cell using viral transduction. In some embodiments, set of exogenous polynucleotides are inserted into at least one allele of the T cell using a lentivirus based viral vector. In some embodiments, the set of exogenous polynucleotides are inserted into a safe harbor or target locus of at least one allele of the cell; and (ii) a pharmaceutically acceptable additive, carrier, diluent or excipient, wherein the pharmaceutical composition is administered in 1-3 doses.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta, the engineered cell further comprising a set of exogenous polynucleotides encoding CD47 and a chimeric antigen receptor (CAR), wherein the set of exogenous polynucleotides are inserted into a safe harbor or target locus of at least one allele of the cell; and (ii) a pharmaceutically acceptable additive, carrier, diluent or excipient, wherein a dose of the pharmaceutical composition is administered for a duration of time of about 60 minutes or less.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta; and (ii) a pharmaceutically acceptable additive, carrier, diluent or excipient, wherein the pharmaceutical composition comprises up to about 6.0×10⁸ cells.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta; and (ii) a pharmaceutically acceptable additive, carrier, diluent or excipient, wherein the pharmaceutical composition is administered in 1-3 doses.

Provided herein is a dosage regimen for treating a disease or disorder in a subject comprising administering a pharmaceutical composition comprising (i) an engineered cell comprising reduced expression of HLA-A, HLA-B, HLA-C, HLA-DP, HLA-DQ, HLA-DR, B2M, CIITA, TCR-alpha, and/or TCR-beta; and (ii) a pharmaceutically acceptable additive, carrier, diluent or excipient, wherein a dose of the pharmaceutical composition is administered for a duration of 60 minutes or less.

In some embodiments, provided is a method of treating an autoimmune diseases/disorders and/or inflammatory diseases/disorders in a subject comprising administration of any of the engineered cells described herein or any of the pharmaceutical compositions described herein or any of the dosage regimens described herein to the subject. In some embodiments, the autoimmune diseases/disorders and/or inflammatory diseases/disorders are at least partially B cell and/or plasma cell mediated autoimmune diseases/disorders and/or inflammatory diseases/disorders. In some embodiments, the autoimmune diseases/disorders and/or inflammatory diseases/disorders are B cell and/or plasma cell mediated autoimmune diseases/disorders and/or inflammatory diseases/disorders. In some embodiments, the B cells and/or plasma cells express CD19, CD20, or a combination thereof. In some embodiments, autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes. Further examples of “autoimmune disease” or “autoimmune disorder” or “inflammatory disease” or “inflammatory disorder” can be found in Section Z below.

In some embodiments, provided is a method of preventing T cell exhaustion in a subject comprising administration of any of the engineered cells described herein to the subject, wherein the CAR is a CD19/CD20-bispecific CAR. In some embodiments, provided is a method of preventing T cell exhaustion in a subject comprising administration of any of the engineered cells described herein to the subject, wherein the CAR is a CD19/CD22-bispecific CAR.

In some embodiments, provided herein is a method of preventing T cell exhaustion or treating a disease in a subject comprising: (i) administration of a first dosage regimen comprising a first population of any of the engineered cells described herein to the subject at a first timepoint, and (ii) administration of a second dosage regimen comprising a second population of any of the engineered cells described herein to the subject at a second timepoint, wherein the first dosage regimen and the second dosage regimen are different.

In some embodiments, provided herein is a method of preventing T cell exhaustion or treating a disease in a subject comprising: (i) administration of a first dosage regimen comprising a first population of any of the engineered cells described herein to the subject at a first timepoint, and (ii) administration of a second dosage regimen comprising a second population of any of the engineered cells described herein to the subject at a second timepoint, wherein the first population of engineered cells and the second population of engineered cells both comprise the same chimeric antigen receptor.

In some embodiments, provided herein is a method of preventing T cell exhaustion or treating a disease in a subject comprising: (i) administration of a first dosage regimen comprising a first population of any of the engineered cells described herein to the subject at a first timepoint, and (ii) administration of a second dosage regimen comprising a second population of any of the engineered cells described herein to the subject at a second timepoint, wherein the first population of engineered cells and the second population of engineered cells both comprise different chimeric antigen receptors.

In some embodiments, provided herein is a method of preventing T cell exhaustion or treating a disease in a subject comprising: (i) administration of a first dosage regimen comprising a first population of any of the engineered cells described herein to the subject at a first timepoint, and (ii) administration of a second dosage regimen comprising a second population of any of the engineered cells described herein to the subject at a second timepoint, wherein the engineered cells of the first population comprise a first chimeric antigen receptor that binds a first antigen and the engineered cells of the second population comprise a second chimeric antigen receptor that binds a second antigen, and wherein the first antigen and the second antigen are the same.

In some embodiments, provided herein is a method of preventing T cell exhaustion or treating a disease in a subject comprising: (i) administration of a first dosage regimen comprising a first population of any of the engineered cells described herein to the subject at a first timepoint, and (ii) administration of a second dosage regimen comprising a second population of any of the engineered cells described herein to the subject at a second timepoint, wherein the engineered cells of the first population comprise a first chimeric antigen receptor that binds a first antigen and the engineered cells of the second population comprise a second chimeric antigen receptor that binds a second antigen, and wherein the first antigen and the second antigen are different.

Provided herein are non-activated T cells comprising reduced expression of HLA-A, HLA-B, HLA-C, HLA-DP, HLA-DQ, HLA-DR, B2M, CIITA, TCR-alpha, and/or TCR-beta relative to a wild-type T cell, and a first exogenous polynucleotide encoding a chimeric antigen receptor (CAR).

In some embodiments, the non-activated T cell is a primary T cell. In other embodiments, the non-activated T cell is differentiated from the engineered cells of the present technology.

In some embodiments, the T cell is a CD8⁺ T cell.

In some embodiments, the non-activated T cell has not been treated with an anti-CD3 antibody, an anti-CD28 antibody, a T cell activating cytokine, or a soluble T cell costimulatory molecule.

In some embodiments, the anti-CD3 antibody is OKT3. In some embodiments, the anti-CD28 antibody is CD28.2. In some embodiments, the T cell activating cytokine is selected from the group of T cell activating cytokines consisting of IL-2, IL-7, IL-15, and IL-21. In some embodiments, the soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the non-activated T cell does not express activation markers.

In some embodiments, the non-activated T cell expresses CD3 and CD28, and wherein the CD3 and/or CD28 are inactive.

In some embodiments, the first exogenous polynucleotide is carried by a lentiviral vector comprising a CD8 binding agent.

In some embodiments, the non-activated T cell further comprises a second exogenous polynucleotide encoding CD47.

In some embodiments, the first and/or second exogenous polynucleotides are inserted into a specific locus of at least one allele of the T cell. In some embodiments, the first and/or second exogenous polynucleotides are inserted into at least one allele of the T cell using viral transduction. In some embodiments, the first and/or second exogenous polynucleotides are inserted into at least one allele of the T cell using a lentivirus based viral vector. In some embodiments, the vector is a pseudotyped, self-inactivating lentiviral vector that carries the first and/or second exogenous polynucleotides. In some embodiments, the vector is a self-inactivating lentiviral vector pseudotyped with a vesicular stomatitis virus glycoprotein (VSV-G) envelope, and which carries the first and/or second exogenous polynucleotides. In some embodiments, the specific locus is selected from the group consisting of a safe harbor or target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus. In some embodiments, the second exogenous polynucleotide encoding CD47 and the first exogenous polynucleotide encoding the CAR are inserted into the CIITA locus. In some embodiments, the second exogenous polynucleotide encoding CD47 and the first exogenous polynucleotide encoding the CAR are inserted into the TRAC locus. In some embodiments, the second exogenous polynucleotide encoding CD47 and the first exogenous polynucleotide encoding the CAR are inserted into the TRB locus. In some embodiments, the second exogenous polynucleotide encoding CD47 and the first exogenous polynucleotide encoding the CAR are inserted into the safe harbor or target locus. In some embodiments, the safe harbor or target locus is selected from the group consisting of a CCR5 gene locus, a CXCR4 gene locus, a PPP1R12C (also known as AAVS1) gene locus, an albumin gene locus, a SHS231 gene locus, a CLYBL gene locus, a Rosa gene locus (e.g., ROSA26 gene locus), an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. In some embodiments, the safe harbor or target locus is selected from the group consisting of the AAVS1 locus, the CCR5 locus, and the ROSA26 locus.

In some embodiments, the CAR is selected from the group consisting of a CD19-specific CAR, a CD22-specific CAR, and a CD20-specific CAR. In some embodiments, the CAR is a bispecific CAR. In some embodiments, the bispecific CAR is a CD19/CD20-bispecific CAR. In some embodiments, the bispecific CAR is a CD19/CD22-bispecific CAR.

In some embodiments, the non-activated T cell does not express HLA-A, HLA-B, and/or HLA-C antigens. In some embodiments, the non-activated T cell does not express B2M. In some embodiments, the non-activated T cell does not express HLA-DP, HLA-DQ, and/or HLA-DR antigens. In some embodiments, the non-activated T cell does not express CIITA. In some embodiments, the non-activated T cell does not express TCR-alpha and TCR-beta.

In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the TRAC locus.

In some embodiments, the non-activated T cell is a B2M^{indel/indel}, CIITA^{indel/indel} TRAC^{indel/indel} cell comprising the second exogenous polynucleotide encoding CD47 and the first exogenous polynucleotide encoding CAR inserted into the TRAC locus.

In some embodiments, the non-activated T cell is a B2M^{indel/indel}, CIITA^{indel/indel} TRAC^{indel/indel} cell comprising second exogenous polynucleotide encoding CD47 and/or the first exogenous polynucleotide encoding CAR inserted into the TRB locus.

In some embodiments, the non-activated T cell is a B2M^{indel/indel}, CIITA^{indel/indel} TRAC^{indel/indel} cell comprising the second exogenous polynucleotide encoding CD47 and the first exogenous polynucleotide encoding CAR inserted into the TRB locus.

In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the B2M locus.

In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a B2M locus.

In some embodiments, the non-activated T cell is a B2M^{indel/indel}, CIITA^{indel/indel} TRAC^{indel/indel} cell comprising second exogenous polynucleotide encoding CD47 and/or the first exogenous polynucleotide encoding CAR inserted into the CIITA locus.

In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a CIITA locus.

Provided herein are engineered T cells comprising reduced expression of HLA-A, HLA-B, HLA-C, HLA-DP, HLA-DQ, HLA-DR, B2M, CIITA, TCR-alpha, and/or TCR-beta relative to a wild-type T cell, wherein the engineered T cell further comprises a first exogenous polynucleotide encoding a chimeric antigen receptor (CAR) carried by a lentiviral vector. Provided herein are engineered T cells comprising reduced expression of HLA-A, HLA-B, HLA-C, HLA-DP, HLA-DQ, HLA-DR, B2M, CIITA, TCR-alpha, and/or TCR-beta relative to a wild-type T cell, wherein the engineered T cell further comprises a first exogenous polynucleotide encoding a chimeric antigen receptor (CAR) carried by a lentiviral vector that comprises a CD8 binding agent.

In some embodiments, the engineered T cell is a primary T cell. In other embodiments, the engineered T cell is differentiated from the engineered cell of the present technology. In some embodiments, the T cell is a CD8⁺ T cell.

In some embodiments, the engineered T cell has not been treated with an anti-CD3 antibody, an anti-CD28 antibody, a T cell activating cytokine, or a soluble T cell costimulatory molecule. In some embodiments, the anti-CD3 antibody is OKT3, wherein the anti-CD28 antibody is CD28.2, wherein the T cell activating cytokine is selected from the group of T cell activating cytokines consisting of IL-2, IL-7, IL-15, and IL-21, and wherein soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the engineered T cell does not express activation markers. In some embodiments, the engineered T cell expresses CD3 and CD28, and wherein the CD3 and/or CD28 are inactive.

In some embodiments, the engineered T cell further comprises a second exogenous polynucleotide encoding CD47. In some embodiments, the first and/or second exogenous polynucleotides are inserted into a specific locus of at least one allele of the T cell. In some embodiments, the specific locus is selected from the group consisting of a safe harbor or target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus, the CIITA locus, the TRAC locus, the TRB locus, or the safe harbor or target locus. In some embodiments, the safe harbor or target locus is selected from the group consisting of a CCR5 gene locus, a CXCR4 gene locus, a PPP1R12C (also known as AAVS1) gene locus, an albumin gene locus, a SHS231 gene locus, a CLYBL gene locus, a Rosa gene locus (e.g., ROSA26 gene locus), an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. In some embodiments, the safe harbor or target locus is selected from the group consisting of the AAVS1 locus, the CCR5 locus, and the ROSA26 locus.

In some embodiments, the CAR is selected from the group consisting of a CD19-specific CAR, a CD22-specific CAR, and a CD20-specific CAR.

In some embodiments, the engineered T cell does not express HLA-A, HLA-B, and/or HLA-C antigens, wherein the engineered T cell does not express B2M, wherein the engineered T cell does not express HLA-DP, HLA-DQ, and/or HLA-DR antigens, wherein the engineered T cell does not express CIITA, and/or wherein the engineered T cell does not express TCR-alpha and TCR-beta.

In some embodiments, the engineered T cell is a B2M^{indel/indel}, CIITA^{indel/indel}, TRAC^{indel/indel} cell comprising the second exogenous polynucleotide encoding CD47 and/or the first exogenous polynucleotide encoding CAR inserted into the TRAC locus, into the TRB locus, into the B2M locus, or into the CIITA locus.

In some embodiments, the non-activated T cell and/or the engineered T cell of the present technology are in a subject. In other embodiments, the non-activated T cell and/or the engineered T cell of the present technology are in vitro.

In some embodiments, the non-activated T cell and/or the engineered T cell of the present technology express a CD8 binding agent. In some embodiments, the CD8 binding agent is an anti-CD8 antibody. In some embodiments, the anti-CD8 antibody is selected from the group consisting of a mouse anti-CD8 antibody, a rabbit anti-CD8 antibody, a human anti-CD8 antibody, a humanized anti-CD8 antibody, a camelid (e.g., llama, alpaca, camel) anti-CD8 antibody, and a fragment thereof. In some embodiments, the fragment thereof is an scFV or a VHH. In some embodiments, the CD8 binding agent binds to a CD8 alpha chain and/or a CD8 beta chain.

In some embodiments, the CD8 binding agent is fused to a transmembrane domain incorporated in the viral envelope. In some embodiments, the lentivirus vector is pseudotyped with a viral fusion protein. In some embodiments, the viral fusion protein comprises one or more modifications to reduce binding to its native receptor.

In some embodiments, the viral fusion protein is fused to the CD8 binding agent. In some embodiments, the viral fusion protein comprises Nipah virus F glycoprotein and Nipah virus G glycoprotein fused to the CD8 binding agent. In some embodiments, the lentivirus vector does not comprise a T cell activating molecule or a T cell costimulatory molecule. In some embodiments, the lentivirus vector encodes the first exogenous polynucleotide and/or the second exogenous polynucleotide.

In some embodiments, following transfer into a first subject, the non-activated T cell or the engineered T cell exhibits one or more responses selected from the group consisting of (a) a T cell response, (b) an NK cell response, and (c) a macrophage response, that are reduced as compared to a wild-type cell following transfer into a second subject. In some embodiments, the first subject and the second subject are different subjects. In some embodiments, the macrophage response is engulfment.

In some embodiments, following transfer into a subject, the non-activated T cell or the engineered T cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject.

In some embodiments, following transfer into a subject, the non-activated T cell or the engineered T cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the non-activated T cell or the engineered T cell is transduced with a lentivirus vector comprising a CD8 binding agent within the subject. In some embodiments, the lentivirus vector carries a gene encoding the CAR and/or CD47.

Provided herein are pharmaceutical compositions comprising a population of the non-activated T cells and/or the engineered T cells of the present technology and a pharmaceutically acceptable additive, carrier, diluent or excipient.

Provided herein are methods comprising administering to a subject a composition comprising a population of the non-activated T cells and/or the engineered T cells of the present technology, or one or more the pharmaceutical compositions of the present technology.

In some embodiments, the subject is not administered a T cell activating treatment before, after, and/or concurrently with administration of the composition. In some embodiments, the T cell activating treatment comprises lymphodepletion.

Provided herein are methods of treating a subject suffering from autoimmune diseases/disorders and/or inflammatory diseases/disorders, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are methods for expanding T cells capable of recognizing and killing tumor cells in a subject in need thereof within the subject, comprising administering to a subject a composition comprising a population of the non-activated T cells and/or the engineered T cells of the present technology, or one or more the pharmaceutical compositions of the present technology, wherein the subject is not administered a T cell activating treatment before, after, and/or concurrently with administration of the composition. In some embodiments, the T cell activating treatment comprises lymphodepletion.

Provided herein are dosage regimens for treating a disease or disorder in a subject comprising administration of a pharmaceutical composition comprising a population of the non-activated T cells and/or the engineered T cells of the present technology, or one or more the pharmaceutical compositions of the present technology, and a pharmaceutically acceptable additive, carrier, diluent or excipient, wherein the pharmaceutical composition is administered in about 1-3 doses.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:117, with the following components: CD8α signal peptide, FMC63 scFv (VL-Whitlow linker-VH), CD8α hinge domain, CD8α transmembrane domain, 4-1BB costimulatory domain, and CD3ζ signaling domain.

In some embodiments, the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:45.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise an exogenous polynucleotide encoding one or more chimeric antigen receptors (CARs) wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of one or more major histocompatibility complex (MHC) class I and/or class II human leukocyte antigen molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

In some embodiments, provided herein is a method of treating an autoimmune disease in a recipient patient that is suspected of having the autoimmune disease or has been diagnosed with the autoimmune disease comprising evaluating the patient for and/or diagnosing the patient with the autoimmune disease, and administering a population of engineered T cells to the patient, wherein the engineered T cells comprise reduced expression of one or more major histocompatibility complex (MHC) class I and/or class II human leukocyte antigen molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOS: 32, 34, 36, and 117, or wherein the one or more CARs have an scFv sequence of any one of SEQ ID NOs: 19, 29, or 37.

In some embodiments, the CAR has an scFv sequence of any one of SEQ ID NOS: 19, 29, and 37.

In some embodiments, the CAR has a sequence of any one of SEQ ID NOs: 32, 34, 36, and 117.

In some embodiments, the engineered T cells comprise a CD19-specific CAR and a CD20-specific CAR.

In some embodiments, the CD19-specific CAR and the CD20-specific CAR are encoded by a single bicistronic polynucleotide.

In some embodiments, the CD19-specific CAR and the CD20-specific CAR are encoded by two separate polynucleotides.

In some embodiments, the CD19 CAR T cells and CD20 CAR T cells are administered concomitantly.

In some embodiments, the CD19 CAR+ T cells and CD20 CAR+ T cells are administered sequentially.

In some embodiments, the CD19 CAR+ T cells are administered prior to administration of the CD20 CAR+ T cells.

In some embodiments, the CD20 CAR+ T cells are administered prior to administration of the CD19 CAR+ T cells.

In some embodiments, the number of cells administered as a therapeutically effective amount of the CD19 and/or CD20 CAR T cells is greater than the number of cells administered as a therapeutically effective amount of CD19 CAR T cells or CD20 CAR T cells alone.

In some embodiments, the number of cells administered to as a therapeutically effective amount of the CD19 and/or CD20 CAR T cells is less than the number of cells administered as a therapeutically effective amount of CD19 CAR T cells or CD20 CAR T cells alone.

In some embodiments, the engineered T cells are propagated from a primary T cell or a progeny thereof, or are derived from a T cell differentiated from an iPSC or a progeny thereof.

In some embodiments, the engineered T cells are differentiated cells derived from an induced pluripotent stem cell or a progeny thereof.

In some embodiments, the differentiated cells are a T cells or natural killer (NK) cells.

In some embodiments, the engineered T cells are a progeny of primary immune cells, optionally wherein the progeny of primary immune cells are T cells or NK cells.

In some embodiments, the engineered T cells comprise reduced expression of beta-2-microglobulin (B2M) and/or MHC class II transactivator (CIITA) relative to an unaltered or unmodified wild-type or control cell.

In some embodiments, the engineered T cells do not express B2M and/or CIITA.

In some embodiments, the engineered T cells comprise reduced expression of TCR-alpha and/or TCR-beta.

In some embodiments, the engineered T cells do not express TCR-alpha and/or TCR-beta.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9.

In some embodiments, the one or more tolerogenic factors comprise CD47.

In some embodiments, the CD19-specific CAR and the one or more tolerogenic factors are encoded by a single bicistronic polynucleotide.

In some embodiments, the CD19-specific CAR and the CD47 are encoded by a single bicistronic polynucleotide.

In some embodiments, the CD20-specific CAR and the one or more tolerogenic factors are encoded by a single bicistronic polynucleotide.

In some embodiments, the CD20-specific CAR and the CD47 are encoded by a single bicistronic polynucleotide.

In some embodiments, one or more of the first, second, and/or third exogenous polynucleotides or the bicistronic polynucleotide is inserted into a first, second, and/or third specific locus of at least one allele of the cell.

In some embodiments, the first, second and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

In some embodiments, the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.

In some embodiments, the gene therapy vector is a retrovirus or a fusosome.

In some embodiments, the first, second, and/or third exogenous polynucleotide or the bicistronic polynucleotide is introduced into the engineered T cells using CRISPR/Cas gene editing.

In some embodiments, the CRISPR/Cas system comprises a Cas effector protein selected from the group consisting of Cas9, Cas12a, and Cas12b.

In some embodiments, the CRISPR/Cas system comprises a Cas effector protein selected from the group consisting of: optionally selected from the group consisting of Cas3, Cas8a, Cas5, Cas8b, Cas8c, Cas10d, Cse1, Cse2, Csy1, Csy2, Csy3, and GSU0054; optionally selected from the group consisting of Cas9, Csn2, and Cas4; optionally selected from the group consisting of Cas10, Csm2, Cmr5, Cas10, Csx11, and Csx10; optionally Csf1; optionally selected from the group consisting of Cas12a, Cas12b, Cas12c, C2c4, C2c8, C2c5, C2c10, C2c9, CasX (Cas12e), and CasY (Cas12d); and optionally selected from the group consisting of Cas13, Cas13a, C2c2, Cas13b, Cas13c, and Cas13d.

In some embodiments, the CRISPR/Cas gene editing is carried out ex vivo from a donor subject.

In some embodiments, the first, second, and/or third exogenous polynucleotide or the bicistronic polynucleotide is introduced into the engineered T cells using a lentiviral vector.

In some embodiments, the engineered T cells evade NK cell mediated cytotoxicity upon administration to the recipient patient.

In some embodiments, the engineered T cells are protected from cell lysis by mature NK cells upon administration to the recipient patient.

In some embodiments, the engineered T cells evade macrophage-mediated cytotoxicity, optionally wherein the macrophage-mediated cytotoxicity involves phagocytosis and/or reactive oxygen species.

In some embodiments, the engineered T cells do not induce an immune response to the cell upon administration to the recipient patient.

In some embodiments, the autoimmune disease is selected from the group consisting of lupus, systemic lupus erythematosus, multiple sclerosis, systemic sclerosis, vasculitis, Crohn's disease, Myasthenia Gravis, Stiff-Person syndrome and pulmonary conditions.

In some embodiments, the administration is selected from the group consisting of intravenous injection, intramuscular injection, intravascular injection, and transplantation.

In some embodiments, the engineered T cells are administered before, during or after starting a different treatment regimen for the patient.

In some embodiments, the different treatment regimen is selected from the group consisting of re-dosing of the same or different cells, and pre-treatment, concurrent treatment, or subsequent treatment with an additional agent.

In some embodiments, the different cells are autologous T or NK cells or CAR-T cells expressing a first CAR that is different from a second CAR expressed by the engineered CAR-T cells.

In some embodiments, the patient was treated with an immunodepleting therapy prior to administering the engineered T cells.

In some embodiments, the immunodepleting therapy comprises administration of fludarabine and/or cyclophosphamide.

In some embodiments, the patient has undergone a prior antibody therapy.

In some embodiments, the antibody therapy is rituximab.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 4 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, about 1000 mg/m² of cyclophosphamide for about 1, about 2, about 3, about 4, about 5, about 6, or about 7 days.

In some embodiments, the immunodepleting therapy comprises IV infusion of about 500 mg/m² of cyclophosphamide for about 2 days.

In some embodiments, at least about 40×10⁴ engineered T cells are administered to the patient.

In some embodiments, at least about 40×10⁵ engineered T cells are administered to the patient.

In some embodiments, the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the wild type cell or the control cell is a starting material.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:117, with the following components: CD8α signal peptide, FMC63 scFv (VL-Whitlow linker-VH), CD8α hinge domain, CD8α transmembrane domain, 4-1BB costimulatory domain, and CD3ζ signaling domain.

In some embodiments, the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:45.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise an exogenous polynucleotide encoding one or more chimeric antigen receptors (CARs) wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of one or more major histocompatibility complex (MHC) class I and/or class II human leukocyte antigen molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a recipient patient that is suspected of having the autoimmune disease or has been diagnosed with the autoimmune disease, wherein the engineered T cells comprise reduced expression of one or more major histocompatibility complex (MHC) class I and/or class II human leukocyte antigen molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, the CAR has an scFv sequence of any one of SEQ ID NOS: 19, 29, and 37.

In some embodiments, the CAR has a sequence of any one of SEQ ID NOs: 32, 34, 36, and 117.

In some embodiments, the engineered T cells comprise a CD19-specific CAR and a CD20-specific CAR.

In some embodiments, the CD19-specific CAR and the CD20-specific CAR are encoded by a single bicistronic polynucleotide.

In some embodiments, the CD19-specific CAR and the CD20-specific CAR are encoded by two separate polynucleotides.

In some embodiments, the CD19 CAR T cells and CD20 CAR T cells are administered concomitantly.

In some embodiments, the CD19 CAR+ T cells and CD20 CAR+ T cells are administered sequentially.

In some embodiments, the CD19 CAR+ T cells are administered prior to administration of the CD20 CAR+ T cells.

In some embodiments, the CD20 CAR+ T cells are administered prior to administration of the CD19 CAR+ T cells.

In some embodiments, the number of cells administered as a therapeutically effective amount of the CD19 and/or CD20 CAR T cells is greater than the number of cells administered as a therapeutically effective amount of CD19 CAR T cells or CD20 CAR T cells alone.

In some embodiments, the number of cells administered to as a therapeutically effective amount of the CD19 and/or CD20 CAR T cells is less than the number of cells administered as a therapeutically effective amount of CD19 CAR T cells or CD20 CAR T cells alone.

In some embodiments, the engineered T cells are propagated from a primary T cell or a progeny thereof, or are derived from a T cell differentiated from an iPSC or a progeny thereof.

In some embodiments, the engineered T cells are differentiated cells derived from an induced pluripotent stem cell or a progeny thereof.

In some embodiments, the differentiated cells are a T cells or natural killer (NK) cells.

In some embodiments, the engineered T cells are a progeny of primary immune cells, optionally wherein the progeny of primary immune cells are T cells or NK cells.

In some embodiments, the engineered T cells comprise reduced expression of beta-2-microglobulin (B2M) and/or MHC class II transactivator (CIITA) relative to an unaltered or unmodified wild-type or control cell.

In some embodiments, the engineered T cells do not express B2M and/or CIITA.

In some embodiments, the engineered T cells comprise reduced expression of TCR-alpha and/or TCR-beta.

In some embodiments, the engineered T cells do not express TCR-alpha and/or TCR-beta.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9.

In some embodiments, the one or more tolerogenic factors comprise CD47.

In some embodiments, the CD19-specific CAR and the one or more tolerogenic factors are encoded by a single bicistronic polynucleotide.

In some embodiments, the CD19-specific CAR and the CD47 are encoded by a single bicistronic polynucleotide.

In some embodiments, the CD20-specific CAR and the one or more tolerogenic factors are encoded by a single bicistronic polynucleotide.

In some embodiments, the CD20-specific CAR and the CD47 are encoded by a single bicistronic polynucleotide.

In some embodiments, one or more of the first, second, and/or third exogenous polynucleotides or the bicistronic polynucleotide is inserted into a first, second, and/or third specific locus of at least one allele of the cell.

In some embodiments, the first, second and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

In some embodiments, the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.

In some embodiments, the first, second, and/or third exogenous polynucleotide or the bicistronic polynucleotide is introduced into the engineered T cells using CRISPR/Cas gene editing.

In some embodiments, the CRISPR/Cas system comprises a Cas effector protein selected from the group consisting of Cas9, Cas12a, and Cas2b.

In some embodiments, the CRISPR/Cas system comprises a Cas effector protein selected from the group consisting of: optionally selected from the group consisting of Cas3, Cas8a, Cas5, Cas8b, Cas8c, Cas10d, Cse1, Cse2, Csy1, Csy2, Csy3, and GSU0054; optionally selected from the group consisting of Cas9, Csn2, and Cas4; optionally selected from the group consisting of Cas10, Csm2, Cmr5, Cas10, Csx11, and Csx10; optionally Csf1; optionally selected from the group consisting of Cas12a, Cas12b, Cas12c, C2c4, C2c8, C2c5, C2c10, C2c9, CasX (Cas12e), and CasY (Cas12d); and optionally selected from the group consisting of Cas13, Cas13a, C2c2, Cas13b, Cas13c, and Cas13d.

In some embodiments, the CRISPR/Cas gene editing is carried out ex vivo from a donor subject.

In some embodiments, the first, second, and/or third exogenous polynucleotide or the bicistronic polynucleotide is introduced into the engineered T cells using a lentiviral vector.

In some embodiments, the engineered T cells evade NK cell mediated cytotoxicity upon administration to the recipient patient.

In some embodiments, the engineered T cells are protected from cell lysis by mature NK cells upon administration to the recipient patient.

In some embodiments, the engineered T cells evade macrophage-mediated cytotoxicity, optionally wherein the macrophage-mediated cytotoxicity involves phagocytosis and/or reactive oxygen species.

In some embodiments, the engineered T cells do not induce an immune response to the cell upon administration to the recipient patient.

In some embodiments, the autoimmune disease is selected from the group consisting of lupus, systemic lupus erythematosus, multiple sclerosis, systemic sclerosis, vasculitis, Crohn's disease, Myasthenia Gravis, Stiff-Person syndrome and pulmonary conditions.

In some embodiments, the administration is selected from the group consisting of intravenous injection, intramuscular injection, intravascular injection, and transplantation.

In some embodiments, the engineered T cells are administered before, during or after starting a different treatment regimen for the patient.

In some embodiments, the different treatment regimen is selected from the group consisting of re-dosing of the same or different cells, and pre-treatment, concurrent treatment, or subsequent treatment with an additional agent.

In some embodiments, the different cells are autologous T or NK cells or CAR-T cells expressing a first CAR that is different from a second CAR expressed by the engineered CAR-T cells.

In some embodiments, the patient was treated with an immunodepleting therapy prior to administering the engineered T cells.

In some embodiments, the immunodepleting therapy comprises administration of fludarabine and/or cyclophosphamide.

In some embodiments, the patient has undergone a prior antibody therapy.

In some embodiments, the antibody therapy is rituximab.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 4 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, about 1000 mg/m² of cyclophosphamide for about 1, about 2, about 3, about 4, about 5, about 6, or about 7 days.

In some embodiments, the immunodepleting therapy comprises IV infusion of about 500 mg/m² of cyclophosphamide for about 2 days.

In some embodiments, at least about 40×10⁴ engineered T cells are administered to the patient.

In some embodiments, at least about 40×10⁵ engineered T cells are administered to the patient.

In some embodiments, the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the wild type cell or the control cell is a starting material.

In some embodiments, the unaltered or unmodified wild-type or control cell is a starting T cell isolated from a donor.

In some embodiments, provided herein is a method of treating a patient with an Epstein Barr Virus (EBV) infection comprising administering a population of engineered T cells to the patient, wherein the engineered T cells comprise reduced expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs) wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating a patient with an EBV infection comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs wherein the one or more CARs comprise an extracellular ligand-binding domain having specificity for CD19 or CD22, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of beta-2-microglobulin (B2M) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a method of treating multiple sclerosis in a patient that is suspected of having multiple sclerosis or has been diagnosed with multiple sclerosis comprising evaluating the patient for and/or diagnosing the patient with EBV infection and optionally multiple sclerosis, and administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, the one or more CARs comprise a CD8α hinge domain, a CD28 hinge domain, or an IgG4 hinge domain.

In some embodiments, the one or more CARs comprise a CD8α hinge domain having the amino acid sequence of SEQ ID NO: 9.

In some embodiments, the one or more CARs comprise a CD28 hinge domain having the amino acid sequence of SEQ ID NO: 10 or 113.

In some embodiments, the one or more CARs comprise a IgG4 hinge domain having the amino acid sequence of SEQ ID NO: 11 or 12.

In some embodiments, the one or more CARs comprise a CD8α transmembrane domain or a CD28 transmembrane domain.

In some embodiments, the one or more CARs comprise a CD8α transmembrane domain having the amino acid sequence of SEQ ID NO: 14.

In some embodiments, the one or more CARs comprise a CD28 transmembrane domain having the amino acid sequence of SEQ ID NO: 15 or 114.

In some embodiments, the one or more CARs comprise a 4-1BB costimulatory domain, a CD28 costimulatory domain, or a CD3ζ signaling domain.

In some embodiments, the one or more CARs comprise a 4-1BB costimulatory domain having the amino acid sequence of SEQ ID NO: 16.

In some embodiments, the one or more CARs comprise a CD28 costimulatory domain having the amino acid sequence of SEQ ID NO: 17.

In some embodiments, the one or more CARs comprise a CD3ζ signaling domain having the amino acid sequence of SEQ ID NO: 18 or 115.

In some embodiments, the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the one or more CARs have a sequence of any one of SEQ ID NOs: 32, 34, 36, 117, 91, 92, 92, 128, 133, or 134.

In some embodiments, provided herein is a method of treating multiple sclerosis in a patient that is suspected of having multiple sclerosis or has been diagnosed with multiple sclerosis and administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD20 CAR having the CDR sequences of SEQ ID NO: 37.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a EBV antigen CAR having the CDR sequences of SEQ ID NO: 133 or 134, and/or the CDR sequences from the VH/VL sequences of SEQ ID NOs: 129-132 or 135-172.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD22 CAR having the CDR sequences of SEQ ID NO: 45.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45.

In some embodiments, provided herein is a method of treating multiple sclerosis in a patient that is suspected of having multiple sclerosis or has been diagnosed with multiple sclerosis comprising administering a population of engineered T cells to the patient, wherein the engineered T cells comprise an exogenous polynucleotide encoding one or more CARs wherein the one or more CARs have a sequence of any one of SEQ ID NOs: 32, 34, 36, 117, 91, 92, 92, 128, 133, or 134, or wherein the one or more CARs have an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the method further comprises evaluating the patient for and/or diagnosing the patient with EBV infection and optionally multiple sclerosis prior to administering the population of engineered T cells to the patient.

In some embodiments, the diagnosis comprises evaluating the patient for EBV infection.

In some embodiments, the diagnosis comprises evaluating the patient for multiple sclerosis.

In some embodiments, the treatment prevents multiple sclerosis.

In some embodiments, the treatment treats multiple sclerosis.

In some embodiments, the patient with the EBV infection has been diagnosed with multiple sclerosis.

In some embodiments, the multiple sclerosis is relapsing-remitting multiple sclerosis, primary progressive multiple sclerosis, or secondary progressive multiple sclerosis.

In some embodiments, the patient undergoes remission of multiple sclerosis following administration of the engineered T cells.

In some embodiments, the patient with the EBV infection is undergoing treatment for the EBV infection.

In some embodiments, the patient with the EBV infection has an active EBV infection.

In some embodiments, the patient with the EBV infection has an inactive EBV infection.

In some embodiments, the patient undergoes a reduced EBV infection following administration of the engineered T cells, optionally wherein the reduced EBV infection is characterized by reduced viral load.

In some embodiments, the treatment prevents an EBV infection change from an inactive to an active EBV infection.

In some embodiments, the method results in B cell depletion.

In some embodiments, the engineered T cells comprise one or more of a CD19-specific CAR, a CD20-specific CAR, a CD22-specific CAR, a BCMA-specific CAR, a GPRC5D-specific CAR, a CD38-specific CAR, a CD70-specific CAR, a CD79b-specific CAR, and an EBV antigen-specific CAR.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise an exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the one or more CARs comprise a CD8α hinge domain, a CD28 hinge domain, or an IgG4 hinge domain.

In some embodiments, the one or more CARs comprise a CD8α hinge domain having the amino acid sequence of SEQ ID NO: 9.

In some embodiments, the one or more CARs comprise a CD28 hinge domain having the amino acid sequence of SEQ ID NO: 10 or 113.

In some embodiments, the one or more CARs comprise a IgG4 hinge domain having the amino acid sequence of SEQ ID NO: 11 or 12.

In some embodiments, the one or more CARs comprise a CD8α transmembrane domain or a CD28 transmembrane domain.

In some embodiments, the one or more CARs comprise a CD8α transmembrane domain having the amino acid sequence of SEQ ID NO: 14.

In some embodiments, the one or more CARs comprise a CD28 transmembrane domain having the amino acid sequence of SEQ ID NO: 15 or 114.

In some embodiments, the one or more CARs comprise a 4-1BB costimulatory domain, a CD28 costimulatory domain, or a CD3ζ signaling domain.

In some embodiments, the one or more CARs comprise a 4-1BB costimulatory domain having the amino acid sequence of SEQ ID NO: 16.

In some embodiments, the one or more CARs comprise a CD28 costimulatory domain having the amino acid sequence of SEQ ID NO: 17.

In some embodiments, the one or more CARs comprise a CD3ζ signaling domain having the amino acid sequence of SEQ ID NO: 18 or 115.

In some embodiments, the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the one or more CARs have a sequence of any one of SEQ ID NOs: 32, 34, 36, 117, 91, 92, 92, 128, 133, or 134.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the method further comprises evaluating the patient for and/or diagnosing the patient with the autoimmune disease.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8α signal peptide, FMC63 scFv (VL-Whitlow linker-VH), CD8α hinge domain, CD8α transmembrane domain, 4-1BB costimulatory domain, and CD3ζ signaling domain.

In some embodiments, the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a EBV antigen-specific CAR having the CDR sequences of SEQ ID NO: 133 or 134, and/or the CDR sequences from the VH/VL sequences of SEQ ID NOs: 129-132 or 135-172, and wherein the autoimmune disease is multiple sclerosis.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise an exogenous polynucleotide encoding one or more CARs wherein the one or more CARs have a sequence of any one of SEQ ID NOS: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, the method further comprises evaluating the patient for and/or diagnosing the patient with the autoimmune disease prior to administering the population of engineered T cells to the patient.

In some embodiments, the autoimmune disease is selected from the group consisting of lupus, systemic lupus erythematosus, multiple sclerosis, systemic sclerosis, vasculitis, Crohn's disease, Myasthenia Gravis, Stiff-Person syndrome and a pulmonary condition.

In some embodiments, the patient is suspected of having an EBV infection or has been diagnosed as having an EBV infection.

In some embodiments, the patient is suspected of having multiple sclerosis or has been diagnosed with multiple sclerosis.

In some embodiments, the method further comprises administering a second, third, fourth, fifth, or sixth dose of the engineered T cells to the patient.

In some embodiments, the same in one or more of the first, second, third, fourth, fifth, and/or sixth dose of engineered T cells.

In some embodiments, the CAR is different in one or more of the first, second, third, fourth, fifth, and/or sixth dose of engineered T cells.

In some embodiments, the CAR has an scFv sequence of any one of SEQ ID NOS: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the CAR has a sequence of any one of SEQ ID NOs: 32, 34, 36, 117, 91, 92, 92, 128, 133, and 134.

The method of any one of claims 1-78, wherein the engineered T cells comprise a CD19-specific CAR and a CD20-specific CAR.

In some embodiments, the CD19-specific CAR has the CDR sequences of SEQ ID NO: 117 and the CD22 CAR has the CDR sequences of SEQ ID NO: 45.

In some embodiments, the CD19-specific CAR and the CD20-specific CAR are encoded by a single bicistronic polynucleotide.

In some embodiments, the CD19-specific CAR and the CD20-specific CAR are encoded by a single bispecific CAR.

In some embodiments, the CD19-specific CAR and the CD20-specific CAR are encoded by two separate polynucleotides.

In some embodiments, the CD19 CAR T cells and CD20 CAR T cells are administered concomitantly.

In some embodiments, the CD19 CAR+ T cells and CD20 CAR+ T cells are administered sequentially.

In some embodiments, the CD19 CAR+ T cells are administered prior to administration of the CD20 CAR+ T cells.

In some embodiments, the CD20 CAR+ T cells are administered prior to administration of the CD19 CAR+ T cells.

In some embodiments, the number of cells administered as a therapeutically effective amount of the CD19 and/or CD20 CAR T cells is greater than the number of cells administered as a therapeutically effective amount of CD19 CAR T cells or CD20 CAR T cells alone.

In some embodiments, the number of cells administered to as a therapeutically effective amount of the CD19 and/or CD20 CAR T cells is less than the number of cells administered as a therapeutically effective amount of CD19 CAR T cells or CD20 CAR T cells alone.

In some embodiments, the engineered T cells comprise an EBV antigen-specific CAR and a CD20-specific CAR.

In some embodiments, the EBV antigen-specific CAR and the CD20-specific CAR are encoded by a single bicistronic polynucleotide.

In some embodiments, the EBV antigen-specific CAR and the CD20-specific CAR are encoded by a single bispecific CAR.

In some embodiments, the EBV antigen-specific CAR and the CD20-specific CAR are encoded by two separate polynucleotides.

In some embodiments, the EBV antigen CAR T cells and CD20 CAR T cells are administered concomitantly.

In some embodiments, the EBV antigen CAR+ T cells and CD20 CAR+ T cells are administered sequentially.

In some embodiments, the EBV antigen CAR+ T cells are administered prior to administration of the CD20 CAR+ T cells.

In some embodiments, the CD20 CAR+ T cells are administered prior to administration of the EBV antigen CAR+ T cells.

In some embodiments, the number of cells administered as a therapeutically effective amount of the EBV antigen and/or CD20 CAR T cells is greater than the number of cells administered as a therapeutically effective amount of EBV antigen CAR T cells or CD20 CAR T cells alone.

In some embodiments, the number of cells administered to as a therapeutically effective amount of the EBV antigen and/or CD20 CAR T cells is less than the number of cells administered as a therapeutically effective amount of EBV antigen CAR T cells or CD20 CAR T cells alone.

In some embodiments, the engineered T cells comprise an EBV antigen-specific CAR and a CD19-specific CAR.

In some embodiments, the EBV antigen-specific CAR and the CD19-specific CAR are encoded by a single bicistronic polynucleotide.

In some embodiments, the EBV antigen-specific CAR and the CD19-specific CAR are encoded by a single bispecific CAR.

In some embodiments, the EBV antigen-specific CAR and the CD19-specific CAR are encoded by two separate polynucleotides.

In some embodiments, the EBV antigen CAR T cells and CD19 CAR T cells are administered concomitantly.

In some embodiments, the EBV antigen CAR+ T cells and CD19 CAR+ T cells are administered sequentially.

In some embodiments, the EBV antigen CAR+ T cells are administered prior to administration of the CD19 CAR+ T cells.

In some embodiments, the CD19 CAR+ T cells are administered prior to administration of the EBV antigen CAR+ T cells.

In some embodiments, the number of cells administered as a therapeutically effective amount of the EBV antigen and/or CD19 CAR T cells is greater than the number of cells administered as a therapeutically effective amount of EBV antigen CAR T cells or CD19 CAR T cells alone.

In some embodiments, the number of cells administered to as a therapeutically effective amount of the EBV antigen and/or CD19 CAR T cells is less than the number of cells administered as a therapeutically effective amount of EBV antigen CAR T cells or CD19 CAR T cells alone.

In some embodiments, the engineered T cells comprise an EBV antigen-specific CAR and a CD22-specific CAR.

In some embodiments, the EBV antigen-specific CAR and the CD22-specific CAR are encoded by a single bicistronic polynucleotide.

In some embodiments, the EBV antigen-specific CAR and the CD22-specific CAR are encoded by a single bispecific CAR.

In some embodiments, the EBV antigen-specific CAR and the CD22-specific CAR are encoded by two separate polynucleotides.

In some embodiments, the EBV antigen CAR T cells and CD22 CAR T cells are administered concomitantly.

In some embodiments, the EBV antigen CAR+ T cells and CD22 CAR+ T cells are administered sequentially.

In some embodiments, the EBV antigen CAR+ T cells are administered prior to administration of the CD22 CAR+ T cells.

In some embodiments, the CD22 CAR+ T cells are administered prior to administration of the EBV antigen CAR+ T cells.

In some embodiments, the number of cells administered as a therapeutically effective amount of the EBV antigen and/or CD22 CAR T cells is greater than the number of cells administered as a therapeutically effective amount of EBV antigen CAR T cells or CD22 CAR T cells alone.

In some embodiments, the number of cells administered to as a therapeutically effective amount of the EBV antigen and/or CD22 CAR T cells is less than the number of cells administered as a therapeutically effective amount of EBV antigen CAR T cells or CD22 CAR T cells alone.

In some embodiments, the engineered T cells are primary T cells, are propagated from a primary T cell or a progeny thereof, or are derived from a T cell differentiated from an iPSC or a progeny thereof.

In some embodiments, the engineered T cells are differentiated cells derived from an induced pluripotent stem cell or a progeny thereof.

In some embodiments, the differentiated cells are a T cells or natural killer (NK) cells.

In some embodiments, the engineered T cells are primary T cells or are progeny of primary immune cells, optionally wherein the progeny of primary immune cells are T cells or NK cells.

In some embodiments, the engineered T cells comprise reduced expression of one or more MHC HLA class I molecules relative to an unaltered or unmodified wild-type or control cell.

In some embodiments, the engineered T cells comprise reduced expression of one or more MHC HLA class II molecules relative to an unaltered or unmodified wild-type or control cell.

In some embodiments, the engineered T cells comprise reduced expression of one or more MHC HLA class I molecules and of one or more MHC HLA class II molecules relative to an unaltered or unmodified wild-type or control cell.

In some embodiments, the engineered T cells comprise reduced expression of B2M and/or CIITA relative to an unaltered or unmodified wild-type or control cell.

In some embodiments, the engineered T cells do not express B2M and/or CIITA.

In some embodiments, the engineered T cells comprise reduced expression of TRAC and/or TRB.

In some embodiments, the engineered T cells do not express TRAC and/or TRB.

In some embodiments, the engineered T cells comprise reduced expression of TRAC.

In some embodiments, the engineered T cells do not express TRAC.

In some embodiments, the engineered T cells comprise reduced expression of TRB.

In some embodiments, the engineered T cells do not express TRB.

In some embodiments, the engineered T cells comprise reduced expression of TRAC and TRB.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, and MANF, and Serpinb9, optionally wherein the one or more tolerogenic factors comprise CD47.

In some embodiments, the CD19-specific CAR and the one or more tolerogenic factors are encoded by a single bicistronic polynucleotide.

In some embodiments, the CD19-specific CAR and the CD47 are encoded by a single bicistronic polynucleotide.

In some embodiments, the CD20-specific CAR and the one or more tolerogenic factors are encoded by a single bicistronic polynucleotide.

In some embodiments, the CD20-specific CAR and the CD47 are encoded by a single bicistronic polynucleotide.

In some embodiments, one or more of the first, second, and/or third exogenous polynucleotides or the bicistronic polynucleotide is inserted into a first, second, and/or third specific locus of at least one allele of the cell.

In some embodiments, the first, second and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

In some embodiments, the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons. In some embodiments, the gene therapy vector is a retrovirus or a fusosome.

In some embodiments, the first, second, and/or third exogenous polynucleotide or the bicistronic polynucleotide is introduced into the engineered T cells using CRISPR/Cas gene editing.

In some embodiments, the CRISPR/Cas system comprises a Cas effector protein selected from the group consisting of Cas9, Cas12a, and Cas12b.

In some embodiments, the CRISPR/Cas system comprises a Cas effector protein selected from the group consisting of:
(a) optionally selected from the group consisting of Cas3, Cas8a, Cas5, Cas8b, Cas8c, Cas10d, Cse1, Cse2, Csy1, Csy2, Csy3, and GSU0054;
(b) optionally selected from the group consisting of Cas9, Csn2, and Cas4;
(c) optionally selected from the group consisting of Cas10, Csm2, Cmr5, Cas10, Csx11, and Csx10;
(d) optionally Csf1;
(e) optionally selected from the group consisting of Cas12a, Cas12b, Cas12c, C2c4, C2c8, C2c5, C2c10, C2c9, CasX (Cas12e), and CasY (Cas12d); and
(f) optionally selected from the group consisting of Cas13, Cas13a, C2c2, Cas13b, Cas13c, and Cas13d.

In some embodiments, the CRISPR/Cas gene editing is carried out ex vivo from a donor subject.

In some embodiments, the first, second, and/or third exogenous polynucleotide or the bicistronic polynucleotide is introduced into the engineered T cells using a lentiviral vector.

In some embodiments, the engineered T cells evade NK cell mediated cytotoxicity upon administration to the patient.

In some embodiments, the engineered T cells are protected from cell lysis by mature NK cells upon administration to the patient.

In some embodiments, the engineered T cells evade macrophage-mediated cytotoxicity, optionally wherein the macrophage-mediated cytotoxicity involves phagocytosis and/or reactive oxygen species.

In some embodiments, the engineered T cells do not induce an immune response to the cell upon administration to the patient.

In some embodiments, the administration is selected from the group consisting of intravenous injection, intramuscular injection, intravascular injection, and transplantation.

In some embodiments, the engineered T cells are administered before, during or after starting a different treatment regimen for the patient.

In some embodiments, the different treatment regimen is selected from the group consisting of re-dosing of the same or different cells, and pre-treatment, concurrent treatment, or subsequent treatment with an additional agent.

In some embodiments, the different cells are autologous T or NK cells or CAR-T cells expressing a first CAR that is different from a second CAR expressed by the engineered CAR-T cells.

In some embodiments, the patient was treated with an immunodepleting therapy prior to administering the engineered T cells.

In some embodiments, the immunodepleting therapy comprises administration of fludarabine and/or cyclophosphamide.

In some embodiments, the patient has undergone a prior antibody therapy.

In some embodiments, the antibody therapy is rituximab.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 4 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, about 1000 mg/m² of cyclophosphamide for about 1, about 2, about 3, about 4, about 5, about 6, or about 7 days.

In some embodiments, the immunodepleting therapy comprises IV infusion of about 500 mg/m² of cyclophosphamide for about 2 days.

In some embodiments, at least about 40×10⁴ engineered T cells are administered to the patient.

In some embodiments, at least about 40×10⁵ engineered T cells are administered to the patient.

In some embodiments, the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the wild type cell or the control cell is a starting material.

In some embodiments, provided herein is a use of a population of engineered T cells for treating an EBV infection in a patient that is suspected of having an EBV infection or has been diagnosed with an EBV infection, wherein the engineered T cells comprise reduced expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs) wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating an EBV infection in a patient that is suspected of having an EBV infection or has been diagnosed with an EBV infection, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs wherein the one or more CARs comprise an extracellular ligand-binding domain having specificity for CD19 or CD22, a hinge domain, a transmembrane domain, a co-stimulatory domain, and an intracellular signaling domain.

In some embodiments, provided herein is a use of a population of engineered T cells for treating an EBV infection in a patient that is suspected of having an EBV infection or has been diagnosed with an EBV infection, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

In some embodiments, the one or more CARs comprise a CD8α hinge domain, a CD28 hinge domain, or an IgG4 hinge domain.

In some embodiments, the one or more CARs comprise a CD8α hinge domain having the amino acid sequence of SEQ ID NO: 9.

In some embodiments, the one or more CARs comprise a CD28 hinge domain having the amino acid sequence of SEQ ID NO: 10 or 113.

In some embodiments, the one or more CARs comprise a IgG4 hinge domain having the amino acid sequence of SEQ ID NO: 11 or 12.

In some embodiments, the one or more CARs comprise a CD8α transmembrane domain or a CD28 transmembrane domain.

In some embodiments, the one or more CARs comprise a CD8α transmembrane domain having the amino acid sequence of SEQ ID NO: 14.

In some embodiments, the one or more CARs comprise a CD28 transmembrane domain having the amino acid sequence of SEQ ID NO: 15 or 114.

In some embodiments, the one or more CARs comprise a 4-1BB costimulatory domain, a CD28 costimulatory domain, or a CD3ζ signaling domain.

In some embodiments, the one or more CARs comprise a 4-1BB costimulatory domain having the amino acid sequence of SEQ ID NO: 16.

In some embodiments, the one or more CARs comprise a CD28 costimulatory domain having the amino acid sequence of SEQ ID NO: 17.

In some embodiments, the one or more CARs comprise a CD3ζ signaling domain having the amino acid sequence of SEQ ID NO: 18 or 115.

In some embodiments, the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the one or more CARs have a sequence of any one of SEQ ID NOs: 32, 34, 36, 117, 91, 92, 92, 128, 133, or 134.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD20 CAR having the CDR sequences of SEQ ID NO: 37.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a EBV antigen CAR having the CDR sequences of SEQ ID NO: 133 or 134, and/or the CDR sequences from the VH/VL sequences of SEQ ID NOs: 129-132 or 135-172.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD22 CAR having the CDR sequences of SEQ ID NO: 45.

In some embodiments, provided herein is a use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45.

In some embodiments, provided herein is a use of a population of engineered T cells for treating multiple sclerosis in a patient that is suspected of having multiple sclerosis or has been diagnosed with multiple sclerosis, wherein the engineered T cells comprise an exogenous polynucleotide encoding one or more CARs wherein the one or more CARs have a sequence of any one of SEQ ID NOs: 32, 34, 36, 117, 91, 92, 92, 128, 133, or 134, or wherein the one or more CARs have an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the use further comprises evaluating the patient for and/or diagnosing the patient with EBV infection and optionally multiple sclerosis prior to administering the population of engineered T cells to the patient.

In some embodiments, the diagnosis comprises evaluating the patient for EBV infection.

In some embodiments, the diagnosis comprises evaluating the patient for multiple sclerosis.

In some embodiments, the treatment prevents multiple sclerosis.

In some embodiments, the treatment treats multiple sclerosis.

In some embodiments, the patient with the EBV infection has been diagnosed with multiple sclerosis.

In some embodiments, the multiple sclerosis is relapsing-remitting multiple sclerosis, primary progressive multiple sclerosis, or secondary progressive multiple sclerosis.

In some embodiments, patient undergoes remission of multiple sclerosis following administration of the engineered T cells.

In some embodiments, the patient with the EBV infection is undergoing treatment for the EBV infection.

In some embodiments, the patient with the EBV infection has an active EBV infection.

In some embodiments, the patient with the EBV infection has an inactive EBV infection.

In some embodiments, the patient undergoes a reduced EBV infection following administration of the engineered T cells, optionally wherein the reduced EBV infection is characterized by reduced viral load.

In some embodiments, the treatment prevents an EBV infection change from an inactive to an active EBV infection.

In some embodiments, the use results in B cell depletion.

In some embodiments, the engineered T cells comprise one or more of a CD19-specific CAR, a CD20-specific CAR, a CD22-specific CAR, a BCMA-specific CAR, a GPRC5D-specific CAR, a CD38-specific CAR, a CD70-specific CAR, a CD79b-specific CAR, and an EBV antigen-specific CAR.

In some embodiments, provided herein is a use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the one or more CARs comprise a CD8α hinge domain, a CD28 hinge domain, or an IgG4 hinge domain.

In some embodiments, the one or more CARs comprise a CD8α hinge domain having the amino acid sequence of SEQ ID NO: 9.

In some embodiments, the one or more CARs comprise a CD28 hinge domain having the amino acid sequence of SEQ ID NO: 10 or 113.

In some embodiments, the one or more CARs comprise a IgG4 hinge domain having the amino acid sequence of SEQ ID NO: 11 or 12.

In some embodiments, the one or more CARs comprise a CD8α transmembrane domain or a CD28 transmembrane domain.

In some embodiments, the one or more CARs comprise a CD8α transmembrane domain having the amino acid sequence of SEQ ID NO: 14.

In some embodiments, the one or more CARs comprise a CD28 transmembrane domain having the amino acid sequence of SEQ ID NO: 15 or 114.

In some embodiments, the one or more CARs comprise a 4-1BB costimulatory domain, a CD28 costimulatory domain, or a CD3ζ signaling domain.

In some embodiments, the one or more CARs comprise a 4-1BB costimulatory domain having the amino acid sequence of SEQ ID NO: 16.

In some embodiments, the one or more CARs comprise a CD28 costimulatory domain having the amino acid sequence of SEQ ID NO: 17.

In some embodiments, the one or more CARs comprise a CD3ζ signaling domain having the amino acid sequence of SEQ ID NO: 18 or 115.

In some embodiments, the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the one or more CARs have a sequence of any one of SEQ ID NOs: 32, 34, 36, 117, 91, 92, 92, 128, 133, or 134.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, use further comprises evaluating the patient for and/or diagnosing the patient with the autoimmune disease.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

In some embodiments, the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:117, with the following components: CD8α signal peptide, FMC63 scFv (VL-Whitlow linker-VH), CD8α hinge domain, CD8α transmembrane domain, 4-1BB costimulatory domain, and CD3ζ signaling domain.

In some embodiments, the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:45.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a EBV antigen-specific CAR having the CDR sequences of SEQ ID NO: 133 or 134, and/or the CDR sequences from the VH/VL sequences of SEQ ID NOs: 129-132 or 135-172, and wherein the autoimmune disease is multiple sclerosis.

In some embodiments, provided herein is a use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, provided herein is a use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

In some embodiments, the use further comprises evaluating the patient for and/or diagnosing the patient with the autoimmune disease prior to administering the population of engineered T cells to the patient.

In some embodiments, the autoimmune disease is selected from the group consisting of lupus, systemic lupus erythematosus, multiple sclerosis, systemic sclerosis, vasculitis, Crohn's disease, Myasthenia Gravis, Stiff-Person syndrome and a pulmonary condition.

In some embodiments, the patient is suspected of having an EBV infection or has been diagnosed as having an EBV infection.

In some embodiments, the patient is suspected of having multiple sclerosis or has been diagnosed with multiple sclerosis.

In some embodiments, the use further comprises administering a second, third, fourth, fifth, or sixth dose of the engineered T cells to the patient.

In some embodiments, the same in one or more of the first, second, third, fourth, fifth, and/or sixth dose of engineered T cells.

In some embodiments, the CAR is different in one or more of the first, second, third, fourth, fifth, and/or sixth dose of engineered T cells.

In some embodiments, the CAR has an scFv sequence of any one of SEQ ID NOS: 19, 29, 37, 45, 54, 85, 63, 72, or 118, or wherein the CARs have an scFv sequence comprising the heavy and light chain sequences of any one of SEQ ID NOs: 129-132 or 135-172.

In some embodiments, the CAR has a sequence of any one of SEQ ID NOs: 32, 34, 36, 117, 91, 92, 92, 128, 133, and 134.

In some embodiments, the engineered T cells comprise a CD19-specific CAR and a CD20-specific CAR.

In some embodiments, the CD19-specific CAR has the CDR sequences of SEQ ID NO: 117 and the CD22 CAR has the CDR sequences of SEQ ID NO: 45.

In some embodiments, the CD19-specific CAR and the CD20-specific CAR are encoded by a single bicistronic polynucleotide.

In some embodiments, the CD19-specific CAR and the CD20-specific CAR are encoded by a single bispecific CAR.

In some embodiments, the CD19-specific CAR and the CD20-specific CAR are encoded by two separate polynucleotides.

In some embodiments, the CD19 CAR T cells and CD20 CAR T cells are administered concomitantly.

In some embodiments, the CD19 CAR+ T cells and CD20 CAR+ T cells are administered sequentially.

In some embodiments, the CD19 CAR+ T cells are administered prior to administration of the CD20 CAR+ T cells.

In some embodiments, the CD20 CAR+ T cells are administered prior to administration of the CD19 CAR+ T cells.

In some embodiments, the number of cells administered as a therapeutically effective amount of the CD19 and/or CD20 CAR T cells is greater than the number of cells administered as a therapeutically effective amount of CD19 CAR T cells or CD20 CAR T cells alone.

In some embodiments, the number of cells administered to as a therapeutically effective amount of the CD19 and/or CD20 CAR T cells is less than the number of cells administered as a therapeutically effective amount of CD19 CAR T cells or CD20 CAR T cells alone.

In some embodiments, the engineered T cells comprise an EBV antigen-specific CAR and a CD20-specific CAR.

In some embodiments, the EBV antigen-specific CAR and the CD20-specific CAR are encoded by a single bicistronic polynucleotide.

In some embodiments, the EBV antigen-specific CAR and the CD20-specific CAR are encoded by a single bispecific CAR.

In some embodiments, the EBV antigen-specific CAR and the CD20-specific CAR are encoded by two separate polynucleotides.

In some embodiments, the EBV antigen CAR T cells and CD20 CAR T cells are administered concomitantly.

In some embodiments, the EBV antigen CAR+ T cells and CD20 CAR+ T cells are administered sequentially.

In some embodiments, the EBV antigen CAR+ T cells are administered prior to administration of the CD20 CAR+ T cells.

In some embodiments, the CD20 CAR+ T cells are administered prior to administration of the EBV antigen CAR+ T cells.

In some embodiments, the number of cells administered as a therapeutically effective amount of the EBV antigen and/or CD20 CAR T cells is greater than the number of cells administered as a therapeutically effective amount of EBV antigen CAR T cells or CD20 CAR T cells alone.

In some embodiments, the number of cells administered to as a therapeutically effective amount of the EBV antigen and/or CD20 CAR T cells is less than the number of cells administered as a therapeutically effective amount of EBV antigen CAR T cells or CD20 CAR T cells alone.

In some embodiments, the engineered T cells comprise an EBV antigen-specific CAR and a CD19-specific CAR.

In some embodiments, the EBV antigen-specific CAR and the CD19-specific CAR are encoded by a single bicistronic polynucleotide.

In some embodiments, the EBV antigen-specific CAR and the CD19-specific CAR are encoded by a single bispecific CAR.

In some embodiments, the EBV antigen-specific CAR and the CD19-specific CAR are encoded by two separate polynucleotides.

In some embodiments, the EBV antigen CAR T cells and CD19 CAR T cells are administered concomitantly.

In some embodiments, the EBV antigen CAR+ T cells and CD19 CAR+ T cells are administered sequentially.

In some embodiments, the EBV antigen CAR+ T cells are administered prior to administration of the CD19 CAR+ T cells.

In some embodiments, the CD19 CAR+ T cells are administered prior to administration of the EBV antigen CAR+ T cells.

In some embodiments, the number of cells administered as a therapeutically effective amount of the EBV antigen and/or CD19 CAR T cells is greater than the number of cells administered as a therapeutically effective amount of EBV antigen CAR T cells or CD19 CAR T cells alone.

In some embodiments, the number of cells administered to as a therapeutically effective amount of the EBV antigen and/or CD19 CAR T cells is less than the number of cells administered as a therapeutically effective amount of EBV antigen CAR T cells or CD19 CAR T cells alone.

In some embodiments, the engineered T cells comprise an EBV antigen-specific CAR and a CD22-specific CAR.

In some embodiments, the EBV antigen-specific CAR and the CD22-specific CAR are encoded by a single bicistronic polynucleotide.

In some embodiments, the EBV antigen-specific CAR and the CD22-specific CAR are encoded by a single bispecific CAR.

In some embodiments, the EBV antigen-specific CAR and the CD22-specific CAR are encoded by two separate polynucleotides.

In some embodiments, the EBV antigen CAR T cells and CD22 CAR T cells are administered concomitantly.

In some embodiments, the EBV antigen CAR+ T cells and CD22 CAR+ T cells are administered sequentially.

In some embodiments, the EBV antigen CAR+ T cells are administered prior to administration of the CD22 CAR+ T cells.

In some embodiments, the CD22 CAR+ T cells are administered prior to administration of the EBV antigen CAR+ T cells.

In some embodiments, the number of cells administered as a therapeutically effective amount of the EBV antigen and/or CD22 CAR T cells is greater than the number of cells administered as a therapeutically effective amount of EBV antigen CAR T cells or CD22 CAR T cells alone.

In some embodiments, the number of cells administered to as a therapeutically effective amount of the EBV antigen and/or CD22 CAR T cells is less than the number of cells administered as a therapeutically effective amount of EBV antigen CAR T cells or CD22 CAR T cells alone.

In some embodiments, the engineered T cells are primary T cells, are propagated from a primary T cell or a progeny thereof, or are derived from a T cell differentiated from an iPSC or a progeny thereof.

In some embodiments, the engineered T cells are differentiated cells derived from an induced pluripotent stem cell or a progeny thereof.

In some embodiments, the differentiated cells are a T cells or natural killer (NK) cells.

In some embodiments, the engineered T cells are primary T cells, are progeny of primary immune cells, optionally wherein the progeny of primary immune cells are T cells or NK cells.

In some embodiments, the engineered T cells comprise reduced expression of one or more MHC HLA class I molecules relative to an unaltered or unmodified wild-type or control cell.

In some embodiments, the engineered T cells comprise reduced expression of one or more MHC HLA class II molecules relative to an unaltered or unmodified wild-type or control cell.

In some embodiments, the engineered T cells comprise reduced expression of one or more MHC HLA class I molecules and of one or more MHC HLA class II molecules relative to an unaltered or unmodified wild-type or control cell.

In some embodiments, the engineered T cells comprise reduced expression of B2M and/or CIITA relative to an unaltered or unmodified wild-type or control cell.

In some embodiments, the engineered T cells do not express B2M and/or CIITA.

In some embodiments, the engineered T cells comprise reduced expression of TRAC and/or TRB.

In some embodiments, the engineered T cells do not express TRAC and/or TRB.

In some embodiments, the engineered T cells comprise reduced expression of TRAC.

In some embodiments, the engineered T cells do not express TRAC.

In some embodiments, the engineered T cells comprise reduced expression of TRB.

In some embodiments, the engineered T cells do not express TRB.

In some embodiments, the engineered T cells comprise reduced expression of TRAC and TRB.

In some embodiments, the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9, optionally wherein the one or more tolerogenic factors comprise CD47.

In some embodiments, the CD19-specific CAR and the one or more tolerogenic factors are encoded by a single bicistronic polynucleotide.

In some embodiments, the CD19-specific CAR and the CD47 are encoded by a single bicistronic polynucleotide.

In some embodiments, the CD20-specific CAR and the one or more tolerogenic factors are encoded by a single bicistronic polynucleotide.

In some embodiments, the CD20-specific CAR and the CD47 are encoded by a single bicistronic polynucleotide.

In some embodiments, one or more of the first, second, and/or third exogenous polynucleotides or the bicistronic polynucleotide is inserted into a first, second, and/or third specific locus of at least one allele of the cell.

In some embodiments, the first, second and/or third specific loci are selected from the group consisting of a safe harbor locus, a target locus, an RHD locus, a B2M locus, a CIITA locus, a TRAC locus, and a TRB locus.

In some embodiments, the safe harbor locus is selected from the group consisting of a CCR5 locus, a PPP1R12C locus, a CLYBL locus, and a Rosa locus.

In some embodiments, the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

In some embodiments, the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.

In some embodiments, the gene therapy vector is a retrovirus or a fusosome.

In some embodiments, the first, second, and/or third exogenous polynucleotide or the bicistronic polynucleotide is introduced into the engineered T cells using CRISPR/Cas gene editing.

In some embodiments, the CRISPR/Cas system comprises a Cas effector protein selected from the group consisting of Cas9, Cas12a, and Cas12b.

In some embodiments, the CRISPR/Cas system comprises a Cas effector protein selected from the group consisting of:
(a) optionally selected from the group consisting of Cas3, Cas8a, Cas5, Cas8b, Cas8c, Cas10d, Cse1, Cse2, Csy1, Csy2, Csy3, and GSU0054;
(b) optionally selected from the group consisting of Cas9, Csn2, and Cas4;
(c) optionally selected from the group consisting of Cas10, Csm2, Cmr5, Cas10, Csx11, and Csx10;
(d) optionally Csf1;
(e) optionally selected from the group consisting of Cas12a, Cas12b, Cas12c,
C2c4, C2c8, C2c5, C2c10, C2c9, CasX (Cas12e), and CasY (Cas12d); and
(f) optionally selected from the group consisting of Cas13, Cas13a, C2c2, Cas13b, Cas13c, and Cas13d.

In some embodiments, the CRISPR/Cas gene editing is carried out ex vivo from a donor subject.

In some embodiments, the first, second, and/or third exogenous polynucleotide or the bicistronic polynucleotide is introduced into the engineered T cells using a lentiviral vector.

In some embodiments, the engineered T cells evade NK cell mediated cytotoxicity upon administration to the patient.

In some embodiments, the engineered T cells are protected from cell lysis by mature NK cells upon administration to the patient.

In some embodiments, the engineered T cells evade macrophage-mediated cytotoxicity, optionally wherein the macrophage-mediated cytotoxicity involves phagocytosis and/or reactive oxygen species.

In some embodiments, the engineered T cells do not induce an immune response to the cell upon administration to the patient.

In some embodiments, the administration is selected from the group consisting of intravenous injection, intramuscular injection, intravascular injection, and transplantation.

In some embodiments, the engineered T cells are administered before, during or after starting a different treatment regimen for the patient.

In some embodiments, the different treatment regimen is selected from the group consisting of re-dosing of the same or different cells, and pre-treatment, concurrent treatment, or subsequent treatment with an additional agent.

In some embodiments, the different cells are autologous T or NK cells or CAR-T cells expressing a first CAR that is different from a second CAR expressed by the engineered CAR-T cells.

In some embodiments, the patient was treated with an immunodepleting therapy prior to administering the engineered T cells.

In some embodiments, the immunodepleting therapy comprises administration of fludarabine and/or cyclophosphamide.

In some embodiments, the patient has undergone a prior antibody therapy.

In some embodiments, the antibody therapy is rituximab.

In some embodiments, the immunodepleting therapy comprises IV infusion of about 1-50 mg/m² of fludarabine for about 1-7 days.

In some embodiments, the immunodepleting therapy comprises IV infusion of about 1, about 5, about 10, about 20, about 30, about 40, or about 50 mg/m² of fludarabine for about 1, about 2, about 3, about 4, about 5, about 6, or about 7 days.

In some embodiments, the immunodepleting therapy comprises IV infusion of about 30 mg/m² of fludarabine for about 4 days.

In some embodiments, the immunodepleting therapy comprises IV infusion of about 100-1000 mg/m² of cyclophosphamide for about 1-7 days.

In some embodiments, the immunodepleting therapy comprises IV infusion of about 100, about 200, about 300, about 400, about 500, about 600, about 700, about 800, about 900, about 1000 mg/m² of cyclophosphamide for about 1, about 2, about 3, about 4, about 5, about 6, or about 7 days.

In some embodiments, the immunodepleting therapy comprises IV infusion of about 500 mg/m² of cyclophosphamide for about 2 days.

In some embodiments, at least about 40×10⁴ engineered T cells are administered to the patient.

In some embodiments, at least about 40×10⁵ engineered T cells are administered to the patient.

In some embodiments, the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

In some embodiments, the wild type cell or the control cell is a starting material.

The present disclosure is related to U.S. Provisional Application filed on Dec. 31, 2020 (Attorney Docket No. 112864-5057-PR) and U.S. Provisional Application filed on Jan. 11, 2021 filed by Morrison and Foerester having Attorney Docket No. 18615-30046.00, the contents of which are hereby incorporated by reference in their entirety. Detailed descriptions of engineered and/or hypoimmunogenic cells, methods of producing thereof, and methods of using thereof are found in U.S. Provisional Application No. 63/065,342 filed on Aug. 13, 2020, WO2016/183041 filed May 9, 2015, WO2018/132783 filed Jan. 14, 2018, WO2020/018615 filed Jul. 17, 2019, WO2020/018620 filed Jul. 17, 2019, WO2020/168317 filed Feb. 16, 2020, the disclosures of which including the examples, sequence listings and figures are incorporated herein by reference in their entireties.

### DETAILED DESCRIPTION

### I. Introduction

Described herein are engineered or modified immune evasive cells based, in part, on the hypoimmune editing platform described in WO2018132783, and PCT/US21/65157 filed Dec. 23, 2021, each of which is incorporated herein by reference in its entirety, including but not limited to human immune evasive cells. To overcome the problem of a subject's immune rejection of these primary and/or stem cell-derived transplants, the inventors have developed and describe herein hypoimmunogenic cells (e.g., hypoimmunogenic pluripotent cells, differentiated cells derived from such, and primary cells) that represent a viable source for any transplantable cell type. Such cells are protected from adaptive and/or innate immune rejection upon administration to a recipient subject. Advantageously, the cells disclosed herein are not rejected by the recipient subject's immune system, regardless of the subject's genetic make-up, as they are protected from adaptive and innate immune rejection upon administration to a recipient subject. In some embodiments, the engineered and/or hypoimmunogenic cells do not express major histocompatibility complex (MHC) class I and class II antigen molecules and/or T-cell receptors. In certain embodiments, the engineered and/or hypoimmunogenic cells do not express MHC I and II antigen molecules and/or T-cell receptors and overexpress CD47 proteins. In certain embodiments, the engineered and/or hypoimmunogenic cells such as engineered and/or hypoimmunogenic T cells do not express MHC I and II antigen molecules and/or T-cell receptors, overexpress CD47 proteins and express exogenous CARs.

In some embodiments, hypoimmunogenic cells outlined herein are not subject to an innate immune cell rejection. In some instances, hypoimmunogenic cells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation, including, e.g., pluripotency, as well as being capable of engraftment and functioning similarly to a corresponding native cell.

The technology disclosed herein utilizes expression of tolerogenic factors and modulation (e.g., reduction or elimination) of MHC I molecules, MHC II molecules, and/or TCR expression in human cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in the cells. In some embodiments, genome editing technologies or other gene modulation technologies are used to insert tolerance-inducing (tolerogenic) factors in human cells, rendering the cells and their progeny (include any differentiated cells prepared therefrom) able to evade immune recognition upon engrafting into a recipient subject. As such, the cells described herein exhibit modulated expression of one or more genes and factors that affect MHC I molecules, MHC II molecules, and/or TCR expression and evade the recipient subject's immune system.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the numerous embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### II. Definitions

As described in the present disclosure, the following terms will be employed, and are defined as indicated below.

The term “antigen”, as used herein, refers to a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign.

The terms “autoimmune disease” or “autoimmune disorder” or “inflammatory disease” or “inflammatory disorder” refer to any disease or disorder in which the subject mounts an immune response against its own tissues and/or cells. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

In some embodiments, autoimmune or inflammatory disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis (such as plaque psoriasis, gutatte psoriasis, pustular psoriasis, and psoriasis of the nails), atopy (including atopic diseases such as hay fever and Job's syndrome), dermatitis (including contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis), x-linked hyper IgM syndrome, allergic intraocular inflammatory diseases, urticaria (such as chronic allergic urticaria, chronic idiopathic urticaria, chronic autoimmune urticaria), myositis, polymyositis/dermatomyositis, juvenile dermatomyositis, toxic epidermal necrolysis, scleroderma (including systemic scleroderma), sclerosis (such as systemic sclerosis; multiple sclerosis (MS), MS associated with Epstein Barr Virus (EBV) infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, and ataxic sclerosis), neuromyelitis optica spectrum disorder (NMO, also known as Devic's Disease or Devic's Syndrome), inflammatory bowel disease (IBD) including Crohn's disease; autoimmune-mediated gastrointestinal diseases; colitis such as ulcerative colitis, colitis ulcerosa, microscopic colitis, collagenous colitis, colitis polyposa, necrotizing enterocolitis, and transmural colitis; and autoimmune inflammatory bowel disease), bowel inflammation, pyoderma gangrenosum, erythema nodosum, primary sclerosing cholangitis, respiratory distress syndrome (including adult or acute respiratory distress syndrome (ARDS)), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis (such as anterior uveitis, acute anterior uveitis, granulomatous uveitis, nongranulomatous uveitis, phacoantigenic uveitis, posterior uveitis, or autoimmune uveitis), glomerulonephritis (GN) with and without nephrotic syndrome (such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, or proliferative nephritis), autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema (including allergic or atopic eczema, asteatotic eczema, dyshidrotic eczema, and vesicular palmoplantar eczema), asthma (such as asthma bronchiale, bronchial asthma, and auto-immune asthma), conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus (including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE or subacute cutaneous SLE, neonatal lupus syndrome (NLE), and lupus erythematosus disseminatus, Type I diabetes, Type II diabetes, and latent autoimmune diabetes in adults (or Type 1.5 diabetes), juvenile onset (Type I) diabetes mellitus, including pediatric insulin-dependent diabetes mellitus (IDDM), adult onset diabetes mellitus (Type II diabetes), idiopathic diabetes, insipidus, diabetic retinopathy, diabetic nephropathy, and diabetic large-artery disorder; immune responses associated with acute and delayed hypersensitivity mediated by cytokines and T-lymphocytes; tuberculosis, sarcoidosis, granulomatosis (including lymphomatoid granulomatosis, Wegener's granulomatosis, or agranulocytosis), vasculitides (including vasculitis, large-vessel vasculitis, polymyalgia rheumatica and giant cell (Takayasu's) arteritis, medium-vessel vasculitis, Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis (such as Churg-Strauss vasculitis or syndrome (CSS) and ANCA-associated small-vessel vasculitis)), temporal arteritis, aplastic anemia, autoimmune aplastic anemia, Coombs positive anemia, Diamond Blackfan anemia, hemolytic anemia, immune hemolytic anemia including autoimmune hemolytic anemia (AIHA), pernicious anemia (anemia perniciosa), Addison's disease, pure red cell anemia or aplasia (PRCA); Factor VIII deficiency; hemophilia A; autoimmune neutropenia, pancytopenia, leukopenia, diseases involving leukocyte diapedesis, CNS inflammatory disorders, Alzheimer's disease, Parkinson's disease, multiple organ injury syndrome (such as those secondary to septicemia, trauma, or hemorrhage), antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP), post-transfusion purpura (PTP), heparin-induced thrombocytopenia, autoimmune or immune-mediated thrombocytopenia such as idiopathic thrombocytopenia purpura (ITP) including chronic or acute ITP, acquired thrombocytopeniarpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases, including thyroiditis autoimmune thyroiditis, Hashimoto's disease, chronic thyroiditis (Hashimoto's thyroiditis), or subacute thyroiditis), autoimmune thyroid disease, idiopathic hypothyroidism, or Grave's disease), polyglandular syndromes, autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, hepatitis, including autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility (e.g., due to anti-spermatozoan antibodies) mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, endometriosis, and pulmonary hemosiderosis.

As used herein, “B cell depletion” refers to a reduction in B cell levels in an animal or human after cell or antibody treatment, as compared to the B cell level before treatment. B cell levels are measurable using well known assays such as by getting a complete blood count, or by FACS analysis staining for known B cell markers. B cell depletion can be partial or complete. In one embodiment, the depletion of CD20 expressing B cells is at least 25%. In one embodiment, the depletion of CD19 expressing B cells is at least 20%, 25%, 30%, 35%, 40%, 45%, 50%, 55%, 60%, 65%, 70%, 75%, 80%, 82.5%, 85%, 87.5%, 90%, 92.5%, 95%, 97.5%, or 100%. In one embodiment, the depletion of CD22 expressing B cells is at least 20%, 25%, 30%, 35%, 40%, 45%, 50%, 55%, 60%, 65%, 70%, 75%, 80%, 82.5%, 85%, 87.5%, 90%, 92.5%, 95%, 97.5%, or 100%. In one embodiment, the depletion of CD19 and CD20 expressing B cells is at least 20%, 25%, 30%, 35%, 40%, 45%, 50%, 55%, 60%, 65%, 70%, 75%, 80%, 82.5%, 85%, 87.5%, 90%, 92.5%, 95%, 97.5%, or 100%. In one embodiment, the depletion of CD19 and CD22 expressing B cells is at least 20%, 25%, 30%, 35%, 40%, 45%, 50%, 55%, 60%, 65%, 70%, 75%, 80%, 82.5%, 85%, 87.5%, 90%, 92.5%, 95%, 97.5%, or 100%. In one embodiment, the depletion of CD20 and CD22 expressing B cells is at least 20%, 25%, 30%, 35%, 40%, 45%, 50%, 55%, 60%, 65%, 70%, 75%, 80%, 82.5%, 85%, 87.5%, 90%, 92.5%, 95%, 97.5%, or 100%. In some embodiments, depletion methods include those as described in Ercoli, G. et al., Front Immunol. 11:611661 (2020), incorporate herein by reference in its entirety.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

As used herein, “clinically effective amount” refers to an amount sufficient to provide a clinical benefit in the treatment and/or management of a disease, disorder, or condition. In some embodiments, a clinically effective amount is an amount that has been shown to produce at least one improved clinical endpoint to the standard of care for the disease, disorder, or condition. In some embodiments, a clinically effective amount is an amount that has been demonstrated, for example in a clinical trial, to be sufficient to provide statistically significant and meaningful effectiveness for treating the disease, disorder, or condition. In some embodiments, the clinically effective amount is also a therapeutically effective amount. In other embodiments, the clinically effective amount is not a therapeutically effective amount.

In some embodiments, an alteration or modification (including, for example, genetic alterations or modifications) described herein results in reduced expression of a target or selected polynucleotide sequence. In some embodiments, an alteration or modification described herein results in reduced expression of a target or selected polypeptide sequence. In some embodiments, an alteration or modification described herein results in increased expression of a target or selected polynucleotide sequence. In some embodiments, an alteration or modification described herein results in increased expression of a target or selected polypeptide sequence.

In additional or alternative embodiments, the present disclosure contemplates altering target polynucleotide sequences in any manner which is available to the skilled artisan, e.g., utilizing a TALEN system or RNA-guided transposases. It should be understood that although examples of methods utilizing CRISPR/Cas (e.g., Cas9 and Cas12a) and TALEN are described in detail herein, the present disclosure is not limited to the use of these methods/systems. Other methods of targeting, e.g., B2M, to reduce or ablate expression in target cells known to the skilled artisan can be utilized herein.

The term “comparable”, as used herein, refers to two or more agents, entities, situations, sets of conditions, etc. that may not be identical to one another but that are sufficiently similar to permit comparison there between so that conclusions may reasonably be drawn based on differences or similarities observed. Persons of ordinary skill in the art will understand, in context, what degree of identity is required in any given circumstance for two or more such agents, entities, situations, sets of conditions, etc. to be considered comparable. A control cell, for example, can be a comparable cell (e.g., same cell type) that does not comprise the relative modifications.

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. In some embodiments, the cells are engineered to have reduced expression of one or more targets relative to an unaltered or unmodified wild-type cell.

In some embodiments, the engineered and hypoimmunogenic cells described are derived from an iPSC or a progeny thereof. As used herein, the term “derived from an iPSC or a progeny thereof” encompasses the initial iPSC that is generated and any subsequent progeny thereof. As used herein, the term “progeny” encompasses, e.g., a first-generation progeny, i.e., the progeny is directly derived from, obtained from, obtainable from or derivable from the initial iPSC by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial iPSC or a progeny thereof.

The term “donor subject” refers to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor subject” also encompasses any vertebrate including but not limited to mammals, reptiles, amphibians and fish. However, advantageously, the donor subject is a mammal such as a human, or other mammals such as a domesticated mammal, e.g., dog, cat, horse, and the like, or production mammal, e.g., cow, sheep, pig, and the like. A “donor subject” can also refer to more than one donor, for example one or more humans or non-human animals or non-human mammals.

The term “endogenous” refers to a referenced molecule or polypeptide that is naturally present in the cell. Similarly, the term when used in reference to expression of an encoding nucleic acid refers to expression of an encoding nucleic acid naturally contained within the cell and not exogenously introduced. Similarly, the term when used in reference to a promoter sequence refers to a promoter sequence naturally contained within the cell and not exogenously introduced.

The term “engineered cell” as used herein refers to a cell that has been altered in at least some way by human intervention, including, for example, by genetic alterations or modifications such that the engineered cell differs from a wild-type cell.

As used herein, the term “exogenous” in the context of a polynucleotide or polypeptide being expressed is intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

As used herein, a “fusosome” includes to a gene therapy vector comprising retroviral vector pseudotyped with an engineered fusogen comprising a G protein modified to include a targeting moiety and an F protein blinded to no longer recognize its cognate receptor. In some embodiments, the fusogen protein complex is from a paraymyxovirus, optionally wherein the paraymyxovirus is a Nipah virus. In some embodiments, the retroviral vector is a lentiviral vector.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and/or locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and/or glycosylation.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences Genetic modifications also include, for example, reduced or increased transcription, reduced or increased mRNA stability, reduced or increased translation, and reduced or increased protein stability.

As used herein, the terms “grafting”, “administering,” “introducing”, “implanting” and “transplanting” as well as grammatical variations thereof are used interchangeably in the context of the placement of cells (e.g., cells described herein) into a subject, by a method or route which results in localization or at least partial localization of the introduced cells at a desired site or systemic introduction (e.g., into circulation). The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e.g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

By “HLA” or “human leukocyte antigen” or “HLA molecules” or “human leukocyte antigen molecules” complex is a gene complex encoding the MHC proteins in humans. These cell-surface proteins that make up the HLA complex are responsible for the regulation of the immune response to antigens. In humans, there are two MHCs, class I molecules and class II molecules, “HLA-I” and “HLA-II”, or “HLA-I molecules” and “HLA-II molecules”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein to characterize a cell, the term “hypoimmunogenic” generally means that such cell is less prone to innate or adaptive immune rejection by a subject into which such cells are transplanted, e.g., the cell is less prone to allorejection by a subject into which such cells are transplanted. For example, relative to a cell of the same cell type that does not comprise the modifications, such a hypoimmunogenic cell may be about 2.5%, 5%, 10%, 20%, 30%, 40%, 50%, 60%, 70%, 80%, 90%, 95%, 97.5%, 99% or more less prone to innate or adaptive immune rejection by a subject into which such cells are transplanted. In some embodiments, genome editing technologies are used to modulate the expression of MHC I and MHC II genes, and thus, contribute to generation of a hypoimmunogenic cell. In some embodiments, a hypoimmunogenic cell evades immune rejection in an MHC-mismatched allogeneic recipient. In some instance, differentiated cells produced from the hypoimmunogenic stem cells outlined herein evade immune rejection when administered (e.g., transplanted or grafted) to an MHC-mismatched allogeneic recipient. In some embodiments, a hypoimmunogenic cell is protected from T cell-mediated adaptive immune rejection and/or innate immune cell rejection. Detailed descriptions of hypoimmunogenic cells, methods of producing thereof, and methods of using thereof are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; PCT/US2020/44635 filed Jul. 31, 2020; WO2021022223 filed Jul. 31, 2020; WO2021041316 filed Aug. 24, 2020; WO2021222285 filed Apr. 27, 2021, 2020; and WO2021222285 filed Apr. 27, 2021, the disclosures including the examples, sequence listings and figures are incorporated herein by reference in their entirety.

Hypoimmunogenicity of a cell can be determined by evaluating the immunogenicity of the cell such as the cell's ability to elicit adaptive and innate immune responses or to avoid eliciting such adaptive and innate immune responses. Such immune response can be measured using assays recognized by those skilled in the art. In some embodiments, an immune response assay measures the effect of a hypoimmunogenic cell on T cell proliferation, T cell activation, T cell killing, donor specific antibody generation, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wild-type cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

“Immune signaling factor” as used herein refers to, in some cases, a molecule, protein, peptide and the like that activates immune signaling pathways.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment. These may be in combination with additional genetic modifications.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level, also referred to as the basal level, is 0.

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A gene editing (e.g. CRISPR/Cas) system of the present disclosure can be used to induce an indel of any length or a point mutation in a target polynucleotide sequence.

As used herein, “knock down” refers to a reduction in expression of the target mRNA or the corresponding target protein. Knock down is commonly reported relative to levels present following administration or expression of a noncontrol molecule that does not mediate reduction in expression levels of RNA (e.g., a non-targeting control shRNA, siRNA, or miRNA). In some embodiments, knock down of a target gene is achieved by way of conditional or inducible shRNAs, conditional or inducible siRNAs, conditional or inducible miRNAs, or conditional or inducible CRISPR interference (CRISPRi). In some embodiments, knock down of a target gene is achieved by way of a protein-based method, such as a conditional or inducible degron method. In some embodiments, knock down of a target gene is achieved by genetic modification, including shRNAs, siRNAs, miRNAs, or use of gene editing systems (e.g. CRISPR/Cas).

Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use the gene editing systems (e.g., CRISPR/Cas) of the present disclosure to knock out a target polynucleotide sequence or a portion thereof based upon the details described herein.

By “knock in” or “knock-in” herein is meant a genetic modification resulting from the insertion of a DNA sequence into a chromosomal locus in a host cell. This causes initiation of or increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This may be accomplished by modifying a promoter, adding a different promoter, adding an enhancer, adding other regulatory elements, or modifying other gene expression sequences.

As used herein, “knock out” or “knock-out” includes deleting all or a portion of a target polynucleotide sequence in a way that interferes with the translation or function of the target polynucleotide sequence. For example, a knock out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g. CRISPR/Cas) of the present disclosure to knock out a target polynucleotide sequence or a portion thereof based upon the details described herein.

In some embodiments, a genetic modification or alteration results in a knock out or knock down of the target polynucleotide sequence or a portion thereof. Knocking out a target polynucleotide sequence or a portion thereof using a gene editing system (e.g. CRISPR/Cas) of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell.

“Modulation” of gene expression refers to a change in the expression level of a gene. Modulation of expression can include, but is not limited to, gene activation and gene repression. Modulation may also be complete, i.e., wherein gene expression is totally inactivated or is activated to wild-type levels or beyond; or it may be partial, wherein gene expression is partially reduced, or partially activated to some fraction of wild-type levels.

In additional or alternative aspects, the present disclosure contemplates altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cas12a) and TALEN are described in detail herein, the disclosure is not limited to the use of these methods/systems. Other methods of targeting to reduce or ablate expression in target cells known to the skilled artisan can be utilized herein. The methods provided herein can be used to alter a target polynucleotide sequence in a cell. The present disclosure contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas) systems of the present disclosure. In other instances, a “mutant cell” exhibits a wild-type phenotype, for example when a gene editing system (e.g., CRISPR/Cas) system of the present disclosure is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

The term “native cell” as used herein refers to a cell that is not otherwise modified (e.g., engineered). In some embodiments, a native cell is a naturally occurring wild-type or a control cell.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

As used herein, “promoter,” “promoter sequence,” or “promoter region” refers to a DNA regulatory region/sequence capable of binding RNA polymerase and involved in initiating transcription of a downstream coding or non-coding sequence. In some examples, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

In some embodiments, the engineered and hypoimmunogenic cells described are propagated from a primary T cell or a progeny thereof. As used herein, the term “propagated from a primary T cell or a progeny thereof” encompasses the initial primary T cell that is isolated from the donor subject and any subsequent progeny thereof. As used herein, the term “progeny” encompasses, e.g., a first-generation progeny, i.e., the progeny is directly derived from, obtained from, obtainable from or derivable from the initial primary T cell by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial primary T cell or a progeny thereof.

The term “recipient patient” refers to an animal, for example, a human to whom treatment, including prophylactic treatment, with the cells as described herein, is provided. For treatment of those infections, conditions or disease states, which are specific for a specific animal such as a human patient, the term patient refers to that specific animal. The term “recipient patient” also encompasses any vertebrate including but not limited to mammals, reptiles, amphibians and fish. However, advantageously, the recipient patient is a mammal such as a human, or other mammals such as a domesticated mammal, e.g. dog, cat, horse, and the like, or production mammal, e.g. cow, sheep, pig, and the like.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example but are not limited to, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. It is recognized that since in most cases the exact boundaries of regulatory sequences have not been completely defined, nucleotide sequences of different lengths may have identical regulatory or promoter activity.

“Safe harbor locus” as used herein refers to a gene locus that allows expression of a transgene or an exogenous gene in a manner that enables the newly inserted genetic elements to function predictably and that also may not cause alterations of the host genome in a manner that poses a risk to the host cell. Exemplary “safe harbor” loci include, but are not limited to, a CCR5 gene, a PPP1R12C (also known as AAVS1) gene, a CLYBL gene, and/or a Rosa gene (e.g., ROSA26).

“Target locus” as used herein refers to a gene locus that allows expression of a transgene or an exogenous gene. Exemplary “target loci” include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, and/or a KDM5D gene (also known as HY). The exogenous polynucleotide encoding the exogenous gene can be inserted in the CDS region for B2M, CIITA, TRAC, TRBC, CCR5, F3 (i.e., CD142), MICA, MICB, LRP1, HMGB1, ABO, RHD, FUT1, KDM5D (i.e., HY), PDGFRa, OLIG2, and/or GFAP. The exogenous polynucleotide encoding the exogenous gene can be inserted in introns 1 or 2 for PPP1R12C (i.e., AAVS1) or CCR5. The exogenous polynucleotide encoding the exogenous gene can be inserted in exons 1 or 2 or 3 for CCR5. The exogenous polynucleotide encoding the exogenous gene can be inserted in intron 2 for CLYBL. The exogenous polynucleotide encoding the exogenous gene can be inserted in a 500 bp window in Ch-4:58,976,613 (i.e., SHS231). The exogenous polynucleotide encoding the exogenous gene can be insert in any suitable region of the aforementioned safe harbor or target loci that allows for expression of the exogenous, including, for example, an intron, an exon or a coding sequence region in a safe harbor or target locus.

As used herein, a “target” can refer to a gene, a portion of a gene, a portion of the genome, or a protein that is subject to regulatable reduced expression by the methods described herein.

As used herein, “therapeutically effective amount” refers to an amount sufficient to provide a therapeutic benefit in the treatment and/or management of a disease, disorder, or condition. In some embodiments, a therapeutically effective amount is an amount sufficient to ameliorate, palliate, stabilize, reverse, slow, attenuate or delay the progression of a disease, disorder, or condition, or of a symptom or side effect of the disease, disorder, or condition. In some embodiments, the therapeutically effective amount is also a clinically effective amount. In other embodiments, the therapeutically effective amount is not a clinically effective amount.

As used herein, the term “treating” and “treatment” includes administering to a subject a therapeutically or clinically effective amount of cells described herein so that the subject has a reduction in at least one symptom of the disease or an improvement in the disease, for example, beneficial or desired therapeutic or clinical results. For purposes of this technology, beneficial or desired therapeutic or clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

For purposes of this technology, beneficial or desired therapeutic or clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

In some embodiments, the cells are engineered to have reduced or increased expression of one or more targets relative to an unaltered or unmodified wild-type cell. In some embodiments, the cells are engineered to have constitutive reduced or increased expression of one or more targets relative to an unaltered or unmodified wild-type cell. In some embodiments, the cells are engineered to have regulatable reduced or increased expression of one or more targets relative to an unaltered or unmodified wild-type cell. In some embodiments, the cells comprise increased expression of CD47 relative to a wild-type cell or a control cell of the same cell type. By “wild-type” or “wt” or “control” in the context of a cell means any cell found in nature. Examples of wild type or control cells include primary cells and T cells found in nature. However, by way of example, in the context of an engineered cell, as used herein, “wild-type” or “control” can also mean an engineered cell that may contain nucleic acid changes resulting in reduced expression of one or more MHC class I molecules and/or class II molecules and/or T-cell receptors, but did not undergo the gene editing procedures to result in overexpression of CD47 proteins. For example, as used herein, “wild-type” or “control” means an engineered cell that comprises reduced or knocked out expression of B2M, CIITA, and/or TRAC. Also as used herein, “wild-type” or “control” means an engineered cell that comprises reduced or knocked out expression of B2M, CIITA, TRAC, and/or TRBC. As used herein, “wild-type” or “control” also means an engineered cell that may contain nucleic acid changes resulting in overexpression of CD47 proteins, but did not undergo the gene editing procedures to result in reduced expression of one or more MHC class I molecules and/or class II molecules and/or T-cell receptors. In the context of an iPSC or a progeny thereof, “wild-type” or “control” also means an iPSC or progeny thereof that may contain nucleic acid changes resulting in pluripotency but did not undergo the gene editing procedures of the present disclosure to achieve reduced expression of one or more MHC class I molecules and/or class II molecules and/or T-cell receptors, and/or overexpression of CD47 proteins. For example, as used herein, “wild-type” or “control” means an iPSC or progeny thereof that comprises reduced or knocked out expression of B2M, CIITA, and/or TRAC. Also as used herein, “wild-type” or “control” means an iPSC or progeny thereof that comprises reduced or knocked out expression of B2M, CIITA, TRAC, and/or TRBC. In the context of a primary T cell or a progeny thereof, “wild-type” or “control” also means a primary T cell or progeny thereof that may contain nucleic acid changes resulting in reduced expression of one or more MHC class I molecules and/or class II molecules and/or T-cell receptors, but did not undergo the gene editing procedures to result in overexpression of CD47 proteins. For example, as used herein, “wild-type” or “control” means a primary T cell or progeny thereof that comprises reduced or knocked out expression of B2M, CIITA, and/or TRAC. Also as used herein, “wild-type” or “control” means a primary T cell or progeny thereof that comprises reduced or knocked out expression of B2M, CIITA, TRAC, and/or TRBC. Also in the context of a primary T cell or a progeny thereof, “wild-type” or “control” also means a primary T cell or progeny thereof that may contain nucleic acid changes resulting in overexpression of CD47 proteins, but did not undergo the gene editing procedures to result in reduced expression of one or more MHC class I molecules and/or class II molecules and/or T-cell receptors. In some embodiments, the cells are engineered to have regulatable reduced or increased expression of one or more targets relative to a cell of the same cell type that does not comprise the modifications. In some embodiments, the wild-type cell or the control cell is a starting material. In some embodiments, the starting material is otherwise modified or engineered to have altered expression of one or more genes to generate the engineered cell.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present disclosure. Any recited method may be carried out in the order of events recited or in any other order that is logically possible. Although any methods and materials similar or equivalent to those described herein may also be used in the practice or testing of the present disclosure, representative illustrative methods and materials are now described.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges and are also encompassed within the present disclosure, subject to any specifically excluded limit in the stated range. Where the stated range includes one or both of the limits, ranges excluding either or both of those included limits are also included in the present disclosure. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number. The term about is used herein to mean plus or minus ten percent (10%) of a value. For example, “about 100” refers to any number between 90 and 110.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### III. Detailed Description

### A. Hypoimmunogenic Cells

In some embodiments, the present disclosure is directed to pluripotent stem cells (e.g., pluripotent stem cells and induced pluripotent stem cells (iPSCs)), differentiated cells derived from such pluripotent stem cells (such as, but not limited to, T cells and NK cells), and primary cells (such as, but not limited to, primary T cells and primary NK cells). In some embodiments, the pluripotent stem cells, differentiated cells derived therefrom, such as T cells and NK cells, and primary cells such as primary T cells and primary NK cells, are engineered for reduced expression or lack of expression of one or more MHC class I and/or MHC class II human leukocyte antigen molecules, and in some instances, for reduced expression or lack of expression of a T-cell receptor (TCR) complex. In some embodiments, the hypoimmune (HIP) T cells and primary T cells overexpress CD47 and a chimeric antigen receptor (CAR) in addition to reduced expression or lack of expression of one or more MHC class I and/or MHC class II human leukocyte antigen molecules, and have reduced expression or lack expression of a T-cell receptor (TCR) complex. In some embodiments, the CAR comprises an antigen binding domain that binds to any one selected from the group consisting of CD19, CD22, CD20, BCMA, an EBV antigen, CD27, CD30, EBNA1, EBNA3A, BRLF1, BALF4, EBNA3C, LMP1, LMP2, LMP2A, LMP2B, BZLF1, BMLF1, gp350, and gH/gL. In some embodiments, the CAR is a CD19-specific CAR. In some embodiments, the CAR is a CD20-specific CAR. In some instances, the CAR is a BCMA-specific CAR. In some embodiments, the CAR is an EBV antigen-specific CAR. In some embodiments, the CAR is a CD27-specific CAR. In some embodiments, the CAR is a CD30-specific CAR. In some embodiments, the CAR is a EBNA1-specific CAR. In some embodiments, the CAR is a EBNA3A-specific CAR. In some embodiments, the CAR is a BRLF1-specific CAR. In some embodiments, the CAR is a BALF4-specific CAR. In some embodiments, the CAR is a EBNA3C-specific CAR. In some embodiments, the CAR is a LMP1-specific CAR. In some embodiments, the CAR is a LMP2-specific CAR. In some embodiments, the CAR is a LMP2A-specific CAR. In some embodiments, the CAR is a LMP2B-specific CAR. In some embodiments, the CAR is a BZLF1-specific CAR. In some embodiments, the CAR is a BMLF1-specific CAR. In some embodiments, the CAR is a gp350-specific CAR. In some embodiments, the CAR is a gH/gL-specific CAR. In some embodiments, the CAR is a bispecific CAR. In some embodiments, the bispecific CAR is a CD19/CD20-bispecific CAR. In some embodiments, the bispecific CAR is a CD19/CD22-bispecific CAR. In some embodiments, the bispecific CAR is an EBV antigen/CD20-bispecific CAR. In some embodiments, the bispecific CAR is an EBV antigen/CD19-bispecific CAR. In some embodiments, the bispecific CAR is an EBV antigen/CD22-bispecific CAR. In some embodiments, the cells described express a CD19-specific CAR and a different CAR, such as, but not limited to a CD20-specific CAR, a BCMA-specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the cells described express a CD20-specific CAR and a different CAR, such as, but not limited to a CD19-specific CAR, a BCMA-specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the cells described express an EBV antigen-specific CAR and a different CAR, such as, but not limited to a CD19-specific CAR, a CD20-specific CAR, a CD22-specific CAR, a BCMA-specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the cells described express a CD22-specific CAR and a different CAR, such as, but not limited to a CD19-specific CAR, a BCMA-specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the cells described express a BCMA-specific CAR and a different CAR, such as, but not limited to a CD20-specific CAR, and a CD19-specific CAR. In some embodiments, the cells described express a BCMA-specific CAR and a different CAR, such as, but not limited to a CD22-specific CAR, and a CD19-specific CAR. In some embodiments, the cells described express a BCMA-specific CAR and a different CAR, such as, but not limited to a CD20-specific CAR, a CD19-specific CAR, a CD22-specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

In some embodiments, engineered and/or hypoimmune (HIP) T cells and primary T cells overexpress CD47 and a chimeric antigen receptor (CAR), and include a genomic modification of the B2M gene. In some embodiments, engineered and/or hypoimmune (HIP) T cells and primary T cells overexpress CD47 and include a genomic modification of the CIITA gene. In some embodiments, engineered and/or hypoimmune (HIP) T cells and primary T cells overexpress CD47 and a CAR, and include a genomic modification of the TRAC gene. In some embodiments, engineered and/or hypoimmune (HIP) T cells and primary T cells overexpress CD47 and a CAR, and include a genomic modification of the TRB gene. In some embodiments, engineered and/or hypoimmune (HIP) T cells and primary T cells overexpress CD47 and a CAR, and include one or more genomic modifications selected from the group consisting of the B2M, CIITA, TRAC, and TRB genes. In some embodiments, engineered and/or hypoimmune (HIP) T cells and primary T cells overexpress CD47 and a CAR, and include genomic modifications of the B2M, CIITA, TRAC, and TRB genes. In some embodiments, the cells are B2M^{-/-}, CIITA^{-/-}, TRAC^{-/-}, CD47tg cells that also express CARs. In some embodiments, engineered and/or hypoimmune (HIP) T cells are produced by differentiating induced pluripotent stem cells such as engineered and/or hypoimmunogenic induced pluripotent stem cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

In some embodiments, the engineered and/or hypoimmune (HIP) T cells and primary T cells are B2M^{-/-}, CIITA^{-/-}, TRB^{-/-}, CD47tg cells that also express CARs. In some embodiments, the cells are B2M^{-/-}, CIITA^{-/-}, TRAC^{-/-}, TRB^{-/-}, CD47tg cells that also express CARs. In certain embodiments, the cells are B2M^{indel/indel}, CIITA^{indel/indel}, TRAC^{indel/indel}, CD47tg cells that also express CARs. In certain embodiments, the cells are B2M^{indel/indel}, CIITA^{indel/indel}, TRB^{indel/indel}, CD47tg cells that also express CARs. In certain embodiments, the cells are B2M^{indel/indel} CIITA^{indel/indel}, TRAC^{indel/indel}, TRB^{indel/indel}, CD47tg cells that also express CARs. In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, or primary T cells. Non-limiting examples of primary T cells include CD3+ T cells, CD4+ T cells, CD8⁺ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

In some embodiments, the primary T cells are from a pool of primary T cells from one or more donor subjects that are different than the recipient subject (e.g., the patient administered the cells). The primary T cells can be obtained from 1, 2, 3, 4, 5, 6, 7, 8, 9, 10, 20, 50, 100 or more donor subjects and pooled together. The primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In certain embodiments, the primary T cells or the pool of primary T cells are engineered to express a chimeric antigen receptor (CAR). The CAR can be any known to those skilled in the art. Useful CARs include those that bind an antigen selected from a group that includes CD19, CD20, CD22, BCMA, an EBV antigen, CD27, CD30, EBNA1, EBNA3A, BRLF1, BALF4, EBNA3C, LMP1, LMP2, LMP2A, LMP2B, BZLF1, BMLF1, gp350, and gH/gL. In some cases, the CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. In certain embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the CAR-T cells comprise a CAR selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

In some embodiments, the CAR-T cells comprise a CAR comprising an antigen binding domain, a transmembrane, and one or more signaling domains. In some embodiments, the CAR also comprises a linker. In some embodiments, the CAR comprises a CD19 antigen binding domain. In some embodiments, the CAR comprises a EBV antigen binding domain. In some embodiments, the CAR comprises a CD27 binding domain. In some embodiments, the CAR comprises a CD30 binding domain. In some embodiments, the CAR comprises a EBNA1 binding domain. In some embodiments, the CAR comprises a EBNA3A binding domain. In some embodiments, the CAR comprises a BRLF1 binding domain. In some embodiments, the CAR comprises a BALF4 binding domain. In some embodiments, the CAR comprises a EBNA3C binding domain. In some embodiments, the CAR comprises a LMP1 binding domain. In some embodiments, the CAR comprises a LMP2 binding domain. In some embodiments, the CAR comprises a LMP2A binding domain. In some embodiments, the CAR comprises a LMP2B binding domain. In some embodiments, the CAR comprises a BZLF1 binding domain. In some embodiments, the CAR comprises a BMLF1 binding domain. In some embodiments, the CAR comprises a gp350 binding domain. In some embodiments, the CAR comprises a gH/gL binding domain. In some embodiments, the CAR comprises a CD28 or a CD8α transmembrane domain. In some embodiments, the CAR comprises a CD8α signal peptide. In some embodiments, the CAR comprises a Whitlow linker GSTSGSGKPGSGEGSTKG (SEQ ID NO: 24). In some embodiments, the antigen binding domain of the CAR is selected from a group including, but not limited to, (a) an antigen binding domain targets an antigen characteristic of a neoplastic cell; (b) an antigen binding domain that targets an antigen characteristic of a T cell; (c) an antigen binding domain targets an antigen characteristic of an autoimmune diseases/disorders and/or inflammatory diseases/disorders; (d) an antigen binding domain that targets an antigen characteristic of senescent cells; (e) an antigen binding domain that targets an antigen characteristic of an infectious disease; and (f) an antigen binding domain that binds to a cell surface antigen of a cell.

In some embodiments, the CAR further comprises one or more linkers. The format of an scFv is generally two variable domains linked by a flexible peptide sequence, or a “linker,” either in the orientation VH-linker-VL or VL-linker-VH. Any suitable linker known to those in the art in view of the specification can be used in the CARs. Examples of suitable linkers include, but are not limited to, a GS based linker sequence, and a Whitlow linker GSTSGSGKPGSGEGSTKG (SEQ ID NO: 24). In some embodiments, the linker is a GS or a gly-ser linker. Exemplary gly-ser polypeptide linkers comprise the amino acid sequence Ser (Gly₄Ser)ₙ (SEQ ID NO:173), as well as (Gly₄Ser)ₙ (SEQ ID NO:174) and/or (Gly₄Ser₃)ₙ (SEQ ID NO:175). In some embodiments, n=1. In some embodiments, n=2. In some embodiments, n=3, i.e., Ser (Gly₄Ser)₃ (SEQ ID NO:176). In some embodiments, n=4, i.e., Ser (Gly₄Ser)₄ (SEQ ID NO:177). In some embodiments, n=5. In some embodiments, n=6. In some embodiments, n=7. In some embodiments, n=8. In some embodiments, n=9. In some embodiments, n=10. Another exemplary gly-ser polypeptide linker comprises the amino acid sequence Ser (Gly₄Ser)ₙ (SEQ ID NO:173). In some embodiments, n=1. In some embodiments, n=2. In some embodiments, n=3. In another embodiment, n=4. In some embodiments, n=5. In some embodiments, n=6. Another exemplary gly-ser polypeptide linker comprises (Gly₄Ser)ₙ (SEQ ID NO: 174). In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ (SEQ ID NO: 178). In some embodiments, n=1. In some embodiments, n=2. In some embodiments, n=3. In some embodiments, n=4. In another embodiment, n=5. In yet another embodiment, n=6. Another exemplary gly-ser polypeptide linker comprises (Gly₄Ser₃)ₙ (SEQ ID NO:175). In some embodiments, n=1. In some embodiments, n=2. In some embodiments, n=3. In some embodiments, n=4. In some embodiments, n=5. In some embodiments, n=6. Another exemplary gly-ser polypeptide linker comprises (Gly₃Ser)ₙ (SEQ ID NO:178). In some embodiments, n=1. In some embodiments, n=2. In some embodiments, n=3. In some embodiments, n=4. In another embodiment, n=5. In yet another embodiment, n=6.

In some embodiments, the antigen binding domain is selected from a group that includes an antibody, an antigen-binding portion or fragment thereof, an scFv, and a Fab. In some embodiments, the antigen binding domain binds to CD19, CD20, CD22, BCMA, an EBV antigen, CD27, CD30, EBNA1, EBNA3A, BRLF1, BALF4, EBNA3C, LMP1, LMP2, LMP2A, LMP2B, BZLF1, BMLF1, gp350, or gH/gL. In some embodiments, the antigen binding domain is an anti-CD19 scFv such as but not limited to FMC63. In some embodiments, the antigen binding domain is an anti-CD20 scFv. In some embodiments, the antigen binding domain is an anti-CD22 scFv. In some embodiments, the antigen binding domain is an anti-BCMA scFv. In some embodiments, the antigen binding domain is an anti-EBV antigen scFv. In some embodiments, the antigen binding domain is an anti-CD27 scFv. In some embodiments, the antigen binding domain is an anti-CD30 scFv. In some embodiments, the antigen binding domain is an anti-EBNA1 scFv. In some embodiments, the antigen binding domain is an anti-EBNA3A scFv. In some embodiments, the antigen binding domain is an anti-BRLF1 scFv. In some embodiments, the antigen binding domain is an anti-BALF4 scFv. In some embodiments, the antigen binding domain is an anti-EBNA3C scFv. In some embodiments, the antigen binding domain is an anti-LMP1 scFv. In some embodiments, the antigen binding domain is an anti-LMP2 scFv. In some embodiments, the antigen binding domain is an anti-LMP2A scFv. In some embodiments, the antigen binding domain is an anti-LMP2B scFv. In some embodiments, the antigen binding domain is an anti-BZLF1 scFv. In some embodiments, the antigen binding domain is an anti-BMLF1 scFv. In some embodiments, the antigen binding domain is an anti-gp350 scFv. In some embodiments, the antigen binding domain is an anti-gH/gL scFv.

In some embodiments, the transmembrane domain comprises one selected from a group that includes a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD3ζ, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the signaling domain(s) of the CAR comprises a costimulatory domain(s). For instance, a signaling domain can contain a costimulatory domain. Or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some cases, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR comprises a CD3 zeta (CD3ζ) domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In other embodiments, the CAR comprises (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In certain embodiments, the CAR comprises (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene. In some embodiments, the CAR comprises a (i) an anti-CD19 scFv; (ii) a CD8α hinge and transmembrane domain or functional variant thereof; (iii) a 4-1BB costimulatory domain or functional variant thereof; and (iv) a CD3ζ signaling domain or functional variant thereof.

Methods for introducing a CAR construct or producing a CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus.

In some embodiments, a CD47 transgene is inserted into a pre-selected locus of the cell. In some embodiments, a transgene encoding a CAR is inserted into a pre-selected locus of the cell. In certain embodiments, a CD47 transgene and a transgene encoding a CAR are inserted into a pre-selected locus of the cell. The pre-selected locus can be a safe harbor or a target locus. Non-limiting examples of a safe harbor or target locus include, but are not limited to, a CCR5 gene locus, a PPP1R12C (also known as AAVS1) gene locus, a CLYBL gene locus, and a Rosa gene locus (e.g., ROSA26 gene locus). Non-limiting examples of a target locus include, but are not limited to, a CXCR4 gene locus, an albumin gene locus, a SHS231 gene locus, an F3 gene locus (also known as CD142), a MICA gene locus, a MICB gene locus, a LRP1 gene locus (also known as a CD91 gene locus), a HMGB1 gene locus, an ABO gene locus, a RHD gene locus, a FUT1 locus, and a KDM5D gene locus. The CD47 transgene can be inserted in Introns 1 or 2 for PPP1R12C (i.e., AAVS1) or CCR5. The CD47 transgene can be inserted in Exons 1 or 2 or 3 for CCR5. The CD47 transgene can be inserted in intron 2 for CLYBL. The CD47 transgene can be inserted in a 500 bp window in Ch-4:58,976,613 (i.e., SHS231). The CD47 transgene can be insert in any suitable region of the aforementioned safe harbor or target loci that allows for expression of the exogenous, including, for example, an intron, an exon or a coding sequence region in a safe harbor or target locus. In some embodiments, the pre-selected locus is selected from the group consisting of the B2M locus, the CIITA locus, the TRAC locus, and the TRB locus. In some embodiments, the pre-selected locus is the B2M locus. In some embodiments, the pre-selected locus is the CIITA locus. In some embodiments, the pre-selected locus is the TRAC locus. In some embodiments, the pre-selected locus is the TRB locus.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or target locus. In many instances, a transgene encoding a CAR is inserted into a safe harbor or target locus. In some instances, a CD47 transgene is inserted into a B2M locus. In some instances, a transgene encoding a CAR is inserted into a B2M locus. In certain instances, a CD47 transgene is inserted into a CIITA locus. In certain instances, a transgene encoding a CAR is inserted into a CIITA locus. In particular instances, a CD47 transgene is inserted into a TRAC locus. In particular instances, a transgene encoding a CAR is inserted into a TRAC locus. In many other instances, a CD47 transgene is inserted into a TRB locus. In many other instances, a transgene encoding a CAR is inserted into a TRB locus. In some embodiments, a CD47 transgene and a transgene encoding a CAR are inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In certain embodiments, a CD47 transgene and a transgene encoding a CAR are inserted into a safe harbor or target locus. In certain embodiments, a CD47 transgene and a transgene encoding a CAR are controlled by a single promoter and are inserted into a safe harbor or target locus. In certain embodiments, a CD47 transgene and a transgene encoding a CAR are controlled by their own promoters and are inserted into a safe harbor or target locus. In certain embodiments, a CD47 transgene and a transgene encoding a CAR are inserted into a TRAC locus. In certain embodiments, a CD47 transgene and a transgene encoding a CAR are controlled by a single promoter and are inserted into a TRAC locus. In certain embodiments, a CD47 transgene and a transgene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2M locus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1α promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1α promoter and a transgene encoding a CAR is controlled by an EF1α promoter. In some embodiments, a CD47 transgene is controlled by a CAG promoter and a transgene encoding a CAR is controlled by a CAG promoter. In some embodiments, a CD47 transgene is controlled by a CAG promoter and a transgene encoding a CAR is controlled by an EF1α promoter. In some embodiments, a CD47 transgene is controlled by an EF1α promoter and a transgene encoding a CAR is controlled by a CAG promoter. In some embodiments, expression of both a CD47 transgene and a transgene encoding a CAR is controlled by a single EF1α promoter. In some embodiments, expression of both a CD47 transgene and a transgene encoding a CAR is controlled by a single CAG promoter.

In another embodiment, the present disclosure disclosed herein is directed to pluripotent stem cells, (e.g., pluripotent stem cells and induced pluripotent stem cells (iPSCs)), differentiated cells derived from such pluripotent stem cells (e.g., hypoimmune (HIP) T cells), and primary T cells that overexpress CD47 (such as exogenously express CD47 proteins), have reduced expression or lack expression of one or more MHC class I and/or MHC class II human leukocyte antigen molecules, and have reduced expression or lack expression of a T-cell receptor (TCR) complex. In some embodiments, the hypoimmune (HIP) T cells and primary T cells overexpress CD47 (such as exogenously express CD47 proteins), have reduced expression or lack expression of one or more MHC class I and/or MHC class II human leukocyte antigen molecules, and have reduced expression or lack expression of a T-cell receptor (TCR) complex.

In some embodiments, pluripotent stem cells, (e.g., pluripotent stem cells and induced pluripotent stem cells (iPSCs)), differentiated cells derived from such pluripotent stem cells (e.g., hypoimmune (HIP) T cells), and primary T cells overexpress CD47 and include a genomic modification of the B2M gene. In some embodiments, pluripotent stem cells, differentiated cell derived from such pluripotent stem cells and primary T cells overexpress CD47 and include a genomic modification of the CIITA gene. In some embodiments, pluripotent stem cells, T cells differentiated from such pluripotent stem cells and primary T cells overexpress CD47 and include a genomic modification of the TRAC gene. In some embodiments, pluripotent stem cells, T cells differentiated from such pluripotent stem cells and primary T cells overexpress CD47 and include a genomic modification of the TRB gene. In some embodiments, pluripotent stem cells, T cells differentiated from such pluripotent stem cells and primary T cells overexpress CD47 and include one or more genomic modifications selected from the group consisting of the B2M, CIITA, TRAC and TRB genes. In some embodiments, pluripotent stem cells, T cells differentiated from such pluripotent stem cells and primary T cells overexpress CD47 and include genomic modifications of the B2M, CIITA and TRAC genes. In some embodiments, pluripotent stem cells, T cells differentiated from such pluripotent stem cells and primary T cells overexpress CD47 and include genomic modifications of the B2M, CIITA and TRB genes. In some embodiments, pluripotent stem cells, T cells differentiated from such pluripotent stem cells and primary T cells overexpress CD47 and include genomic modifications of the B2M, CIITA, TRAC and TRB genes. In certain embodiments, the pluripotent stem cells, differentiated cell derived from such pluripotent stem cells and primary T cells are B2M^{-/-}, CIITA^{-/-}, TRAC^{-/-}, CD47tg cells. In certain embodiments, the cells are B2M^{-/-}, CIITA^{-/-}, TRB^{-/-}, CD47tg cells. In certain embodiments, the cells are B2M^{-/-}, CIITA^{-/-}, TRAC^{-/-}, TRB^{-/-}, CD47tg cells. In some embodiments, the cells are B2M^{indel/indel}, CIITA^{indel/indel}, TRAC^{indel/indel}, CD47tg cells. In some embodiments, the cells are B2M^{indel/indel}, CIITA^{indel/indel}, TRB^{indel/indel}, CD47tg cells. In some embodiments, the cells are B2M^{indel/indel}, CIITA^{indel/indel}, TRAC^{indel/indel}, TRB^{indel/indel}, CD47tg cells. In some embodiments, the engineered or modified cells described are pluripotent stem cells, T cells differentiated from such pluripotent stem cells or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

In some embodiments, a CD47 transgene is inserted into a pre-selected locus of the cell. The pre-selected locus can be a safe harbor or target locus. Non-limiting examples of a safe harbor or target locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, the pre-selected locus is the TRAC locus. In some embodiments, a CD47 transgene is inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In certain embodiments, a CD47 transgene is inserted into the B2M locus. In certain embodiments, a CD47 transgene is inserted into the B2M locus. In certain embodiments, a CD47 transgene is inserted into the TRAC locus. In certain embodiments, a CD47 transgene is inserted into the TRB locus.

In some instances, expression of a CD47 transgene is controlled by a constitutive promoter. In other instances, expression of a CD47 transgene is controlled by an inducible promoter. In some embodiments, the promoter is an EF1alpha (EF1α) promoter. In some embodiments, the promoter a CAG promoter.

In yet another embodiment, the present disclosure disclosed herein is directed to pluripotent stem cells, (e.g., pluripotent stem cells and induced pluripotent stem cells (iPSCs)), T cells derived from such pluripotent stem cells (e.g., hypoimmune (HIP) T cells), and primary T cells that have reduced expression or lack expression of one or more MHC class I and/or MHC class II human leukocyte antigen molecules and have reduced expression or lack expression of a T-cell receptor (TCR) complex. In some embodiments, the cells have reduced or lack expression of one or more MHC class I human leukocyte antigen molecules, MHC class II human leukocyte antigen molecules, and TCR complexes.

In some embodiments, pluripotent stem cells (e.g., iPSCs), differentiated cells derived from such (e.g., T cells differentiated from such), and primary T cells include a genomic modification of the B2M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs), T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In certain embodiments, the cells including iPSCs, T cells differentiated from such, and primary T cells are B2M^{-/-}, CIITA^{-/-}, TRAC/cells. In certain embodiments, the cells including iPSCs, T cells differentiated from such, and primary T cells are B2M^{-/-}, CIITA^{-/-}, TRB^{-/-} cells. In some embodiments, the cells including iPSCs, T cells differentiated from such, and primary T cells are B2M^{indel/indel}, CIITA^{indel/indel}, TRAC^{indel/indel} cells. In some embodiments, the cells including iPSCs, T cells differentiated from such, and primary T cells are B2M^{indel/indel}. CIITA^{indel/indel}, TRB^{indel/indel} cells. In some embodiments, the cells including iPSCs, T cells differentiated from such, and primary T cells are B2M^{indel/indel}, CIITA^{indel/indel}, TRAC^{indel/indel} TRB^{indel/indel} cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

Cells of the present disclosure exhibit reduced or lack expression of MHC class I human leukocyte antigen molecules, MHC class II human leukocyte antigen molecules, and/or TCR complexes. Reduction of one or more MHC class I and/or class II HLA molecules expression can be accomplished, for example, by one or more of the following: (1) targeting the polymorphic HLA alleles (HLA-A, HLA-B, HLA-C) and MHC-II genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRFI, NF-Y (including NFY-A, NFY-B, NFY-C), and CIITA that are critical for HLA expression.

In some embodiments, HLA expression is interfered with by targeting individual HLAs (e.g., knocking out, knocking down, or reducing expression of HLA-A, HLA-B, HLA-C, HLA-DP, HLA-DQ, and/or HLA-DR), targeting transcriptional regulators of HLA expression (e.g., knocking out, knocking down, or reducing expression of NLRC5, CIITA, RFX5, RFXAP, RFXANK, NFY-A, NFY-B, NFY-C and/or IRF-1), blocking surface trafficking of MHC class I molecules (e.g., knocking out, knocking down, or reducing expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I molecules and/or MHC-II molecules and are thus characterized as being hypoimmunogenic. For example, in certain embodiments, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In some embodiments, guide RNAs, shRNAs, siRNAs, or miRNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide, siRNA, shRNA, or miRNA molecule that targets a conserved region in HLAs can act as an HLA Razor.

Methods provided are useful for inactivation or ablation of MHC class I molecule expression and/or MHC class II molecule expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I molecule and MHC II molecule expression. In some embodiments, the cells are nonimmunogenic (e.g., do not induce an innate and/or an adaptive immune response) in a recipient subject.

In some embodiments, the cell includes a modification to increase expression of CD47 and one or more factors selected from the group consisting of DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, A20/TNFAIP3, CD39, CR1, HLA-F, IL15-RF, MANF, and/or Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M, CIITA and NLRC5 genes. In certain embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is otherwise modified or engineered to have altered expression of one or more genes to generate the engineered cell.

In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of one or more MHC I molecules and/or MHC II molecules (including one or more MHC class I and/or class II HLA molecules) is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens/molecules, (b) MHC II antigens/molecules, (c) TCR complexes, (d) both MHC I and II antigens/molecules, and (e) MHC I and II antigens and TCR complexes. In certain embodiments, the modification includes increasing expression of CD47. In some embodiments, the cells include an exogenous or recombinant CD47 polypeptide. In certain embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of one or more MHC I antigens/molecules, MHC II antigens/molecules and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In certain embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen, HLA-DQ antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In certain embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of one or more MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, HLA-DP, HLA-DQ, HLA-DR, B2M, CIITA, TCR-alpha, and TCR-beta relative to a wild-type stem cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Also provided herein are hypoimmunogenic primary T cells including any subtype of primary T cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR), wherein the first and/or second exogenous polynucleotides are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered eis protected from cell lysis by NK cells, including immature and/or mature NK cells upon administration to a recipient patient. In some embodiments, the population of engineered cells evades macrophage engulfment upon administration to a recipient patient. In some embodiments, the population of engineered cells does not induce an innate and/or an adaptive immune response to the cell upon administration to a recipient patient.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a safety switch.

In some embodiments, the safety switch comprises a therapeutic agent that inhibits or blocks the interaction of CD47 and SIRPα. In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the cells described herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a suicide gene.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### B. CIITA

In some embodiments, the technologies disclosed herein modulate (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II molecules are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### C. B2M

In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS: 81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene.

Assays to test whether the B2M gene has been inactivated are known and described herein. In some embodiments, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### D. NLRC5

In many embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules: HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS: 36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

### E. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS: 532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

### F. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. P0DSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. P0DSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS: 610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

### G. CD142

In many embodiments, the technologies disclosed herein modulate (e.g., reduce or eliminate) the expression of CD142, which is also known as tissue factor, factor III, and F3. In some embodiments, the modulation occurs using a gene editing system (e.g. CRISPR/Cas).

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In some embodiments, the resulting genetic modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, reverse transcriptase polymerase chain reactions (RT-PCR) are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### H. CTLA-4

In some embodiments, the target polynucleotide sequence is CTLA-4 or a variant of CTLA-4. In some embodiments, the target polynucleotide sequence is a homolog of CTLA-4. In some embodiments, the target polynucleotide sequence is an ortholog of CTLA-4.

In some embodiments, the cells outlined herein comprise a genetic modification targeting the CTLA-4 gene. In certain embodiments, primary T cells comprise a genetic modification targeting the CTLA-4 gene. The genetic modification can reduce expression of CTLA-4 polynucleotides and CTLA-4 polypeptides in T cells includes primary T cells and CAR-T cells. In some embodiments, the genetic modification targeting the CTLA-4 gene by the rare-cutting endonuclease comprises a Cas protein or a polynucleotide encoding a Cas protein, and at least one guide ribonucleic acid (gRNA) sequence for specifically targeting the CTLA-4 gene. Useful methods for identifying gRNA sequences to target CTLA-4 are described below.

Assays to test whether the CTLA-4 gene has been inactivated are known and described herein. In some embodiments, the resulting genetic modification of the CTLA-4 gene by PCR and the reduction of CTLA-4 expression can be assays by FACS analysis. In another embodiment, CTLA-4 protein expression is detected using a Western blot of cells lysates probed with antibodies to the CTLA-4 protein. In another embodiment, reverse transcriptase polymerase chain reactions (RT-PCR) are used to confirm the presence of the inactivating genetic modification.

Useful genomic, polynucleotide and polypeptide information about the human CTLA-4 are provided in, for example, the GeneCard Identifier GC02P203867, HGNC No. 2505, NCBI Gene ID 1493, NCBI RefSeq Nos. NM_005214.4, NM_001037631.2, NP_001032720.1 and NP_005205.2, UniProt No. P16410, and the like.

### I. PD-1

In some embodiments, the target polynucleotide sequence is PD-1 or a variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In certain embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression can be assays by FACS analysis. In another embodiment, PD-1 protein expression is detected using a Western blot of cells lysates probed with antibodies to the PD-1 protein. In another embodiment, reverse transcriptase polymerase chain reactions (RT-PCR) are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

### J. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of an CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 1.

TABLE 1
Amino acid sequences of human CD47

| | SEQ | | Amino |
|---|---|---|---|
| | ID | | acid |
| Protein | NO: | Sequence | residues |
| Human | 13 | QLLFNKTKSVEFTFCNDTVV | aa 19-323 |
| CD47 | | IPCFVTNMEAQNTTEVYVKW | |
| (without | | KFKGRDIYTFDGALNKSTVP | |
| signal | | TDFSSAKIEVSQLLKGDASL | |
| sequence) | | KMDKSDAVSHTGNYTCEVTE | |
| | | LTREGETIIELKYRVVSWFS | |
| | | PNENILIVIFPIFAILLFWG | |
| | | QFGIKTLKYRSGGMDEKTIA | |
| | | LLVAGLVITVIVIVGAILFV | |
| | | PGEYSLKNATGLGLIVTSTG | |
| | | ILILLHYYVFSTAIGLTSFV | |
| | | IAILVIQVIAYILAVVGLSL | |
| | | CIAACIPMHGPLLISGLSIL | |
| | | ALAQLLGLVYMKFVASNQKT | |
| | | IQPPRKAVEEPLNAFKESKG | |
| | | MMNDE | |
| Human | 14 | MWPLVAALLLGSACCGSAQL | aa 1-323 |
| CD47 (with | | LFNKTKSVEFTFCNDTVVIP | |
| signal | | CFVTNMEAQNTTEVYVKWKF | |
| sequence) | | KGRDIYTFDGALNKSTVPTD | |
| | | FSSAKIEVSQLLKGDASLKM | |
| | | DKSDAVSHTGNYTCEVTELT | |
| | | REGETIIELKYRVVSWFSPN | |
| | | ENILIVIFPIFAILLFWGQF | |
| | | GIKTLKYRSGGMDEKTIALL | |
| | | VAGLVITVIVIVGAILFVPG | |
| | | EYSLKNATGLGLIVTSTGIL | |
| | | ILLHYYVFSTAIGLTSFVIA | |
| | | ILVIQVIAYILAVVGLSLCI | |
| | | AACIPMHGPLLISGLSILAL | |
| | | AQLLGLVYMKFVASNQKTIQ | |
| | | PPRKAVEEPLNAFKESKGMM | |
| | | NDE | |

In some embodiments, the cell comprises a CD47 polypeptide having at least 95% sequence identity (e.g., 95%, 96%, 97%, 98%, 99%, or more) to the amino acid sequence of SEQ ID NO: 13. In some embodiments, the cell comprises a CD47 polypeptide having the amino acid sequence of SEQ ID NO:13. In some embodiments, the cell comprises a CD47 polypeptide having at least 95% sequence identity (e.g., 95%, 96%, 97%, 98%, 99%, or more) to the amino acid sequence of SEQ ID NO:14. In some embodiments, the cell comprises a CD47 polypeptide having the amino acid sequence of SEQ ID NO:14.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:13. In some embodiments, the cell comprises a nucleotide sequence encoding a CD47 polypeptide having the amino acid sequence of SEQ ID NO: 13. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:14. In some embodiments, the cell comprises a nucleotide sequence encoding a CD47 polypeptide having the amino acid sequence of SEQ ID NO: 14. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some cases, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is inserted into any one of the gene loci depicted in Table 30 provided herein. In certain embodiments, the polynucleotide encoding CD47 is operably linked to a promoter.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### K. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 30 provided herein. In certain embodiments, the polynucleotide encoding CD24 is operably linked to a promoter.

In another embodiment, CD24 protein expression is detected using a Western blot of cells lysates probed with antibodies against the CD24 protein. In another embodiment, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some cases, the polynucleotide encoding CD24 is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is inserted into any one of the gene loci depicted in Table 30 provided herein. In certain embodiments, the polynucleotide encoding CD24 is operably linked to a promoter.

### L. DUX4

In some embodiments, the present disclosure provides a cell (e.g., stem cell, induced pluripotent stem cell, differentiated cell, hematopoietic stem cell, primary T cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4. In some embodiments, the disclosure provides a cell or population thereof comprising exogenously expressed DUX4 proteins. In some embodiments, increased expression of DUX4 suppresses, reduces or eliminates expression of one or more of the following MHC I molecules: HLA-A, HLA-B, and HLA-C.

DUX4 is a transcription factor that is active in embryonic tissues and induced pluripotent stem cells, and is silent in normal, healthy somatic tissues (Feng et al., 2015, ELife4; De Iaco et al., 2017, Nat Genet, 49, 941-945; Hendrickson et al., 2017, Nat Genet, 49, 925-934; Snider et al., 2010, PLOS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLOS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In many embodiments, at least one or more polynucleotides may be utilized to facilitate the exogenous expression of DUX4 by a cell, e.g., a stem cell, induced pluripotent stem cell, differentiated cell, hematopoietic stem cell, primary T cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some cases, the polynucleotide encoding DUX4 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is inserted into any one of the gene loci depicted in Table 30 provided herein. In certain embodiments, the polynucleotide encoding DUX4 is operably linked to a promoter.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO: 3, SEQ ID NO: 4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO:12, SEQ ID NO: 13, SEQ ID NO:14, SEQ ID NO:15, SEQ ID NO: 16, SEQ ID NO:17, SEQ ID NO:18, SEQ ID NO: 19, SEQ ID NO:20, SEQ ID NO:21, SEQ ID NO: 22, SEQ ID NO: 23, SEQ ID NO: 24, SEQ ID NO:25, SEQ ID NO: 26, SEQ ID NO:27, SEQ ID NO:28, and SEQ ID NO: 29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO: 7, SEQ ID NO:8, SEQ ID NO:9, SEQ ID NO: 10, SEQ ID NO:11, SEQ ID NO: 12, SEQ ID NO: 13, SEQ ID NO:14, SEQ ID NO:15, SEQ ID NO:16, SEQ ID NO:17, SEQ ID NO:18, SEQ ID NO: 19, SEQ ID NO: 20, SEQ ID NO: 21, SEQ ID NO: 22, SEQ ID NO: 23, SEQ ID NO: 24, SEQ ID NO:25, SEQ ID NO:26, SEQ ID NO:27, SEQ ID NO:28, and SEQ ID NO:29. Amino acid sequences set forth as SEQ ID NOS: 2-29 are shown in FIG. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. P0CJ85.1 or an amino acid sequence set forth in UniProt No. P0CJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. P0CJ87.1 or an amino acid sequence of set forth in UniProt No. P0CJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some cases, the codon altered sequence of DUX4 comprises SEQ ID NO:1 of PCT/US2020/44635. In some cases, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO: 7, SEQ ID NO: 8, SEQ ID NO:9, SEQ ID NO: 10, SEQ ID NO: 11, SEQ ID NO:12, SEQ ID NO: 13, SEQ ID NO:14, SEQ ID NO:15, SEQ ID NO: 16, SEQ ID NO:17, SEQ ID NO:18, SEQ ID NO: 19, SEQ ID NO: 20, SEQ ID NO: 21, SEQ ID NO: 22, SEQ ID NO: 23, SEQ ID NO:24, SEQ ID NO:25, SEQ ID NO: 26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO:15, SEQ ID NO:16, SEQ ID NO:17, SEQ ID NO:18, SEQ ID NO:19, SEQ ID NO: 20, SEQ ID NO: 21, SEQ ID NO: 22, SEQ ID NO:23, SEQ ID NO:24, SEQ ID NO: 25, SEQ ID NO:26, SEQ ID NO:27, SEQ ID NO:28, and SEQ ID NO:29 of PCT/US2020/44635.

An increase of DUX4 expression can be assayed using known techniques, such as Western blots, ELISA assays, FACS assays, immunoassays, and the like.

### M. CD7

In many embodiments, the technologies disclosed herein modulate (e.g., reduce or eliminate) the expression of CD7. In some embodiments, the modulation occurs using a CRISPR/Cas system. CD7 is transmembrane protein and is a member of the immunoglobulin superfamily. CD7 is found on thymocytes and mature T cells. CD7 plays a role in T-cell interactions and also in T-cell/B-cell interaction during early lymphoid development.

In some embodiments, the target polynucleotide sequence encodes a variant of CD7. In some embodiments, the target polynucleotide sequence encodes a homolog of CD7. In some embodiments, the target polynucleotide sequence encodes an ortholog of CD7.

In some embodiments, the cells outlined herein comprise a genetic modification targeting the CD7 gene. In some embodiments, the genetic modification targeting the CD7 gene by the rare-cutting endonuclease comprises a Cas protein or a polynucleotide encoding a Cas protein, and at least one guide ribonucleic acid sequence for specifically targeting the CD7 gene.

Assays to test whether the CD7 gene has been inactivated are known and described herein. In some embodiments, the resulting genetic modification of the CD7 gene can be assayed by PCR and the reduction of CD7 expression can be assayed by FACS analysis. In another embodiment, CD7 protein expression is detected using a Western blot of cell lysates probed with antibodies that bind to the CD7 protein. In another embodiment, reverse transcriptase polymerase chain reactions (RT-PCR) are used to confirm the presence of the inactivating genetic modification.

### N. CD52

In many embodiments, the technologies disclosed herein modulate (e.g., reduce or eliminate) the expression of CD52 (also known as CAMPATH-1). In some embodiments, the modulation occurs using a CRISPR/Cas system. Typically, CD52 is present on the surface of mature lymphocytes, but not on the stem cells from which these lymphocytes are derived. CD52 is also found on monocytes and dendritic cells. CD52 is associated with cancer, and in particular, certain types of lymphoma. A reduction or elimination of CD52 can lead to a reduction or depletion of B cells and/or T cells.

In some embodiments, the target polynucleotide sequence encodes a variant of CD52. In some embodiments, the target polynucleotide sequence encodes a homolog of CD52. In some embodiments, the target polynucleotide sequence encodes an ortholog of CD52.

In some embodiments, the cells outlined herein comprise a genetic modification targeting the CD52 gene. In some embodiments, the genetic modification targeting the CD52 gene by the rare-cutting endonuclease comprises a Cas protein or a polynucleotide encoding a Cas protein, and at least one guide ribonucleic acid sequence for specifically targeting the CD52 gene.

Assays to test whether the CD52 gene has been inactivated are known and described herein. In some embodiments, the resulting genetic modification of the CD52 gene can be assayed by PCR and the reduction of CD52 expression can be assayed by FACS analysis. In another embodiment, CD52 protein expression is detected using a Western blot of cells lysates probed with antibodies that bind to the CD52 protein. In another embodiment, reverse transcriptase polymerase chain reactions (RT-PCR) are used to confirm the presence of the inactivating genetic modification.

### O. CD70

In many embodiments, the technologies disclosed herein modulate (e.g., reduce or eliminate) the expression of CD70. In some embodiments, the modulation occurs using a CRISPR/Cas system. CD70 is a member of the TNF family and its expression is upregulated on dendritic cells upon maturation. Its receptor, CD27, is expressed on T cells and NK cells. The CD70/CD27 pathway promotes effector CD8⁺ T cells responses by sustaining survival of cytotoxic T cells and influences polarization of CD4⁺ T cells. Overexpression of CD70 has been associated with spontaneous activation of T cells, leading to fatal immunopathologies.

In some embodiments, the target polynucleotide sequence encodes a variant of CD70. In some embodiments, the target polynucleotide sequence encodes a homolog of CD70. In some embodiments, the target polynucleotide sequence encodes an ortholog of CD70.

In some embodiments, the cells outlined herein comprise a genetic modification targeting the CD70 gene. In some embodiments, the genetic modification targeting the CD70 gene by the rare-cutting endonuclease comprises a Cas protein or a polynucleotide encoding a Cas protein, and at least one guide ribonucleic acid sequence for specifically targeting the CD70 gene.

Assays to test whether the CD70 gene has been inactivated are known and described herein. In some embodiments, the resulting genetic modification of the CD70 gene can be assayed by PCR and the reduction of CD70 expression can be assayed by FACS analysis. In another embodiment, CD70 protein expression is detected using a Western blot of cells lysates probed with antibodies that bind to the CD70 protein. In another embodiment, reverse transcriptase polymerase chain reactions (RT-PCR) are used to confirm the presence of the inactivating genetic modification.

### P. BCL11A

In many embodiments, the technologies disclosed herein modulate (e.g., reduce or eliminate) the expression of Transcription factor B-cell lymphoma/leukemia 11A (BCL11A). In some embodiments, the modulation occurs using a CRISPR/Cas system. BCL11A is a zinc-finger protein that is predominantly expressed in brain and hematopoietic tissue. BCL11A functions mainly as a transcriptional repressor that can be crucial in brain, hematopoietic system development, as well as fetal-to-adult hemoglobin switching. Studies indicate that BCL11A is involved in, e.g., β-hemoglobinopathies, hematological malignancies, malignant solid tumors, 2p15-p16.1 microdeletion syndrome, and Type II diabetes.

In some embodiments, the target polynucleotide sequence encodes a variant of BCL11A. In some embodiments, the target polynucleotide sequence encodes a homolog of BCL11A. In some embodiments, the target polynucleotide sequence encodes an ortholog of BCL11A.

In some embodiments, the cells outlined herein comprise a genetic modification targeting the BCL11A gene. In some embodiments, the genetic modification targeting the BCL11A gene by the rare-cutting endonuclease comprises a Cas protein or a polynucleotide encoding a Cas protein, and at least one guide ribonucleic acid sequence for specifically targeting the BCL11A gene.

Assays to test whether the BCL11A gene has been inactivated are known and described herein. In some embodiments, the resulting genetic modification of the BCL11A gene can be assayed by PCR. In another embodiment, BCL11A protein expression is detected using a Western blot of cell lysates probed with antibodies that bind to the BCL11A protein. In another embodiment, reverse transcriptase polymerase chain reactions (RT-PCR) are used to confirm the presence of the inactivating genetic modification.

### Q. Tolerogenic Factors

In many embodiments, one or more tolerogenic factors can be inserted or reinserted into genome-edited cells to create immune-privileged universal donor cells, such as universal donor stem cells, universal donor T cells, or universal donor cells. In certain embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, and Serpinb9, A20/TNFAIP3, CD39, CR1, HLA-F, IL15-RF, and MANF. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, Serpinb9, A20/TNFAIP3, CD39, CR1, HLA-F, IL15-RF, and MANF.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3.

Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, and, RNA or protein expression technologies and the like. For all of these technologies, well known recombinant techniques are used, to generate recombinant nucleic acids as outlined herein.

In some embodiments, the cells (e.g., stem cell, induced pluripotent stem cell, differentiated cell, hematopoietic stem cell, primary T cell or CAR-T cell) possess genetic modifications that inactivate the B2M and CIITA genes and express a plurality of exogenous polypeptides selected from the group including CD47 and DUX4, CD47 and CD24, CD47 and CD27, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, and CD47 Serpinb9, CD47 and A20/TNFAIP3, CD47 and CD39, CD47 and CR1, CD47 and HLA-F, CD47 and IL15-RF, and CD47 and MANF, and any combination thereof. In some instances, such cells also possess a genetic modification that inactivates the CD142 gene.

In some instances, a gene editing system such as the CRISPR/Cas system is used to facilitate the insertion of tolerogenic factors, such as the tolerogenic factors into a safe harbor or target locus, such as the AAVS1 locus, to actively inhibit immune rejection. In some instances, the tolerogenic factors are inserted into a safe harbor or target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, expression of a target gene (e.g., DUX4, CD47, or another tolerogenic factor gene) is increased by expression of fusion protein or a protein complex containing (1) a site-specific binding domain specific for the endogenous target gene (e.g., DUX4, CD47, or another tolerogenic factor gene) and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (-1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31 (7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; Mckenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, CI, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rttl09 (Bemdsen and Denu (2008) Curr Opin Struct Biol 18 (6): 682-689). In other instances the domain is a histone deacetylase (HD AC) such as the class I (HDAC-I, 2, 3, and 8), class II (HDAC IIA (HDAC-4, 5, 7 and 9), HD AC IIB (HDAC 6 and 10)), class IV (HDAC-I 1), class III (also known as sirtuins (SIRTs); SIRT1-7) (see Mottamal et al., (2015) Molecules 20 (3): 3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In certain embodiments, at least one ribonucleic acid or at least one pair of ribonucleic acids may be utilized to facilitate the insertion of CD47 into a cell line. In certain embodiments, the at least one ribonucleic acid or the at least one pair of ribonucleic acids is selected from the group consisting of SEQ ID NOS: 200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In certain embodiments, at least one ribonucleic acid or at least one pair of ribonucleic acids may be utilized to facilitate the insertion of HLA-C into a cell line. In certain embodiments, the at least one ribonucleic acid or the at least one pair of ribonucleic acids is selected from the group consisting of SEQ ID NOS: 3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In certain embodiments, at least one ribonucleic acid or at least one pair of ribonucleic acids may be utilized to facilitate the insertion of HLA-E into a cell line. In certain embodiments, the at least one ribonucleic acid or the at least one pair of ribonucleic acids is selected from the group consisting of SEQ ID NOS: 189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In certain embodiments, at least one ribonucleic acid or at least one pair of ribonucleic acids may be utilized to facilitate the insertion of HLA-F into a cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In certain embodiments, at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS: 188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In certain embodiments, at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS: 193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In certain embodiments, at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express Cl-inhibitor. In some embodiments, the present disclosure provides a method for altering a cell genome to express Cl-inhibitor. In certain embodiments, at least one ribonucleic acid or at least one pair of ribonucleic acids may be utilized to facilitate the insertion of Cl-inhibitor into a stem cell line. In certain embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In certain embodiments, at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing CD47 in a cell comprises a polynucleotide sequence encoding CD47. The expression vector can be an inducible expression vector. The expression vector can be a viral vector, such as but not limited to, a lentiviral vector. In some embodiments, the tolerogenic factors are introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain embodiments, at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosure is incorporated herein by references.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some cases, the polynucleotide encoding the tolerogenic factor is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into a B2M gene locus, a CIITA gene locus, a TRAC gene locus, or a TRB gene locus. In some embodiments, the polynucleotide encoding the tolerogenic factor is inserted into any one of the gene loci depicted in Table 30 provided herein. In certain embodiments, the polynucleotide encoding the tolerogenic factor is operably linked to a promoter.

In some embodiments, the cells are engineered to expresses an increased amount of one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, CD16, CD52, H2-M3, CD16 Fc receptor, IL15-RF, Serpinb9, A20/TNFAIP3, CD39, CR1, HLA-F, IL15-RF, and/or MANF relative to a cell of the same cell type that does not comprise the modifications.

### R. Chimeric Antigen Receptors

Provided herein are hypoimmunogenic cells comprising a chimeric antigen receptor (CAR). In some embodiments, the CAR binds to CD19. In some embodiments, the CAR binds to CD22. In some embodiments, the CAR binds to CD20. In some embodiments, the CAR binds to BCMA. In some embodiments, the CAR binds to an EBV antigen. In some embodiments, the CAR binds to CD27. In some embodiments, the CAR binds to CD30. In some embodiments, the CAR binds to CD19 and CD20. In some embodiments, the CAR binds to CD19 and CD22. In some embodiments, the CAR binds to CD19 and CD27. In some embodiments, the CAR binds to EBNA1. In some embodiments, the CAR binds to EBNA3A. In some embodiments, the CAR binds to BRLF1. In some embodiments, the CAR binds to BALF4. In some embodiments, the CAR binds to EBNA3C. In some embodiments, the CAR binds to LMP1. In some embodiments, the CAR binds to LMP2. In some embodiments, the CAR binds to LMP2A. In some embodiments, the CAR binds to LMP2B. In some embodiments, the CAR binds to BZLF1. In some embodiments, the CAR binds to BMLF1. In some embodiments, the CAR binds to gp350. In some embodiments, the CAR binds to gH/gL. In some embodiments, the CAR binds to EBNA1 and LMP1. In some embodiments, the CAR binds to EBNA1 and LMP2A. In some embodiments, the CAR binds to EBNA1, LMP1 and LMP2A. In some embodiments, the CAR binds to LMP, BARF1 and EBNA1. In some embodiments, the CAR binds to CD19 and an EBV antigen. In some embodiments, the CAR binds to CD20 and an EBV antigen. In some embodiments, the CAR binds to CD22 and an EBV antigen. In some embodiments, the CAR is selected from the group consisting of a first generation CAR, a second generation CAR, a third generation CAR, and a fourth generation CAR. In some embodiments, the CAR includes a single binding domain that binds to a single target antigen. In some embodiments, the CAR includes a single binding domain that binds to more than one target antigen, e.g., 2, 3, or more target antigens. In some embodiments, the CAR includes two binding domains such that each binding domain binds to a different target antigens. In some embodiments, the CAR includes two binding domains such that each binding domain binds to the same target antigen. Detailed descriptions of exemplary CARs including CD19-specific, CD20-specific and CD19/CD20-bispecific CARs can be found in WO2012/079000, WO2016/149578 and WO2020/014482, the disclosures including the sequence listings and figures are incorporated herein by reference in their entirety. In some embodiments, the CAR includes two binding domains such that each binding domain binds to the same target antigen. Detailed descriptions of exemplary CARs including CD19-specific, CD22-specific and CD19/CD22-bispecific CARs can be found in WO2012/079000, WO2016/149578 and WO2020/014482, the disclosures including the sequence listings and figures are incorporated herein by reference in their entirety. Detailed descriptions of exemplary CARs, TCRs or scFvs including CD27-specific, CD30-specific, EBNA1-specific, EBNA3C-specific, LMP1-specific, LMP2-specific, LMP2A-specific, gp350-specific CARs, gH/gL-specific CARs can be found in EP 2 520 589, U.S. Pat. Nos. 9,403,914, 11,180,566, US 2021/0009706, EP 2 558 498 B1, WO 2021/222929, US 2021/10206863, WO 2015/199617A1, WO 2012/109659A1, U.S. Pat. No. 7,786,269B2, WO 2021/211455A1, US 2016/0199479, WO2019/201995A1, and U.S. Pat. No. 11,116,835, the disclosures including the sequence listings and figures are incorporated herein by reference in their entireties. In some embodiments, the CAR includes two binding domains such that each binding domain binds to the same target antigen.

In some embodiments, the CD19 specific CAR includes an anti-CD19 single-chain antibody fragment (scFv), a transmembrane domain such as one derived from human CD8α, a 4-1BB (CD137) co-stimulatory signaling domain, and a CD3ζ signaling domain. In some embodiments, the CD20 specific CAR includes an anti-CD20 scFv, a transmembrane domain such as one derived from human CD8α, a 4-1BB (CD137) co-stimulatory signaling domain, and a CD3ζ signaling domain. In some embodiments, the CD19/CD20-bispecific CAR includes an anti-CD19 scFv, an anti-CD20 scFv, a transmembrane domain such as one derived from human CD8α, a 4-1BB (CD137) co-stimulatory signaling domain, and a CD3ζ signaling domain. In some embodiments, the CD22 specific CAR includes an anti-CD22 scFv, a transmembrane domain such as one derived from human CD8α, a 4-1BB (CD137) co-stimulatory signaling domain, and a CD3ζ signaling domain. In some embodiments, the CD19/CD22-bispecific CAR includes an anti-CD19 scFv, an anti-CD22 scFv, a transmembrane domain such as one derived from human CD8α, a 4-1BB (CD137) co-stimulatory signaling domain, and a CD3ζ signaling domain. In some embodiments, the EBNA1 specific CAR includes an anti-EBNA1 scFv, a transmembrane domain such as one derived from human CD8α, a 4-1BB (CD137) co-stimulatory signaling domain, and a CD3ζ signaling domain. In some embodiments, the EBNA3A CAR includes an anti-EBNA3A scFv, a transmembrane domain such as one derived from human CD8α, a 4-1BB (CD137) co-stimulatory signaling domain, and a CD3ζ signaling domain. In some embodiments, the EBNA3C CAR includes an anti-EBNA3C scFv, a transmembrane domain such as one derived from human CD8α, a 4-1BB (CD137) co-stimulatory signaling domain, and a CD3ζ signaling domain. In some embodiments, the LMP1 specific CAR includes an anti-LMP1 scFv, a transmembrane domain such as one derived from human CD8α, a 4-1BB (CD137) co-stimulatory signaling domain, and a CD3ζ signaling domain. In some embodiments, the LMP2 specific CAR includes an anti-LMP2 scFv, a transmembrane domain such as one derived from human CD8α, a 4-1BB (CD137) co-stimulatory signaling domain, and a CD3ζ signaling domain. In some embodiments, the LMP2A CAR includes an anti-LMP2A scFv, a transmembrane domain such as one derived from human CD8α, a 4-1BB (CD137) co-stimulatory signaling domain, and a CD3ζ signaling domain. In some embodiments, the BZLF1 CAR includes an anti-BZLF1 scFv, a transmembrane domain such as one derived from human CD8α, a 4-1BB (CD137) co-stimulatory signaling domain, and a CD37 signaling domain. In some embodiments, the BMLF1 CAR includes an anti-BMLF1 scFv, a transmembrane domain such as one derived from human CD8α, a 4-1BB (CD137) co-stimulatory signaling domain, and a CD3ζ signaling domain. In some embodiments, the gp350 CAR includes an anti-gp350 scFv, a transmembrane domain such as one derived from human CD8α, a 4-1BB (CD137) co-stimulatory signaling domain, and a CD3ζ signaling domain. In some embodiments, the gH/gL specific CAR includes an anti-gH/gL scFv, a transmembrane domain such as one derived from human CD8α, a 4-1BB (CD137) co-stimulatory signaling domain, and a CD37 signaling domain.

In some embodiments, the CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL119 from Novartis, P-BMCA-101 from Poseida Therapeutics, PBCAR19B and PBCAR269A from Precision Biosciences, FT819 from Fate Therapeutics, and CYAD-211 from Clyad Oncology.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

1. ABD Targets an Antigen Characteristic of an Autoimmune Diseases/Disorders and/or Inflammatory Diseases/Disorders

In some embodiments, the antigen binding domain targets an antigen characteristic of an autoimmune diseases/disorders and/or inflammatory diseases/disorders, including those described in Section Z below. In some embodiments, the ABD binds an antigen associated with an autoimmune or inflammatory disorder. In some instances, the antigen is expressed by a cell associated with an autoimmune diseases/disorders and/or inflammatory diseases/disorders. In some embodiments, the autoimmune or inflammatory disorder is selected from chronic graft-vs-host disease (GVHD), lupus, arthritis, immune complex glomerulonephritis, goodpasture syndrome, uveitis, hepatitis, systemic sclerosis or scleroderma, type I diabetes, multiple sclerosis, EBV infection, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cryoglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15 (4): 931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, or a virus-derived antigen, such as a nuclear antigen or an envelope protein.

In some embodiments, an antigen binding domain of a CAR binds to a ligand expressed on B cells, plasma cells, or plasmablasts. In some embodiments, an antigen binding domain of a CAR binds to CD19, CD20, CD22, or BCMA, CD27, CD30, EBNA1, LMP1, LMP2, LMP2A, EBNA3A, EBNA3C, BZLF1, BMLF1, gp350, or gH/gL. See, e.g., US 2003/0077249; WO 2017/058753; WO 2017/058850; US 2021/0230245; WO 2019/201995A1; EP 3 781 590 A1, the contents of which are herein incorporated by reference.

In some embodiments, autoimmune or inflammatory disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis (such as plaque psoriasis, gutatte psoriasis, pustular psoriasis, and psoriasis of the nails), atopy (including atopic diseases such as hay fever and Job's syndrome), dermatitis (including contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis), x-linked hyper IgM syndrome, allergic intraocular inflammatory diseases, urticaria (such as chronic allergic urticaria, chronic idiopathic urticaria, chronic autoimmune urticaria), myositis, polymyositis/dermatomyositis, juvenile dermatomyositis, toxic epidermal necrolysis, scleroderma (including systemic scleroderma), sclerosis (such as systemic sclerosis; multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, and ataxic sclerosis), neuromyelitis optica spectrum disorder (NMO, also known as Devic's Disease or Devic's Syndrome), inflammatory bowel disease (IBD) including Crohn's disease; autoimmune-mediated gastrointestinal diseases; colitis such as ulcerative colitis, colitis ulcerosa, microscopic colitis, collagenous colitis, colitis polyposa, necrotizing enterocolitis, and transmural colitis; and autoimmune inflammatory bowel disease), bowel inflammation, pyoderma gangrenosum, erythema nodosum, primary sclerosing cholangitis, respiratory distress syndrome (including adult or acute respiratory distress syndrome (ARDS)), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis (such as anterior uveitis, acute anterior uveitis, granulomatous uveitis, nongranulomatous uveitis, phacoantigenic uveitis, posterior uveitis, or autoimmune uveitis), glomerulonephritis (GN) with and without nephrotic syndrome (such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, or proliferative nephritis), autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema (including allergic or atopic eczema, asteatotic eczema, dyshidrotic eczema, and vesicular palmoplantar eczema), asthma (such as asthma bronchiale, bronchial asthma, and auto-immune asthma), conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus (including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE or subacute cutaneous SLE, neonatal lupus syndrome (NLE), and lupus erythematosus disseminatus, Type I diabetes, Type II diabetes, and latent autoimmune diabetes in adults (or Type 1.5 diabetes), juvenile onset (Type I) diabetes mellitus, including pediatric insulin-dependent diabetes mellitus (IDDM), adult onset diabetes mellitus (Type II diabetes), idiopathic diabetes, insipidus, diabetic retinopathy, diabetic nephropathy, and diabetic large-artery disorder; immune responses associated with acute and delayed hypersensitivity mediated by cytokines and T-lymphocytes; tuberculosis, sarcoidosis, granulomatosis (including lymphomatoid granulomatosis, Wegener's granulomatosis, or agranulocytosis), vasculitides (including vasculitis, large-vessel vasculitis, polymyalgia rheumatica and giant cell (Takayasu's) arteritis, medium-vessel vasculitis, Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis (such as Churg-Strauss vasculitis or syndrome (CSS) and ANCA-associated small-vessel vasculitis)), temporal arteritis, aplastic anemia, autoimmune aplastic anemia, Coombs positive anemia, Diamond Blackfan anemia, hemolytic anemia, immune hemolytic anemia including autoimmune hemolytic anemia (AIHA), pernicious anemia (anemia perniciosa), Addison's disease, pure red cell anemia or aplasia (PRCA); Factor VIII deficiency; hemophilia A; autoimmune neutropenia, pancytopenia, leukopenia, diseases involving leukocyte diapedesis, CNS inflammatory disorders, Alzheimer's disease, Parkinson's disease, multiple organ injury syndrome (such as those secondary to septicemia, trauma, or hemorrhage), antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP), post-transfusion purpura (PTP), heparin-induced thrombocytopenia, autoimmune or immune-mediated thrombocytopenia such as idiopathic thrombocytopenia purpura (ITP) including chronic or acute ITP, acquired thrombocytopenia purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases, including thyroiditis autoimmune thyroiditis, Hashimoto's disease, chronic thyroiditis (Hashimoto's thyroiditis), or subacute thyroiditis), autoimmune thyroid disease, idiopathic hypothyroidism, or Grave's disease), polyglandular syndromes, autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, hepatitis, including autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility (e.g., due to anti-spermatozoan antibodies) mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, endometriosis, and pulmonary hemosiderosis.

### 2. ABD Binds to a Cell Surface or Intracellular Antigen of a Cell

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell. In some embodiments, an antigen binding domain (ABD) binds to an intracellular antigen. In some embodiments, an intracellular antigen is characteristic of a cell infected by a pathogen. In some embodiments, an intracellular antigen is characteristic of a cell infected by a virus. In some embodiments, an intracellular antigen is characteristic of a cell infected by EBV. In some embodiments, an intracellular antigen is characteristic of a B cell infected with a pathogen. In some embodiments, an intracellular antigen is characteristic of a B cell infected with a virus. In some embodiments, an intracellular antigen is characteristic of a B cell infected with EBV. In some embodiments, an intracellular pathogen is processed endogenously by infected cells and presented to T cells through MHC class I molecules.

In some embodiments, a CAR antigen binding domain binds a cell surface antigen characteristic of a T cell, such as a cell surface antigen on a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, a CAR antigen binding domain binds a cell surface antigen characteristic of a B cell, such as a cell surface antigen on a B cell. In some embodiments, an antigen characteristic of a B cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a B cell. In some embodiments, an antigen characteristic of a B cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

### 3. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### 4. Signaling Domain or Plurality of Signaling Domains

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thyl; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

5. Domain which Upon Successful Signaling of the CAR Induces Expression of a Cytokine Gene

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, IL-4, IL-10, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and/or CAR-T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR-T cell proliferation, and or CAR-T cell persistence during T cell activation.

### 6. ABD Comprising an Antibody or Antigen-Binding Portion Thereof.

In some embodiments, a CAR antigen binding domain is or comprises an antibody or antigen-binding portion thereof. In some embodiments, a CAR antigen binding domain is or comprises an scFv or Fab. In some embodiments, a CAR antigen binding domain comprises an scFv or Fab fragment of a CD19 antibody; CD22 antibody; a CD20 antibody; a CD27 antibody; a CD30 antibody; a BRLF1 antibody; a BALF4 antibody; a EBNA1 antibody; a EBNA3A antibody; a EBNA3C antibody; a LMP1 antibody; a LMP2 antibody; a LMP2A antibody; a BZLF1 antibody; a BMLF1 antibody; a gp350 antibody; or a gH/gL antibody.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

### 7. Additional Descriptions of CARS

In certain embodiments, the cell may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimalization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ ID | | |
|---|---|---|
| NO: | Sequence | Description |
| 6 | MALPVTALLLPLA | CD8α signal peptide |
| | LLLHAARP | |
| 7 | METDTLLLWVLLL | IgK signal peptide |
| | WVPGSTG | |
| 8 | MLLLVTSLLLCEL | GMCSFR-α (CSF2RA) |
| | PHPAFLLIP | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G4S)ₙ (SEQ ID NO: 179) (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD19, CD20, CD22, and B cell maturation agent (BCMA). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ | | |
|---|---|---|
| ID | | |
| NO: | Sequence | Description |
| 9 | TTTPAPRPPTPAPTIASQPLSL | CD8α hinge |
| | RPEACRPAAGGAVHTRGLDFAC | domain |
| | D | |
| 10 | IEVMYPPPYLDNEKSNGTIIHV | CD28 hinge |
| | KGKHLCPSPLFPGPSKP | domain |
| 113 | AAAIEVMYPPPYLDNEKSNGTI | CD28 hinge |
| | IHVKGKHLCPSPLFPGPSKP | domain |
| 11 | ESKYGPPCPPCP | IgG4 hinge |
| | | domain |
| 12 | ESKYGPPCPSCP | IgG4 hinge |
| | | domain |
| 180 | ESKYGPPCPPCPAPEFLGGPSV | IgG4 hinge- |
| | FLFPPKPKDTLMISRTPEVTCV | CH2-CH3 |
| | VVDVSQEDPEVQFNWYVDGVEV | domain |
| | HNAKTKPREEQFNSTYRVVSVL | |
| | TVLHQDWLNGKEYKCKVSNKGL | |
| | PSSIEKTISKAKGQPREPQVYT | |
| | LPPSQEEMTKNQVSLTCLVKGF | |
| | YPSDIAVEWESNGQPENNYKTT | |
| | PPVLDSDGSFFLYSRLTVDKSR | |
| | WQEGNVFSCSVMHEALHNHYTQ | |
| | KSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of transmembrane domains

| SEQ ID NO: | Sequence | Description |
|---|---|---|
| 181 | IYIWAPLAGTCGVL | CD8α transmembrane |
| | LLSLVITLYC | domain |
| 15 | FWVLVVVGGVLACY | CD28 transmembrane |
| | SLLVTVAFIIFWV | domain |
| 114 | MFWVLVVVGGVLAC | CD28 transmembrane |
| | YSLLVTVAFIIFWV | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO: 115).

TABLE 5
Exemplary sequences of intracellular
costimulatory and/or signaling domains

| SEQ | | |
|---|---|---|
| ID | | |
| NO: | Sequence | Description |
| 16 | KRGRKKLLYIFKQPFMRPVQTTQEEDG | 4-1BB costimulatory domain |
| | CSCRFPEEEEGGCEL | |
| 17 | RSKRSRLLHSDYMNMTPRRPGPTRKHY | CD28 costimulatory domain |
| | QPYAPPRDFAAYRS | |
| 18 | RVKFSRSADAPAYQQGQNQLYNELNLG | CD3ζ signaling domain |
| | RREEYDVLDKRRGRDPEMGGKPRRKN | |
| | PQEGLYNELQKDKMAEAYSEIGMKGER | |
| | RRGKGHDGLYQGLSTATKDTYDALHM | |
| | QALPPR | |
| 115 | RVKFSRSADAPAYKQGQNQLYNELNLG | CD35 signaling domain (with |
| | RREEYDVLDKRRGRDPEMGGKPRRKN | Q to K mutation at |
| | PQEGLYNELQKDKMAEAYSEIGMKGER | position 14) |
| | RRGKGHDGLYQGLSTATKDTYDALHM | |
| | QALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO:19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, the CD19-specific scFv may comprise one or more CDRs having amino acid sequences set forth in SEQ ID NOs: 21-23 and 26-28. In some embodiments, the CD19-specific scFv may comprise a light chain with one or more CDRs having amino acid sequences set forth in SEQ ID NOs: 21-23. In some embodiments, the CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:24. In some embodiments, the Whitlow linker may be replaced by a different linker, for example, a 3xG₄S (SEQ ID NO: 30) linker having an amino acid sequence set forth in SEQ ID NO:30, which gives rise to a different FMC63-derived scFv having an amino acid sequence set forth in SEQ ID NO: 29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 29.

TABLE 6
Exemplary sequences of anti-CD19 scFv and components

| SEQ ID NO: | Amino Acid Sequence | Description |
|---|---|---|
| 19 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISNL | Whitlow linker |
| | EQEDIATYFCQQGNTLPYTFGGGTK | |
| | LEITGSTSGSGKPGSGEGSTKGEVKL | |
| | QESGPGLVAPSQSLSVTCTVSGVSLP | |
| | DYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 20 | DIQMTQTTSSLSASLGDRVTISCRAS | Anti-CD19 FMC63 scFv |
| | QDISKYLNWYQQKPDGTVKLLIYHT | light chain variable |
| | SRLHSGVPSRFSGSGSGTDYSLTISNL | region |
| | EQEDIATYFCQQGNTLPYTFGGGTK | |
| | LEIT | |
| 21 | QDISKY | Anti-CD19 FMC63 scFv |
| | | light chain CDR1 |
| 22 | HTS | Anti-CD19 FMC63 scFv |
| | | light chain CDR2 |
| 23 | QQGNTLPYT | Anti-CD19 FMC63 scFv |
| | | light chain CDR3 |
| 24 | GSTSGSGKPGSGEGSTKG | Whitlow linker |
| 25 | EVKLQESGPGLVAPSQSLSVTCTVSG | Anti-CD19 FMC63 scFv |
| | VSLPDYGVSWIRQPPRKGLEWLGVI | heavy chain variable |
| | WGSETTYYNSALKSRLTIIKDNSKSQ | region |
| | VFLKMNSLQTDDTAIYYCAKHYYY | |
| | GGSYAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISNL | 3xG₄S (SEQ ID NO: 30) |
| | EQEDIATYFCQQGNTLPYTFGGGTK | linker |
| | LEITGGGGSGGGGSGGGGSEVKLQE | |
| | SGPGLVAPSQSLSVTCTVSGVSLPDY | |
| | GVSWIRQPPRKGLEWLGVIWGSETT | |
| | YYNSALKSRLTIIKDNSKSQVFLKM | |
| | NSLQTDDTAIYYCAKHYYYGGSYA | |
| | MDYWGQGTSVTVSS | |
| 30 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD37 signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO:29, the CD28 hinge domain of SEQ ID NO:10, the CD28 transmembrane domain of SEQ ID NO:15, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:116 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:116 (see Table 7). The encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:117, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3xG₄S (SEQ ID NO: 30) linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 7, with annotations of the sequences provided in Table 8.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 32, 34, or 36, respectively.

TABLE 7
Exemplary sequences of CD19 CARs

| SEQ ID NO: | Sequence | Description |
|---|---|---|
| 116 | atggccttaccagtgaccgccttgctcctgccgctggccttgctgct | Exemplary CD19 |
| | ccacgccgccaggccggacatccagatgacacagactacatcctc | CAR nucleotide |
| | cctgtctgcctctctgggagacagagtcaccatcagttgcagggca | sequence |
| | agtcaggacattagtaaatatttaaattggtatcagcagaaaccagat | |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggctccacctctggatccggcaagcccggatctg | |
| | gcgagggatccaccaagggcgaggtgaaactgcaggagtcagg | |
| | acctggcctggtggcgccctcacagagcctgtccgtcacatgcact | |
| | gtctcaggggtctcattacccgactatggtgtaagctggattcgcca | |
| | gcctccacgaaagggtctggagtggctgggagtaatatggggtag | |
| | tgaaaccacatactataattcagctctcaaatccagactgaccatcat | |
| | caaggacaactccaagagccaagttttcttaaaaatgaacagtctgc | |
| | aaactgatgacacagccatttactactgtgccaaacattattactacg | |
| | gtggtagctatgctatggactactggggccaaggaacctcagtcac | |
| | cgtctcctcaaccacgacgccagcgccgcgaccaccaacaccgg | |
| | cgcccaccatcgcgtcgcagcccctgtccctgcgcccagaggcgt | |
| | gccggccagcggcggggggcgcagtgcacacgagggggctgg | |
| | acttcgcctgtgatatctacatctgggcgcccttggccgggacttgt | |
| | ggggtccttctcctgtcactggttatcaccctttactgcaaacggggc | |
| | agaaagaaactcctgtatatattcaaacaaccatttatgagaccagta | |
| | caaactactcaagaggaagatggctgtagctgccgatttccagaag | |
| | aagaagaaggaggatgtgaactgagagtgaagttcagcaggagc | |
| | gcagacgcccccgcgtaccagcagggccagaaccagctctataa | |
| | cgagctcaatctaggacgaagagaggagtacgatgttttggacaa | |
| | gagacgtggccgggaccctgagatggggggaaagccgagaag | |
| | gaagaaccctcaggaaggcctgtacaatgaactgcagaaagataa | |
| | gatggcggaggcctacagtgagattgggatgaaaggcgagcgcc | |
| | ggaggggcaaggggcacgatggcctttaccagggtctcagtaca | |
| | gccaccaaggacacctacgacgcccttcacatgcaggccctgccc | |
| | cctcgc | |
| 117 | MALPVTALLLPLALLLHAARPDIQMTQTTS | Exemplary CD19 |
| | SLSASLGDRVTISCRASQDISKYLNWYQQK | CAR amino acid |
| | PDGTVKLLIYHTSRLHSGVPSRFSGSGSGT | sequence |
| | DYSLTISNLEQEDIATYFCQQGNTLPYTFGG | |
| | GTKLEITGSTSGSGKPGSGEGSTKGEVKLQ | |
| | ESGPGLVAPSQSLSVTCTVSGVSLPDYGVS | |
| | WIRQPPRKGLEWLGVIWGSETTYYNSALK | |
| | SRLTIIKDNSKSQVFLKMNSLQTDDTAIYYC | |
| | AKHYYYGGSYAMDYWGQGTSVTVSSTTT | |
| | PAPRPPTPAPTIASQPLSLRPEACRPAAGGA | |
| | VHTRGLDFACDIYIWAPLAGTCGVLLLSLVI | |
| | TLYCKRGRKKLLYIFKQPFMRPVQTTQEED | |
| | GCSCRFPEEEEGGCELRVKFSRSADAPAYQ | |
| | QGQNQLYNELNLGRREEYDVLDKRRGRDP | |
| | EMGGKPRRKNPQEGLYNELQKDKMAEAY | |
| | SEIGMKGERRRGKGHDGLYQGLSTATKDT | |
| | YDALHMQALPPR | |
| 31 | atggccttaccagtgaccgccttgctcctgccgctggccttgctgct | Tisagenlecleucel |
| | ccacgccgccaggccggacatccagatgacacagactacatcctc | CD19 CAR |
| | cctgtctgcctctctgggagacagagtcaccatcagttgcagggca | nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggtggcggtggctcgggcggtggtgggtcgggt | |
| | ggcggcggatctgaggtgaaactgcaggagtcaggacctggcct | |
| | ggtggcgccctcacagagcctgtccgtcacatgcactgtctcagg | |
| | ggtctcattacccgactatggtgtaagctggattcgccagcctccac | |
| | gaaagggtctggagtggctgggagtaatatggggtagtgaaacca | |
| | catactataattcagctctcaaatccagactgaccatcatcaaggac | |
| | aactccaagagccaagttttcttaaaaatgaacagtctgcaaactga | |
| | tgacacagccatttactactgtgccaaacattattactacggtggtag | |
| | ctatgctatggactactggggccaaggaacctcagtcaccgtctcct | |
| | caaccacgacgccagcgccgcgaccaccaacaccggcgcccac | |
| | catcgcgtcgcagcccctgtccctgcgcccagaggcgtgccggc | |
| | cagcggcggggggcgcagtgcacacgagggggctggacttcgc | |
| | ctgtgatatctacatctgggcgcccttggccgggacttgtggggtcc | |
| | ttctcctgtcactggttatcaccctttactgcaaacggggcagaaag | |
| | aaactcctgtatatattcaaacaaccatttatgagaccagtacaaact | |
| | actcaagaggaagatggctgtagctgccgatttccagaagaagaa | |
| | gaaggaggatgtgaactgagagtgaagttcagcaggagcgcaga | |
| | cgcccccgcgtacaagcagggccagaaccagctctataacgagc | |
| | tcaatctaggacgaagagaggagtacgatgttttggacaagagac | |
| | gtggccgggaccctgagatggggggaaagccgagaaggaaga | |
| | accctcaggaaggcctgtacaatgaactgcagaaagataagatgg | |
| | cggaggcctacagtgagattgggatgaaaggcgagcgccggag | |
| | gggcaaggggcacgatggcctttaccagggtctcagtacagccac | |
| | caaggacacctacgacgcccttcacatgcaggccctgccccctcg | |
| | c | |
| 32 | MALPVTALLLPLALLLHAARPDIQMTQTTS | Tisagenlecleucel |
| | SLSASLGDRVTISCRASQDISKYLNWYQQK | CD19 CAR amino |
| | PDGTVKLLIYHTSRLHSGVPSRFSGSGSGT | acid sequence |
| | DYSLTISNLEQEDIATYFCQQGNTLPYTFGG | |
| | GTKLEITGGGGSGGGGSGGGGSEVKLQES | |
| | GPGLVAPSQSLSVTCTVSGVSLPDYGVSWI | |
| | RQPPRKGLEWLGVIWGSETTYYNSALKSR | |
| | LTIIKDNSKSQVFLKMNSLQTDDTAIYYCA | |
| | KHYYYGGSYAMDYWGQGTSVTVSSTTTP | |
| | APRPPTPAPTIASQPLSLRPEACRPAAGGAV | |
| | HTRGLDFACDIYIWAPLAGTCGVLLLSLVIT | |
| | LYCKRGRKKLLYIFKQPFMRPVQTTQEEDG | |
| | CSCRFPEEEEGGCELRVKFSRSADAPAYKQ | |
| | GQNQLYNELNLGRREEYDVLDKRRGRDPE | |
| | MGGKPRRKNPQEGLYNELQKDKMAEAYS | |
| | EIGMKGERRRGKGHDGLYQGLSTATKDTY | |
| | DALHMQALPPR | |
| 33 | atgctgctgctggtgaccagcctgctgctgtgcgagctgccccacc | Lisocabtagene |
| | ccgcctttctgctgatccccgacatccagatgacccagaccacctc | maraleucel CD19 |
| | cagcctgagcgccagcctgggcgaccgggtgaccatcagctgcc | CAR nucleotide |
| | gggccagccaggacatcagcaagtacctgaactggtatcagcag | sequence |
| | aagcccgacggcaccgtcaagctgctgatctaccacaccagccg | |
| | gctgcacagcggcgtgcccagccggtttagcggcagcggctccg | |
| | gcaccgactacagcctgaccatctccaacctggaacaggaagata | |
| | tcgccacctacttttgccagcagggcaacacactgccctacaccttt | |
| | ggcggcggaacaaagctggaaatcaccggcagcacctccggca | |
| | gcggcaagcctggcagcggcgagggcagcaccaagggcgagg | |
| | tgaagctgcaggaaagcggccctggcctggtggcccccagccag | |
| | agcctgagcgtgacctgcaccgtgagcggcgtgagcctgcccga | |
| | ctacggcgtgagctggatccggcagccccccaggaagggcctgg | |
| | aatggctgggcgtgatctggggcagcgagaccacctactacaaca | |
| | gcgccctgaagagccggctgaccatcatcaaggacaacagcaag | |
| | agccaggtgttcctgaagatgaacagcctgcagaccgacgacacc | |
| | gccatctactactgcgccaagcactactactacggcggcagctacg | |
| | ccatggactactggggccagggcaccagcgtgaccgtgagcagc | |
| | gaatctaagtacggaccgccctgccccccttgccctatgttctgggt | |
| | gctggtggtggtcggaggcgtgctggcctgctacagcctgctggt | |
| | caccgtggccttcatcatcttttgggtgaaacggggcagaaagaaa | |
| | ctcctgtatatattcaaacaaccatttatgagaccagtacaaactactc | |
| | aagaggaagatggctgtagctgccgatttccagaagaagaagaag | |
| | gaggatgtgaactgcgggtgaagttcagcagaagcgccgacgcc | |
| | cctgcctaccagcagggccagaatcagctgtacaacgagctgaac | |
| | ctgggcagaagggaagagtacgacgtcctggataagcggagag | |
| | gccgggaccctgagatgggcggcaagcctcggcggaagaaccc | |
| | ccaggaaggcctgtataacgaactgcagaaagacaagatggccg | |
| | aggcctacagcgagatcggcatgaagggcgagcggaggcggg | |
| | gcaagggccacgacggcctgtatcagggcctgtccaccgccacc | |
| | aaggatacctacgacgccctgcacatgcaggccctgcccccaag | |
| | g | |
| 34 | MLLLVTSLLLCELPHPAFLLIPDIQMTQTTS | Lisocabtagene |
| | SLSASLGDRVTISCRASQDISKYLNWYQQK | maraleucel CD19 |
| | PDGTVKLLIYHTSRLHSGVPSRFSGSGSGT | CAR amino acid |
| | DYSLTISNLEQEDIATYFCQQGNTLPYTFGG | sequence |
| | GTKLEITGSTSGSGKPGSGEGSTKGEVKLQ | |
| | ESGPGLVAPSQSLSVTCTVSGVSLPDYGVS | |
| | WIRQPPRKGLEWLGVIWGSETTYYNSALK | |
| | SRLTIIKDNSKSQVFLKMNSLQTDDTAIYYC | |
| | AKHYYYGGSYAMDYWGQGTSVTVSSESK | |
| | YGPPCPPCPMFWVLVVVGGVLACYSLLVT | |
| | VAFIIFWVKRGRKKLLYIFKQPFMRPVQTT | |
| | QEEDGCSCRFPEEEEGGCELRVKFSRSADA | |
| | PAYQQGQNQLYNELNLGRREEYDVLDKRR | |
| | GRDPEMGGKPRRKNPQEGLYNELQKDKM | |
| | AEAYSEIGMKGERRRGKGHDGLYQGLSTA | |
| | TKDTYDALHMQALPPR | |
| 35 | atgcttctcctggtgacaagccttctgctctgtgagttaccacaccca | Axicabtagene |
| | gcattcctcctgatcccagacatccagatgacacagactacatcctc | ciloleucel CD19 |
| | cctgtctgcctctctgggagacagagtcaccatcagttgcagggca | CAR nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggactaagtt | |
| | ggaaataacaggctccacctctggatccggcaagcccggatctgg | |
| | cgagggatccaccaagggcgaggtgaaactgcaggagtcagga | |
| | cctggcctggtggcgccctcacagagcctgtccgtcacatgcactg | |
| | tctcaggggtctcattacccgactatggtgtaagctggattcgccag | |
| | cctccacgaaagggtctggagtggctgggagtaatatggggtagt | |
| | gaaaccacatactataattcagctctcaaatccagactgaccatcatc | |
| | aaggacaactccaagagccaagttttcttaaaaatgaacagtctgca | |
| | aactgatgacacagccatttactactgtgccaaacattattactacgg | |
| | tggtagctatgctatggactactggggtcaaggaacctcagtcacc | |
| | gtctcctcagcggccgcaattgaagttatgtatcctcctccttaccta | |
| | gacaatgagaagagcaatggaaccattatccatgtgaaagggaaa | |
| | cacctttgtccaagtcccctatttcccggaccttctaagcccttttggg | |
| | tgctggtggtggttgggggagtcctggcttgctatagcttgctagta | |
| | acagtggcctttattattttctgggtgaggagtaagaggagcaggct | |
| | cctgcacagtgactacatgaacatgactccccgccgccccgggcc | |
| | cacccgcaagcattaccagccctatgccccaccacgcgacttcgc | |
| | agcctatcgctccagagtgaagttcagcaggagcgcagacgccc | |
| | ccgcgtaccagcagggccagaaccagctctataacgagctcaatc | |
| | taggacgaagagaggagtacgatgttttggacaagagacgtggcc | |
| | gggaccctgagatggggggaaagccgagaaggaagaaccctca | |
| | ggaaggcctgtacaatgaactgcagaaagataagatggcggagg | |
| | cctacagtgagattgggatgaaaggcgagcgccggaggggcaa | |
| | ggggcacgatggcctttaccagggtctcagtacagccaccaagga | |
| | cacctacgacgcccttcacatgcaggccctgccccctcgc | |
| 36 | MLLLVTSLLLCELPHPAFLLIPDIQMTQTTS | Axicabtagene |
| | SLSASLGDRVTISCRASQDISKYLNWYQQK | ciloleucel CD19 |
| | PDGTVKLLIYHTSRLHSGVPSRFSGSGSGT | CAR amino acid |
| | DYSLTISNLEQEDIATYFCQQGNTLPYTFGG | sequence |
| | GTKLEITGSTSGSGKPGSGEGSTKGEVKLQ | |
| | ESGPGLVAPSQSLSVTCTVSGVSLPDYGVS | |
| | WIRQPPRKGLEWLGVIWGSETTYYNSALK | |
| | SRLTIIKDNSKSQVFLKMNSLQTDDTAIYYC | |
| | AKHYYYGGSYAMDYWGQGTSVTVSSAAA | |
| | IEVMYPPPYLDNEKSNGTIIHVKGKHLCPSP | |
| | LFPGPSKPFWVLVVVGGVLACYSLLVTVAF | |
| | IIFWVRSKRSRLLHSDYMNMTPRRPGPTRK | |
| | HYQPYAPPRDFAAYRSRVKFSRSADAPAYQ | |
| | QGQNQLYNELNLGRREEYDVLDKRRGRDP | |
| | EMGGKPRRKNPQEGLYNELQKDKMAEAY | |
| | SEIGMKGERRRGKGHDGLYQGLSTATKDT | |
| | YDALHMQALPPR | |

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S(SEQ ID NO: 30) | | |
| linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 32, 34, or 36, respectively.

### CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In some embodiments, the CD20 CAR is derived from a CAR specific to CD20, including, for example, MB-106, UCART20, or C-CAR066, as detailed in Table 11A. In any of these embodiments, the extracellular binding domain of the CD20 CAR can comprise or consist of the V_{H}, the V_{L}, and/or one or more CDRs of any of the antibodies or CARS detailed in Table 11A.

TABLE 11A
Exemplary CD20 -specific CARs

| CAR | | | |
|---|---|---|---|
| Name | Antigen | Company | Reference |
| MB-106 | CD20 | Fred Hutchinson | Shadman et al., Blood |
| | | Cancer | 134 (Suppl._1): 3235 |
| | | Research Center | (2019) |
| UCART20 | CD20 | Cellectis | www.cellbiomedgroup.com |
| C-CAR066 | CD20 | Cellular | Liang et al., J Clin |
| | | Biomedicine | Oncol |
| | | Group | 39(15)_suppl: 2508-2508 |
| | | | (2021) |

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14 (12): 1025-1033 (2001). In some embodiments, the linker is a 3xG₄S (SEQ ID NO: 30) linker. In other embodiments, the linker is a Whitlow linker as described herein. In some embodiments, the amino acid sequences of different portions of the entire Leu16-derived scFv (also referred to as Leu16 scFv) and its different portions are provided in Table 11B below. In some embodiments, the CD20-specific scFv comprises or consists of an amino acid sequence set forth in SEQ ID NO:37, 38, or 42, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37, 38, or 42. In some embodiments, the CD20-specific scFv may comprise one or more CDRs having amino acid sequences set forth in SEQ ID NOs: 39-41, 43 and 44. In some embodiments, the CD20-specific scFv may comprise a light chain with one or more CDRs having amino acid sequences set forth in SEQ ID NOs: 39-41. In some embodiments, the CD20-specific scFv may comprise a heavy chain with one or more CDRs having amino acid sequences set forth in SEQ ID NOs: 43-44. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11B
Exemplary sequences of anti-CD20 scFv and components

| SEQ ID NO: | Amino Acid Sequence | Description |
|---|---|---|
| 37 | DIVLTQSPAILSASPGEKVTMTCRAS | Anti-CD20 Leu16 scFv |
| | SSVNYMDWYQKKPGSSPKPWIYATS | entire sequence, with |
| | NLASGVPARFSGSGSGTSYSLTISRVE | Whitlow linker |
| | AEDAATYYCQQWSFNPPTFGGGTK | |
| | LEIKGSTSGSGKPGSGEGSTKGEVQL | |
| | QQSGAELVKPGASVKMSCKASGYT | |
| | FTSYNMHWVKQTPGQGLEWIGAIY | |
| | PGNGDTSYNQKFKGKATLTADKSSS | |
| | TAYMQLSSLTSEDSADYYCARSNYY | |
| | GSSYWFFDVWGAGTTVTVSS | |
| 38 | DIVLTQSPAILSASPGEKVTMTCRAS | Anti-CD20 Leu16 scFv |
| | SSVNYMDWYQKKPGSSPKPWIYATS | light chain variable |
| | NLASGVPARFSGSGSGTSYSLTISRVE | region |
| | AEDAATYYCQQWSFNPPTFGGGTK | |
| | LEIK | |
| 39 | RASSSVNYMD | Anti-CD20 Leu16 scFv |
| | | light chain CDR1 |
| 40 | ATSNLAS | Anti-CD20 Leu16 scFv |
| | | light chain CDR2 |
| 41 | QQWSFNPPT | Anti-CD20 Leu16 scFv |
| | | light chain CDR3 |
| 42 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTAD | |
| | KSSSTAYMQLSSLTSEDSADYYCARS | |
| | NYYGSSYWFFDVWGAGTTVTVSS | |
| 43 | SYNMH | Anti-CD20 Leu16 scFv |
| | | heavy chain CDR1 |
| 44 | AIYPGNGDTSYNQKFKG | Anti-CD20 Leu16 scFv |
| | | heavy chain CDR2 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the CD28 hinge domain of SEQ ID NO: 10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:37, the IgG4 hinge domain of SEQ ID NO:11 or SEQ ID NO:1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3xG₄S (SEQ ID NO: 30) linker. In other embodiments, the Whitlow linker may be used instead. In some embodiments, the amino acid sequences of the entire m971-derived scFv (also referred to as m971 scFv) and its different portions are provided in Table 12 below. In some embodiments, the CD22-specific scFv comprises or consists of an amino acid sequence set forth in SEQ ID NO:45, 46, or 50, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:45, 46, or 50. In some embodiments, the CD22-specific scFv may comprise one or more CDRs having amino acid sequences set forth in SEQ ID NOs: 47-49 and 51-53. In some embodiments, the CD22-specific scFv may comprise a heavy chain with one or more CDRs having amino acid sequences set forth in SEQ ID NOs: 47-49. In some embodiments, the CD22-specific scFv may comprise a light chain with one or more CDRs having amino acid sequences set forth in SEQ ID NOs: 51-53. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3xG₄S (SEQ ID NO: 30) linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:54, 55, or 59, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:54, 55, or 59. In some embodiments, the CD22-specific scFv may comprise one or more CDRs having amino acid sequences set forth in SEQ ID NOs: 56-58 and 60-62. In some embodiments, the CD22-specific scFv may comprise a heavy chain with one or more CDRs having amino acid sequences set forth in SEQ ID NOs: 56-58. In some embodiments, the CD22-specific scFv may comprise a light chain with one or more CDRs having amino acid sequences set forth in SEQ ID NOs: 60-62. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22 scFv and components

| SEQ ID NO: | Amino Acid Sequence | Description |
|---|---|---|
| 45 | QVQLQQSGPGLVKPSQTLSLTCAISG | Anti-CD22 m971 scFv |
| | DSVSSNSAAWNWIRQSPSRGLEWLG | entire sequence, with |
| | RTYYRSKWYNDYAVSVKSRITINPD | 3xG4S(SEQ ID NO: 30) |
| | TSKNQFSLQLNSVTPEDTAVYYCAR | linker |
| | EVTGDLEDAFDIWGQGTMVTVSSG | |
| | GGGSGGGGSGGGGSDIQMTQSPSSL | |
| | SASVGDRVTITCRASQTIWSYLNWY | |
| | QQRPGKAPNLLIYAASSLQSGVPSRF | |
| | SGRGSGTDFTLTISSLQAEDFATYYC | |
| | QQSYSIPQTFGQGTKLEIK | |
| 46 | QVQLQQSGPGLVKPSQTLSLTCAISG | Anti-CD22 m971 scFv |
| | DSVSSNSAAWNWIRQSPSRGLEWLG | heavy chain variable |
| | RTYYRSKWYNDYAVSVKSRITINPD | region |
| | TSKNQFSLQLNSVTPEDTAVYYCAR | |
| | EVTGDLEDAFDIWGQGTMVTVSS | |
| 47 | GDSVSSNSAA | Anti-CD22 m971 scFv |
| | | heavy chain CDR1 |
| 48 | TYYRSKWYN | Anti-CD22 m971 scFv |
| | | heavy chain CDR2 |
| 49 | AREVTGDLEDAFDI | Anti-CD22 m971 scFv |
| | | heavy chain CDR3 |
| 50 | DIQMTQSPSSLSASVGDRVTITCRAS | Anti-CD22 m971 scFv |
| | QTIWSYLNWYQQRPGKAPNLLIYAA | light chain |
| | SSLQSGVPSRFSGRGSGTDFTLTISSL | |
| | QAEDFATYYCQQSYSIPQTFGQGTK | |
| | LEIK | |
| 51 | QTIWSY | Anti-CD22 m971 scFv |
| | | light chain CDR1 |
| 52 | AAS | Anti-CD22 m971 scFv |
| | | light chain CDR2 |
| 53 | QQSYSIPQT | Anti-CD22 m971 scFv |
| | | light chain CDR3 |
| 54 | QVQLQQSGPGMVKPSQTLSLTCAIS | Anti-CD22 m971-L7 |
| | GDSVSSNSVAWNWIRQSPSRGLEWL | scFv entire sequence, |
| | GRTYYRSTWYNDYAVSMKSRITINP | with 3xG4S (SEQ ID |
| | DTNKNQFSLQLNSVTPEDTAVYYCA | NO: 30) linker |
| | REVTGDLEDAFDIWGQGTMVTVSS | |
| | GGGGSGGGGSGGGGSDIQMIQSPSS | |
| | LSASVGDRVTITCRASQTIWSYLNW | |
| | YRQRPGEAPNLLIYAASSLQSGVPSR | |
| | FSGRGSGTDFTLTISSLQAEDFATYY | |
| | CQQSYSIPQTFGQGTKLEIK | |
| 55 | QVQLQQSGPGMVKPSQTLSLTCAIS | Anti-CD22 m971-L7 |
| | GDSVSSNSVAWNWIRQSPSRGLEWL | scFv heavy chain |
| | GRTYYRSTWYNDYAVSMKSRITINP | variable region |
| | DTNKNQFSLQLNSVTPEDTAVYYCA | |
| | REVTGDLEDAFDIWGQGTMVTVSS | |
| 56 | GDSVSSNSVA | Anti-CD22 m971-L7 |
| | | scFv heavy chain CDR1 |
| 57 | TYYRSTWYN | Anti-CD22 m971-L7 |
| | | scFv heavy chain CDR2 |
| 58 | AREVTGDLEDAFDI | Anti-CD22 m971-L7 |
| | | scFv heavy chain CDR3 |
| 59 | DIQMIQSPSSLSASVGDRVTITCRAS | Anti-CD22 m971-L7 |
| | QTIWSYLNWYRQRPGEAPNLLIYAA | scFv light chain variable |
| | SSLQSGVPSRFSGRGSGTDFTLTISSL | region |
| | QAEDFATYYCQQSYSIPQTFGQGTK | |
| | LEIK | |
| 60 | QTIWSY | Anti-CD22 m971-L7 |
| | | scFv light chain CDR1 |
| 61 | AAS | Anti-CD22 m971-L7 |
| | | scFv light chain CDR2 |
| 62 | QQSYSIPQT | Anti-CD22 m971-L7 |
| | | scFv light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280 (1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:45 or SEQ ID NO:54, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of sBCMA has been shown to be significantly increased in rheumatoid arthritis (RA) and systemic lupus erythematosus (SLE) patients and correlates with disease activity (Salazar-Camarena, et la., Scientific Reports, 10:6236 (2020)). In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19 (8): 2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 13 below. In some embodiments, the BCMA-specific extracellular binding domain comprises or consists of an amino acid sequence set forth in SEQ ID NO:63, 64, or 68, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:63, 64, or 68. In some embodiments, the BCMA-specific extracellular binding domain may comprise one or more CDRs having amino acid sequences set forth in SEQ ID NOs: 65-67 and 69-71. In some embodiments, the BCMA-specific extracellular binding domain may comprise a light chain with one or more CDRs having amino acid sequences set forth in SEQ ID NOs: 65-67. In some embodiments, the BCMA-specific extracellular binding domain may comprise a heavy chain with one or more CDRs having amino acid sequences set forth in SEQ ID NOs: 69-71. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19 (8): 2048-2060 (2013) and PCT Application Publication No. WO2010/104949, the amino acid sequence of which is also provided in Table 13 below. In some embodiments, the BCMA-specific extracellular binding domain comprises or consists of an amino acid sequence set forth in SEQ ID NO:72, 73, or 77, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:72, 73, or 77. In some embodiments, the BCMA-specific extracellular binding domain may comprise one or more CDRs having amino acid sequences set forth in SEQ ID NOs: 74-76 and 78-80. In some embodiments, the BCMA-specific extracellular binding domain may comprise a light chain with one or more CDRs having amino acid sequences set forth in SEQ ID NOs: 74-76. In some embodiments, the BCMA-specific extracellular binding domain may comprise a heavy chain with one or more CDRs having amino acid sequences set forth in SEQ ID NOs: 78-80. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29 (5): 585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11 (1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11 (1): 283 (2020), also referred to as FHVH33. See also, PCT Application Publication No. WO2019/006072. The amino acid sequences of FHVH33 and its CDRs are provided in Table 13 below. In some embodiments, the BCMA-specific extracellular binding domain comprises or consists of an amino acid sequence set forth in SEQ ID NO:81 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:81. In some embodiments, the BCMA-specific extracellular binding domain may comprise one or more CDRs having amino acid sequences set forth in SEQ ID NOs: 82-84. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO:118, 119, or 123, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 118, 119, or 123. In some embodiments, the BCMA-specific extracellular binding domain may comprise one or more CDRs having amino acid sequences set forth in SEQ ID NOs: 120-122 and 124-126. In some embodiments, the BCMA-specific extracellular binding domain may comprise a light chain with one or more CDRs having amino acid sequences set forth in SEQ ID NOs: 120-122. In some embodiments, the BCMA-specific extracellular binding domain may comprise a heavy chain with one or more CDRs having amino acid sequences set forth in SEQ ID NOs: 124-126. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA binder and components

| SEQ ID NO: | Amino Acid Sequence | Description |
|---|---|---|
| 63 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGGG | |
| | TKLEIKGSTSGSGKPGSGEGSTKGQI | |
| | QLVQSGPELKKPGETVKISCKASGY | |
| | TFTDYSINWVKRAPGKGLKWMGWI | |
| | NTETREPAYAYDFRGRFAFSLETSAS | |
| | TAYLQINNLKYEDTATYFCALDYSY | |
| | AMDYWGQGTSVTVSS | |
| 64 | DIVLTQSPASLAMSLGKRATISCRAS | Anti-BCMA C11D5.3 |
| | ESVSVIGAHLIHWYQQKPGQPPKLLI | scFv light chain variable |
| | YLASNLETGVPARFSGSGSGTDFTLT | region |
| | IDPVEEDDVAIYSCLQSRIFPRTFGGG | |
| | TKLEIK | |
| 65 | RASESVSVIGAHLIH | Anti-BCMA C11D5.3 |
| | | scFv light chain CDR1 |
| 66 | LASNLET | Anti-BCMA C11D5.3 |
| | | scFv light chain CDR2 |
| 67 | LQSRIFPRT | Anti-BCMA C11D5.3 |
| | | scFv light chain CDR3 |
| 68 | QIQLVQSGPELKKPGETVKISCKASG | Anti-BCMA C11D5.3 |
| | YTFTDYSINWVKRAPGKGLKWMG | scFv heavy chain |
| | WINTETREPAYAYDFRGRFAFSLETS | variable region |
| | ASTAYLQINNLKYEDTATYFCALDYS | |
| | YAMDYWGQGTSVTVSS | |
| 69 | DYSIN | Anti-BCMA C11D5.3 |
| | | scFv heavy chain CDR1 |
| 70 | WINTETREPAYAYDFRG | Anti-BCMA C11D5.3 |
| | | scFv heavy chain CDR2 |
| 71 | DYSYAMDY | Anti-BCMA C11D5.3 |
| | | scFv heavy chain CDR3 |
| 72 | DIVLTQSPPSLAMSLGKRATISCRASE | Anti-BCMA C12A3.2 |
| | SVTILGSHLIYWYQQKPGQPPTLLIQ | scFv entire sequence, |
| | LASNVQTGVPARFSGSGSRTDFTLTI | with Whitlow linker |
| | DPVEEDDVAVYYCLQSRTIPRTFGGG | |
| | TKLEIKGSTSGSGKPGSGEGSTKGQI | |
| | QLVQSGPELKKPGETVKISCKASGY | |
| | TFRHYSMNWVKQAPGKGLKWMGR | |
| | INTESGVPIYADDFKGRFAFSVETSA | |
| | STAYLVINNLKDEDTASYFCSNDYLY | |
| | SLDFWGQGTALTVSS | |
| 73 | DIVLTQSPPSLAMSLGKRATISCRASE | Anti-BCMA C12A3.2 |
| | SVTILGSHLIYWYQQKPGQPPTLLIQ | scFv light chain variable |
| | LASNVQTGVPARFSGSGSRTDFTLTI | region |
| | DPVEEDDVAVYYCLQSRTIPRTFGGG | |
| | TKLEIK | |
| 74 | RASESVTILGSHLIY | Anti-BCMA C12A3.2 |
| | | scFv light chain CDR1 |
| 75 | LASNVQT | Anti-BCMA C12A3.2 |
| | | scFv light chain CDR2 |
| 76 | LQSRTIPRT | Anti-BCMA C12A3.2 |
| | | scFv light chain CDR3 |
| 77 | QIQLVQSGPELKKPGETVKISCKASG | Anti-BCMA C12A3.2 |
| | YTFRHYSMNWVKQAPGKGLKWMG | scFv heavy chain |
| | RINTESGVPIYADDFKGRFAFSVETS | variable region |
| | ASTAYLVINNLKDEDTASYFCSNDYL | |
| | YSLDFWGQGTALTVSS | |
| 78 | HYSMN | Anti-BCMA C12A3.2 |
| | | scFv heavy chain CDR1 |
| 79 | RINTESGVPIYADDFKG | Anti-BCMA C12A3.2 |
| | | scFv heavy chain CDR2 |
| 80 | DYLYSLDF | Anti-BCMA C12A3.2 |
| | | scFv heavy chain CDR3 |
| 81 | EVQLLESGGGLVQPGGSLRLSCAAS | Anti-BCMA FHVH33 |
| | GFTFSSYAMSWVRQAPGKGLEWVS | entire sequence |
| | SISGSGDYIYYADSVKGRFTISRDISK | |
| | NTLYLQMNSLRAEDTAVYYCAKEG | |
| | TGANSSLADYRGQGTLVTVSS | |
| 82 | GFTFSSYA | Anti-BCMA FHVH33 |
| | | CDR1 |
| 83 | ISGSGDYI | Anti-BCMA FHVH33 |
| | | CDR2 |
| 84 | AKEGTGANSSLADY | Anti-BCMA FHVH33 |
| | | CDR3 |
| 118 | DIQMTQSPSSLSASVGDRVTITCRAS | Anti-BCMA CT103A |
| | QSISSYLNWYQQKPGKAPKLLIYAA | scFv entire sequence, |
| | SSLQSGVPSRFSGSGSGTDFTLTISSL | with Whitlow linker |
| | QPEDFATYYCQQKYDLLTFGGGTKV | |
| | EIKGSTSGSGKPGSGEGSTKGQLQL | |
| | QESGPGLVKPSETLSLTCTVSGGSISS | |
| | SSYYWGWIRQPPGKGLEWIGSISYS | |
| | GSTYYNPSLKSRVTISVDTSKNQFSL | |
| | KLSSVTAADTAVYYCARDRGDTILD | |
| | VWGQGTMVTVSS | |
| 119 | DIQMTQSPSSLSASVGDRVTITCRAS | Anti-BCMA CT103A |
| | QSISSYLNWYQQKPGKAPKLLIYAA | scFv light chain variable |
| | SSLQSGVPSRFSGSGSGTDFTLTISSL | region |
| | QPEDFATYYCQQKYDLLTFGGGTKV | |
| | EIK | |
| 120 | QSISSY | Anti-BCMA CT103A |
| | | scFv light chain CDR1 |
| 121 | AAS | Anti-BCMA CT103A |
| | | scFv light chain CDR2 |
| 122 | QQKYDLLT | Anti-BCMA CT103A |
| | | scFv light chain CDR3 |
| 123 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRGD | |
| | TILDVWGQGTMVTVSS | |
| 124 | GGSISSSSYY | Anti-BCMA CT103A |
| | | scFv heavy chain CDR1 |
| 125 | ISYSGST | Anti-BCMA CT103A |
| | | scFv heavy chain CDR2 |
| 126 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:127 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:127 (see Table 14). The encoded BCMA CAR has a corresponding amino acid sequence set forth in SEQ ID NO:128 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:128, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 14
Exemplary sequences of BCMA CARs

| SEQ ID NO: | Sequence | Description |
|---|---|---|
| 127 | atggccttaccagtgaccgccttgctcctgccgctggccttgctg | Exemplary BCMA |
| | ctccacgccgccaggccggacatccagatgacccagtctccatcc | CAR nucleotide |
| | tccctgtctgcatctgtaggagacagagtcaccatcacttgccgg | sequence |
| | gcaagtcagagcattagcagctatttaaattggtatcagcagaaa | |
| | ccagggaaagcccctaagctcctgatctatgctgcatccagtttg | |
| | caaagtggggtcccatcaaggttcagtggcagtggatctgggaca | |
| | gatttcactctcaccatcagcagtctgcaacctgaagattttgca | |
| | acttactactgtcagcaaaaatacgacctcctcacttttggcgga | |
| | gggaccaaggttgagatcaaaggcagcaccagcggctccggcaag | |
| | cctggctctggcgagggcagcacaaagggacagctgcagctgcag | |
| | gagtcgggcccaggactggtgaagccttcggagaccctgtccctc | |
| | acctgcactgtctctggtggctccatcagcagtagtagttactac | |
| | tggggctggatccgccagcccccagggaaggggctggagtggatt | |
| | gggagtatctcctatagtgggagcacctactacaacccgtccctc | |
| | aagagtcgagtcaccatatccgtagacacgtccaagaaccagttc | |
| | tccctgaagctgagttctgtgaccgccgcagacacggcggtgtac | |
| | tactgcgccagagatcgtggagacaccatactagacgtatggggt | |
| | cagggtacaatggtcaccgtcagctcattcgtgcccgtgttcctg | |
| | cccgccaaacctaccaccacccctgcccctagacctcccacccca | |
| | gccccaacaatcgccagccagcctctgtctctgcggcccgaagcc | |
| | tgtagacctgctgccggcggagccgtgcacaccagaggcctggac | |
| | ttcgcctgcgacatctacatctgggcccctctggccggcacctgt | |
| | ggcgtgctgctgctgagcctggtgatcaccctgtactgcaaccac | |
| | cggaacaaacggggcagaaagaaactcctgtatatattcaaacaa | |
| | ccatttatgagaccagtacaaactactcaagaggaagatggctgt | |
| | agctgccgatttccagaagaagaagaaggaggatgtgaactgaga | |
| | gtgaagttcagcagatccgccgacgcccctgcctaccagcaggga | |
| | cagaaccagctgtacaacgagctgaacctgggcagacgggaagag | |
| | tacgacgtgctggacaagcggagaggccgggaccccgagatgggc | |
| | ggaaagcccagacggaagaacccccaggaaggcctgtataacgaa | |
| | ctgcagaaagacaagatggccgaggcctacagcgagatcggcatg | |
| | aagggcgagcggaggcgcggcaagggccacgatggcctgtaccag | |
| | ggcctgagcaccgccaccaaggacacctacgacgccctgcacatg | |
| | caggccctgccccccaga | |
| 128 | MALPVTALLLPLALLLHAARPDIQMTQSPSSLSASVGDRVTITCR | Exemplary BCMA |
| | ASQSISSYLNWYQQKPGKAPKLLIYAASSLQSGVPSRFSGSGSGT | CAR amino acid |
| | DFTLTISSLQPEDFATYYCQQKYDLLTFGGGTKVEIKGSTSGSGK | sequence |
| | PGSGEGSTKGQLQLQESGPGLVKPSETLSLTCTVSGGSISSSSYY | |
| | WGWIRQPPGKGLEWIGSISYSGSTYYNPSLKSRVTISVDTSKNQF | |
| | SLKLSSVTAADTAVYYCARDRGDTILDVWGQGTMVTVSSFVPVFL | |
| | PAKPTTTPAPRPPTPAPTIASQPLSLRPEACRPAAGGAVHTRGLD | |
| | FACDIYIWAPLAGTCGVLLLSLVITLYCNHRNKRGRKKLLYIFKQ | |
| | PFMRPVQTTQEEDGCSCRFPEEEEGGCELRVKFSRSADAPAYQQG | |
| | QNQLYNELNLGRREEYDVLDKRRGRDPEMGGKPRRKNPQEGLYNE | |
| | LQKDKMAEAYSEIGMKGERRRGKGHDGLYQGLSTATKDTYDALHM | |
| | QALPPR | |

### CD27 CAR

In some embodiments, the CAR is a CD27 CAR (“CD27-CAR”), and in these embodiments, the polycistronic vector comprises an expression cassette that contains a nucleotide sequence encoding a CD27 CAR. CD27 is an antigen found on the surface of memory B cells, until maturation into antibody-producing plasma cells, as well as CD4+ and CD8⁺ T cells, NK cells, and thymocytes. CD27 positive cells are also sometimes found in cases of B cell lymphomas, adult T cell leukemia/lymphoma, and acute myeloid leukemia. In some embodiments, the CD27 CAR may comprise a signal peptide, an extracellular binding domain that specifically binds CD27, a hinge domain, a transmembrane domain, an intracellular costimulatory domain, and/or an intracellular signaling domain in tandem.

In some embodiments, the signal peptide of the CD27 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the CD27 CAR is specific to CD27, for example, human CD27, as described in clinical trial NCT01460134. The extracellular binding domain of the CD27 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD27 CAR is derived from an antibody specific to CD27, including, for example, varlilumab/CDX1127, or from any of the antibodies or CARs disclosed in Table 15, optionally comprising the heavy chain variable region (V_{H}) and the light chain variable region (V_{L}) of one of the antibodies or CARS, connected by a linker. In any of these embodiments, the extracellular binding domain of the CD27 CAR can comprise or consist of the V_{H}, the V_{L}, and/or one or more CDRs of any of the antibodies.

TABLE 15
Exemplary CD27 antigen binding domains

| Ab/CAR | | | |
|---|---|---|---|
| Name | Antigen | Company | Reference |
| | CD27 | Kyowa Hakko Kirin Co | EP 2 520 589 |
| | | Ltd | |
| | CD27 | Kyowa Hakko Kirin Co | U.S. Pat. |
| | | Ltd | No. 9,403,914 |
| | CD27 | Celldex Therapeutics, | U.S. Pat. |
| | | Inc. | No. 11,180,566 |
| | CD27 | Jiangsu Hengrui | US 2021/0009706 |
| | | Medicine Co., Ltd | |
| | CD27 | Celldex Therapeutics, | EP 2 558 498 B1 |
| | | Inc. | |
| Varlilumab | CD27 | Celldex Therapeutics, | |
| | | Inc. | |

In some embodiments, the extracellular binding domain of the CD27 CAR comprises an scFv derived from the varlilumab monoclonal antibody, which comprises the heavy chain variable region (V_{H}) and the light chain variable region (V_{L}) of varlilumab connected by a linker. See Wu et al., Protein Engineering. 14 (12): 1025-1033 (2001). In some embodiments, the linker is a 3xG₄S (SEQ ID NO: 30) linker. In other embodiments, the linker is a Whitlow linker as described herein. In some embodiments, the amino acid sequences of different portions of the varlilumab-derived scFv (also referred to as varlilumab scFv) and its different portions are provided in Table 16 below. In some embodiments, the CD27-specific scFv comprises or consists of the heavy and light chain sequences sequence set forth in SEQ ID NOs: 129 and 130, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 129 and 130. In some embodiments, the CD27-specific scFv may comprise one or more CDRs having amino acid sequences set forth in SEQ ID NOs: 129 and 130. In some embodiments, the CD27-specific scFv may comprise a light chain with one or more CDRs having amino acid sequences set forth in SEQ ID NO: 130. In some embodiments, the CD27-specific scFv may comprise a heavy chain with one or more CDRs having amino acid sequences set forth in SEQ ID NOs: 129. In any of these embodiments, the CD27-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD27 CAR comprises or consists of the one or more CDRs as described herein.

TABLE 16
Exemplary sequences of anti-CD27 scFv components

| SEQ ID NO: | Amino Acid Sequence | Description |
|---|---|---|
| 129 | QVQLVESGGGVVQPGRSLRLSCAASGFTFSS | Varlilumab heavy chain |
| | YDMHWVRQAPGKGLEWVAVIWYDGSNKY | |
| | YADSVKGRFTISRDNSKNTLYLQMNSLRAED | |
| | TAVYYCARGSGNWGFFDYWGQGTLVTVSS | |
| 130 | DIQMTQSPSSLSASVGDRVTITCRASQGISR | Varlilumab light chain |
| | WLAWYQQKPEKAPKSLIYAASSLQSGVPSRF | |
| | SGSGSGTDFTLTISSLQPEDFATYYCQQYNTY | |
| | PRTFGQGTKVEIK | |

In some embodiments, the hinge domain of the CD27 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the CD27 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD27 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD27 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD27 CAR, including, for example, a CD27 CAR comprising the CD27-specific scFv having heavy and light chain sequences set forth in SEQ ID NOs: 129 and 130, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD27 CAR, including, for example, a CD27 CAR comprising the CD27-specific scFv having heavy and light chain sequences set forth in SEQ ID NOs: 129 and 130, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD27 CAR, including, for example, a CD27 CAR comprising the CD27-specific scFv heavy and light chain sequences set forth in SEQ ID NOs: 129 and 130, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD27 CAR, including, for example, a CD27 CAR comprising the CD27-specific scFv heavy and light chain sequences set forth in SEQ ID NOs: 129 and 130, the CD8α hinge domain of SEQ ID NO:9, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD27 CAR, including, for example, a CD27 CAR comprising the CD27-specific scFv heavy and light chain sequences set forth in SEQ ID NOs: 129 and 130, the CD28 hinge domain of SEQ ID NO:10, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD27 CAR, including, for example, a CD27 CAR comprising the CD27-specific scFv heavy and light chain sequences set forth in SEQ ID NOs: 129 and 130, the IgG4 hinge domain of SEQ ID NO:11 or SEQ ID NO:1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### CD30 CAR

In some embodiments, the CAR is a CD30 CAR (“CD30-CAR”), and in these embodiments, the polycistronic vector comprises an expression cassette that contains a nucleotide sequence encoding a CD30 CAR. CD30 is a transmembrane glycoprotein receptor found on the surface of a small subset of activated T and B lymphocytes, as well as a variety of lymphoid neoplasms such as Hodgkin's lymphoma, anaplastic large cell lymphoma, peripheral T-cell leukemia not otherwise specified (PTCL-NOS), adult-T-cell leukemia/lymphoma, cutaneous T-cell lymphoma, extra-nodal NK-T-cell lymphoma, diffuse large B-cell lymphoma, very often EBV-positive. CD30-positive cells are also occasionally found in advanced systemic mastocytosis and certain non-hematopoietic malignancies such as germ cell tumors and testicular embryonic carcinomas. In some embodiments, the CD30 CAR may comprise a signal peptide, an extracellular binding domain that specifically binds CD30, a hinge domain, a transmembrane domain, an intracellular costimulatory domain, and/or an intracellular signaling domain in tandem.

In some embodiments, the signal peptide of the CD30 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the CD30 CAR is specific to CD30, for example, human CD30. The extracellular binding domain of the CD30 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD30 CAR is derived from an antibody specific to CD30, including, for example, brentuximab (see clinical trials NCT02388490; NCT01805037; NCT04138875), BerH2 (as described in van der Weyden et al., Blood Cancer Journal 7, e603 (2017)) or others, as described in U.S. Pat. No. 7,387,776 Keler et al. (2003); U.S. Pat. No. 8,088,377, Keler et al., (2008), incorporated herein by reference in their entireties). In any of these embodiments, the extracellular binding domain of the CD30 CAR can comprise or consist of the V_{H}, the V_{L}, and/or one or more CDRs of any of the antibodies.

In some embodiments, the extracellular binding domain of the CD30 CAR comprises an scFv derived from the brentuximab monoclonal antibody, which comprises the heavy chain variable region (V_{H}) and the light chain variable region (V_{L}) of brentuximab connected by a linker. See Wu et al., Protein Engineering. 14 (12): 1025-1033 (2001). In some embodiments, the linker is a 3xG₄S (SEQ ID NO: 30) linker. In other embodiments, the linker is a Whitlow linker as described herein. In some embodiments, the amino acid sequences of different portions of the brentuximab-derived scFv (also referred to as brentuximab scFv) and its different portions are provided in Table 16 below. In some embodiments, the CD30-specific scFv comprises or consists of the heavy and light chain sequences set forth in SEQ ID NOs: 131 and 132, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 131 and 132. In some embodiments, the CD30-specific scFv may comprise one or more CDRs having amino acid sequences set forth in SEQ ID NOs: 131 and 132. In some embodiments, the CD30-specific scFv may comprise a light chain with one or more CDRs having amino acid sequences set forth in SEQ ID NO: 132. In some embodiments, the CD30-specific scFv may comprise a heavy chain with one or more CDRs having amino acid sequences set forth in SEQ ID NO: 131. In any of these embodiments, the CD30-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD30 CAR comprises or consists of the one or more CDRs as described herein.

In some embodiments, the CD30-specific CAR comprises or consists of the sequences set forth in SEQ ID NOs: 133 or 134, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 133 or 134.

TABLE 17
Exemplary sequences of anti-CD30 scFv components

| SEQ ID NO: | Amino Acid Sequence | Description |
|---|---|---|
| 131 | QIQLQQSGPEVVKPGASVKISCKASGYTFTD | Brentuximab heavy chain |
| | YYITWVKQKPGQGLEWIGWIYPGSGNTKYN | |
| | EKFKGKATLTVDTSSSTAFMQLSSLTSEDTAVY | |
| | FCANYGNYWFAYWGQGTQVTVSA | |
| 132 | DIVLTQSPASLAVSLGQRATISCKASQSVDFD | Brentuximab light chain |
| | GDSYMNWYQQKPGQPPKVLIYAASNLESGI | |
| | PARFSGSGSGTDFTLNIHPVEEEDAATYYCQ | |
| | QSNEDPWTFGGGTKLEIK | |

TABLE 18
Exemplary sequences of CD30 CARS

| SEQ ID NO: | Sequence | Description |
|---|---|---|
| 133 | QVQLQQSGAELARPGASVKMSCKASGYTFTTYTIH | CD30 CAR (lacking |
| | VWRRRPGHDLEWIGYINPSSGCSDYNQNFKGKTTL | signal peptide) |
| | TADKSSNTAYMQLNSLTSEDSAVYYCARRADYGNYE | (SEQ ID NO: 35 |
| | YTWFAYVVGQGTTVTVSSSGGGSGGGGSGGGGSV | from WO |
| | IELTQSPKFMSTSVGDRVNVTYKASQNVGTNVAWF | 2021/222929) |
| | QQKPGQSPKVLIYSASYRYSGVPDRFTGSGSGTDFTL | |
| | TISNVQSEDLAEYFCQQYHTYPLTFGGGTKLEIKRSD | |
| | PAEPKSPDKTHTCPPCPAPELLGGPSVFLFPPKPKDT | |
| | LMISRTPEVTCVVVDVSHEDPEVKFNWYVDGVEVH | |
| | NAKTKPREEQYNSTYRVVSVLTVLHQDWLNGKEYK | |
| | CKVSNKALPAPIEKTISKAKGQPREPQVYTLPPSRDEL | |
| | TKNQVSLTCLVKGFYPSDIAVEWESNGQPENNYKTT | |
| | PPVLDSDGSFFLYSKLTVDKSRWQQGNVFSCSVMH | |
| | EALHNHYTQKSLSLSPGKKDPKFVWLVVVGGVLAC | |
| | YSLLVTVAFIIFWVRSKRSRLLHSDYMNMTPRRPGP | |
| | TRKHYQPYAPPRDFAAYRSRVKFSRSADAPAYQQGQ | |
| | NQLYNELNLGRREEYDVLDKRRGRDPEMGGKPRRK | |
| | NPQEGLYNELQKDKMAEAYSEIGMKGERRRGKGH | |
| | DGLYQGLSTATKDTYDALHMQALPPR | |
| 134 | MEFGLSWLFLVAILKGVQCSRDIVLTQSPASLAVSLG | CD30 CAR |
| | QRATISCKASQSVDFDGDSYMNWYQQKPGQPPKV | (SEQ ID NO: 26 |
| | LIYAASNLESGIPARFSGSGSGTDFTLNIHPVEEEDAA | from |
| | TYYCQQSNEDPWTFGGGTKLEIKGGGSGGGGQIQL | US20210206863) |
| | QQSGPEVVKPGASVKISCKASGYTFTDYYITWVKQK | |
| | PGQGLEWIGWIYPGSGNTKYNEKFKGKATLTVDTSS | |
| | STAFMQLSSLTSEDTAVYFCANYGNYWFAYWGQGT | |
| | QVTVSAGSELPTQGTFSNVSTNVSPAPRPPTPAPTIA | |
| | SQPLSLRPEACRPAAGGAVHTRGLDFACDIYIWAPL | |
| | AGTCGVLLLSLVITLYCNHRNEFRSKRSRLLHSDYMN | |
| | MTPRRPGPTRKHYQPYAPPRDFAAYRSRDQRLPPD | |
| | AHKPPGGGSFRTPIQEEQADAHSTLAKIRVKFSRSA | |
| | DAPAYQQGQNQLYNELNLGRREEYDVLDKRRGRDP | |
| | EMGGKPRRKNPQEGLYNELQKDKMAEAYSEIGMK | |
| | GERRRGKGHDGLYQGLSTATKDTYDALHMQALPPR | |

In some embodiments, the hinge domain of the CD30 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the CD30 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the CD30 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the CD30 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the CD8α hinge domain of SEQ ID NO:9, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the CD28 hinge domain of SEQ ID NO:10, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the IgG4 hinge domain of SEQ ID NO:11 or SEQ ID NO:12, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the CD8α hinge domain of SEQ ID NO:9, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the CD28 hinge domain of SEQ ID NO:10, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD30 CAR, including, for example, a CD30 CAR comprising the CD30-specific scFv heavy and light chain sequences set forth in SEQ ID NOs: 131 and 132, the IgG4 hinge domain of SEQ ID NO:11 or SEQ ID NO:1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### EBV CARS

In some embodiments, the CAR is specific for an EBV antigen (“EBV antigen-specific CAR” or “EBV antigen CAR”). Examples of EBV antigens include, but are not limited to, BRLF1, BALF4, EBNA1, EBNA3A, EBNA3C, LMP1, LMP2, LMP2A, LMP2B, BZLF1, BMLF1, Gp350, and gH/gL. Accordingly, in some embodiments, an EBV antigen CAR described herein is directed to BRLF1, BALF4, EBNA1, EBNA3A, EBNA3C, LMP1, LMP2, LMP2A (which includes antigens that bind to LMP2B), BZLF1, BMLF1, Gp350, and/or gH/gL.

### EBNA1 CAR

In some embodiments, the CAR is a EBNA1 CAR (“EBNA1-CAR”), and in these embodiments, the polycistronic vector comprises an expression cassette that contains a nucleotide sequence encoding a EBNA1 CAR. EBNA1, which is an Epstein-Barr Virus (EBV) nuclear protein having functions in maintaining the viral genome, and is expressed during the viral latency stages. It is found on Burkitt's lymphoma cells, Hodgkin's lymphoma cells, nasopharyngeal carcinoma cells, gastric carcinoma cells, lymphomas in immunosuppressed patients, as well as T/NK cell lymphomas, aggressive NK-cell leukemia, and T cell lymphoproliferative disorder of childhood. Patients suffering multiple sclerosis often display high numbers of EBNA1-specific T cells. In some embodiments, the EBNA1 CAR may comprise a signal peptide, an extracellular binding domain that specifically binds EBNA1, a hinge domain, a transmembrane domain, an intracellular costimulatory domain, and/or an intracellular signaling domain in tandem.

In some embodiments, the signal peptide of the EBNA1 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the EBNA1 CAR is specific to EBNA1, for example, viral EBNA1. The binding domain of the EBNA1 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the EBNA1 CAR is derived from an antibody specific to EBNA1, including, for example, D810H, 3EB7, 10C124, 2Q1914, 10C123, M5042521, 7E124 (as described in WIPO Patent Application No. WO1986001210, Vaughan et al. (1985), incorporated herein by reference in their entireties, anti-EBNA1-16D2 (as described in Yadav et al. Immun. Inflamm. Dis. 4 (3): 362-375 (2016), incorporated herein by reference in their entireties), or anti-EBNA1-3D4 (as described in Yadav et al., Plos One 6 (1):e14488 (2011), incorporated herein by reference in their entireties). In any of these embodiments, the extracellular binding domain of the EBNA1 CAR can comprise or consist of the V_{H}, the V_{L}, and/or one or more CDRs of any of the antibodies.

In some embodiments, the extracellular binding domain of the EBNA1 CAR comprises an scFv derived from the HLA-A0201/EBNA1 monoclonal antibody. In some embodiments, the amino acid sequences of the HLA-A0201/EBNA1-derived scFv and its different portions are provided in Table 19 below. In some embodiments, the EBNA1-specific scFv comprises or consists of the heavy and light chain sequences sequence set forth in SEQ ID NOs: 135 and 136, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 135 and 136. In some embodiments, the EBNA1-specific scFv may comprise one or more CDRs having amino acid sequences set forth in SEQ ID NOs: 135 and 136. In some embodiments, the EBNA1-specific scFv may comprise a heavy chain with one or more CDRs having amino acid sequences set forth in SEQ ID NO: 135. In some embodiments, the EBNA1-specific scFv may comprise a light chain with one or more CDRs having amino acid sequences set forth in SEQ ID NO: 136. In any of these embodiments, the EBNA1-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the EBNA1 CAR comprises or consists of the one or more CDRs as described herein.

In some embodiments, the extracellular binding domain of the EBNA1 CAR comprises an scFv derived from a TCR-like monoclonal antibody targeting EBNA1 amino acid sequence 562-570 (EBNA1₅₆₂₋₅₆₀), as described in Lai et al., Blood (2016) 128 (10): 1396-1407, and in Sim et al., Sci Rep 2013; 3:3232. In some embodiments, the scFv derived from this TCR-like monoclonal antibody comprises the V_{H} and the V_{L} of EBNA1₅₆₂₋₅₇₀ connected by a 3xG₄S (SEQ ID NO: 30) linker. In other embodiments, the Whitlow linker may be used instead. In some embodiments, the amino acid sequences of the EBNA1₅₆₂₋₅₇₀ derived scFv (also referred to as EBNA1₅₆₂₋₅₇₀ sCFv) and its different portions are provided in Table 19 below. In some embodiments, the EBNA1-specific scFv comprises or consists of an amino acid sequence set forth in SEQ ID NO: 135 or 136, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:135 or 136. In some embodiments, the EBNA1-specific scFv may comprise one or more CDRs having amino acid sequences set forth in SEQ ID NOs: 135 and 136 (bolded). In some embodiments, the EBNA1-specific scFv may comprise a heavy chain with one or more CDRs having amino acid sequences set forth in SEQ ID NOs: 135. In some embodiments, the EBNA1-specific scFv may comprise a light chain with one or more CDRs having amino acid sequences set forth in SEQ ID NOs: 136. In any of these embodiments, the EBNA1-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the EBNA1 CAR comprises or consists of the one or more CDRs as described herein.

In some embodiments, the CAR is a CD20 CAR (“CD20-CAR”), and in these signaling domains in tandem.

TABLE 19
Exemplary sequences of anti-EBNA scFv components

| SEQ ID NO: | Amino Acid Sequence | Description |
|---|---|---|
| 135 | QVQLKESGPGLVAPSQSLSITCTVSGFSLTGY | HLA-A0201/EBNA1 |
| | GVNWVRQPPGKGLEWLGMIWGDGSTDY | hybridoma, |
| | NSALKSRLSISKDNSKSQVFLKMNSLQTDDT | Heavy chain |
| | ARYYCARDPYGYIFDYWGQGTTLTVSSAKTT | Bolded sequences are |
| | PPSVYPLAPGSAAQTNSMVTLGCLVKGYFPE | CDRs |
| | PVTVTWNSGSLSSGVHTFPA | (SEQ ID NO: 31 from |
| | VLQSDLYTLSSSVTVPSSTWPSETVTCNVAHP | WO2015199617A1) |
| | ASSTKVDKKIVPRDC | |
| 136 | DIVMTQSQKFMSTSVGDRVSITCKASQNVH | HLA-A0201/EBNA1 |
| | TAVAEWYQQKAGQSPKALIYLASNRHTGVP | hybridoma, |
| | DRFTGSGSGTDFTL | Light chain |
| | TISNVQSEDLADYFCLQHWNNPLTFGAGTK | Bolded sequences are |
| | LELKRADAAPTVSIFPPSSEQLTSGGASVVCFL | CDRs |
| | NNFYPKDINVKWKIDGSERQNGVLNSWTD | (SEQ ID NO: 36 from |
| | QDSKDSTYSMSSTLTLTKDEYERHNSYTCEAT | WO2015199617A1) |
| | HKTSTSPIVKSFNRNEC | |

In some embodiments, the hinge domain of the EBNA1 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the EBNA1 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the EBNA1 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the EBNA1 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA1 CAR, including, for example, a EBNA1 CAR comprising the EBNA1-specific scFv heavy and light chain sequences set forth in SEQ ID NOs: 135 and 136, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In some embodiments, the hinge domain of the CD20 CAR comprises a CD8α hinge identical) to the amino acid sequence set forth in SEQ ID NO:13.

### EBNA3A CAR

In some embodiments, the CAR is a EBNA3A CAR (“EBNA3A-CAR”), and in these embodiments, the polycistronic vector comprises an expression cassette that contains a nucleotide sequence encoding a EBNA3A CAR. EBNA3A is a nuclear antigen expressed during the full latency of EBV-infected naïve B cells. EBNA3A is involved in the differentiation of naïve B cells into premalignant precursor cells in diseases such as DLBCL, Hodgkin's lymphoma and Burkitt's lymphoma. The expression of EBNA3A has been shown to be related to most EBV-associated lymphoproliferative disorders and malignancies. Patients suffering multiple sclerosis often display high numbers of EBNA3A-specific T cells. In some embodiments, the EBNA3A CAR may comprise a signal peptide, an extracellular binding domain that specifically binds EBNA3A, a hinge domain, a transmembrane domain, an intracellular costimulatory domain, and/or an intracellular signaling domain in tandem.

In some embodiments, the signal peptide of the EBNA3A CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the EBNA3A CAR is specific to EBNA3A, for example, viral EBNA3A. The binding domain of the EBNA3A CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the EBNA3A CAR is derived from an antibody specific to EBNA3A, including, for example, any of the antibodies or CARs disclosed in Table 20, the references cited in which are incorporated by reference in their entireties herein. In any of these embodiments, the extracellular binding domain of the EBNA3A CAR can comprise or consist of the V_{H}, the V_{L}, and/or one or more CDRs of any of the antibodies disclosed in Table 20.

In some embodiments, the extracellular binding domain of the EBNA3A CAR comprises an scFv derived from the any of the antibodies or CARs disclosed in Table 20, optionally comprising the heavy chain variable region (V_{H}) and the light chain variable region (V_{L}) of one of the antibodies or CARs, connected by a linker. In some embodiments, the linker is a 3xG₄S (SEQ ID NO: 30) linker. In other embodiments, the Whitlow linker may be used instead. In some embodiments, the EBNA3A-specific scFv comprises or consists of the scFv of an antibody or CAR disclosed in Table 20, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 20. In some embodiments, the EBNA3A-specific scFv may comprise one or more CDRs having amino acid sequences of the CDRs of an antibody or CAR disclosed in Table 20. In some embodiments, the EBNA3A-specific scFv may comprise a heavy chain with one or more CDRs having amino acid sequences of the CDRs of an antibody or CAR disclosed in Table 20. In some embodiments, the EBNA3A-specific scFv may comprise a light chain with one or more CDRs having amino acid sequences of the CDRs of an antibody or CAR disclosed in Table 20. In any of these embodiments, the EBNA3A-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the EBNA3A CAR comprises or consists of the one or more CDRs as described herein, including in Table 20.

TABLE 20
Exemplary EBNA3A antigen binding domains

| Ab/CAR | | | |
|---|---|---|---|
| Name | Antigen | Company | Reference |
| Ab16126 | EBNA3A | Abcam | Styles et al., Plos One |
| | | | 15(8): e2001992 (2017) |
| T2.78 | EBNA3A | N/A | Rowe et al., J. Virol. |
| | | | 63(3): 1031-1039 (1989) |
| BS-0820R | EBNA3A | Bioss | Biossusa.com/products/bs- |
| | | | 0820r |
| F115P | EBNA3A | Exalpha | Maruo et al., J. Virol. |
| | | Biologicals | 77(9): 10437-10447 |
| | | | (2003) |
| ABIN728623 | EBNA3A | Antibodies- | Antibodies-online.com |
| | | online | |
| TCR-YC0337 | EBNA3A | Creative | Creative- |
| | | Biolabs | biolabs.com/car-t/anti- |
| | | | ebna-3a-t-cell-receptor- |
| | | | yc04-pctcr1-77873 |

In some embodiments, the hinge domain of the EBNA3A CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the EBNA3A CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the EBNA3A CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the EBNA3A CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a EBNA3A CAR, including, for example, a EBNA3A CAR comprising any of the EBNA3A-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA3A CAR, including, for example, a EBNA3A CAR comprising any of the EBNA3A-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA3A CAR, including, for example, a EBNA3A CAR comprising the EBNA3A-specific scFv having sequences of an antibody or CAR disclosed in Table 20, the CD8α hinge domain of SEQ ID NO:9, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof

In some embodiments, the polycistronic vector comprises an expression cassette that contains a nucleotide sequence encoding a EBNA3A CAR, including, for example, a EBNA3A CAR comprising the EBNA3A-specific scFv having sequences of an antibody or CAR disclosed in Table 20, the CD28 hinge domain of SEQ ID NO:10, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a EBNA3A CAR, including, for example, a EBNA3A CAR comprising the EBNA3A-specific scFv having sequences of an antibody or CAR disclosed in Table 20, the IgG4 hinge domain of SEQ ID NO:11 or SEQ ID NO:12, the CD28 transmembrane domain of SEQ ID NO: 15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### EBNA3C CAR

In some embodiments, the CAR is a EBNA3C CAR (“EBNA3C-CAR”), and in these embodiments, the polycistronic vector comprises an expression cassette that contains a nucleotide sequence encoding a EBNA3C CAR. Similarly to EBNA3A, EBNA3C is a nuclear antigen expressed during the full latency of EBV-infected naïve B cells. EBNA3C is involved in the differentiation of naïve B cells into premalignant precursor cells in diseases such as DLBCL, Hodgkin's lymphoma and Burkitt's lymphoma. The expression of EBNA3C has been shown to be related to most EBV-associated lymphoproliferative disorders and malignancies. Patients suffering multiple sclerosis often display high numbers of EBNA3C-specific T cells. In some embodiments, the EBNA3C CAR may comprise a signal peptide, an extracellular binding domain that specifically binds EBNA3C, a hinge domain, a transmembrane domain, an intracellular costimulatory domain, and/or an intracellular signaling domain in tandem.

In some embodiments, the signal peptide of the EBNA3C CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the EBNA3C CAR is specific to EBNA3C, for example, viral EBNA3C. The binding domain of the EBNA3C CAR can be codon-optimized for expression in a host cell or to have variant sequences to increase functions of the extracellular binding domain.

In some embodiments, the binding domain comprises an immunogenically active portion of an immunoglobulin molecule, for example, an scFv. In some embodiments, the extracellular binding domain of the EBNA3C CAR is derived from an antibody specific to EBNA3C, including, for example, TÜ165 (from Dragon et al.), or fragments of T-cell receptor TCR-C055Z, or of CAR MZ043 (commercially available through Creative-Biolabs). In any of these embodiments, the extracellular binding domain of the EBNA3C CAR can comprise or consist of the V_{H}, the V_{L}, and/or one or more CDRs of any of the antibodies.

In some embodiments, the extracellular binding domain of the EBNA3C CAR comprises an scFv derived from TÜ165, a monoclonal antibody as described in Dragon et al., J Immunother Cancer 8:e000736 (2020).

In some embodiments, the EBNA3C-specific extracellular binding domain comprises or consists of an amino acid sequence set forth in SEQ ID NO:139, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:139. In some embodiments, the EBNA3C-specific extracellular binding domain may comprise one or more CDRs having amino acid sequences set forth in SEQ ID NO: 139. In some embodiments, the EBNA3C-specific extracellular binding domain may comprise a light chain with one or more CDRs having amino acid sequences set forth in SEQ ID NO: 139. In some embodiments, the EBNA3C-specific extracellular binding domain may comprise a heavy chain with one or more CDRs having amino acid sequences set forth in SEQ ID NO: 139. In any of these embodiments, the EBNA3C-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the EBNA3C CAR comprises or consists of the one or more CDRs as described herein.

In some embodiments, the EBNA3C-specific extracellular binding domain comprises or consists of the heavy and light chain sequences sequence set forth in SEQ ID NOS: 137 and 138, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 137 and 138. In some embodiments, the EBNA3C-specific extracellular binding domain may comprise one or more CDRs having amino acid sequences set forth in SEQ ID NOs: 137 and 138. In some embodiments, the EBNA3C-specific extracellular binding domain may comprise a light chain with one or more CDRs having amino acid sequences set forth in SEQ ID NO: 138. In some embodiments, the EBNA3C-specific extracellular binding domain may comprise a heavy chain with one or more CDRs having amino acid sequences set forth in SEQ ID NO: 139. In any of these embodiments, the EBNA3C-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the EBNA3C CAR comprises or consists of the one or more CDRs as described herein.

Additionally, CARs and binders directed to EBNA3C have been described in PCT application WO 2012/109659 A1, the entire contents of each of which are incorporated by reference herein.

TABLE 20
Exemplary sequences of anti-EBNA3C scFv components

| SEQ ID NO: | Amino Acid Sequence | Description |
|---|---|---|
| 137 | EVQLLESGGGLVQPGGSLRLSCAASGFTFSG | Anti-EBNA3C full VH |
| | YAMSWVRQAPGKGLEWVSEIAPPGLNTRY | (CDRs are bolded) |
| | ADSVKGRFTISRDNSKNTLYLQMNSLRAEDT | (SEQ ID NO: 24 from |
| | AVYYCAKSDTAFDYWGQGTLVTVS | WO2012109659A1) |
| 138 | STDIQMTQSPSSLSASVGDRVTITCRASQSIS | Anti-EBNA3C full VL |
| | SYLNWYQQKPGKAPKLLIYLASNLQSGVPSR | (CDRs are bolded) |
| | FSGSGSGTDFTLTISSLQPEDFATYYCQQAEY | (SEQ ID NO: 25 from |
| | MPLTFGQGTKVEIKRA | WO2012109659A1) |
| 139 | EVQLLESGGGLVQPGGSLRLSCAASGFTFSG | Anti-EBNA3C scFv clone |
| | YAMSWVRQAPGKGLEWVSEIAPPGLNTRYA | 315 |
| | DSVKGRFTISRDNSKNTLYLQMNSLRAEDTA | (SEQ ID NO: 5 from |
| | VYYCAKSDTAFDYWGQGTLVTVSSGGGGSG | WO201209759A1) |
| | GGGSGGGGSTDIQMTQSPSSLSASVGDRVT | |
| | ITCRASQSISSYLNWYQQKPGKAPKLLIYLAS | |
| | NLQSGVPSRFSGSGSGTDFTLTISSLQPEDFA | |
| | TYYCQQAEYMPLTFGQGTKVEIKRA | |

In some embodiments, the hinge domain of the EBNA3C CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the EBNA3C CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the EBNA3C CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the EBNA3C CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a EBNA3C CAR, including, for example, a EBNA3C CAR comprising any of the EBNA3C-specific extracellular binding domains as described, the CD8α hinge domain of SEQ ID NO:9, the CD8α transmembrane domain of SEQ ID NO: 14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the EBNA3C CAR may additionally comprise a signal peptide (e.g., a CD8α signal peptide) as described.

In some embodiments, the polycistronic vector comprises an expression cassette that contains a nucleotide sequence encoding a EBNA3C CAR, including, for example, a EBNA3C CAR comprising any of the EBNA3C-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the EBNA3C CAR may additionally comprise a signal peptide as described.

### LMP1 CAR

In some embodiments, the CAR is a LMP1 CAR (“LMP1-CAR”), and in these embodiments, the polycistronic vector comprises an expression cassette that contains a nucleotide sequence encoding a LMP1 CAR. LMP1 is a large multipass transmembrane oncoprotein that immortalizes B cells and non-lymphoid tissues, leading to survival of EBV-infected cells. LMP1 is involved in the latency stage of EBV infection, as well as in cell transformation and immortalization, cytokine and chemokine production, immune modulation, tumor angiogenesis, and cell migration. The expression of LMP1 has been shown to be related to most EBV-associated lymphoproliferative disorders and malignancies. Patients suffering multiple sclerosis often display high numbers of LMP1-specific T cells. In some embodiments, the LMP1 CAR may comprise a signal peptide, an extracellular binding domain that specifically binds LMP1, a hinge domain, a transmembrane domain, an intracellular costimulatory domain, and/or an intracellular signaling domain in tandem.

In some embodiments, the signal peptide of the LMP1 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the LMP1 CAR is specific to LMP1, for example, viral LMP1. The binding domain of the LMP1 CAR can be codon-optimized for expression in a host cell or to have variant sequences to increase functions of the extracellular binding domain.

In some embodiments, the binding domain comprises an immunogenically active portion of an immunoglobulin molecule, for example, an scFv. In some embodiments, the binding domain of the LMP1 CAR is derived from an antibody specific to LMP1, including, for example, anti-TES1-A3H5, anti-TES1-B10E7, anti-TES1-H11F6, anti-TES1-B10B7, or anti-TES1-B2A4 (as described in Gennari et al., J. Mol. Biol. 335:193-207 (2003), incorporated herein by reference in its entirety), anti-LMP1-B8, anti-LMP1-G5, anti-LMP1-A4, anti-LMP1-F5, anti-LMP1-E2, or anti-LMP1-H3, as detailed in Table 21. In any of these embodiments, the extracellular binding domain of the LMP1 CAR can comprise or consist of the V_{H}, the V_{L}, and/or one or more CDRs of any of the antibodies.

TABLE 21
Exemplary LMP1 antigen binding domains

| Ab/CAR | Antigen | Company | Reference |
|---|---|---|---|
| Anti-LMP1-B8 | LMP1 | Creative Biolabs | Creative-biolabs.net |
| Anti-LMP1-G5 | LMP1 | Creative Biolabs | Creative-biolabs.net |
| Anti-LMP1-A4 | LMP1 | Creative Biolabs | Creative-biolabs.net |
| Anti-LMP1-F5 | LMP1 | Creative Biolabs | Creative-biolabs.net |
| Anti-LMP1-E2 | LMP1 | Creative Biolabs | Creative-biolabs.net |
| Anti-LMP1-H3 | LMP1 | Creative Biolabs | Creative-biolabs.net |

In some embodiments, the binding domain of the LMP1 CAR comprises an scFv derived from the antibodies as described in U.S. Pat. No. 7,786,269; PCT Application Publication No. WO2010/015705; and European Patent No EP1470159, incorporated herein by reference in their entireties). The scFv may comprise the heavy chain variable region (V_{H}) and the light chain variable region (V_{L}) of said antibodies connected by the Whitlow linker, the amino acid sequences of which is provided in Table 22 below. In some embodiments, the LMP1-specific extracellular binding domain comprises or consists of an amino acid sequence set forth in SEQ ID NO: 140, 141, or 142, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 140, 141, or 142. In some embodiments, the LMP1-specific extracellular binding domain may comprise one or more CDRs having amino acid sequences set forth in SEQ ID NO: 140, 141, or 142. In some embodiments, the LMP1-specific extracellular binding domain may comprise a light chain with one or more CDRs having amino acid sequences set forth in SEQ ID NO: 140, 141, or 142. In some embodiments, the LMP1-specific extracellular binding domain may comprise a heavy chain with one or more CDRs having amino acid sequences set forth in SEQ ID NO: 140, 141, or 142. In any of these embodiments, the LMP1-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the LMP1 CAR comprises or consists of the one or more CDRs as described herein.

Additionally, T-cells expressing a LMP1-specific CAR have been described in Tang et al. J Biomed Res 28 (6): 468-475 (2014), the entire contents of each of which are incorporated by reference herein.

TABLE 22
Exemplary sequences of anti-LMP1 scFv components

| SEQ ID NO: | Amino Acid Sequence | Description |
|---|---|---|
| 140 | QVQLVQSGSELKKPGSSVKVSCKASGGTFSS | LMP1-specific antibody |
| | YAISWVRQAPGQGLEWMGGIIPIFGTANYA | (SEQ ID NO: 3 from US |
| | QKFQGRVTITADKSTSTAYMELSSLRSEDTAV | 7,786,269) |
| | YYCARGRDGMDVWGQGTLVTVSSGGGGS | (CDRs are bolded) |
| | GGGGSGGGGSQPGLTQPPSVSVSPGQTASI | |
| | TCSGDELGNRYAYWYQQKPGQSPVLVIYQD | |
| | RKRPSGIPERFSGSNSGNTATLTISGTQAMD | |
| | EADYYCQAWASGTGVFGTGTKVTVL | |
| 141 | VQLVQSGAEVKKPGSSVKVSCKASGVTFSSY | LMP1 antibody |
| | GINWVRQAPGQGLEWMGGIIPIFGTGNYA | (SEQ ID NO: 5 from US |
| | QKFQGRLTISADESTSTAYMELNSLRSEDTAV | 7,786,269) |
| | YYCARGNPFGQTWGQGTLVTVSSGGGGSG | (CDRs are bolded) |
| | GGGSGGGGSQPGLTQPPSVSPGQTASITCS | |
| | GDKLGDKYASWYQLKPGQSPLLVIYQDVKR | |
| | PSGIPERFSGSNSGNTATLTISGTQAMDEADY | |
| | YCQAWDSGTAVFGGGTKLTVL | |
| 142 | QVQLVQSGAEVKKPGSSVKVSCKASGGTFSS | LMP1 antibody |
| | YAISWVRQAPGQGLEWMGGIIPIFGTANYA | (SEQ ID NO: 7 from US |
| | QKFQGRVTITADESTSTAYMELSSLRSEDTAV | 7,786,269) |
| | YYCARPYLGWGQGTMVTVSSGGGGSGGG | (CDRs are bolded) |
| | GSGGGGSNFMLTQPHSVSESPGKTVNISCT | |
| | RSSGSIASHYVQWFQQRPGSAPATVIYEDK | |
| | QRPSGVPDRFSGSIDSSSNSASLTISGLRTEDE | |
| | ADYYCQSYDTGTWVFGGGTKLTVL | |

In some embodiments, the hinge domain of the LMP1 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the LMP1 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the LMP1 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the LMP1 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a LMP1 CAR, including, for example, a LMP1 CAR comprising any of the LMP1-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP1 CAR may additionally comprise a signal peptide (e.g., a CD8α signal peptide) as described.

In some embodiments, the polycistronic vector comprises an expression cassette that contains a nucleotide sequence encoding a LMP1 CAR, including, for example, a LMP1 CAR comprising any of the LMP1-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP1 CAR may additionally comprise a signal peptide as described.

In some embodiments, the LMP1 CAR comprises an anti-LMP1 scFv sequence as set forth in SEQ ID NO: 140, 141, or 142, or an scFv sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 140, 141, or 142, with the following components: CD8α signal peptide, anti-LMP1 scFv (V_{L}-Whitlow linker-V_{H}), CD8α hinge domain, CD8α transmembrane domain, 4-1BB costimulatory domain, and CD37 signaling domain.

### LMP2 CAR

In some embodiments, the CAR is a LMP2 CAR (“LMP2-CAR”), and in these embodiments, the polycistronic vector comprises an expression cassette that contains a nucleotide sequence encoding a LMP2 CAR. Similarly to LMP1, LMP2 is a large multipass transmembrane oncoprotein that immortalizes B cells and non-lymphoid tissues, leading to survival of EBV-infected cells. LMP2 is involved in the latency stage of EBV infection, and is able to redirect BCR signaling. The expression of LMP2 has been shown to be related to most EBV-associated lymphoproliferative disorders and malignancies. Patients suffering multiple sclerosis often display high numbers of LMP2-specific T cells. In some embodiments, the LMP2 CAR may comprise a signal peptide, an extracellular binding domain that specifically binds LMP2, a hinge domain, a transmembrane domain, an intracellular costimulatory domain, and/or an intracellular signaling domain in tandem.

In some embodiments, the signal peptide of the LMP2 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the LMP2 CAR is specific to LMP2, for example, viral LMP2. The binding domain of the LMP2 CAR can be codon-optimized for expression in a host cell or to have variant sequences to increase functions of the extracellular binding domain.

In some embodiments, the binding domain comprises an immunogenically active portion of an immunoglobulin molecule, for example, an scFv. In some embodiments, the binding domain of the LMP2 CAR is derived from an antibody specific to LMP2, including, for example, MHC-CN0186, MHC-CN0185, MHC-CN0144, MHC-CN0143, MHC-CN0142, MHC-CN0141, MHC-CN0140, MHC-CN0139, MHC-CN0120, MHC-CN0119, MHC-CN0092, MHC-CN0091, MHC-CN0080, MHC-CN0079, MHC-CN0249, MHC-CN-0250, MHC-CN0251, MHC-CN0252, MHC-CN0282, MHC-CN0281, all commercially available through Creative-Biolabs. In any of these embodiments, the extracellular binding domain of the LMP2 CAR can comprise or consist of the V_{H}, the V_{L}, and/or one or more CDRs of any of the antibodies.

In some embodiments, the extracellular binding domain of the LMP2 CAR comprises an scFv derived from an LMP2-specific TCR as described in PCT Patent Application No WO 2021/211455 A1. The LMP2-specific TCR-derived scFv may comprise the LMP2-specific binding sequences of SEQ ID NOs: 143-148, provided in Table 22 below. In some embodiments, the LMP2-specific extracellular binding domain comprises or consists of an amino acid sequence set forth in SEQ ID NOs: 143-148, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 143-148. In some embodiments, the LMP2-specific extracellular binding domain may comprise one or more CDRs having amino acid sequences set forth in SEQ ID NOs: 143-148. In some embodiments, the LMP2-specific extracellular binding domain may comprise a light chain with one or more CDRs having amino acid sequences set forth in SEQ ID NOs: 143-148. In some embodiments, the LMP2-specific extracellular binding domain may comprise a heavy chain with one or more CDRs having amino acid sequences set forth in SEQ ID NOs: 143-148. In any of these embodiments, the LMP2-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the LMP2 CAR comprises or consists of the one or more CDRs as described herein.

In some embodiments, the extracellular binding domain of the LMP2 CAR comprises an scFv derived from HLA class I-restricted T cell receptors directed against LMP2 as described in PCT Patent Application No. WO2021211455, Hinrichs and Liu, the amino acid sequence of which is provided in Table 23 below. In some embodiments, the LMP2-specific extracellular binding domain comprises or consists of an amino acid sequence set forth in SEQ ID NO: 143 and 144, or 145 and 145, or 147 and 148, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 143 and 144, or 145 and 145, or 147 and 148. In some embodiments, the LMP2-specific extracellular binding domain may comprise one or more CDRs having amino acid sequences bolded in SEQ ID NOs: 143 and 144, or 145 and 145, or 147 and 148. In some embodiments, the LMP2-specific extracellular binding domain may comprise a light (alpha) chain with one or more CDRs having amino acid sequences set forth in SEQ ID NOs: 143, 145, or 147. In some embodiments, the LMP2-specific extracellular binding domain may comprise a heavy (beta) chain with one or more CDRs having amino acid sequences set forth in SEQ ID NOs: 144, 146 or 148. In any of these embodiments, the LMP2-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the LMP2 CAR comprises or consists of the one or more CDRs as described herein.

TABLE 23
Exemplary sequences of anti-LMP2 binders

| SEQ ID NO: | Amino Acid Sequence | Description |
|---|---|---|
| 143 | METLLGLLILWLQLQWVSSKQEVTQIPAALS | TCR D6 LMP2-specific |
| | VPEGENVLVLNCSFTDSAIYNLQWFRQDPG | alpha variable domain |
| | KGLTSLLLIQSSQREQTSGRLNASLDKSSGRS | with N-terminal signal |
| | TLYIAASQPGDSATYLCAVLMDSNYQLIWGA | peptide |
| | GTKLIIKP | (SEQ ID NO: 20 from WO |
| | | 2021/211455A1) |
| | | (CDRs are bolded) |
| 144 | MATRLFFYVALCLLWAGHRDAGITQSPRYKIT | TCR D6 LMP2-specific |
| | ETGRQVTLMCHQTWSHSYMFWYRQDLGH | beta variable domain |
| | GLRLIYYSAAADITDKGEVPDGYVVSRSKTEN | with N-terminal signal |
| | FPLTLESATRSQTSVYFCASSEDGMNTEAFF | peptide |
| | GQGTRLTVV | (SEQ ID NO: 21 from WO |
| | | 2021/211455A1) |
| | | (CDRs are bolded) |
| 145 | MLLITSMLVLWMQLSQVNGQQVMQIPQY | TCR D2 LMP2-specific |
| | QHVQEGEDFTTYCNSSTTLSNIQWYKQRPG | alpha chain variable |
| | GHPVFLIQLVKSGEVKKQKRLTFQFGEAKKN | domain with N-terminal |
| | SSLHITATQTTDVGTYFCAGSGAGSYQLTFGK | signal peptide |
| | GTKLSVIP | (SEQ ID NO: 22 from |
| | | WO2021211455A1) |
| | | (CDRs are bolded) |
| 146 | MASRLLCWVLLCLLGAGPVKAGVTQTPRYLI | TCR D2 LMP2-specific |
| | KTRGQQVTLSCSPISGHRSVSWYQQTPGQG | beta chain variable |
| | LQFLFEYFSETQRNKGNFPGRFSGRQFSNSR | domain with N-terminal |
| | SEMNVSTLELGDSALYLCASSLEGQASSYEQ | signal peptide |
| | YFGPGTRLTVT | (SEQ ID NO: 23 from |
| | | WO2021211455A1) |
| | | (CDRs are bolded) |
| 147 | MLLITSMLVLWMQLSQVNGQQVMQIPQY | TCR D29 LMP2-specific |
| | QHVQEGEDFTTYCNSSTTLSNIQWYKQRPG | alpha variable domain |
| | GHPVFLIQLVKSGEVKKQKRLTFQFGEAKKN | with N-terminal signal |
| | SSLHITATQTTDVGTYFCAATLWVFGNEKLTF | peptide |
| | GTGTRLTIIP | (SEQ ID NO: 24 from |
| | | WO2021211455A1) |
| | | (CDRs are bolded) |
| 148 | MALLLLLLGPGSGLGAVVSQHPSWVICKSGT | TCR D29 LMP2-specific |
| | SVKIECRSLDFQATTMFWYRQFPKQSLMLM | beta variable domain |
| | ATSNEGSKATYEQGVEKDKFLINHASLTLSTL | with N-terminal signal |
| | TVTSAHPEDSSFYICSARDVRGTDVYEQYFG | peptide |
| | PGTRLTVT | (SEQ ID NO: 25 from |
| | | WO2021211455A1) |
| | | (CDRs are bolded) |

In some embodiments, the hinge domain of the LMP2 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the LMP2 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the LMP2 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the LMP2 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a LMP2 CAR, including, for example, a LMP2 CAR comprising any of the LMP2-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP2 CAR may additionally comprise a signal peptide (e.g., a CD8α signal peptide) as described.

In some embodiments, the polycistronic vector comprises an expression cassette that contains a nucleotide sequence encoding a LMP2 CAR, including, for example, a LMP2 CAR comprising any of the LMP2-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP2 CAR may additionally comprise a signal peptide as described.

In some embodiments, the LMP2 CAR comprise the LMP2-specific binding sequences of SEQ ID NOs: 143-148 or LMP2-specific binding sequences at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 143-148, with the following components: CD8α signal peptide, LMP2-specific scFv (V_{L}-Whitlow linker-V_{H}), CD8α hinge domain, CD8α transmembrane domain, 4-1BB costimulatory domain, and CD3ζ signaling domain.

### LMP2A CAR

In some embodiments, the CAR is a LMP2A CAR (“LMP2A-CAR”), and in these embodiments, the polycistronic vector comprises an expression cassette that contains a nucleotide sequence encoding a LMP2A CAR. Similarly to LMP1, LMP2A is a large multipass transmembrane oncoprotein that immortalizes B cells and non-lymphoid tissues, leading to survival of EBV-infected cells. LMP2A is involved in the latency stage of EBV infection, and is able to redirect BCR signaling. The expression of LMP2A has been shown to be related to most EBV-associated lymphoproliferative disorders and malignancies. Patients suffering multiple sclerosis often display high numbers of LMP2A-specific T cells. In some embodiments, the LMP2A CAR may comprise a signal peptide, an extracellular binding domain that specifically binds LMP2A, a hinge domain, a transmembrane domain, an intracellular costimulatory domain, and/or an intracellular signaling domain in tandem.

In some embodiments, the signal peptide of the LMP2A CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the LMP2A CAR is specific to LMP2A, for example, viral LMP2A. The binding domain of the LMP2A CAR can be codon-optimized for expression in a host cell or to have variant sequences to increase functions of the extracellular binding domain.

In some embodiments, the binding domain comprises an immunogenically active portion of an immunoglobulin molecule, for example, an scFv. In some embodiments, the binding domain of the LMP2A CAR is derived from an antibody or CTR specific to LMP2A, including, for example, TCR-LA-ZP153, TCR-LA-ZP154, TLA-CA-151PZ, TLA-CA-154PZ, TLA-CA-339PZ, TLZ-CA-153PZ, or TLA-CA-338PZ, all commercially available through Creative Biolabs. In any of these embodiments, the extracellular binding domain of the LMP2A CAR can comprise or consist of the V_{H}, the V_{L}, and/or one or more CDRs of any of the antibodies.

In some embodiments, the extracellular binding domain of the LMP2A CAR comprises an scFv derived from an LMP2A-specific TCR. The LMP2A-specific TCR-derived scFv may comprise the LMP2A-specific binding sequences of SEQ ID NOs: 149-156, provided in Table 23 below. In some embodiments, the LMP2A-specific extracellular binding domain comprises or consists of an amino acid sequence set forth in SEQ ID NOs: 149-156, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 149-156. In some embodiments, the LMP2A-specific extracellular binding domain may comprise one or more CDRs having amino acid sequences set forth in SEQ ID NOS: 149-156. In some embodiments, the LMP2A-specific extracellular binding domain may comprise a light chain with one or more CDRs having amino acid sequences set forth in SEQ ID NOs: 149-156. In some embodiments, the LMP2A-specific extracellular binding domain may comprise a heavy chain with one or more CDRs having amino acid sequences set forth in SEQ ID NOs: 149-156. In any of these embodiments, the LMP2A-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the LMP2A CAR comprises or consists of the one or more CDRs as described herein. In some embodiments, LMP2A-specific extracellular binding domains can also bind to LMP2B.

In some embodiments, the extracellular binding domain of the LMP2A CAR comprises an scFv derived from HLA-A2 restricted LMP2A peptides as described in US Patent Application 2016/0199479, the amino acid sequence of which is provided in Table 24 below. In some embodiments, the LMP2A-specific extracellular binding domain comprises or consists of an amino acid sequence set forth in SEQ ID NO: 149 and 150, or 151 and 152, or 153 and 154, or 155 and 156, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 149 and 150, or 151 and 152, or 153 and 154, or 155 and 156. In some embodiments, the LMP2A-specific extracellular binding domain may comprise one or more CDRs having amino acid sequences bolded in SEQ ID NOS: 149, 150, 151, 152, 153, 154, 155 or 156. In some embodiments, the LMP2A-specific extracellular binding domain may comprise a light (alpha) chain with one or more CDRs having amino acid sequences set forth in SEQ ID NOs: 149, 151, 153 or 155. In some embodiments, the LMP2A-specific extracellular binding domain may comprise a heavy (beta) chain with one or more CDRs having amino acid sequences set forth in SEQ ID NOs: 150, 152, 154 or 156. In any of these embodiments, the LMP2A-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the LMP2A CAR comprises or consists of the one or more CDRs as described herein.

TABLE 24
Exemplary sequences of anti-LMP2A binding domains

| SEQ ID NO: | Amino Acid Sequence | Description |
|---|---|---|
| 149 | QKEVEQDPGPLSVPEGAIVSLNCTYSNSAFQ | EBV LMP2A-specific TCR |
| | YFMWYRQYSRKGPELLMYTYSSGNKEDGRF | alpha chain variable |
| | TAQVDKSSKYISLFIRDSQPSDSATYLCAMSA | domain |
| | EAHQPYRLNFGKGTSLLVTPHIQNPDPAWQ | (SEQ ID NO: 6 from US |
| | LRDSKSSDKSVCLFTDFDSQTNVSQSKDSDW | 2016/0199479) |
| | ITDKCVLDMRSMDFKSNSAVAWSNKSDFAC | (CDRs are bolded) |
| | ANAFNNSIIPEDTFFPSPESS | |
| 150 | QKEVEQDPGPLSVPEGAIVSLNCTYSNSAFQ | EBV LMP2A-specific TCR |
| | YFMWYRQYSRKGPELLMYLPGGGNKEDGR | alpha chain variable |
| | FTAQVDKSSKYISLFIRDSQPSDSATYLCAMS | domain |
| | ADAYGHYALNFGKGTSLLVTPHIQNPDPAVY | (SEQ ID NO: 7 from US |
| | QLRDSKSSDKSVCLFTDFDSQTNVSQSKDSD | 2016/0199479) |
| | VYITDKCVLDMRSMDFKSNSAVAWSNKSDF | (CDRs are bolded) |
| | ACANAFNNSIIPEDTFFPSPESS | |
| 151 | QKEVEQDPGPLSVPEGAIVSLNCTYSNSAFQ | EBV LMP2A-specific TCR |
| | YFMWYRQYSRKGPELLMYQFQDGNKEDG | alpha chain variable |
| | RFTAQVDKSSKYISLFIRDSQPSDSATYLCAM | domain |
| | SAEAHQPYRLNFGKGTSLLVTPHIQNPDPAV | (SEQ ID NO: 8 from US |
| | YQLRDSKSSDKSVCLFTDFDSQTNVSQSKDS | 2016/0199479) |
| | DVYITDKCVLDMRSMDFKSNSAVAWSNKSD | (CDRs are bolded) |
| | FACANAFNNSIIPEDTFFPSPESS | |
| 152 | QKEVEQDPGPLSVPEGAIVSLNCTYSNPHM | EBV LMP2A-specific TCR |
| | AYFMWYRQYSRKGPELLMYTYSSGNKEDG | alpha chain variable |
| | RFTAQVDKSSKYISLFIRDSQPSDSATYLCAM | domain SEQ ID NO: 9 |
| | SADAYGHYALNFGKGTSLLVTPHIQNPDPAV | from US20160199479) |
| | YQLRDSKSSDKSVCLFTDFDSQTNVSQSKDS | (CDRs are bolded) |
| | DVYITDKCVLDMRSMDFKSNSAVAWSNKSD | |
| | FACANAFNNSIIPEDTFFPSPESS | |
| 153 | QKEVEQDPGPLSVPEGAIVSLNCTYSNPHM | EBV LMP2A-specific TCR |
| | AYFMWYRQYSRKGPELLMYTYSSGNKEDG | alpha chain variable |
| | RFTAQVDKSSKYISLFIRDSQPSDSATYLCAM | domain |
| | SAEAHQPYRLNFGKGTSLLVTPHIQNPDPAV | (SEQ ID NO: 10 from |
| | YQLRDSKSSDKSVCLFTDFDSQTNVSQSKDS | US20160199479) |
| | DVYITDKCVLDMRSMDFKSNSAVAWSNKSD | (CDRs are bolded) |
| | FACANAFNNSIIPEDTFFPSPESS | |
| 154 | QKEVEQDPGPLSVPEGAIVSLNCTYSNPHM | EBV LMP2A-specific TCR |
| | AYFMWYRQYSRKGPELLMYQFQDGNKEDG | alpha chain variable |
| | RFTAQVDKSSKYISLFIRDSQPSDSATYLCAM | domain |
| | SADAYGHYALNFGKGTSLLVTPHIQNPDPAV | (SEQ ID NO: 11 from US |
| | YQLRDSKSSDKSVCLFTDFDSQTNVSQSKDS | 2016/0199479) |
| | DVYITDKf.VLDMRSMDFKSNSAVAWSNKSD | (CDRs are bolded) |
| | FACANAFNNSIIPEDTFFPSPESS | |
| 155 | QKEVEQDPGPLSVPEGAIVSLNCTYSNPHM | EBV LMP2A-specific TCR |
| | AYFMWYRQYSRKGPELLMYQFQDGNKEDG | alpha chain variable |
| | RFTAQVDKSSKYISLFIRDSQPSDSATYLCAM | domain |
| | SAEAHQPYRLNFGKGTSLLVTPHIQNPDPAV | (SEQ ID NO: 12 from US |
| | YQLRDSKSSDKSVCLFTDFDSQTNVSQSKDS | 2016/0199479) |
| | DVYITDKf.VLDMRSMDFKSNSAVAWSNKSD | (CDRs are bolded) |
| | FACANAFNNSIIPEDTFFPSPESS | |
| 156 | EAGVAQSPRYKIIEKRQSVAFWCNPISGHATL | EBV LMP2A-specific TCR |
| | YWYQQILGQGPKLLIQFVVAASVDDSQLPK | beta chain variable |
| | DRFSAERLKGVDSTLKIQPAKLEDSAVYLCAS | domain |
| | SLGGYEQYFGPGTRLTVTEDLKNVFPPEVAV | (SEQ ID NO: 13 from |
| | FEPSEAEISHTQKATLVCLATGFYPDHVELSVV | US20160199479) |
| | WVNGKEVHSGVETDPQPLKEQPALNDSRYA | (CDRs are bolded) |
| | LSSRLRVSATFWQDPRNHFRCQVQFYGLSE | |
| | NDEWTQDRAKPVTQIVSAEAWGRAD | |

In some embodiments, the hinge domain of the LMP2A CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the LMP2A CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the LMP2A CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the LMP2A CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a LMP2A CAR, including, for example, a LMP2A CAR comprising any of the LMP2A-specific extracellular binding domains as described, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP2A CAR may additionally comprise a signal peptide (e.g., a CD8α signal peptide) as described.

In some embodiments, the polycistronic vector comprises an expression cassette that contains a nucleotide sequence encoding a LMP2A CAR, including, for example, a LMP2A CAR comprising any of the LMP2A-specific extracellular binding domains as described, the CD8α hinge domain of SEQ ID NO:9, the CD8α transmembrane domain of SEQ ID NO:14, the CD28 costimulatory domain of SEQ ID NO:17, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the LMP2A CAR may additionally comprise a signal peptide as described.

In some embodiments, the LMP2A CAR comprises the LMP2A-specific binding sequences of SEQ ID NOs: 149-156 or LMP2A-specific binding sequences at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 149-156, with the following components: CD8α signal peptide, LMP2-specific scFv (V_{L}-Whitlow linker-V_{H}), CD8α hinge domain, CD8α transmembrane domain, 4-1BB costimulatory domain, and CD3ζ signaling domain.

### BZLF1 CAR

In some embodiments, the CAR is a BZLF1 CAR (“BZLF1-CAR”), and in these embodiments, the polycistronic vector comprises an expression cassette that contains a nucleotide sequence encoding a BZLF1 CAR. BZLF1 is a EBV transcription factor expressed during the lytic phase of the viral cycle leading to the production of infectious viral particles. BZLF1 is involved in the lytic EBV replication from fully methylated DNA. The expression of BZLF1 has been shown to be related to B cell oncogenesis with replication. Patients suffering multiple sclerosis often display high numbers of BZLF1-specific T cells. In some embodiments, the BZLF1 CAR may comprise a signal peptide, an extracellular binding domain that specifically binds BZLF1, a hinge domain, a transmembrane domain, an intracellular costimulatory domain, and/or an intracellular signaling domain in tandem.

In some embodiments, the signal peptide of the BZLF1 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the BZLF1 CAR is specific to BZLF1, for example, viral BZLF1. The extracellular binding domain of the BZLF1 CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BZLF1 CAR is derived from any of the antibodies or CARs disclosed in Table 25. In any of these embodiments, the extracellular binding domain of the BZLF1 CAR can comprise or consist of the V_{H}, the V_{L}, and/or one or more CDRs of any of the antibodies.

In some embodiments, the extracellular binding domain of the BZLF1 CAR comprises an scFv derived from any of the antibodies or CARs disclosed in Table 25, optionally comprising the heavy chain variable region (V_{H}) and the light chain variable region (V_{L}) of one of the antibodies or CARs, connected by a linker. In some embodiments, the linker is a 3xG₄S (SEQ ID NO: 30) linker. In other embodiments, the Whitlow linker may be used instead. In some embodiments, the BZLF1-specific extracellular binding domain comprises or consists of an antibody or CAR disclosed in Table 15, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 25. In some embodiments, the BZLF1-specific extracellular binding domain may comprise one or more CDRs having amino acid sequences of the CDRs of an antibody or CAR disclosed in Table 25. In some embodiments, the BZLF1-specific scFv may comprise a heavy chain with one or more CDRs having amino acid sequences of the CDRs of an antibody or CAR disclosed in Table 25. In some embodiments, the BZLF1-specific scFv may comprise a light chain with one or more CDRs having amino acid sequences of the CDRs of an antibody or CAR disclosed in Table 25. In any of these embodiments, the BZLF1-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BZLF1 CAR comprises or consists of the one or more CDRs as described herein, including in Table 25.

TABLE 25
Exemplary BZLF1 antigen binding domains

| Ab/CAR Name | Antigen | Company | Reference |
|---|---|---|---|
| scTCR-063ZP-S(P) | BZLF1 | Creative Biolabs | Creative-biolabs.net |
| scTCR-062ZP-S(P) | BZLF1 | Creative Biolabs | Creative-biolabs.net |
| scTCR-061ZP-S(P) | BZLF1 | Creative Biolabs | Creative-biolabs.net |
| scTCR-060ZP-S(P) | BZLF1 | Creative Biolabs | Creative-biolabs.net |
| scTCR-059ZP-S(P) | BZLF1 | Creative Biolabs | Creative-biolabs.net |
| scTCR-058ZP-S(P) | BZLF1 | Creative Biolabs | Creative-biolabs.net |
| scTCR-057ZP-S(P) | BZLF1 | Creative Biolabs | Creative-biolabs.net |
| scTCR-056ZP-S(P) | BZLF1 | Creative Biolabs | Creative-biolabs.net |
| scTCR-055ZP-S(P) | BZLF1 | Creative Biolabs | Creative-biolabs.net |
| scTCR-054ZP-S(P) | BZLF1 | Creative Biolabs | Creative-biolabs.net |
| scTCR-053ZP-S(P) | BZLF1 | Creative Biolabs | Creative-biolabs.net |
| scTCR-052ZP-S(P) | BZLF1 | Creative Biolabs | Creative-biolabs.net |
| scTCR-051ZP-S(P) | BZLF1 | Creative Biolabs | Creative-biolabs.net |
| scTCR-050ZP-S(P) | BZLF1 | Creative Biolabs | Creative-biolabs.net |
| scTCR-064ZP-S(P) | BZLF1 | Creative Biolabs | Creative-biolabs.net |
| scTCR-065ZP-S(P) | BZLF1 | Creative Biolabs | Creative-biolabs.net |

In some embodiments, the hinge domain of the BZLF1 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the BZLF1 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BZLF1 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BZLF1 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BZLF1 CAR, including, for example, a BZLF1 CAR comprising any of the BZLF1-specific scFv having sequences of an antibody or CAR disclosed in Table 25, the CD8α hinge domain of SEQ ID NO:9, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BZLF1 CAR, including, for example, a BZLF1 CAR comprising any of the BZLF1-specific scFv having sequences of an antibody or CAR disclosed in Table 25, the CD28 hinge domain of SEQ ID NO:10, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BZLF1 CAR, including, for example, a BZLF1 CAR comprising the BZLF1-specific scFv having sequences of an antibody or CAR disclosed in Table 25, the IgG4 hinge domain of SEQ ID NO:11 or SEQ ID NO:12, the CD8α transmembrane domain of SEQ ID NO: 14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### BMLF1 CAR

In some embodiments, the CAR is a BMLF1 CAR (“BMLF1-CAR”), and in these embodiments, the polycistronic vector comprises an expression cassette that contains a nucleotide sequence encoding a BMLF1 CAR. BMLF1 is a multifunctional RNA-binding viral protein that increases either viral or cellular gene expression during the EBV lytic phase. The expression of BMLF1 has been shown to be related to most EBV-associated malignancies. Patients suffering multiple sclerosis often display high numbers of BMLF1-specific T cells. In some embodiments, the BMLF1 CAR may comprise a signal peptide, an extracellular binding domain that specifically binds BMLF1, a hinge domain, a transmembrane domain, an intracellular costimulatory domain, and/or an intracellular signaling domain in tandem.

In some embodiments, the signal peptide of the BMLF1 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the BMLF1 CAR is specific to BMLF1, for example, viral BMLF1. The extracellular binding domain of the BMLF1 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the BMLF1 CAR is derived from an antibody specific to BMLF1, including, for example, any of the antibodies or CARs disclosed in Table 26, the references cited in which are incorporated by reference in their entireties herein. In any of these embodiments, the extracellular binding domain of the BMLF1 CAR can comprise or consist of the V_{H}, the V_{L}, and/or one or more CDRs of any of the antibodies disclosed in Table 26

In some embodiments, the extracellular binding domain of the BMLF1 CAR comprises an scFv derived from the any of the antibodies or CARs disclosed in Table 26, optionally comprising the heavy chain variable region (V_{H}) and the light chain variable region (V_{L}) of one of the antibodies or CARs, connected by a linker. In some embodiments, the linker is a 3xG₄S (SEQ ID NO: 30) linker. In other embodiments, the Whitlow linker may be used instead. In some embodiments, the BMLF1-specific scFv comprises or consists of the scFv of an antibody or CAR disclosed in Table 26, or an amino acid sequence that is at least 80% identical (e.g., at least 80%, at least 85%, at least 90%, at least 95%, at least 96%, at least 97%, at least 98%, at least 99%, or 100% identical) to the amino acid sequence of the scFv of an antibody or CAR disclosed in Table 26. In some embodiments, the BMLF1-specific scFv may comprise one or more CDRs having amino acid sequences of the CDRs of an antibody or CAR disclosed in Table 26. In some embodiments, the BMLF1-specific scFv may comprise a heavy chain with one or more CDRs having amino acid sequences of the CDRs of an antibody or CAR disclosed in Table 26. In some embodiments, the BMLF1-specific scFv may comprise a light chain with one or more CDRs having amino acid sequences of the CDRs of an antibody or CAR disclosed in Table 26. In any of these embodiments, the BMLF1-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BMLF1 CAR comprises or consists of the one or more CDRs as described herein, including in Table 26.

TABLE 26
Exemplary BMLF1 antigen binding domains

| Ab/CAR Name | Antigen | Company | Reference |
|---|---|---|---|
| scTCR-011CQ-S(P) | BMLF1 | Creative Biolabs | Creative-biolabs.net |
| MHC-LC1640 | BMLF1 | Creative Biolabs | Creative-biolabs.net |
| MHC-LC1642 | BMLF1 | Creative Biolabs | Creative-biolabs.net |
| MHC-LC715 | BMLF1 | Creative Biolabs | Creative-biolabs.net |
| MHC-YC117 | BMLF1 | Creative Biolabs | Creative-biolabs.net |
| MHC-LC225 | BMLF1 | Creative Biolabs | Creative-biolabs.net |

In some embodiments, the hinge domain of the BMLF1 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the BMLF1 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the BMLF1 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the BMLF1 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the CD28 hinge domain of SEQ ID NO:10, the CD8α transmembrane domain of SEQ ID NO: 14, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the IgG4 hinge domain of SEQ ID NO:11 or SEQ ID NO:12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the CD28 hinge domain of SEQ ID NO:10, the CD28 transmembrane domain of SEQ ID NO: 15, the 4-1BB costimulatory domain of SEQ ID NO:16, the CD3ζ signaling domain of SEQ ID NO: 18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a BMLF1 CAR, including, for example, a BMLF1 CAR comprising the BMLF1-specific scFv having sequences of an antibody or CAR disclosed in Table 26, the IgG4 hinge domain of SEQ ID NO:11 or SEQ ID NO:12, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### Gp350 CAR

In some embodiments, the CAR is a gp350 CAR (“gp350-CAR”), and in these embodiments, the polycistronic vector comprises an expression cassette that contains a nucleotide sequence encoding a gp350 CAR. gp350 is the most abundant EBV surface glycoprotein and is important for the attachment of the virus to B cells through interactions with the surface receptors CD21 (a.k.a. CR2) or CD35 (a.k.a. CR1). gp350 positive cells are also sometimes found in cases of B cell lymphomes, adult T cell leukemia/lymphoma, and acute myeloid leukemia. In some embodiments, the gp350 CAR may comprise a signal peptide, an extracellular binding domain that specifically binds gp350, a hinge domain, a transmembrane domain, an intracellular costimulatory domain, and/or an intracellular signaling domain in tandem.

In some embodiments, the signal peptide of the gp350 CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the gp350 CAR is specific to gp350, for example, viral gp350. The extracellular binding domain of the gp350 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the gp350 CAR is derived from an antibody specific to gp350, including, for example, any of the antibodies or CARs disclosed in Table 27, the references cited in which are incorporated by reference in their entireties herein. In any of these embodiments, the extracellular binding domain of the gp350 CAR can comprise or consist of the V_{H}, the V_{L}, and/or one or more CDRs of any of the antibodies in Table 27, namely the 72A1 monoclonal antibody as described by Mutsvunguma et al., Virology 536:1-15 (2019). In any of these embodiments, the extracellular binding domain of the gp350 CAR can comprise or consist of the V_{H}, the V_{L}, and/or one or more CDRs of any of the antibodies disclosed in Table 27.

TABLE 27
Exemplary gp350 antigen binding domains

| Ab/CAR | | |
|---|---|---|
| Name | Antigen | Reference |
| HB1 | Gp350 | Mutsvunguma et al., Virology 536: 1-15 (2019) |
| HB2 | Gp350 | Mutsvunguma et al., Virology 536: 1-15 (2019) |
| HB3 | Gp350 | Mutsvunguma et al., Virology 536: 1-15 (2019) |
| HB4 | Gp350 | Mutsvunguma et al., Virology 536: 1-15 (2019) |
| HB5 | Gp350 | Mutsvunguma et al., Virology 536: 1-15 (2019) |
| HB6 | Gp350 | Mutsvunguma et al., Virology 536: 1-15 (2019) |
| HB7 | Gp350 | Mutsvunguma et al., Virology 536: 1-15 (2019) |
| HB8 | Gp350 | Mutsvunguma et al., Virology 536: 1-15 (2019) |
| HB9 | Gp350 | Mutsvunguma et al., Virology 536: 1-15 (2019) |
| HB10 | Gp350 | Mutsvunguma et al., Virology 536: 1-15 (2019) |
| HB11 | Gp350 | Mutsvunguma et al., Virology 536: 1-15 (2019) |
| HB12 | Gp350 | Mutsvunguma et al., Virology 536: 1-15 (2019) |
| HB13 | Gp350 | Mutsvunguma et al., Virology 536: 1-15 (2019) |
| HB14 | Gp350 | Mutsvunguma et al., Virology 536: 1-15 (2019) |
| HB15 | Gp350 | Mutsvunguma et al., Virology 536: 1-15 (2019) |
| HB16 | Gp350 | Mutsvunguma et al., Virology 536: 1-15 (2019) |
| HB17 | Gp350 | Mutsvunguma et al., Virology 536: 1-15 (2019) |
| HB18 | Gp350 | Mutsvunguma et al., Virology 536: 1-15 (2019) |
| HB19 | Gp350 | Mutsvunguma et al., Virology 536: 1-15 (2019) |
| HB20 | Gp350 | Mutsvunguma et al., Virology 536: 1-15 (2019) |
| HB21 | Gp350 | Mutsvunguma et al., Virology 536: 1-15 (2019) |
| HB22 | Gp350 | Mutsvunguma et al., Virology 536: 1-15 (2019) |
| h72A1 | Gp350 | Mutsvunguma et al., Virology 536: 1-15 (2019) |

In some embodiments, the extracellular binding domain of the gp350 CAR comprises an scFv derived from the 7A1, the 6G4, or the humanized 72A1 monoclonal antibody, which comprise the heavy chain variable region (V_{H}) and the light chain variable region (V_{L}) of 7A1,6G4 or h72A1 connected by a linker. See PCT Application No. WO 2019/201995 A1, Stripecke et al.; Mustvunguma et al., 2019, incorporated herein by reference in their entireties. In some embodiments, the linker is a 3xG₄S (SEQ ID NO: 30) linker. In other embodiments, the linker is a Whitlow linker as described herein. In some embodiments, the amino acid sequences of different portions of the 7A1-derived scFv (also referred to as 7A1 scFv), the 6G4-derived scFv (also referred to as 6G4 scFv), or the humanized 72A1-derived scFv and their different portions are provided in Table 28 below. In some embodiments, the gp350-specific scFv comprises or consists of the heavy and light chain sequences sequence set forth in SEQ ID NOs: 157, 158, 159, 160, 161, or 162, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 157, 158, 159, 160, 161, or 162. In some embodiments, the gp350-specific scFv may comprise one or more CDRs having amino acid sequences bolded in SEQ ID NOs: 157, 158, 159, 160, 161, or 162. In some embodiments, the gp350-specific scFv may comprise a light chain with one or more CDRs having amino acid sequences set forth in SEQ ID NOs: 158, 160, and 162. In some embodiments, the gp350-specific scFv may comprise a heavy chain with one or more CDRs having amino acid sequences set forth in SEQ ID NOs: 157, 159, and 161. In any of these embodiments, the gp350-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the gp350 CAR comprises or consists of the one or more CDRs as described herein.

TABLE 28
Exemplary sequences of anti-gp350 scFv components

| SEQ ID NO: | Amino Acid Sequence | Description |
|---|---|---|
| 157 | QVQLKESGPGLVQPSQTLSLTCTVSGLSLTSN | 7A1 V_{H} |
| | GVSWIRQPPGKGLVHEWLGVIWSNGGTDY | (SEQ ID NO: 15 from WO |
| | NSAIKSRLSFSRDTSKSQVFLKMNSLQTEDTA | 2019/201995A1) |
| | MYFCARPRYNSGYFFDYWGQGVMVTVSS | (CDRs are bolded) |
| 158 | DTVLTQSPALAVSPGERVTISCKASESVSTRM | 7A1 V_{L} |
| | HWYRQKPGQQPKLLIYKTSNLASGVPARFS | (SEQ ID NO: 16 from WO |
| | GSGSGTDFTLTIDPVEADDTATYFCQQSWN | 2019/201995A1) |
| | GPLTFGSGTKLEIKR | (CDRs are bolded) |
| 159 | QVQLKESGPGLVQPSQTLSLTCTVSGFSLTSY | 6G4 V_{H} |
| | HVHWVRQPPGKGLEWMGVMWSDGDTLY | (SEQ ID NO: 17 from WO |
| | NSALKSRLSISRDTSKSQVLLQMDSLQSEDTA | 2019/201995A1) |
| | TYYCARLQVFGYPGIRDYVMDAWGQGASV | (CDRs are bolded) |
| | TVSS | |
| 160 | DLVMTQSPFSLAVSEGEMVTIKCKSSQSLLSS | 6G4 V_{L} |
| | RHQKNFLAWYRQKPGQSPKLLIYHASTRQS | (SEQ ID NO: 18 from WO |
| | GVPDRFIGSGSGTDFTLTISDVQAEDLADYYC | 2019/201995A1) |
| | LQHYTSPYTFGAGTKLELKR | (CDRs bolded) |
| 161 | EVQLVESGGGLVQPGGSLRLSCAASGSSFTD | h72A1 V_{H} |
| | YTMNWMRQSPGKGLEWIGLINPYNGGTRY | (CDRs are bolded) |
| | ADSVKGRATLSLDKSKNTAYLQMNSLRAEDT | (from Mutsvunguma et |
| | AIYYCAGGLRRVNWFAYWGQGTLVTVSS | al., 2019) |
| 162 | DAQLTQSPILLSASVGDRVTITCRSSTGAVTTS | h72A1 V_{L} |
| | NYANWVQERTNGSFRGLIGGTNNRVPGVP | (CDRs are bolded) |
| | SRFSGSLSGDDATLTISSLQPEDEADYFCVLW | (from Mutsvunguma et |
| | HSNHWVFGAGTKVEIKR | al., 2019) |

In some embodiments, the hinge domain of the gp350 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO: 10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the gp350 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the gp350 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the gp350 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOs: 157, 159, 161, 158, 160, and 162, the CD8α hinge domain of SEQ ID NO:9, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOs: 157, 159, 161, 158, 160, and 162, the CD28 hinge domain of SEQ ID NO:10, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOs: 157, 159, 161, 158, 160, and 162, the IgG4 hinge domain of SEQ ID NO:11 or SEQ ID NO:12, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOs: 157, 159, 161, 158, 160, and 162, the CD8α hinge domain of SEQ ID NO:9, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOS: 157, 159, 161, 158, 160, and 162, the CD28 hinge domain of SEQ ID NO:10, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gp350 CAR, including, for example, a gp350 CAR comprising the gp350-specific scFv heavy and light chain sequences set forth in SEQ ID NOs: 157, 159, 161, 158, 160, and 162, the IgG4 hinge domain of SEQ ID NO:11 or SEQ ID NO:1, the CD28 transmembrane domain of SEQ ID NO:15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

gH/gL CAR

In some embodiments, the CAR is a gH/gL CAR (“gH/gL-CAR”), and in these embodiments, the polycistronic vector comprises an expression cassette that contains a nucleotide sequence encoding a gH/gL CAR. gH/gL is a critical EBV protein for the attachment and fusion process during viral entry into cells. In some embodiments, the gH/gL CAR may comprise a signal peptide, an extracellular binding domain that specifically binds gH/gL, a hinge domain, a transmembrane domain, an intracellular costimulatory domain, and/or an intracellular signaling domain in tandem.

In some embodiments, the signal peptide of the gH/gL CAR comprises a CD8α signal peptide. In some embodiments, the CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 6. In some embodiments, the signal peptide comprises an IgK signal peptide. In some embodiments, the IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:7. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:8.

In some embodiments, the extracellular binding domain of the gH/gL CAR is specific to gH/gL, for example, viral gH/gL. The extracellular binding domain of the gH/gL CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the gH/gL CAR is derived from an antibody specific to gH/gL, including, for example, AMMO1, AMMO2, AMMO3, AMMO4 or AMMO5 as described in U.S. Pat. No. 11,116,835, incorporated by reference. In any of these embodiments, the extracellular binding domain of the gp350 CAR can comprise or consist of the V_{H}, the V_{L}, and/or one or more CDRs of any of the antibodies.

In some embodiments, the extracellular binding domain of the gH/gL CAR comprises an scFv derived from the AMMO1 monoclonal antibody, which comprises the heavy chain variable region (V_{H}) and the light chain variable region (V_{L}) of AMMO1 connected by a linker. See U.S. Pat. No. 11,116,835, Mcguire (2018). In some embodiments, the linker is a 3xG₄S (SEQ ID NO: 30) linker. In other embodiments, the linker is a Whitlow linker as described herein. In some embodiments, the amino acid sequences of different portions of the AMMO1-derived scFv (also referred to as AMMO1 scFv) and its different portions are provided in Table 29 below. In some embodiments, the gH/gL-specific scFv comprises or consists of the heavy and light chain sequences sequence set forth in SEQ ID NOs: 164, 166, 168, 170, 172, 163, 165, 167, 169, and 171, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 164, 166, 168, 170, 172, 163, 165, 167, 169, and 171. In some embodiments, the gH/gL-specific scFv may comprise one or more CDRs having amino acid sequences bolded in SEQ ID NOs: 164, 166, 168, 170, 172, 163, 165, 167, 169, and 171. In some embodiments, the gH/gL-specific scFv may comprise a light chain with one or more CDRs having amino acid sequences set forth in SEQ ID NOs: 163, 165, 167, 169, and 171. In some embodiments, the gH/gL-specific scFv may comprise a heavy chain with one or more CDRs having amino acid sequences set forth in SEQ ID NOs: 164, 166, 168, 170, 172. In any of these embodiments, the gH/gL-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the gH/gL CAR comprises or consists of the one or more CDRs as described herein.

TABLE 29
Exemplary sequences of anti-gH/gL scFv components

| SEQ ID NO: | Amino Acid Sequence | Description |
|---|---|---|
| 163 | SVVLTQPPSVSVAPGKTARITCGGNNIGSKSV | VL for AMMO1 |
| | HWYQQKPGQAPVLVIYYDSDRPSGIPERFS | (SEQ ID NO: 65 from US |
| | GSNSGNTATLTISRVEAGDEADVYCQVWDS | 11,116,835 B2) |
| | SSDHPLYVFGTGTKVTVL | (CDRs are bolded) |
| 164 | QVQLVQSGAEVKKPGASVKVSCKASGYTFTS | VH for AMMO1 |
| | YGISWVRQAPGQGLEWMGWISAYNGNTN | (SEQ ID NO: 66 from US |
| | YAQKLQGRVTMTTDTSTSTAYMELRSLRSDD | 11,116,835 B2) |
| | TAVYVCARALEMGYRSGFPFDYWGQGTLV | (CDRs are bolded) |
| | TVSS | |
| 165 | DIQMTQSPSSVSASVGDRVTITCRASQGISS | VL for AMMO2 |
| | WLAWYQQKPGKAPKLLIYAASSLQSGVPSR | (SEQ ID NO: 67 from US |
| | FSGSGSGTDFTLTISSLQPEDFATYVCQQANS | 11,116,835 B2) |
| | FPLTFGGGTKVEIK | (CDRs are bolded) |
| 166 | QVQLVQSGAEVKKPGSSVKVSCKASGGTFSS | VH for AMMO2 |
| | YAISWVRQAPGQGLEWMGGIIPIFGTANYA | (SEQ ID NO: 68 from US |
| | QKFQGRVTITADESTSTAYMELSSLRSEDTAV | 11,116,835 B2) |
| | YVCARDRVLGAHGGNPLNGHVYGMDVW | (CDRs are bolded) |
| | GQGTTVTVSS | |
| 167 | SVELTQPPSVSVSPGQTASITCSGDKLGDKYA | VL for AMMO3 |
| | CWYQQKPGQSPVLVIYQDSKRPSGIPERFSG | (SEQ ID NO: 69 from US |
| | SNSGNTATLTISGTQAMDEADYVCQAWDSS | 11,116,835 B2) |
| | TVVFGGGTKLTVL | (CDRs are bolded) |
| 168 | QVTLKESGPTLVKPTQTLTLTCTFSGFSLSTSG | VH for AMMO3 |
| | VGVGWIRQPPGKALEWLALIYWDDDKRYG | (SEQ ID NO: 70 from US |
| | PSLKSRLTITKDTSKNQVVLTMTNMDPVDTA | 11,116,835 B2) |
| | TYYCAHRDKLYSGYVKFDYWGQGTLVTVSS | (CDRs are bolded) |
| 169 | DVVMTQSPLSLPVTLGQPASISCRSSQSLVYS | VL for AMMO4 |
| | DGNTYLNWFQQRPGQSPRRLIYKVSNRDS | (SEQ ID NO: 71 from US |
| | GVPDRFSGSGSGTDFTLKISRVEAEDVGVYV | 11,116,835 B2) |
| | CMQGTHWPPMYTFGQGTKLEIK | (CDRs are bolded) |
| 170 | QVQLVQSGAEVKKPGSSVKVSCKASGGTFSS | VH for AMMO4 |
| | YAISWVRQAPGQGLEWMGGIIPILGIANYA | (SEQ ID NO: 72 from US |
| | QKFQGRVTITADKSTSTAYMELSSLRSEDTAV | 11,116,835 B2) |
| | YYCARDVPGQCTRTSCYNFSSQWGQGTLVT | (CDRs are bolded) |
| | VSS | |
| 171 | EIVLTQSPGTLSLSPGERATLSCRASQSVSSSY | VL for AMMO5 |
| | LAWYQQKPGQAPRLLIYGASSRATGIPDRFS | (SEQ ID NO: 73 from US |
| | GSGSGTDFTLTISRLEPEDFAVYYCQQYGSSP | 11,116,835 B2) |
| | PRTFGQGTKVEIK | (CDRs are bolded) |
| 172 | EVQLLESGGGLVQPGGSLRLSCAASGFTFSSY | VH for AMMO5 |
| | AMSWVRQAPGKGLEWVSAISGSGGSTYYA | (SEQ ID NO: 74 from US |
| | DSVKGRFTISRDNSKNTLYLQMNSLRAEDTA | 11,116,835 B2) |
| | VYYCAKDGAGDYLGWFDPWGQGTLVTVSS | (CDRs are bolded) |

In some embodiments, the hinge domain of the gH/gL CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:13.

In some embodiments, the transmembrane domain of the gH/gL CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of the gH/gL CAR comprises a 4-1BB costimulatory domain, for example, a human 4-1BB costimulatory domain. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17.

In some embodiments, the intracellular signaling domain of the gH/gL CAR comprises a CD3 zeta (ζ) signaling domain, for example, a human CD3ζ signaling domain. In some embodiments, the CD3ζ signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, the polycistronic vector comprises an expression cassette that contains a nucleotide sequence encoding a gH/gL CAR, including, for example, a gp350 CAR comprising the gH/gL-specific scFv heavy and light chain sequences set forth in SEQ ID NOs: 164, 166, 168, 170, 172, 163, 165, 167, 169, and 171, the CD8α hinge domain of SEQ ID NO:9, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gH/gL CAR, including, for example, a gH/gL CAR comprising the gp350-specific scFv heavy and light chain sequences set forth in SEQ ID NOs: 164, 166, 168, 170, 172, 163, 165, 167, 169, and 171, the CD28 hinge domain of SEQ ID NO:10, the CD8α transmembrane domain of SEQ ID NO:14, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gH/gL CAR, including, for example, a gH/gL CAR comprising the gH/gL-specific scFv heavy and light chain sequences set forth in SEQ ID NOs: 164, 166, 168, 170, 172, 163, 165, 167, 169, and 171, the IgG4 hinge domain of SEQ ID NO:11 or SEQ ID NO: 12, the CD8α transmembrane domain of SEQ ID NO:14, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gH/gL CAR, including, for example, a gH/gL CAR comprising the gH/gL-specific scFv heavy and light chain sequences set forth in SEQ ID NOs: 164, 166, 168, 170, 172, 163, 165, 167, 169, and 171, the CD8α hinge domain of SEQ ID NO:9, the CD28 transmembrane domain of SEQ ID NO:15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gH/gL CAR, including, for example, a gH/gL CAR comprising the gH/gL-specific scFv heavy and light chain sequences set forth in SEQ ID NOs: 164, 166, 168, 170, 172, 163, 165, 167, 169, and 171, the CD28 hinge domain of SEQ ID NO:10, the CD28 transmembrane domain of SEQ ID NO:15, the 4-1BB costimulatory domain of SEQ ID NO: 16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a gH/gL CAR, including, for example, a gH/gL CAR comprising the gH/gL-specific scFv heavy and light chain sequences set forth in SEQ ID NOs: 164, 166, 168, 170, 172, 163, 165, 167, 169, and 171, the IgG4 hinge domain of SEQ ID NO:11 or SEQ ID NO: 1, the CD28 transmembrane domain of SEQ ID NO:15, the 4-1BB costimulatory domain of SEQ ID NO:16, the CD3ζ signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### S. Chimeric Autoantibody Receptors

Provided herein are hypoimmunogenic cells comprising a chimeric autoantibody receptor (CAAR). A CAAR recognizes and binds to the target autoantibodies, e.g., expressed on autoreactive cells (e.g., autoreactive B-cells).

In some embodiments, a CAAR comprises an antigen, e.g., an autoantigen that can be bound by autoantibodies. In some embodiments, a CAAR comprises a transmembrane domain. In some embodiments, a CAAR comprises a signaling domain. In some embodiments, a CAAR comprises one or more signaling domains. In some embodiments, a CAAR comprises an antigen, a transmembrane domain, and a signaling domain. In some embodiments, a CAAR comprises an antigen, a transmembrane domain, and one or more signaling domains.

A CAAR can be expressed by, e.g., a hypoimmunogenic T-cell. Thus, the present disclosure provides CAAR-T cells. CAAR T-cells can recognize and can bind target autoantibodies expressed on autoreactive cells via an antigen of a CAAR. Once a CAAR T-cell binds a target autoantibody expressed on an autoreactive cell, the CAAR T-cell can destroy the autoreactive cell.

A CAAR can be expressed by, e.g., a hypoimmunogenic NK-cell. Thus, the present disclosure provides CAAR NK-cells. CAAR NK-cells can recognize and can bind target autoantibodies expressed on autoreactive cells via an antigen of a CAAR. Once a CAAR NK-cell binds a target autoantibody expressed on an autoreactive cell, the CAAR NK-cell can destroy the autoreactive cell.

### 1. CAAR Antigens

As discussed above, provided herein are CAARs comprising an antigen. Antigens in CAARs as provided are generally known to be bound by autoantibodies. In some embodiments, autoantibodies bind autoantigens associated with an autoimmune disease. Autoantigens associated with various autoimmune diseases can be determined. For example, certain autoantibodies and the associated autoimmune disease are provided in Table 35 below:

TABLE 35
Exemplary Autoantibodies

| Disease | Autoantigen(s) |
|---|---|
| Diabetes | Pancreatic β-cell Antigen |
| Rheumatoid Arthritis | Synovial Joint Antigen |
| Expermental Autoimmune | Myelin Basic Protein, Proteolipid Protein, |
| Encephalomyelities | Myelin Oligodendritic Glycoprotein |
| Multiple Sclerosis | Myelin Basic Protein, Proteolipid Protein, |
| | Myelin Oligodendritic Glycoprotein |
| Myastenia gravis | MuSK |
| Pemphigus Vulgaris | Keratinocyte Adhesion Protien Desmoglein 3 |
| | (Dsg3) |
| Sjogren's Syndrome | Ro-RNP Complex, La antigen |
| Vasculitis | Myeloperoxidase, proteinase 3, Cardiolipin |
| Wegener's Granulomatosis | Myeloperoxidase, proteinase 3, Cardiolipin |
| Rheumatoid Arthritis | Citrullinated Proteins, Carbamylated Proteins |
| Goodpasture's Syndrome | α3 Chain of basement membrane collagen |

### 2. Transmembrane Domain

In some embodiments, a CAAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, a transmembrane domain comprises at least a transmembrane region(s) of CD8α, CD83, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### 3. Signaling Domain or Plurality of Signaling Domains

In some embodiments, a CAAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thyl; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAAR comprises a CD3 zeta domain or an immunoreceptor tyrosine-based activation motif (ITAM), or functional variant thereof. In some embodiments, the CAAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

T. Chimeric B-cell-targeting Antibody Receptors

Provided herein are hypoimmunogenic cells comprising a chimeric B-cell autoantibody receptor (BAR). A BAR recognizes and binds to certain antibody-expressing B cells.

In some embodiments, a BAR comprises an antigen. An antigen of a BAR can be bound by neutralizing antibodies. The neutralizing antibodies may be undesirable because they can block or inhibit an effect or function of antigen to which they bind. For example, hemophilia patients can receive therapeutic factor VIII (FVIII) as part of their treatment. However, a patient's body may develop an immune response against the FVIII, including the production of anti-FVIII antibodies from B cells. When the patient produces anti-FVIII antibodies that bind to FVIII, FVIII is not able to perform its therapeutic functions. Accordingly, it may be beneficial to remove the anti-FVIII antibodies and/or the B-cells producing those antibodies from the patient. A BAR, which includes an FVIII antigen, can be used for this purpose.

In some embodiments, a BAR comprises a transmembrane domain. In some embodiments, a BAR comprises a signaling domain. In some embodiments, a BAR comprises one or more signaling domains.

In some embodiments, a BAR comprises an antigen, a transmembrane domain, and a signaling domain. In some embodiments, a BAR comprises an antigen, a transmembrane domain, and one or more signaling domains.

A BAR can be expressed by, e.g., a hypoimmunogenic T-cell. Thus, the present disclosure provides BAR T-cells. BAR T-cells can recognize and can bind target select antibodies and/or the B cells producing those antibodies. Once a BAR T-cell binds a target antibody, the BAR T-cell can destroy the antibodies and/or the B cells producing those antibodies. In some embodiments, a BAR T-cell is a BAR T-cell (Treg), e.g., a regulatory T-cell (Treg) comprising a BAR.

A BAR can be expressed by, e.g., a hypoimmunogenic NK-cell. Thus, the present disclosure provides BAR NK-cells. BAR NK-cells can recognize and can bind target select antibodies and/or the B cells producing those antibodies. Once a BAR NK-cell binds a target antibody, the BAR NK-cell can destroy the antibodies and/or the B cells producing those antibodies.

### 1. BAR Antigens

As discussed above, provided herein are BARs comprising an antigen. Antigens in BARs as provided are generally known to be bound by autoantibodies. An antigen of a BAR can be bound by neutralizing antibodies. The neutralizing antibodies may be undesirable because they can block or inhibit an effect or function of antigen to which they bind.

### 2. Transmembrane Domain

In some embodiments, a BAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, a transmembrane domain comprises at least a transmembrane region(s) of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

### 3. Signaling Domain or Plurality of Signaling Domains

In some embodiments, a BAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thyl; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof. In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the BAR comprises a CD3 zeta domain or an immunoreceptor tyrosine-based activation motif (ITAM), or functional variant thereof. In some embodiments, the BAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the BAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the BAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the BAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

### U. Characteristics of Hypoimmunogenic Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

V. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient.

In some embodiments, the hypoimmunogenic cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present disclosure is directed to hypoimmunogenic primary T cells that overexpress CD47 and CARs, and have reduced expression or lack expression of one or more MHC class I and/or MHC class II human leukocyte antigen molecules and have reduced expression or lack expression of TCR complex molecules. The cells outlined herein overexpress CD47 and CARs and evade immune recognition. In some embodiments, the primary T cells display reduced levels or activity of MHC class I antigens/molecules, MHC class II antigens/molecules, and/or TCR complex molecules. In certain embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments, T cells overexpress CD47 and CARs and harbor genomic modifications in one or more of the following genes: the B2M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In certain embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some cases, the modified T cell comprise a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, the hypoimmunogenic cells described herein comprise T cells that are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In certain embodiments, the T cells described herein such as the engineered or modified T cells include reduced expression of CTLA-4 and PD-1. Methods of reducing or eliminating expression of CTLA-4, PD-1 and both CTLA-4 and PD-1 can include any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA-4 and/or PD-1 gene locus.

In some embodiments, the T cells described herein such as the engineered or modified T cells include enhanced expression of PD-L1.

In some embodiments, the hypoimmunogenic T cell includes a polynucleotide encoding a CAR, wherein the polynucleotide is inserted in a genomic locus. In some embodiments, the polynucleotide is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1 or CTLA-4 gene.

In some embodiments, the hypoimmunogenic T cell includes a polynucleotide encoding a CAR that is expressed in a cell using an expression vector. In some embodiments, the CAR is introduced to the cell using a viral expression vector that mediates integration of the CAR sequence using an into the genome of the cell. For example, the expression vector for expressing the CAR in a cell comprises a polynucleotide sequence encoding the CAR. The expression vector can be an inducible expression vector. The expression vector can be a viral vector, such as but not limited to, a lentiviral vector.

Hypoimmunogenic T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

W. Therapeutic Cells Differentiated from Hypoimmunogenic Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigen molecules. In other embodiments, the pluripotent stem cell and any cell differentiated from such a pluripotent stem cell is modified to exhibit reduced expression of MHC class II human leukocyte antigen molecules. In certain embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of one or more MHC class I and II human leukocyte antigen molecules. In some embodiments, the pluripotent stem cell and any cell differentiated from such a pluripotent stem cell is modified to exhibit reduced expression of one or more MHC class I and II human leukocyte antigen molecules and TCR complexes.

In some embodiments, the pluripotent stem cell and any cell differentiated from such a pluripotent stem cell is modified to exhibit reduced expression of one or more MHC class I and/or II human leukocyte antigen molecules and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of one or more MHC class I and II human leukocyte antigen molecules and exhibit increased CD47 expression. In some embodiments, the pluripotent stem cell and any cell differentiated from such a pluripotent stem cell is modified to exhibit reduced expression of one or more MHC class I and II human leukocyte antigen molecules and TCR complexes and exhibit increased CD47 expression.

In some embodiments, the pluripotent stem cell and any cell differentiated from such a pluripotent stem cell is modified to exhibit reduced expression of one or more MHC class I and/or II human leukocyte antigen molecules, to exhibit increased CD47 expression, and to exogenously express a chimeric antigen receptor. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of one or more MHC class I and II human leukocyte antigen molecules, exhibit increased CD47 expression, and to exogenously express a chimeric antigen receptor. In some embodiments, the pluripotent stem cell and any cell differentiated from such a pluripotent stem cell is modified to exhibit reduced expression of one or more MHC class I and II human leukocyte antigen molecules and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

Any of the pluripotent stem cells described herein can be differentiated into any cells of an organism and tissue. In some embodiments, the cells exhibit reduced expression of one or more MHC class I and/or II human leukocyte antigen molecules and reduced expression of TCR complexes. In some instances, expression of one or more MHC class I and/or II human leukocyte antigen molecules is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of one or more MHC class I and/or II human leukocyte antigen molecules and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, an inflammatory disorder, a neurological disorder, and the like.

1. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16 (4): 357-366 (2015); Themeli et al., Nature Biotechnology 31:928-933 (2013).

T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR). Any suitable CAR can be included in the hyHIP-derived T cell, including the CARS described herein. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. Any suitable method can be used to insert the CAR into the genomic locus of the hypoimmunogenic cell including the gene editing methods described herein (e.g., a CRISPR/Cas system).

HIP-derived T cells provided herein are useful for the treatment of suitable autoimmune diseases/disorders and/or inflammatory diseases/disorders including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, MS associated with EBV infection, Myasthenia gravis and Diabetes. Further examples of “autoimmune disease” or “autoimmune disorder” or “inflammatory disease” or “inflammatory disorder” can be found in Section Z below.

2. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins/molecules. On contact with a target cell NK cells release proteins such as perforin, and enzymes such as proteases (granzymes). Perforin can form pores in the cell membrane of a target cell, inducing apoptosis or cell lysis.

There are a number of techniques that can be used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that can be used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9 (4): 493-504, all of which are incorporated herein by reference in their entirety and specifically for the methodologies and reagents for differentiation. Differentiation can be assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. Any suitable CAR can be included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, PD1 or CTLA4 gene. Any suitable method can be used to insert the CAR into the genomic locus of the NK cell including the gene editing methods described herein (e.g., a CRISPR/Cas system).

### X. Methods of Genetic Modifications

In some embodiments, a vector herein is a nucleic acid molecule capable transferring or transporting another nucleic acid molecule, including into the cell or into genome of a cell. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. Non-viral vectors may require a delivery vehicle to facilitate entry of the nucleic acid molecule into a cell.

A viral vector can comprise a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively. Retroviruses may also contain additional genes which code for proteins other than gag, pol and env. Examples of additional genes include (in HIV), one or more of vif, vpr, vpx, vpu, tat, rev and nef. ElAV has (amongst others) the additional gene S2.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretro virus. In some embodiments the retrovirus is a Deltaretro virus. In some embodiments the retrovirus is a Spumaretrovirus. In some embodiments the retrovirus is an endogenous retrovirus. In some embodiments the retrovirus is a lentivirus.

In some embodiments, a retroviral or lentivirus vector further comprises one or more insulator elements, e.g., an insulator element described herein. In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE. In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used. A lentivirus vector can comprise a viral vector or plasmid containing structural and functional genetic elements, or portions thereof, including LTRs that are primarily derived from a lentivirus.

In embodiments, a lentivirus vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In embodiments, a lentivirus vector is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentivirus vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included.

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The present disclosure contemplates altering target polynucleotide sequences in any manner which is available to the skilled artisan utilizing a gene editing system (e.g. CRISPR/Cas) of the present disclosure. Any CRISPR/Cas system that is capable of altering a target polynucleotide sequence in a cell can be used. Such CRISPR-Cas systems can employ a variety of Cas proteins (Haft et al. PLOS Comput Biol. 2005; 1 (6) e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR Type I system. In some embodiments, the CRISPR/Cas system is a CRISPR Type II system. In some embodiments, the CRISPR/Cas system is a CRISPR Type V system.

The CRISPR/Cas systems of the present disclosure can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present disclosure can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present disclosure includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein, isoform thereof, or any Cas-like protein with similar function or activity of any Cas protein or isoform thereof. In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises type V Cas protein. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019). Examples of Cas proteins include, but are not limited to: Cas3, Cas8a, Cas5, Cas8b, Cas8c, Cas10d, Cse1, Cse2, Csy1, Csy2, Csy3, and/or GSU0054. In some embodiments, a Cas protein comprises Cas3, Cas8a, Cas5, Cas8b, Cas8c, Cas10d, Cse1, Cse2, Csy1, Csy2, Csy3, and/or GSU0054. Examples of Cas proteins include, but are not limited to: Cas9, Csn2, and/or Cas4. In some embodiments, a Cas protein comprises Cas9, Csn2, and/or Cas4. In some embodiments, Examples of Cas proteins include, but are not limited to: Cas10, Csm2, Cmr5, Cas10, Csx11, and/or Csx10. In some embodiments, a Cas protein comprises a Cas10, Csm2, Cmr5, Cas10, Csx11, and/or Csx10. In some embodiments, examples of Cas proteins include, but are not limited to: Csf1. In some embodiments, a Cas protein comprises Csf1. In some embodiments, examples of Cas proteins include, but are not limited to: Cas12a, Cas12b, Cas12c, C2c4, C2c8, C2c5, C2c10, and C2c9; as well as CasX (Cas12e) and CasY (Cas12d). Also see, e.g., Koonin et al., Curr Opin Microbiol. 2017; 37:67-78: “Diversity, classification and evolution of CRISPR-Cas systems.” In some embodiments, a Cas protein comprises Cas12a, Cas12b, Cas12c, Cas12d, Cas12e, Cas12d, and/or Cas12e. In some embodiments, a Cas protein comprises In some embodiments, a Cas protein comprises Cas13, Cas13a, C2c2, Cas13b, Cas13c, and/or Cas13d.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In many embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5 (8): 747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods of the present disclosure contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids of the present disclosure can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 30. The sequences can be found in WO2016183041 filed May 9, 2016, the disclosure including the Tables, Appendices, and Sequence Listing is incorporated herein by reference in its entirety.

TABLE 30
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |
| Gene Name | SEQ ID NO: | US20160348073 |
| TRAC | SEQ ID NOS: 532-609 and | |
| | 9102-9797 | |
| TRB (also | SEQ ID NOS: 610-765 and | |
| TCRB and | 9798-10532 | |
| TRBC) | | |

Other exemplary gRNA sequences useful for CRISPR/Cas-based targeting of genes described herein are provided in U.S. Provisional Patent Application No. 63/190,685, filed May 19, 2021, and in U.S. Provisional Patent Application No. 63/221,887, filed Jul. 14, 2021, the disclosures of which, including the Tables, Appendices, and Sequence Listings, are incorporated herein by reference in their entireties.

In some embodiments, the cells of the technology are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In numerous embodiments, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells of the present disclosure are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the technology may for example correspond to a LAGLIDADG endonuclease, to a HNH endonuclease, or to a GIY-YIG endonuclease. Preferred homing endonuclease according to the present disclosure can be an I-CreI variant.

In some embodiments, the cells of the technology are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells of the technology are made using RNA silencing or RNA interference (RNAi) to knock down (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knock down methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, NLRC5, TCR-alpha, and TCR-beta.

In some embodiments, the cells provided herein are genetically modified to reduce expression of one or more immune factors (including target polypeptides) to create immune-privileged or hypoimmunogenic cells. In certain embodiments, the cells (e.g., stem cells, induced pluripotent stem cells, differentiated cells, hematopoietic stem cells, primary T cells and CAR-T cells) disclosed herein comprise one or more genetic modifications to reduce expression of one or more target polynucleotides. Non-limiting examples of such target polynucleotides and polypeptides include CIITA, B2M, NLRC5, CTLA-4, PD-1, HLA-A, HLA-BM, HLA-C, RFX-ANK, NFY-A, RFX5, RFX-AP, NFY-B, NFY-C, IRF1, and TAP1.

In some embodiments, the genetic modification occurs using a CRISPR/Cas system. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

### 1. Gene Editing Systems

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems, as well as nickase systems, base editing systems, prime editing systems, and gene writing systems known in the art.

i. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

ii. TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

iii. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

iv. Transposases

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

v. CRISPR/Cas Systems

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAS (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 30 below.

TABLE 30
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas12a (Cpf1) | Acidaminococcus sp. | TTTV |
| AacCas12b | Alicyclobacillus acidiphilus | TTN |
| BhCas12b v4 | Bacillus hisashii | ATTN, TTTN, or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity. 6. Nickases

Nuclease domains of the Cas, in particular the Cas9, nuclease can be mutated independently to generate enzymes referred to as DNA “nickases”. Nickases are capable of introducing a single-strand cut with the same specificity as a regular CRISPR/Cas nuclease system, including for example CRISPR/Cas9. Nickases can be employed to generate double-strand breaks which can find use in gene editing systems (Mali et al., Nat Biotech, 31 (9): 833-838 (2013); Mali et al. Nature Methods, 10:957-963 (2013); Mali et al., Science, 339 (6121): 823-826 (2013)). In some instances, when two Cas nickases are used, long overhangs are produced on each of the cleaved ends instead of blunt ends which allows for additional control over precise gene integration and insertion (Mali et al., Nat Biotech, 31 (9): 833-838 (2013); Mali et al. Nature Methods, 10:957-963 (2013); Mali et al., Science, 339 (6121): 823-826 (2013)). As both nicking Cas enzymes must effectively nick their target DNA, paired nickases can have lower off-target effects compared to the double-strand-cleaving Cas-based systems (Ran et al., Cell, 155 (2): 479-480 (2013); Mali et al., Nat Biotech, 31 (9): 833-838 (2013); Mali et al. Nature Methods, 10:957-963 (2013); Mali et al., Science, 339 (6121): 823-826 (2013)).

Y. Methods of Recombinant Expression of Tolerogenic Factors and/or Chimeric Antigen Receptors

For all of these technologies, well-known recombinant techniques are used, to generate recombinant nucleic acids as outlined herein. In certain embodiments, the recombinant nucleic acids encoding a tolerogenic factor or a chimeric antigen receptor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. Some embodiments, include an expression vector comprising a nucleotide sequence encoding a variant polypeptide operably linked to at least one regulatory sequence. Regulatory sequence for use herein include promoters, enhancers, and other expression control elements. In some embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, CAG promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al., Nature 273:113-120 (1978)). The immediate early promoter of the human cytomegalovirus is conveniently obtained as a HindIII restriction enzyme fragment (Greenaway et al., Gene 18:355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., AAV transduction, lentiviral transduction) or otherwise delivered on a viral vector (e.g., fusogen-mediated delivery). In some embodiments, the polynucleotides are introduced into a cell via a fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system). In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Unlike certain methods of introducing the polynucleotides described herein into cells which generally involve activating cells, such as activating T cells (e.g., CD8⁺ T cells), suitable techniques can be utilized to introduce polynucleotides into non-activated T cells. Suitable techniques include, but are not limited to, activation of T cells, such as CD8⁺ T cells, with one or more antibodies which bind to CD3, CD8, and/or CD28, or fragments or portions thereof (e.g., scFv and VHH) that may or may not be bound to beads. Surprisingly, fusogen-mediated introduction of polynucleotides into T cells is performed in non-activated T cells (e.g., CD8⁺ T cells) that have not been previously contacted with one or more activating antibodies or fragments or portions thereof (e.g., CD3, CD8, and/or CD28). In some embodiments, fusogen-mediated introduction of polynucleotides into T cells is performed in vivo (e.g., after the T cells have been administered to a subject). In other embodiments, fusogen-mediated introduction of polynucleotides into T cells is performed in vitro (e.g., before the T cells are been administered to a subject).

Provided herein are non-activated T cells comprising reduced expression of HLA-A, HLA-B, HLA-C, HLA-DP, HLA-DQ, HLA-DR, B2M, CIITA, TCR-alpha, and/or TCR-beta relative to a wild-type T cell, wherein the non-activated T cell further comprises a first exogenous polynucleotide encoding a chimeric antigen receptor (CAR).

In some embodiments, the non-activated T cell has not been treated with an anti-CD3 antibody, an anti-CD28 antibody, a T cell activating cytokine, or a soluble T cell costimulatory molecule. In some embodiments, the non-activated T cell does not express activation markers. In some embodiments, the non-activated T cell expresses CD3 and CD28, and wherein the CD3 and/or CD28 are inactive.

In some embodiments, the anti-CD3 antibody is OKT3. In some embodiments, the anti-CD28 antibody is CD28.2. In some embodiments, the T cell activating cytokine is selected from the group of T cell activating cytokines consisting of IL-2, IL-7, IL-15, and IL-21. In some embodiments, the soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody.

In some embodiments, the non-activated T cell is a primary T cell. In other embodiments, the non-activated T cell is differentiated from the hypoimmunogenic cells of the present disclosure. In some embodiments, the T cell is a CD8⁺ T cell.

In some embodiments, the first exogenous polynucleotide encodes a CAR selected from the group consisting of a CD19-specific CAR, a CD22-specific CAR, a CD20-specific CAR, a BCMA specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR.

In some embodiments, the first and/or second exogenous polynucleotide is carried by a viral vector, including a lentiviral vector. In some embodiments, the first and/or second exogenous polynucleotide is carried by a lentiviral vector that comprises a CD8 binding agent. In some embodiments, the first and/or second exogenous polynucleotide is introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the non-activated T cell further comprises a second exogenous polynucleotide encoding CD47. In some embodiments, the first and/or second exogenous polynucleotides are inserted into a specific locus of at least one allele of the T cell. In some embodiments, the specific locus is selected from the group consisting of a safe harbor or target locus, a target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus. In some embodiments, the second exogenous polynucleotide encoding CD47 and the first exogenous polynucleotide encoding the CAR are inserted into the CIITA locus. In some embodiments, the second exogenous polynucleotide encoding CD47 and the first exogenous polynucleotide encoding the CAR are inserted into the TRAC locus. In some embodiments, the second exogenous polynucleotide encoding CD47 and the first exogenous polynucleotide encoding the CAR are inserted into the TRB locus. In some embodiments, the second exogenous polynucleotide encoding CD47 and the first exogenous polynucleotide encoding the CAR are inserted into the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, the non-activated T cell does not express HLA-A, HLA-B, and/or HLA-C antigens. In some embodiments, the non-activated T cell does not express B2M. In some embodiments, the non-activated T cell does not express HLA-DP, HLA-DQ, and/or HLA-DR antigens. In some embodiments, the non-activated T cell does not express CIITA. In some embodiments, the non-activated T cell does not express TCR-alpha. In some embodiments, the non-activated T cell does not express TCR-beta. In some embodiments, the non-activated T cell does not express TCR-alpha and TCR-beta.

In some embodiments, the non-activated T cell is a B2M^{indel/indel}, CIITA^{indel/indel}, TRAC^{indel/indel} cell comprising first exogenous polynucleotide encoding CAR and/or the second exogenous polynucleotide encoding CD47. In some embodiments, the first and/or second exogenous polynucleotides are inserted into at least one allele of the T cell using viral transduction. In some embodiments, the first and/or second exogenous polynucleotides are inserted into at least one allele of the T cell using a lentivirus based viral vector. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the TRAC locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into the TRAC locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the TRB locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into the TRB locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the B2M locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a B2M locus. In some embodiments, the non-activated T cell is a B2M^{indel/indel}, CIITA^{indel/indel}, TRAC^{indel/indel} cell comprising second exogenous polynucleotide encoding CD47 and/or the first exogenous polynucleotide encoding CAR inserted into the CIITA locus. In some embodiments, the non-activated T cell is a B2M^{indel/indel}, CIITA^{indel/indel}, TRAC^{indel/indel} cell comprising the second exogenous polynucleotide encoding CD47 and the first exogenous polynucleotide encoding CAR inserted into a CIITA locus.

In some embodiments, the non-activated T cell is a B2M^{indel/indel}, CIITA^{indel/indel} TRB^{indel/indel} cell comprising second exogenous polynucleotide encoding CD47 and/or the first exogenous polynucleotide encoding CAR inserted into the TRAC locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into the TRAC locus. In some embodiments, the non-activated T cell is a B2M^{indel/indel} CIITA^{indel/indel}, TRB^{indel/indel} cell comprising second exogenous polynucleotide encoding CD47 and/or the first exogenous polynucleotide encoding CAR inserted into the TRB locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into the TRB locus. In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the B2M locus. In some embodiments, the non-activated T cell is a B2M^{indel/indel}, CIITA^{indel/indel}, TRB^{indel/indel} cell comprising the second exogenous polynucleotide encoding CD47 and the first exogenous polynucleotide encoding CAR inserted into a B2M locus. In some embodiments, the non-activated T cell is a B2M^{indel/indel}, CIITA^{indel/indel}, TRB^{indel/indel} cell comprising second exogenous polynucleotide encoding CD47 and/or the first exogenous polynucleotide encoding CAR inserted into the CIITA locus. In some embodiments, the non-activated T cell is a B2M^{indel/indel} CIITA^{indel/indel}, TRB^{indel/indel} cell comprising the second exogenous polynucleotide encoding CD47 and the first exogenous polynucleotide encoding CAR inserted into a CIITA locus.

Provided herein are engineered T cells comprising reduced expression of HLA-A, HLA-B, HLA-C, HLA-DP, HLA-DQ, HLA-DR, B2M, CIITA, TCR-alpha, and/or TCR-beta relative to a wild-type T cell, wherein the engineered T cell further comprises a first exogenous polynucleotide encoding a chimeric antigen receptor (CAR) carried by a lentiviral vector that comprises a CD8 binding agent.

In some embodiments, the engineered T cell is a primary T cell. In other embodiments, the engineered T cell is differentiated from the hypoimmunogenic cell of the present disclosure. In some embodiments, the T cell is a CD8⁺ T cell. In some embodiments, the T cell is a CD4⁺ T cell.

In some embodiments, the engineered T cell does not express activation markers. In some embodiments, the engineered T cell expresses CD3 and CD28, and wherein the CD3 and/or CD28 are inactive.

In some embodiments, the engineered T cell has not been treated with an anti-CD3 antibody, an anti-CD28 antibody, a T cell activating cytokine, or a soluble T cell costimulatory molecule. In some embodiments, the anti-CD3 antibody is OKT3, wherein the anti-CD28 antibody is CD28.2, wherein the T cell activating cytokine is selected from the group of T cell activating cytokines consisting of IL-2, IL-7, IL-15, and IL-21, and wherein soluble T cell costimulatory molecule is selected from the group of soluble T cell costimulatory molecules consisting of an anti-CD28 antibody, an anti-CD80 antibody, an anti-CD86 antibody, an anti-CD137L antibody, and an anti-ICOS-L antibody. In some embodiments, the engineered T cell has not been treated with one or more T cell activating cytokines selected from the group consisting of IL-2, IL-7, IL-15, and IL-21. In some instances, the cytokine is IL-2. In some embodiments, the one or more cytokines is IL-2 and another selected from the group consisting of IL-7, IL-15, and IL-21.

In some embodiments, the engineered T cell further comprises a second exogenous polynucleotide encoding CD47. In some embodiments, the first and/or second exogenous polynucleotides are inserted into a specific locus of at least one allele of the T cell. In some embodiments, the specific locus is selected from the group consisting of a safe harbor or target locus, a target locus, a B2M locus, a CIITA locus, a TRAC locus, and a TRB locus. In some embodiments, the second exogenous polynucleotide encoding CD47 is inserted into the specific locus selected from the group consisting of a safe harbor or target locus, a target locus, a B2M locus, a CIITA locus, a TRAC locus and a TRB locus. In some embodiments, the first exogenous polynucleotide encoding the CAR is inserted into the specific locus selected from the group consisting of a safe harbor or target locus, a target locus, a B2M locus, a CIITA locus, a TRAC locus and a TRB locus. In some embodiments, the second exogenous polynucleotide encoding CD47 and the first exogenous polynucleotide encoding the CAR are inserted into different loci. In some embodiments, the second exogenous polynucleotide encoding CD47 and the first exogenous polynucleotide encoding the CAR are inserted into the same locus. In some embodiments, the second exogenous polynucleotide encoding CD47 and the first exogenous polynucleotide encoding the CAR are inserted into the B2M locus, the CIITA locus, the TRAC locus, the TRB locus, or the safe harbor or target locus. In some embodiments, the safe harbor or target locus is selected from the group consisting of a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, the CAR is selected from the group consisting of a CD19-specific CAR, a CD22-specific CAR, a CD20-specific CAR, a BCMA specific CAR, an EBV antigen-specific CAR, a CD27-specific CAR, a CD30-specific CAR, a EBNA1-specific CAR, a EBNA3A-specific CAR, a BRLF1-specific CAR, a BALF4-specific CAR, a EBNA3C-specific CAR, a LMP1-specific CAR, a LMP2-specific CAR, a LMP2A-specific CAR, a LMP2B-specific CAR, a BZLF1-specific CAR, a BMLF1-specific CAR, a gp350-specific CAR, and a gH/gL-specific CAR. In some embodiments, the CAR is a CD19-specific CAR. In some embodiments, the CAR is a CD20-specific CAR. In some embodiments, the CAR is a CD22-specific CAR. In some embodiments, the CAR is a BCMA specific CAR. In some embodiments, the CAR binds to BCMA. In some embodiments, the CAR binds to an EBV antigen. In some embodiments, the CAR binds to CD27. In some embodiments, the CAR binds to CD30. In some embodiments, the CAR binds to EBNA1. In some embodiments, the CAR binds to EBNA3A. In some embodiments, the CAR binds to BRLF1. In some embodiments, the CAR binds to BALF4. In some embodiments, the CAR binds to EBNA3C. In some embodiments, the CAR binds to LMP1. In some embodiments, the CAR binds to LMP2. In some embodiments, the CAR binds to LMP2A. In some embodiments, the CAR binds to LMP2B. In some embodiments, the CAR binds to BZLF1. In some embodiments, the CAR binds to BMLF1. In some embodiments, the CAR binds to gp350. In some embodiments, the CAR binds to gH/gL. In some embodiments, the CAR comprises an antigen binding domain that binds to any one selected from the group consisting of CD19, CD20, CD22, BCMA, an EBV antigen, CD27, CD30, EBNA1, EBNA3A, BRLF1, BALF4, EBNA3C, LMP1, LMP2, LMP2A, LMP2B, BZLF1, BMLF1, gp350, and gH/gL.

In some embodiments, the engineered T cell does not express HLA-A, HLA-B, and/or HLA-C antigens, wherein the engineered T cell does not express B2M, wherein the engineered T cell does not express HLA-DP, HLA-DQ, and/or HLA-DR antigens, wherein the engineered T cell does not express CIITA, and/or wherein the engineered T cell does not express TCR-alpha and TCR-beta.

In some embodiments, the engineered T cell is a B2M^{indel/indel}, CIITA^{indel/indel}, TRAC^{indel/indel} cell comprising the second exogenous polynucleotide encoding CD47 and/or the first exogenous polynucleotide encoding CAR inserted into the TRAC locus, into the TRB locus, into the B2M locus, or into the CIITA locus. In some embodiments, the engineered T cell is a B2M^{indel/indel}, CIITA^{indel/indel}, TRB^{indel/indel} cell comprising the second exogenous polynucleotide encoding CD47 and/or the first exogenous polynucleotide encoding CAR inserted into the TRAC locus, into the TRB locus, into the B2M locus, or into the CIITA locus.

In some embodiments, the non-activated T cell and/or the engineered T cell of the present disclosure are in a subject. In other embodiments, the non-activated T cell and/or the engineered T cell of the present disclosure are in vitro.

In some embodiments, the non-activated T cell and/or the engineered T cell of the present disclosure express a CD8 binding agent. In some embodiments, the CD8 binding agent is an anti-CD8 antibody. In some embodiments, the anti-CD8 antibody is selected from the group consisting of a mouse anti-CD8 antibody, a rabbit anti-CD8 antibody, a human anti-CD8 antibody, a humanized anti-CD8 antibody, a camelid (e.g., llama, alpaca, camel) anti-CD8 antibody, and a fragment thereof. In some embodiments, the fragment thereof is an scFv or a VHH. In some embodiments, the CD8 binding agent binds to a CD8 alpha chain and/or a CD8 beta chain.

In some embodiments, the CD8 binding agent is fused to a transmembrane domain incorporated in the viral envelope. In some embodiments, the lentivirus vector is pseudotyped with a viral fusion protein. In some embodiments, the viral fusion protein comprises one or more modifications to reduce binding to its native receptor.

In some embodiments, the viral fusion protein is fused to the CD8 binding agent. In some embodiments, the viral fusion protein comprises Nipah virus F glycoprotein and Nipah virus G glycoprotein fused to the CD8 binding agent. In some embodiments, the lentivirus vector does not comprise a T cell activating molecule or a T cell costimulatory molecule. In some embodiments, the lentivirus vector encodes the first exogenous polynucleotide and/or the second exogenous polynucleotide.

In some embodiments, following transfer into a first subject, the non-activated T cell or the engineered T cell exhibits one or more responses selected from the group consisting of (a) a T cell response, (b) an NK cell response, and (c) a macrophage response, that are reduced as compared to a wild-type cell following transfer into a second subject. In some embodiments, the first subject and the second subject are different subjects. In some embodiments, the macrophage response is engulfment.

In some embodiments, following transfer into a subject, the non-activated T cell or the engineered T cell exhibits one or more selected from the group consisting of (a) reduced TH1 activation in the subject, (b) reduced NK cell killing in the subject, and (c) reduced killing by whole PBMCs in the subject, as compared to a wild-type cell following transfer into the subject.

In some embodiments, following transfer into a subject, the non-activated T cell or the engineered T cell elicits one or more selected from the group consisting of (a) reduced donor specific antibodies in the subject, (b) reduced IgM or IgG antibodies in the subject, and (c) reduced complement-dependent cytotoxicity (CDC) in a subject, as compared to a wild-type cell following transfer into the subject.

In some embodiments, the non-activated T cell or the engineered T cell is transduced with a lentivirus vector comprising a CD8 binding agent within the subject. In some embodiments, the lentivirus vector carries a gene encoding the CAR and/or CD47.

In some embodiments, the gene encoding the CAR and/or CD47 is introduced into the cells using a gene therapy vector or a transposase system selected from the group consisting of transposases, PiggyBac transposons, Sleeping Beauty (SB11) transposons, Mos1 transposons, and Tol2 transposons. In some embodiments, the gene therapy vector is a retrovirus or a fusosome.

Provided herein are pharmaceutical compositions comprising a population of the non-activated T cells and/or the engineered T cells of the present disclosure and a pharmaceutically acceptable additive, carrier, diluent or excipient.

Provided herein are methods comprising administering to a subject a composition comprising a population of the non-activated T cells and/or the engineered T cells of the present disclosure, or one or more the pharmaceutical compositions of the present disclosure.

In some embodiments, the subject is not administered a T cell activating treatment before, after, and/or concurrently with administration of the composition. In some embodiments, the T cell activating treatment comprises lymphodepletion.

Provided herein are methods of treating a subject suffering from autoimmune diseases/disorders and/or inflammatory diseases/disorders, comprising administering to a subject a composition comprising a population of the non-activated T cells and/or the engineered T cells of the present disclosure, or one or more the pharmaceutical compositions of the present disclosure, wherein the subject is not administered a T cell activating treatment before, after, and/or concurrently with administration of the composition. In some embodiments, the T cell activating treatment comprises lymphodepletion.

Provided herein are methods for expanding T cells capable of recognizing and killing tumor cells in a subject in need thereof within the subject, comprising administering to a subject a composition comprising a population of the non-activated T cells and/or the engineered T cells of the present disclosure, or one or more the pharmaceutical compositions of the present disclosure, wherein the subject is not administered a T cell activating treatment before, after, and/or concurrently with administration of the composition. In some embodiments, the T cell activating treatment comprises lymphodepletion.

Provided herein are dosage regimens for treating a condition, disease or disorder in a subject comprising administration of a pharmaceutical composition comprising a population of the non-activated T cells and/or the engineered T cells of the present disclosure, or one or more the pharmaceutical compositions of the present disclosure, and a pharmaceutically acceptable additive, carrier, diluent or excipient, wherein the pharmaceutical composition is administered in about 1-3 therapeutically effective doses. Provided herein are dosage regimens for treating a condition, disease or disorder in a subject comprising administration of a pharmaceutical composition comprising a population of the non-activated T cells and/or the engineered T cells of the present disclosure, or one or more the pharmaceutical compositions of the present disclosure, and a pharmaceutically acceptable additive, carrier, diluent or excipient, wherein the pharmaceutical composition is administered in about 1-3 clinically effective doses.

Once altered, the presence of expression of any of the molecule described herein can be assayed using known techniques, such as Western blots, ELISA assays, FACS assays, other immunoassays, reverse transcriptase polymerase chain reactions (RT-PCR), and the like.

### Z. Generation of Induced Pluripotent Stem Cells

The technology provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7 (1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### AA. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116 (21), 10441-10446, the disclosures including the figures, figure legends, and description of methods are incorporated herein by reference in their entirety.

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of one or more HLA I and II (or MHC I and II) molecules, the hypoimmunogenic cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

### BB. Exogenous Polynucleotides

In some embodiments, the hypoimmunogenic cells provided herein are genetically modified to include one or more exogenous polynucleotides inserted into one or more genomic loci of the hypoimmunogenic cell. In some embodiments, the exogenous polynucleotide encodes a protein of interest, e.g., a chimeric antigen receptor. Any suitable method can be used to insert the exogenous polynucleotide into the genomic locus of the hypoimmunogenic cell including the gene editing methods described herein (e.g., a CRISPR/Cas system).

The exogenous polynucleotide can be inserted into any suitable genomic loci of the hypoimmunogenic cell. In some embodiments, the exogenous polynucleotide is inserted into a safe harbor or target locus as described herein. Suitable safe harbor and target loci include, but are not limited to, a CCR5 gene, a CXCR4 gene, a PPP1R12C (also known as AAVS1) gene, an albumin gene, a SHS231 locus, a CLYBL gene, a Rosa gene (e.g., ROSA26), an F3 gene (also known as CD142), a MICA gene, a MICB gene, a LRP1 gene (also known as CD91), a HMGB1 gene, an ABO gene, a RHD gene, a FUT1 gene, a PDGFRa gene, an OLIG2 gene, a GFAP gene, and a KDM5D gene (also known as HY). In some embodiments, the exogenous polynucleotide is interested into an intron, exon, or coding sequence region of the safe harbor or target gene locus. In some embodiments, the exogenous polynucleotide is inserted into an endogenous gene wherein the insertion causes silencing or reduced expression of the endogenous gene. In some embodiments, the polynucleotide is inserted in a B2M, CIITA, TRAC, TRB, PD-1 or CTLA-4 gene locus. Exemplary genomic loci for insertion of an exogenous polynucleotide are depicted in Table 31.

TABLE 31
Exemplary genomic loci for insertion of exogenous polynucleotides

| | | | | Target region | |
|---|---|---|---|---|---|
| Number | species | name | Ensembl ID | for cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, | |
| | | | | and CDS | |
| 7 | human | THUMPD3-AS1 | ENSG00000206573 | Intron 1 | ROSA26 |
| 8 | human | Ch- | | 500 bp | SHS231 |
| | | 4: 58,976,613 | | window | |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

TABLE 32
Non-limiting examples of Cas9 guide RNAs

| | SEQ | | | | |
|---|---|---|---|---|---|
| | ID | | | Target | |
| Gene | NO: | guide sequence | PAM | site | gRNA cut location |
| ABO | 1 | UCUCUCCAUGUGCAGUAGGA | AGG | Exon 7 | chr9: 133,257,541 |
| FUT1 | 2 | CUGGAUGUCGGAGGAGUACG | CGG | Exon 4 | chr19: 48,750,822 |
| RH | 3 | GUCUCCGGAAACUCGAGGUG | AGG | Exon 2 | chr1: 25,284,622 |
| F3 (CD142) | 4 | ACAGUGUAGACUUGAUUGAC | GGG | Exon 2 | chr1: 94,540,281 |
| B2M | 5 | CGUGAGUAAACCUGAAUCUU | TGG | Exon 2 | chr15: 44,715,434 |
| CIITA | 182 | GAUAUUGGCAUAAGCCUCCC | TGG | Exon 3 | chr16: 10,895,747 |
| TRAC | 183 | AGAGUCUCUCAGCUGGUACA | CGG | Exon 1 | chr14: 22,5547,533 |

For the Cas9 guides, the spacer sequence for all Cas9 guides is provided in Table 33, with description that the 20nt guide sequence corresponds to a unique guide sequence and can be any of those described herein, including for example those listed in Table 32.

TABLE 33
Cas9 guide RNAs

| | SEQ ID | |
|---|---|---|
| Description | NO: | Sequence |
| 20 nt guide | | NNNNNNNNNNNNNNNNNNNN |
| sequence* | | |
| 12 nt crRNA repeat | 184 | GUUUUAGAGCUA |
| sequence | | |
| 4 nt tetraloop | 10 | GAAA |
| sequence | | |
| 64 nt tracrRNA | 185 | UAGCAAGUUAAAAUAAGGCUAGUCCGUUAUCAACUUGAAAAAG |
| sequence | | UGGCACCGAGUCGGUGCUUU |
| Exemplary full | 186 | NNNNNNNNNNNNNNNNNNNNGUUUUAGAGCUAGAAAUAGCA |
| sequence | | AGUUAAAAUAAGGCUAGUCCGUUAUCAACUUGAAAAAGUGGCA |
| | | CCGAGUCGGUGCUUU |

In some embodiments, the hypoimmunogenic cell that includes the exogenous polynucleotide is derived from a hypoimmunogenic induced pluripotent cell (HIP), for example, as described herein. Such hypoimmunogenic cells include, for example, T cells and NK cells. In some embodiments, the hypoimmunogenic cell that includes the exogenous polynucleotide is a T cell (e.g., a primary T cell), or an NK cell.

In some embodiments, the exogenous polynucleotide encodes an exogenous CD47 polypeptide (e.g., a human CD47 polypeptide) and the exogenous polypeptide is inserted into a safe harbor or target gene loci or a safe harbor or target site as disclosed herein or a genomic locus that causes silencing or reduced expression of the endogenous gene. In some embodiments, the polynucleotide is inserted in a B2M, CIITA, TRAC, TRB, PD1 or CTLA4 gene locus.

In some embodiments, the hypoimmunogenic cell that includes the exogenous polynucleotide is a primary T cell or a T cell derived from a hypoimmunogenic pluripotent cell (e.g., a hypoimmunogenic iPSC). In exemplary embodiments, the exogenous polynucleotide is a chimeric antigen receptor (e.g., any of the CARs described herein). In some embodiments, the exogenous polynucleotide is operably linked to a promoter for expression of the exogenous polynucleotide in the hypoimmunogenic cell.

### CC. Pharmaceutically Acceptable Carriers

In some embodiments, the pharmaceutical composition provided herein further include a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline).

In some embodiments, the pharmaceutical composition includes one or more electrolyte base solutions selected from the group consisting of lactated CryoStor®, Ringer's solution, PlasmaLyte-A™, Iscove's Modified Dulbecco's Medium, Normosol-R™, Veen-D™, Polysal® and Hank's Balanced Salt Solution (containing no phenol red). These base solutions closely approximate the composition of extracellular mammalian physiological fluids.

In some embodiments, the pharmaceutical composition includes one or more cryoprotective agents selected from the group consisting of arabinogalactan, glycerol, polyvinylpyrrolidone (PVP), dextrose, dextran, trehalose, sucrose, raffinose, hydroxyethyl starch (HES), propylene glycol, human serum albumin (HSA), and dimethylsulfoxide (DMSO). In some embodiments, the pharmaceutically acceptable buffer is neutral buffer saline or phosphate buffered saline. In some embodiments, pharmaceutical compositions provided herein include one or more of CryoStor® CSB, Plasma-Lyte-A™, HSA, DMSO, and trehalose.

CryoStor® is an intracellular-like optimized solution containing osmotic/oncotic agents, free radical scavengers, and energy sources to minimize apoptosis, minimize ischemia/reperfusion injury and maximize the post-thaw recovery of the greatest numbers of viable, functional cells. CryoStor® is serum- and protein-free, and non-immunogenic. CryoStor® is cGMP-manufactured from raw materials of USPgrade or higher. CryoStor® is a family of solutions pre-formulated with 0%, 2%, 5% or 10% DMSO. CryoStor® CSB is a DMSO-free version of CryoStor®. In some embodiments, the pharmaceutical composition includes a base solution of CryoStor® CSB at a concentration of about 0-100%, 5-95%, 10-90%, 15-85%, 20-80%, 30-80%, 40-80%, 50-80%, 60-80%, 70-80%, 25-75%, 30-70%, 35-65%, 40-60%, or 45-55% w/w. In some embodiments, the pharmaceutical composition includes a base solution of CryoStor® CSB at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

PlasmaLyte-A™ is a non-polymeric plasma expander and contains essential salts and nutrients similar to those found in culture medium but does not contain additional constituents found in tissue culture medium which are not approved for human infusion, e.g., phenol red, or are unavailable in U.S.P. grade. PlasmaLyte-A™ contains about 140 mEq/liter of sodium (Na), about 5 mEq/liter of potassium (K), about 3 mEq/liter of magnesium (Mg), about 98 mEq/liter of chloride (CI), about 27 mEq/liter of acetate, and about 23 mEq/liter of gluconate. (PlasmaLyte-A™ is commercially available from Baxter, Hyland Division, Glendale Calif., product No. 2B2543). In some embodiments, the pharmaceutical composition includes a base solution of PlasmaLyte-A™ at a concentration of about 0-100%, 5-95%, 10-90%, 15-85%, 15-80%, 15-75%, 15-70%, 15-65%, 15-60%, 15-55%, 15-50%, 15-45%, 15-40%, 15-35%, 15-30%, 15-25%, 20-80%, 20-75%, 20-70%, 20-65%, 20-60%, 20-55%, 20-50%, 20-45%, 20-40%, 20-35%, 20-30%, 25-75%, 30-70%, 35-65%, 40-60%, or 45-55% w/w. In some embodiments, the pharmaceutical composition includes a base solution of PlasmaLyte-A™ at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

In some embodiments, the pharmaceutical composition includes human serum albumin (HSA) at a concentration of about 0-10%, 0.3-9.3%, 0.3-8.3%, 0.3-7.3%, 0.3-6.3%, 0.3-5.3%, 0.3-4.3%, 0.3-3.3%, 0.3-2.3%, 0.3-1.3%, 0.6-8.3%, 0.9-7.3%, 1.2-6.3%, 1.5-5.3%, 1.8-4.3%, or 2.1-3.3% w/v. In some embodiments, the pharmaceutical composition includes HSA at a concentration of about 0%, 0.3%, 0.6%, 0.9%, 1.2%, 1.5%, 1.8%, 2.1%, 2.4%, 2.7%, 3.0%, 3.3%, 3.6%, 3.9%, 4.3%, 4.6%, 4.9%, 5.3%, 5.6%, 5.9%, 6.3%, 6.6%, 6.9%, 7.3%, 7.6%, 7.9%, 8.3%, 8.6%, 8.9%, 9.3%, 9.6%, 9.9%, or 10% w/v.

In some embodiments, the pharmaceutical composition includes dimethylsulfoxide (DMSO) at a concentration of about 0-10%, 0.5-9.5%, 1-9%, 1.5-8.5%, 2-8%, 3-8%, 4-8%, 5-8%, 6-8%, 7-8%, 2.5-7.5%, 3-7%, 3.5-6.5%, 4-6%, or 4.5-5.5% v/v. In some embodiments, the pharmaceutical composition includes HSA at a concentration of about 0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0% v/v.

In some embodiments, the pharmaceutical composition includes trehalose at a concentration of about 0-500 mM, 50-450 mM, 100-400 mM, 150-350 mM, or 200-300 mM. In some embodiments, the pharmaceutical composition includes trehalose at a concentration of about 0 mM, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM.

Exemplary pharmaceutical composition components are shown in Table 34.

TABLE 34
Exemplary pharmaceutical composition components.

| | | | Additional | |
|---|---|---|---|---|
| Formulation | Base Solution | c[DMSO] | c[HSA]* | c[trehalose] |
| A | 75% CroStor ® | 7.5% | 0.3% | |
| B | CSB + 25% | 3.75% | 0.3% | |
| C | PlasmaLyte A ™ + | | 5.3% | |
| D | 1.2% HSA | | 0.3% | 250 mM |
| E | 100% | 7.5% | 0.3% | |
| F | PlasmaLyte A ™ + | 7.5% | 5.3% | |
| G | 1.2% HSA | 7.5% | 5.3% | 250 mM |

*Additional HSA in addition to PlasmaLyte.

In some embodiments, the pharmaceutical composition comprises hypoimmunogenic cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

### DD. Formulations and Dosage Regimens

Any therapeutically effective amount of cells described herein can be included in the pharmaceutical composition, depending on the autoimmune indication being treated. Non-limiting examples of the cells include primary T cells, T cells differentiated from hypoimmunogenic induced pluripotent stem cells, and other cells differentiated from hypoimmunogenic induced pluripotent stem cells described herein. In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, or 1×10¹⁰-5×10¹⁰ cells. In exemplary embodiments, the pharmaceutical composition includes from about 1.0×10⁶ to about 2.5×10⁸ cells. In certain embodiments, the pharmaceutical composition includes from about 2.0×10⁶ to about 2.0×10⁸ cells, such as but not limited to, primary T cells, T cells differentiated from hypoimmunogenic induced pluripotent stem cells.

In some embodiments, the pharmaceutical composition has a volume of at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In certain embodiments, the pharmaceutical composition has a volume that ranges from about 10 ml to about 50 ml.

The specific amount/dosage regimen will vary depending on the weight, gender, age and health of the individual, the formulation, the biochemical nature, bioactivity, bioavailability and the side effects of the cells and the number and identity of the cells in the complete therapeutic regimen.

In some embodiments, a therapeutically effective dose or a clinically effective dose of the pharmaceutical composition includes about 1.0×10⁵ to about 2.5×10⁸ cells at a volume of about 10 ml to 50 ml and the pharmaceutical composition is administered as a single therapeutically effective dose or clinically effective dose. In some cases, the therapeutically effective dose or clinically effective dose includes about 1.0×10⁵ to about 2.5×10⁸ primary T cells described herein at a volume of about 10 ml to 50 ml. In some cases, the therapeutically effective dose or clinically effective dose includes about 1.0×10⁵ to about 2.5×10⁸ primary T cells that have been described above at a volume of about 10 ml to 50 ml. In various cases, the therapeutically effective dose or clinically effective dose includes about 1.0×10⁵ to about 2.5×10⁸ T cells differentiated from hypoimmunogenic induced pluripotent stem cells described herein at a volume of about 10 ml to 50 ml. In some embodiments, the therapeutically effective dose or clinically effective dose is 1.0×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, or 2.5×10⁸ T cells differentiated from hypoimmunogenic induced pluripotent stem cells described herein at a volume of about 10 ml to 50 ml. In other cases, the therapeutically effective dose or clinically effective dose is at a range that is lower than about 1.0×10⁵ to about 2.5×10⁸ T cells, including primary T cells or T cells differentiated from hypoimmunogenic induced pluripotent stem cells. In yet other cases, the therapeutically effective dose or clinically effective dose is at a range that is higher than about 1.0×10⁵ to about 2.5×10⁸ T cells, including primary T cells and T cells differentiated from hypoimmunogenic induced pluripotent stem cells.

In some embodiments, the pharmaceutical composition is administered as a single therapeutically effective dose or clinically effective dose of from about 1.0×10⁵ to about 1.0×10⁷ cells (such as primary T cells and T cells differentiated from hypoimmunogenic induced pluripotent stem cells) per kg body weight for subjects 50 kg or less. In some embodiments, the pharmaceutical composition is administered as a single therapeutically effective dose or clinically effective dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the therapeutically effective dose or clinically effective dose is 0.5×10⁵, 0.6×10⁵, 0.7×10⁵, 0.8×10⁵, 0.9×10⁵, 1.0×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 2.6×10⁵, 2.7×10⁵, 2.8×10⁵, 2.9×10⁵, 3.0×10⁵, 3.1×10⁵, 3.2×10⁵, 3.3×10⁵, 3.4×10⁵, 3.5×10⁵, 3.6×10⁵, 3.7×10⁵, 3.8×10⁵, 3.9×10⁵, 4.0×10⁵, 4.1×10⁵, 4.2×10⁵, 4.3×10⁵, 4.4×10⁵, 4.5×10⁵, 4.6×10⁵, 4.7×10⁵, 4.8×10⁵, 4.9×10⁵, 5.0×10⁵, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 0.5×10⁷, 0.6×10⁷, 0.7×10⁷, 0.8×10⁷, 0.9×10⁷, or 1.0×10⁷ cells per kg body weight for subjects 50 kg or less. In some embodiments, the therapeutically effective dose or clinically effective dose is from about 0.2×10⁶ to about 5.0×10⁶ cells per kg body weight for subjects 50 kg or less. In certain embodiments, the therapeutically effective dose or clinically effective dose is at a range that is lower than from about 0.2×10⁶ to about 5.0×10⁶ cells per kg body weight for subjects 50 kg or less. or clinically effective dose In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the cells are administered in a single therapeutically effective dose of from about 1.0×10⁶ to about 5.0×10⁸ cells (such as primary T cells and T cells differentiated from hypoimmunogenic induced pluripotent stem cells) for subjects above 50 kg. In some embodiments, the pharmaceutical composition is administered as a single therapeutically effective dose or clinically effective dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In some embodiments, the therapeutically effective dose or clinically effective dose is 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, or 5.0×10⁸ cells per kg body weight for subjects 50 kg or less. In certain embodiments, the cells are administered in a single therapeutically effective dose or clinically effective dose of about 1.0×10⁷ to about 2.5×10⁸ cells for subjects above 50 kg. In some embodiments, the cells are administered in a single therapeutically effective dose or clinically effective dose of a range that is less than about 1.0×10⁷ to about 2.5×10⁸ cells for subjects above 50 kg. In some embodiments, the cells are administered in a single therapeutically effective dose or clinically effective dose of a range that is higher than about 1.0×10⁷ to about 2.5×10⁸ cells for subjects above 50 kg. In some embodiments, the dose is administered intravenously. In exemplary embodiments, the single therapeutically effective dose or clinically effective dose is at a volume of about 10 ml to 50 ml. In some embodiments, the therapeutically effective dose or clinically effective dose is administered intravenously.

In exemplary embodiments, the therapeutically effective dose or clinically effective dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single therapeutically effective dose or clinically effective dose of the pharmaceutical composition is present in a single infusion bag. In other embodiments, a single therapeutically effective dose or clinically effective dose of the pharmaceutical composition is divided into 2, 3, 4 or 5 separate infusion bags.

In some embodiments, the cells described herein are administered in a plurality of doses such as 2, 3, 4, 5, 6 or more doses, wherein the plurality of doses together constitute a therapeutically effective dose or clinically effective dose regimen. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In certain embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, the first dosage regimen includes hypoimmune (HIP) T cells or primary T cells expressing a first CAR and the second dosage regimen includes hypoimmune (HIP) T cells or primary T cells expressing a second CAR such that the first CAR and the second CAR are different. For instance, the first CAR and second CAR bind different target antigens. In some cases, the first CAR includes an scFv that binds an antigen and the second CAR includes an scFv that binds a different antigen. In some embodiments, the first dosage regimen includes hypoimmune (HIP) T cell or primary T cells expressing a first CAR and the second dosage regimen includes hypoimmune (HIP) T cell or primary T cells expressing a second CAR such that the first CAR and the second CAR are the same. The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., autoimmune diseases) and at least two of the dosage regimens comprise the same type of hypoimmune (HIP) T cells or primary T cells described herein. In other embodiments, at least two of the plurality of dosage regimens comprise different types of hypoimmune (HIP) T cells or primary T cells described herein.

In some embodiments, the CD19 specific (CD19) CAR-T cells described herein are administered to a subject at a dose of about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the viable CD19 specific CAR-T cells include CD19 specific CAR expressing CD4+ T cells and CD19 specific CAR expressing CD8+ T cells at a ratio of about 1:1. In some embodiments, the CD19 specific CAR of the cells is lisocabtagene maraleucel (BREYANZI®), a structural equivalent thereof, or a functional equivalent thereof.

In some embodiments, a subject is administered about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells described herein. In some embodiments, the dose is a therapeutically effective amount of viable CD19 specific CAR-T cells. In other embodiments, the dose is a clinically effective amount of viable CD19 specific CAR-T cells. In some instances, 50% of the viable CD19 specific CAR-T cells are CD19 specific CAR expressing CD4+ T cells and 50% of the viable CD19 specific CAR-T cells are CD19 specific CAR expressing CD8+ T cells. In some embodiments, the CD19 specific CAR of the cells is lisocabtagene maraleucel (BREYANZI®), a structural equivalent thereof, or a functional equivalent thereof.

In some embodiments, the CD19 specific CAR-T cells described herein are administered to a subject at a dose of about 2×10⁶ per kg of body weight. In some embodiments, a maximum dose administered is about 2×10⁸ viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as axicabtagene ciloleucel (YESCARTA®), a structural equivalent thereof, or a functional equivalent thereof.

In some embodiments, the CD19 specific CAR-T cells described herein are administered to a subject at a dose of about 2×10⁶ per kg of body weight. In some embodiments, a maximum dose of about 2×10⁸ viable CD19 specific CAR-T cells is administered to a patient of about 100 kg of body weight and above. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as brexucabtagene autoleucel (TECARTUS®), a structural equivalent thereof, or a functional equivalent thereof.

In some embodiments, the CD19 specific CAR-T cells described herein are administered to a subject at a dose of up to about 2×10⁸ viable CD19 specific CAR-T cells. In some embodiments, a subject is administered from about 0.2×10⁶ to about 5.0×10⁶ (e.g., about 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, 2.5×10⁶, 2.6×10⁶, 2.8×10⁶, 2.9×10⁶, 3.0×10⁶, 3.2×10⁶, 3.4×10⁶, 3.5×10⁶, 3.6×10⁶, 3.8×10⁶, 3.9×10⁶, 4.0×10⁶, 4.2×10⁶, 4.4×10⁶, 4.5×10⁶, 4.6×10⁶, 4.8×10⁶, 4.9×10⁶, or 5.0×10⁶) viable CD19 specific CAR-T cells per kg of body weight for a subject with a body weight of about 50 kg or less. In some embodiments, a subject is administered from about 0.1×10⁸ to about 2.5×10⁸ (e.g., about 0.1×10⁶, 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, or 2.5×10⁶) viable CD19 specific CAR-T cells for a subject with a body weight of greater than about 50 kg. In some embodiments, a subject is administered from about 0.6×10⁸ to about 6.0×10⁸ (e.g., about 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, 5.0×10⁸, 5.2×10⁸, 5.4×10⁸, 5.5×10⁸, 5.6×10⁸, 5.8×10⁸, 5.9×10⁸, or 6.0×10⁸) viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as tisagenlecleucel (KYMRIAH®), a structural equivalent thereof, or a functional equivalent thereof.

In some embodiments, a single dose of any of the CD19 specific CAR-T cells described herein includes about 50×10⁶ to about 110×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, or 110×10⁶) viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the viable CD19 specific CAR-T cells include CD19 specific CAR expressing CD4+ T cells and CD19 specific CAR expressing CD8+ T cells at a ratio of about 1:1. In some embodiments, the CD19 specific CAR is the same CD19 specific CAR as lisocabtagene maraleucel (BREYANZI″), a structural equivalent thereof, or a functional equivalent thereof.

In some embodiments, a single dose of any of the CD19 specific CAR-T cells described herein includes about 2×10⁸ viable CD19 specific CAR-T cells. In some embodiments, a single infusion bag of any of the CD19 specific CAR-T cells described herein includes about 2×10⁸ viable CD19 specific CAR-T cells in a cell suspension of about 68 mL. In some embodiments, the CD19 specific CAR is the same CD19 specific CAR as axicabtagene ciloleucel (YESCARTA″), a structural equivalent thereof, or a functional equivalent thereof.

In some embodiments, a single dose of any of the CD19 specific CAR-T cells described herein includes about 2×10⁸ viable CD19 specific CAR-T cells. In some embodiments, a single infusion bag of any of the CD19 specific CAR-T cells described herein includes about 2×10⁸ viable CD19 specific CAR-T cells in a cell suspension of about 68 mL. In some embodiments, the CD19 specific CAR is the same CD19 specific CAR as brexucabtagene autoleucel (TECARTUS®), a structural equivalent thereof, or a functional equivalent thereof.

In some embodiments, a single dose of any of the CD19 specific CAR-T cells described herein includes about 0.2×10⁶ to about 5.0×10⁶ (e.g., about 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, or 5.0×10⁶) viable CD19 specific CAR-T cells per kg of body weight for a subject with a body weight of 50 kg or less. In some embodiments, a single dose of any of the CD19 specific CAR-T cells described herein includes about 0.1×10⁸ to about 2.5×10⁸ (e.g., about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, or 2.5×10⁶) viable CD19 specific CAR-T cells per kg of body weight for a subject with a body weight of more than 50 kg. In some embodiments, a single dose of any of the CD19 specific CAR-T cells described herein includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells. In some embodiments, a single infusion bag of any of the CD19 specific CAR-T cells described herein includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells in a cell suspension of from about 10 mL to about 50 mL. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as tisagenlecleucel (KYMRIAH″), a structural equivalent thereof, or a functional equivalent thereof.

In some embodiments, the BCMA specific (BCMA) CAR-T cells described herein are administered to a subject at a dose of about 250×10⁶ to about 500×10⁶ (e.g., 250×10⁶, 255×10⁶, 260×10⁶, 265×10⁶, 270×10⁶, 275×10⁶, 280×10⁶, 285×10⁶, 290×10⁶, 295×10⁶, 300×10⁶, 305×10⁶, 310×10⁶, 315×10⁶, 320×10⁶, 325×10⁶, 330×10⁶, 335×10⁶, 340×10⁶, 345×10⁶, 350×10⁶, 355×10⁶, 360×10⁶, 365×10⁶, 370×10⁶, 375×10⁶, 380×10⁶, 385×10⁶, 390×10⁶, 395×10⁶, 400×10⁶, 405×10⁶, 410×10⁶, 415×10⁶, 420×10⁶, 425×10⁶, 430×10⁶, 435×10⁶, 440×10⁶, 445×10⁶, 450×10⁶, 455×10⁶, 460×10⁶, 465×10⁶, 470×10⁶, 475×10⁶, 480×10⁶, 485×10⁶, 490×10⁶, 495×10⁶, or 500×10⁶) viable BCMA specific CAR-T cells. In some embodiments, the dose is a therapeutically effective amount of viable BCMA specific CAR-T cells. In other embodiments, the dose is a clinically effective amount of viable BCMA specific CAR-T cells. In some embodiments, the viable BCMA specific CAR-T cells include BCMA specific CAR expressing CD4+ T cells and BCMA specific CAR expressing CD8+ T cells at a ratio of about 1:1. In some embodiments, the BCMA specific CAR of the cells is idecabtagene vicleucel (ABECEMA®), a structural equivalent thereof, or a functional equivalent thereof.

In some embodiments, a subject is administered about 250×10⁶ to about 500×10⁶ (e.g., 250×10⁶, 255×10⁶, 260×10⁶, 265×10⁶, 270×10⁶, 275×10⁶, 280×10⁶, 285×10⁶, 290×10⁶, 295×10⁶, 300×10⁶, 305×10⁶, 310×10⁶, 315×10⁶, 320×10⁶, 325×10⁶, 330×10⁶, 335×10⁶, 340×10⁶, 345×10⁶, 350×10⁶, 355×10⁶, 360×10⁶, 365×10⁶, 370×10⁶, 375×10⁶, 380×10⁶, 385×10⁶, 390×10⁶, 395×10⁶, 400×10⁶, 405×10⁶, 410×10⁶, 415×10⁶, 420×10⁶, 425×10⁶, 430×10⁶, 435×10⁶, 440×10⁶, 445×10⁶, 450×10⁶, 455×10⁶, 460×10⁶, 465×10⁶, 470×10⁶, 475×10⁶, 480×10⁶, 485×10⁶, 490×10⁶, 495×10⁶, or 500×10⁶) viable BCMA specific CAR-T cells described herein. In some embodiments, the dose is a therapeutically effective amount of viable BCMA specific CAR-T cells. In other embodiments, the dose is a clinically effective amount of viable BCMA specific CAR-T cells. In some instances, 50% of the viable BCMA specific CAR-T cells are BCMA specific CAR expressing CD4+ T cells and 50% of the viable BCMA specific CAR-T cells are BCMA specific CAR expressing CD8+ T cells. In some embodiments, the BCMA specific CAR of the cells is idecabtagene vicleucel (ABECEMA®), a structural equivalent thereof, or a functional equivalent thereof.

In some embodiments, the BCMA specific CAR-T cells described herein are administered to a subject at a dose of up to about 5×10⁸ viable BCMA specific CAR-T cells. In some embodiments, a subject is administered from about 2.5×10⁸ to about 5.0×10⁸ (e.g., about 0.2×10⁸, 0.4×10⁸, 0.5×10⁸, 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, or 5.0×10⁸) viable BCMA specific CAR-T cells per kg of body weight. In some embodiments, the dose is a therapeutically effective amount of viable BCMA specific CAR-T cells. In other embodiments, the dose is a clinically effective amount of viable BCMA specific CAR-T cells. In some embodiments, the BCMA specific CAR of the cells is the same BCMA specific CAR as idecabtagene vicleucel (ABECEMA″), a structural equivalent thereof, or a functional equivalent thereof.

In some embodiments, a single dose of any of the BCMA specific CAR-T cells described herein includes about 250×10⁶ to about 500×10⁶ (e.g., 250×10⁶, 255×10⁶, 260×10⁶, 265×10⁶, 270×10⁶, 275×10⁶, 280×10⁶, 285×10⁶, 290×10⁶, 295×10⁶, 300×10⁶, 305×10⁶, 310×10⁶, 315×10⁶, 320×10⁶, 325×10⁶, 330×10⁶, 335×10⁶, 340×10⁶, 345×10⁶, 350×10⁶, 355×10⁶, 360×10⁶, 365×10⁶, 370×10⁶, 375×10⁶, 380×10⁶, 385×10⁶, 390×10⁶, 395×10⁶, 400×10⁶, 405×10⁶, 410×10⁶, 415×10⁶, 420×10⁶, 425×10⁶, 430×10⁶, 435×10⁶, 440×10⁶, 445×10⁶, 450×10⁶, 455×10⁶, 460×10⁶, 465×10⁶, 470×10⁶, 475×10⁶, 480×10⁶, 485×10⁶, 490×10⁶, 495×10⁶, or 500×10⁶) viable BCMA specific CAR-T cells. In some embodiments, the dose is a therapeutically effective amount of viable BCMA specific CAR-T cells. In other embodiments, the dose is a clinically effective amount of viable BCMA specific CAR-T cells. In some embodiments, the viable BCMA specific CAR-T cells include BCMA specific CAR expressing CD4+ T cells and BCMA specific CAR expressing CD8+ T cells at a ratio of about 1:1. In some embodiments, the BCMA specific CAR is the same BCMA specific CAR as idecabtagene vicleucel (ABECEMA®), a structural equivalent thereof, or a functional equivalent thereof.

In some embodiments, a single dose of any of the BCMA specific CAR-T cells described herein includes about 250×10⁶ to about 500×10⁶ (e.g., 250×10⁶, 255×10⁶, 260×10⁶, 265×10⁶, 270×10⁶, 275×10⁶, 280×10⁶, 285×10⁶, 290×10⁶, 295×10⁶, 300×10⁶, 305×10⁶, 310×10⁶, 315×10⁶, 320×10⁶, 325×10⁶, 330×10⁶, 335×10⁶, 340×10⁶, 345×10⁶, 350×10⁶, 355×10⁶, 360×10⁶, 365×10⁶, 370×10⁶, 375×10⁶, 380×10⁶, 385×10⁶, 390×10⁶, 395×10⁶, 400×10⁶, 405×10⁶, 410×10⁶, 415×10⁶, 420×10⁶, 425×10⁶, 430×10⁶, 435×10⁶, 440×10⁶, 445×10⁶, 450×10⁶, 455×10⁶, 460×10⁶, 465×10⁶, 470×10⁶, 475×10⁶, 480×10⁶, 485×10⁶, 490×10⁶, 495×10⁶, or 500×10⁶) viable BCMA specific CAR-T cells per kg of body weight. In some embodiments, a single dose of any of the BCMA specific CAR-T cells described herein includes about 2.5×10⁸ to about 5.0×10⁸ (e.g., about 0.2×10⁸, 0.4×10⁸, 0.5×10⁸, 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, or 5.0×10⁸) viable BCMA specific CAR-T cells per kg of body weight. In some embodiments, a single dose of any of the BCMA specific CAR-T cells described herein includes about 2.5×10⁸ to about 5.0×10⁸ (e.g., about 0.2×10⁸, 0.4×10⁸, 0.5×10⁸, 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, or 5.0×10⁸) viable BCMA specific CAR-T cells. In some embodiments, a single infusion bag of any of the BCMA specific CAR-T cells described herein includes about 2.5×10⁸ to about 5.0×10⁸ (e.g., about 0.2×10⁸, 0.4×10⁸, 0.5×10⁸, 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, or 5.0×10⁸) viable BCMA specific CAR-T cells in a cell suspension of from about 10 mL to about 500 mL. In some embodiments, the cell suspension is about 50 mL, 250 mL, or about 500 mL. In some embodiments, the dose is a therapeutically effective amount of viable BCMA specific CAR-T cells. In other embodiments, the dose is a clinically effective amount of viable BCMA specific CAR-T cells. In some embodiments, the BCMA specific CAR of the cells is the same BCMA specific CAR as idecabtagene vicleucel (ABECEMA®), a structural equivalent thereof, or a functional equivalent thereof.

### EE. Methods for Administering Hypoimmunogenic Cells Including T Cells

As is described in further detail herein, provided herein are methods for treating a patient with a condition, disorder, or disorder through administration of hypoimmunogenic cells, particularly hypoimmunogenic T cells. As will be appreciated, for all the multiple embodiments described herein related to the timing and/or combinations of therapies, the administration of the cells is accomplished by a method or route which results in at least partial localization of the introduced cells at a desired site. The cells can be infused, implanted, or transplanted directly to the desired site, or alternatively be administered by any appropriate route which results in delivery to a desired location in the subject where at least a portion of the implanted cells or components of the cells remain viable.

Provided herein are methods for treating a patient with a condition, disorder, or disorder includes administration of a population of hypoimmunogenic cells (e.g., primary T cells, T cells differentiated from hypoimmunogenic induced pluripotent stem cells, or other cells differentiated from hypoimmunogenic induced pluripotent stem cells described herein) to a subject, e.g., a human patient. For instance, a population of hypoimmunogenic primary T cells such as, but limited to, CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γδ T cells, and any other subtype of T cell is administered to a patient to treat a condition, disorder, or disorder. In some embodiments, an immunosuppressive and/or immunomodulatory agent (such as, but not limited to a lymphodepletion agent) is not administered to the patient before the administration of the population of hypoimmunogenic cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the administration of the cells. In numerous embodiments, an immunosuppressive and/or immunomodulatory agent is not administered to the patient after the administration of the cells, or is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the administration of the cells. In some embodiments where an immunosuppressive and/or immunomodulatory agent is administered to the patient before or after the administration of the cells, the administration is at a lower dosage than would be required for cells with one or more MHC I and/or MHC II molecule expression and without exogenous expression of CD47.

Non-limiting examples of an immunosuppressive and/or immunomodulatory agent (such as, but not limited to a lymphodepletion agent) include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In some embodiments, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, where an immunosuppressive and/or immunomodulatory agent is administered to the patient before or after the administration of the cells, the administration is at a lower dosage than would be required for cells with one or more MHC I and/or MHC II molecule expression, TCR expression and without exogenous expression of CD47. In some embodiments, where an immunosuppressive and/or immunomodulatory agent is administered to the patient before or after the first administration of the cells, the administration is at a lower dosage than would be required for cells with one or more MHC I and MHC II molecule expression, TCR expression and without exogenous expression of CD47.

In some embodiments, the cells described are co-administered with a therapeutic agent that that binds to and/or interacts with one or more receptors selected from the group consisting of CD94, KIR2DL4, PD-1, an inhibitory NK cell receptor, and an activating NK receptor. In some instances, the therapeutic agent binds to a receptor on the surface of an NK cell, including one or more subpopulations of NK cells. In some embodiments, the therapeutic agent is selected from the group consisting of an antibody and fragments and variants thereof, an antibody mimetic, a small molecule, a blocking peptide, and a receptor antagonist.

For therapeutic application, cells prepared according to the disclosed methods can typically be supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

In some embodiments, the cells described herein are contraindicated in patients with known Type I hypersensitivity or anaphylactic reactions to murine proteins, Chinese Hamster Ovary (CHO) cell proteins, or to any component of the compositions described herein. In some embodiments, the cells described herein are contraindicated in patients who have or have had progressive multifocal leukoencephalopathy (PML). In some embodiments, the cells described herein are not recommended for use in patients with severe, active infections.

In some embodiments, the cells described herein are administered to a subject with an autoimmune disease/disorder and/or inflammatory disease/disorder who has been previously treated with rituximab (RITUXAN®). In some embodiments, the cells described herein are administered to a subject with an autoimmune disease/disorder and/or inflammatory disease/disorder who has been previously treated with rituximab (RITUXAN®) and has failed and/or not responded to the rituximab treatment. In some embodiments, the patent has rheumatoid arthritis (RA). In some embodiments, the patient has RA and the rituximab treatment is in combination with methotrexate. In some embodiments, the patient is an adult patient that has moderately- to severely-active RA. In some embodiments, the patient is an adult patient that has moderately- to severely-active RA and the rituximab treatment is in combination with methotrexate. In some embodiments, the patient is an adult patient that has moderately- to severely-active RA who has inadequate response to one or more TNF antagonist therapies and the rituximab treatment is in combination with methotrexate. In some embodiments, the rituximab dose for RA in combination with methotrexate is two-1000 mg intravenous infusions separated by 2 weeks (one course) every 24 weeks and/or based on clinical evaluation, but not sooner than every 16 weeks. In some embodiments, the Methylprednisolone 100 mg intravenous or equivalent glucocorticoid is recommended 30 minutes prior to each infusion.

In some embodiments, the cells described herein are administered to a subject with an autoimmune disease/disorder and/or inflammatory disease/disorder who has been previously treated with rituximab (RITUXAN®). In some embodiments, the cells described herein are administered to a subject with an autoimmune disease/disorder and/or inflammatory disease/disorder who has been previously treated with rituximab (RITUXAN®) and has failed and/or not responded to the rituximab treatment. In some embodiments, the patent has granulomatosis with polyangiitis (GPA) (Wegener's Granulomatosis). In some embodiments, the patent has Microscopic polyangiitis (MPA) in adult patients in combination with glucocorticoids. In some embodiments, the rituximab dose for GPA and MPA in combination with glucocorticoids is 375 mg/m² once weekly for 4 weeks. In some embodiments, the rituximab is administered as a 100 mg/10 mL solution in a single-use vial. In some embodiments, the rituximab is administered as a 500 mg/50 mL solution in a single-use vial.

In some embodiments, cells described herein are administered to a subject with an autoimmune disease/disorder and/or inflammatory disease/disorder as part of a combination therapy. In some embodiments, cells described herein are administered to a subject with an autoimmune disease/disorder and/or inflammatory disease/disorder as part of a combination therapy with an anti-B-lymphocyte stimulator (anti-BLyS) therapy. In some embodiments, an anti-BLyS therapy comprises belimumab.

FF. Autoimmune Diseases/Disorders and/or Inflammatory Diseases/Disorders for Treatment

Autoimmune or inflammatory disorders include diseases or disorders arising from and directed against an individual's own tissues or organs or a manifestation thereof or a condition resulting therefrom. In one embodiment, it refers to a condition that results from, or is aggravated by, the production of T cells that are reactive with normal body tissues and antigens. In one embodiment, it refers to a condition that results from, or is aggravated by, the production by antibodies that are reactive with normal body tissues and antigens.

In some embodiments, autoimmune or inflammatory disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis (such as plaque psoriasis, gutatte psoriasis, pustular psoriasis, and psoriasis of the nails), atopy (including atopic diseases such as hay fever and Job's syndrome), dermatitis (including contact dermatitis, chronic contact dermatitis, exfoliative dermatitis, exfoliative psoriatic dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis), x-linked hyper IgM syndrome, allergic intraocular inflammatory diseases, urticaria (such as chronic allergic urticaria, chronic idiopathic urticaria, chronic autoimmune urticaria), myositis, polymyositis/dermatomyositis, juvenile dermatomyositis, toxic epidermal necrolysis, scleroderma (including systemic scleroderma), sclerosis (such as systemic sclerosis; multiple sclerosis (MS), MS associated with EBV infection, spino-optical MS, primary progressive MS (PPMS), relapsing-remitting MS (RRMS), progressive relapsing MS, secondary progressive MS (SPMS), progressive systemic sclerosis, atherosclerosis, arteriosclerosis, sclerosis disseminata, and ataxic sclerosis), neuromyelitis optica spectrum disorder (NMO, also known as Devic's Disease or Devic's Syndrome), inflammatory bowel disease (IBD) including Crohn's disease; autoimmune-mediated gastrointestinal diseases; colitis such as ulcerative colitis, colitis ulcerosa, microscopic colitis, collagenous colitis, colitis polyposa, necrotizing enterocolitis, and transmural colitis; and autoimmune inflammatory bowel disease), bowel inflammation, pyoderma gangrenosum, erythema nodosum, primary sclerosing cholangitis, respiratory distress syndrome (including adult or acute respiratory distress syndrome (ARDS)), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis (such as anterior uveitis, acute anterior uveitis, granulomatous uveitis, nongranulomatous uveitis, phacoantigenic uveitis, posterior uveitis, or autoimmune uveitis), glomerulonephritis (GN) with and without nephrotic syndrome (such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, or proliferative nephritis), autoimmune polyglandular endocrine failure, balanitis including balanitis circumscripta plasmacellularis, balanoposthitis, erythema annulare centrifugum, erythema dyschromicum perstans, erythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema (including allergic or atopic eczema, asteatotic eczema, dyshidrotic eczema, and vesicular palmoplantar eczema), asthma (such as asthma bronchiale, bronchial asthma, and auto-immune asthma), conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus (including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE or subacute cutaneous SLE, neonatal lupus syndrome (NLE), and lupus erythematosus disseminatus, Type I diabetes, Type II diabetes, and latent autoimmune diabetes in adults (or Type 1.5 diabetes), juvenile onset (Type I) diabetes mellitus, including pediatric insulin-dependent diabetes mellitus (IDDM), adult onset diabetes mellitus (Type II diabetes), idiopathic diabetes, insipidus, diabetic retinopathy, diabetic nephropathy, and diabetic large-artery disorder; immune responses associated with acute and delayed hypersensitivity mediated by cytokines and T-lymphocytes; tuberculosis, sarcoidosis, granulomatosis (including lymphomatoid granulomatosis, Wegener's granulomatosis, or agranulocytosis), vasculitides (including vasculitis, large-vessel vasculitis, polymyalgia rheumatica and giant cell (Takayasu's) arteritis, medium-vessel vasculitis, Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis (such as Churg-Strauss vasculitis or syndrome (CSS) and ANCA-associated small-vessel vasculitis)), temporal arteritis, aplastic anemia, autoimmune aplastic anemia, Coombs positive anemia, Diamond Blackfan anemia, hemolytic anemia, immune hemolytic anemia including autoimmune hemolytic anemia (AIHA), pernicious anemia (anemia perniciosa), Addison's disease, pure red cell anemia or aplasia (PRCA); Factor VIII deficiency; hemophilia A; autoimmune neutropenia, pancytopenia, leukopenia, diseases involving leukocyte diapedesis, CNS inflammatory disorders, Alzheimer's disease, Parkinson's disease, multiple organ injury syndrome (such as those secondary to septicemia, trauma, or hemorrhage), antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP), post-transfusion purpura (PTP), heparin-induced thrombocytopenia, autoimmune or immune-mediated thrombocytopenia such as idiopathic thrombocytopenia purpura (ITP) including chronic or acute ITP, acquired thrombocytopenia purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases, including thyroiditis autoimmune thyroiditis, Hashimoto's disease, chronic thyroiditis (Hashimoto's thyroiditis), or subacute thyroiditis), autoimmune thyroid disease, idiopathic hypothyroidism, or Grave's disease), polyglandular syndromes, autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), sensory neuropathy, multifocal motor neuropathy, Sheehan's syndrome, hepatitis, including autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility (e.g., due to anti-spermatozoan antibodies) mixed connective tissue disease, Chagas' disease, rheumatic fever, recurrent abortion, farmer's lung, erythema multiforme, post-cardiotomy syndrome, post myocardial infarction cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Samter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, endometriosis, and pulmonary hemosiderosis.

### 1. Multiple Sclerosis

Multiple Sclerosis (MS) is an inflammatory and demyelinating degenerative disease of the human central nervous system (CNS) which affects approximately 300,000 persons in the United States (see, Anderson et al. Ann Neurology 31 (3): 333-6 (1992); Noonan et al. Neurology 58:136-8 (2002)). MS is a heterogeneous disorder based on clinical course, magnetic resonance imaging (MRI) scan assessment, and pathology analysis of biopsy and autopsy material (see, Lucchinetti et al. Ann Neurol 47:707-17 (2000)). The disease manifests itself in a large number of possible combinations of deficits, including spinal cord, brainstem, cranial nerve, cerebellar, cerebral, and cognitive syndromes. MS can be difficult to diagnose because of the non-specific clinical findings, which led to the development of highly structured diagnostic criteria that include several technological advances, consisting of MRI scans, evoked potentials, and cerebrospinal fluid (CSF) studies. All diagnostic criteria rely upon the general principles of scattered lesions in the central white matter occurring at different times and not explained by other etiologies such as infection, vascular disorder, or autoimmune disorder (see, McDonald et al. Ann Neurol 50:121-7 (2001)). MS has four patterns of disease: relapsing-remitting MS (RRMS; 80%-85% of cases at onset), primary progressive MS (PPMS; 10%-15% at onset), progressive relapsing MS (PRMS; 5% at onset); and secondary progressive MS (SPMS) (see, Kremenchutzky et al. Brain 122 (Pt 10): 1941-50 (1999); Confavreux et al. N Engl J Med 343 (20): 1430-8 (2000)). An estimated 50% of patients with RRMS will develop SPMS in 10 years, and up to 90% of RRMS patients will eventually develop SPMS (Weinshenker et al. Brain 112 (Pt 1): 133-46 (1989)).

In some embodiments, the multiple sclerosis is relapsing-remitting multiple sclerosis, progressive relapsing multiple sclerosis, primary progressive multiple sclerosis, or secondary progressive multiple sclerosis. In some embodiments, the multiple sclerosis is relapsing-remitting multiple sclerosis. In some embodiments, the multiple sclerosis is progressive relapsing multiple sclerosis. In some embodiments, the multiple sclerosis is primary progressive multiple sclerosis. In some embodiments, the multiple sclerosis is secondary progressive multiple sclerosis.

### IV. Examples

### Example 1: In Vivo Study of HIP CAR-T Cells in an SLE Mouse Model

### Systemic Lupus Erythematosus (SLE)

Systemic lupus erythematosus (SLE) is a multifactorial autoimmune disease characterized by poly-systemic inflammation as a result of auto-reactive T and B cells. In mammals, the immune system can sustain prolonged adaptive immune responses to foreign antigens by generating, through genetic recombination, lymphocytes with highly diverse receptors. Due to the randomness of this process, a proportion of T and B cells may have receptors that are directed against self-antigen; however, these lymphocytes can be eliminated, reprogrammed, or inactivated in the lymphoid organs.

In SLE patients, regulatory T cells that remove autoantibody-antigen complexes may be reduced or lacking, and the continued presence of these complexes activate an autoimmune positive feedback loop that allows the disease to progress beyond the inciting autoantigen through “epitope spreading”.

SLE has been recognized in a variety of species including humans, mice, rats, and other domesticated animals. SLE in mice closely resembles SLE in humans, including autoantibody production and renal disease. Mouse models of SLE fall under two main categories, spontaneous and induced, and each model presents its own iterations of lupus-like disease with a subset of symptoms similar to those seen in human SLE.

Two commonly used mouse SLE models are the MRL/MpJ-Fas^{lpr} and NZBWF1/J strains, which develop spontaneous disease, i.e., with no external stimuli required, characterized by hyperactive B and T cells, high titers of several autoantibodies directed against nuclear antigens, defective clearance of immune complexes, and fatal immune glomerulonephritis. Although these mouse models are primarily characterized by the development of nephritis, other lupus-like symptoms may develop in either strain. MRL/MpJ-Fas^{lpr} mice develop nephritis as well as other human disease-relevant manifestations (skin rash, cerebritis, vasculitis, auto-antibodies) within weeks, and brain manifestations of SLE include extrafollicular lymphoid structures.

Different SLE model mice will be subjected to treatment with HIP CAR-T cells described herein.

### In Vivo Study

Mice with spontaneous SLE will be employed to evaluate the efficacy of CD19 hypoimmunogenic CAR-T cells (HLA-/TCR-/CD47tg CD19-specific CAR-T cells) in treatment of SLE.

Standard clinical endpoints for MRL/MpJ-Fas^{lpr} models include proteinuria, body weight changes, anti-dsdna titers, anti-ana titers, and/or lymphadenopathy skin lesions. Standard morphologic endpoints for MRL/MpJ-Fas^{lpr}-models include glomerulonephritis, protein casts, interstitial nephritis, perivascular infiltration, and/or total histopathology.

Standard clinical endpoints for NZBWF1/J models include Proteinuria, body weight changes, anti-dsdna titers, and/or anti-ana titers. Standard morphologic endpoints for NZBWF1/J models include glomerulonephritis, protein casts, interstitial nephritis, perivascular infiltration, and/or total histopathology. Other endpoints for NZBWF1/J models (including a muring CAR-T) include systemic autoimmunity, hemolytic anemia, proteinuria, and/or immune complex glomerulonephritis.

Standard endpoints for MRL/lpr* models include autoantibodies, skin rash, arthritis, active proliferative glomerulonephritis, and/or total knockout of B cells ameliorated disease.

Standard endpoints and markers of disease onset and progression for CD20+ Humanized Mouse models include B cell depletion and/or induction of SLE-like kidney damage (damage induction) in the presence of pristane.

Published data for Rituximab, Ocrelizumab, Obinutuzumab, and CD19 CAR-T (Kansal et al., 2019), all of which have been tested in the MRL/MpJ-Fas^{lpr} model, will serve as a comparison for the results of the study.

### Disease Parameters/Progression:

On study day 0, animals will be randomized into treatment groups based on body weight, proteinuria, clinical DAI, anti-dsDNA (ANA) titers or a combination of outcomes.

Treatment groups will include vehicle control, CAR-T cells, and cyclophosphamide (CPA) positive control. Animals will be assessed weekly for body weight change, proteinuria, skin lesion scoring, blood drawing for autoantibody testing, and clinical scores. Post-study histology and CAR-T detection in organs, including kidney, will be carried out.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

### CERTAIN EMBODIMENTS

Embodiment 1. A method comprising administering a population of engineered T cells to a patient, wherein the engineered T cells comprise reduced expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs), wherein the one or more CARs comprise (i) an extracellular ligand-binding domain having specificity for CD19, CD20, CD22, BCMA, or an EBV antigen, (ii) a hinge domain, (iii) a transmembrane domain, (iv) a co-stimulatory domain, and (v) an intracellular signaling domain.

Embodiment 2. A method comprising administering a population of engineered T cells to a patient, wherein the engineered T cells comprise reduced expression of B2M and MHC class II transactivator (CIITA) relative to an unaltered or unmodified wild-type or control cell, a first exogenous polynucleotide encoding CD47, and a second exogenous polynucleotide encoding one or more CARs, wherein the one or more CARs comprise (i) an extracellular ligand-binding domain having specificity for CD19 or CD22, (ii) a hinge domain, (iii) a transmembrane domain, (iv) a co-stimulatory domain, and (v) an intracellular signaling domain.

Embodiment 3. A method comprising administering a population of engineered T cells to a patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise (i) an extracellular ligand-binding domain having specificity for CD19, CD20, CD22, BCMA, or an EBV antigen, (ii) a hinge domain, (iii) a transmembrane domain, (iv) a co-stimulatory domain, and (v) an intracellular signaling domain.

Embodiment 4. A method comprising administering a population of engineered T cells to a patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise (i) an extracellular ligand-binding domain having specificity for CD19, CD20, CD22, BCMA, or an EBV antigen, (ii) a hinge domain, (iii) a transmembrane domain, (iv) a co-stimulatory domain, and (v) an intracellular signaling domain.

Embodiment 5. A method comprising administering a population of engineered T cells to a patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 6. A method comprising administering a population of engineered T cells to a patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 7. A method comprising administering a population of engineered T cells to a patient, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 8. A method comprising administering a population of engineered T cells to a patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 9. A method comprising administering a population of engineered T cells to a patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 10. A method comprising administering a population of engineered T cells to a patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 11. A method comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 12. A method comprising administering a population of engineered T cells to a patient, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 13. A method comprising evaluating the patient for and/or diagnosing a patient for an autoimmune disease, and administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 14. A method of treating a patient with an Epstein Barr Virus (EBV) infection comprising administering a population of engineered T cells to the patient, wherein the engineered T cells comprise reduced expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs) wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 15. A method of treating a patient with an EBV infection comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs wherein the one or more CARs comprise an extracellular ligand-binding domain having specificity for CD19 or CD22, a hinge domain, a transmembrane domain, a co-stimulatory domain, and an intracellular signaling domain.

Embodiment 16. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 17. A method of treating multiple sclerosis in a patient that is suspected of having multiple sclerosis or has been diagnosed with multiple sclerosis comprising administering a population of engineered T cells to the patient, wherein the engineered T cells comprise reduced expression of beta-2-microglobulin (B2M) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 18. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 19. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and MHC class II transactivator (CIITA) relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 20. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 21. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 22. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 23. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 24. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 25. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 26. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 27. A method of treating multiple sclerosis in a patient that is suspected of having multiple sclerosis or has been diagnosed with multiple sclerosis comprising administering a population of engineered T cells to the patient, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 28. A method of treating multiple sclerosis in a patient that is suspected of having multiple sclerosis or has been diagnosed with multiple sclerosis comprising evaluating the patient for and/or diagnosing the patient with EBV infection and optionally multiple sclerosis, and administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 29. The method according to any one of embodiment embodiments 1-28, wherein the one or more CARs comprise a CD8α hinge domain, a CD28 hinge domain, or an IgG4 hinge domain.

Embodiment 30. The method according to embodiment embodiment 28, wherein the one or more CARs comprise a CD8α hinge domain having the amino acid sequence of SEQ ID NO: 9.

Embodiment 31. The method according to embodiment embodiment 28, wherein the one or more CARs comprise a CD28 hinge domain having the amino acid sequence of SEQ ID NO: 10 or 113.

Embodiment 32. The method according to embodiment embodiment 28, wherein the one or more CARs comprise a IgG4 hinge domain having the amino acid sequence of SEQ ID NO: 11 or 12.

Embodiment 33. The method according to any one of embodiments 1-32, wherein the one or more CARs comprise a CD8α transmembrane domain or a CD28 transmembrane domain.

Embodiment 34. The method according to embodiment 33, wherein the one or more CARs comprise a CD8α transmembrane domain having the amino acid sequence of SEQ ID NO: 14.

Embodiment 35. The method according to embodiment 33, wherein the one or more CARs comprise a CD28 transmembrane domain having the amino acid sequence of SEQ ID NO: 15 or 114.

Embodiment 36. The method according to any one of embodiments 1-35, wherein the one or more CARs comprise a 4-1BB costimulatory domain, a CD28 costimulatory domain, or a CD37 signaling domain.

Embodiment 37. The method according to embodiment 36, wherein the one or more CARs comprise a 4-1BB costimulatory domain having the amino acid sequence of SEQ ID NO: 16.

Embodiment 38. The method according to embodiment 36, wherein the one or more CARs comprise a CD28 costimulatory domain having the amino acid sequence of SEQ ID NO: 17.

Embodiment 39. The method according to embodiment 36, wherein the one or more CARs comprise a CD3ζ signaling domain having the amino acid sequence of SEQ ID NO: 18 or 115.

Embodiment 40. The method according to any one of embodiments 1-39, wherein the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 41. The method according to any one of embodiments 1-40, wherein the one or more CARs have a sequence of any one of SEQ ID NOs: 32, 34, 36, 117, 91, 92, 92, 128, 133, or 134.

Embodiment 42. A method of treating multiple sclerosis in a patient that is suspected of having multiple sclerosis or has been diagnosed with multiple sclerosis and administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD20 CAR having the CDR sequences of SEQ ID NO: 37.

Embodiment 43. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a EBV antigen CAR having the CDR sequences of SEQ ID NO: 133 or 134, and/or the CDR sequences from the VH/VL sequences of SEQ ID NOs: 129-132 or 135-172.

Embodiment 44. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117.

Embodiment 45. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD22 CAR having the CDR sequences of SEQ ID NO: 45.

Embodiment 46. A method of treating multiple sclerosis in a patient that is suspected of having multiple sclerosis or has been diagnosed with multiple sclerosis comprising administering a population of engineered T cells to the patient, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45.

Embodiment 47. A method of treating multiple sclerosis in a patient that is suspected of having multiple sclerosis or has been diagnosed with multiple sclerosis comprising administering a population of engineered T cells to the patient, wherein the engineered T cells comprise an exogenous polynucleotide encoding one or more CARs wherein the one or more CARs have a sequence of any one of SEQ ID NOs: 32, 34, 36, 117, 91, 92, 92, 128, 133, or 134, or wherein the one or more CARs have an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 48. The method of any one of embodiments 1-47 further comprising evaluating the patient for and/or diagnosing the patient with EBV infection and optionally multiple sclerosis prior to administering the population of engineered T cells to the patient.

Embodiment 49. The method of embodiment 48, wherein the diagnosis comprises evaluating the patient for EBV infection.

Embodiment 50. The method of embodiment 48 or 49, wherein the diagnosis comprises evaluating the patient for multiple sclerosis.

Embodiment 51. The method according to any one of embodiments 1-50, wherein the treatment prevents multiple sclerosis.

Embodiment 52. The method according to any one of embodiments 1-50, wherein the treatment treats multiple sclerosis.

Embodiment 53. The method according to any one of embodiments 1-52, wherein the patient with the EBV infection has been diagnosed with multiple sclerosis.

Embodiment 54. The method according to any one of embodiments 1-53, wherein the multiple sclerosis is relapsing-remitting multiple sclerosis, progressive relapsing multiple sclerosis, primary progressive multiple sclerosis, or secondary progressive multiple sclerosis.

Embodiment 55. The method according to any one of embodiments 1-54, wherein the patient undergoes remission of multiple sclerosis following administration of the engineered T cells.

Embodiment 56. The method according to any one of embodiments 1-55, wherein the patient with the EBV infection is undergoing treatment for the EBV infection.

Embodiment 57. The method according to any one of embodiments 1-56, wherein the patient with the EBV infection has an active EBV infection.

Embodiment 58 The method according to any one of embodiments 1-56, wherein the patient with the EBV infection has an inactive EBV infection.

Embodiment 59. The method according to any one of embodiments 1-58, wherein the patient undergoes a reduced EBV infection following administration of the engineered T cells, optionally wherein the reduced EBV infection is characterized by reduced viral load.

Embodiment 60. The method according to any one of embodiments 1-59, wherein the treatment prevents an EBV infection change from an inactive to an active EBV infection.

Embodiment 61. The method of any one of embodiments 1-60, wherein the method results in B cell depletion.

Embodiment 62. The method of any one of embodiments 1-61, wherein the engineered T cells comprise one or more of a CD19-specific CAR, a CD20-specific CAR, a CD22-specific CAR, a BCMA-specific CAR, a GPRC5D-specific CAR, a CD3δ-specific CAR, a CD70-specific CAR, a CD79b-specific CAR, and an EBV antigen-specific CAR.

Embodiment 63. A method of treating an autoimmune disease in a patient that is suspected of having the autoimmune disease or has been diagnosed with the autoimmune disease comprising administering a population of engineered T cells to the patient, wherein the engineered T cells comprise an exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 64. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 65. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 66. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 67. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 68. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 69. The method according to any one of embodiments 63-68, wherein the one or more CARs comprise a CD8α hinge domain, a CD28 hinge domain, or an IgG4 hinge domain.

Embodiment 70. The method according to embodiment 69, wherein the one or more CARs comprise a CD8α hinge domain having the amino acid sequence of SEQ ID NO: 9.

Embodiment 71. The method according to embodiment 69, wherein the one or more CARs comprise a CD28 hinge domain having the amino acid sequence of SEQ ID NO: 10 or 113.

Embodiment 72. The method according to embodiment 69, wherein the one or more CARs comprise a IgG4 hinge domain having the amino acid sequence of SEQ ID NO: 11 or 12.

Embodiment 73. The method according to any one of embodiments 63-72, wherein the one or more CARs comprise a CD8α transmembrane domain or a CD28 transmembrane domain.

Embodiment 74. The method according to embodiment 73, wherein the one or more CARs comprise a CD8α transmembrane domain having the amino acid sequence of SEQ ID NO: 14.

Embodiment 75. The method according to embodiment 73, wherein the one or more CARs comprise a CD28 transmembrane domain having the amino acid sequence of SEQ ID NO: 15 or 114.

Embodiment 76. The method according to any one of embodiments 63-75, wherein the one or more CARs comprise a 4-1BB costimulatory domain, a CD28 costimulatory domain, or a CD3ζ signaling domain.

Embodiment 77. The method according to embodiment 76, wherein the one or more CARs comprise a 4-1BB costimulatory domain having the amino acid sequence of SEQ ID NO: 16.

Embodiment 78. The method according to embodiment 76, wherein the one or more CARs comprise a CD28 costimulatory domain having the amino acid sequence of SEQ ID NO: 17.

Embodiment 79. The method according to embodiment 76, wherein the one or more CARs comprise a CD3ζ signaling domain having the amino acid sequence of SEQ ID NO: 18 or 115.

Embodiment 80. The method according to any one of embodiments 63-79, wherein the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 81. The method according to any one of embodiments 63-80, wherein the one or more CARs have a sequence of any one of SEQ ID NOs: 32, 34, 36, 117, 91, 92, 92, 128, 133, or 134.

Embodiment 82. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 83. The method according to embodiment 82, further comprising evaluating the patient for and/or diagnosing the patient with the autoimmune disease.

Embodiment 84. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 85. The method of any one of embodiments 82-84, wherein the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 117, with the following components: CD8α signal peptide, FMC63 scFv (VL-Whitlow linker-VH), CD8α hinge domain, CD8α transmembrane domain, 4-1BB costimulatory domain, and CD3ζ signaling domain.

Embodiment 86. The method of any one of embodiments 82-85, wherein the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 45.

Embodiment 87. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a EBV antigen-specific CAR having the CDR sequences of SEQ ID NO: 133 or 134, and/or the CDR sequences from the VH/VL sequences of SEQ ID NOs: 129-132 or 135-172, and wherein the autoimmune disease is multiple sclerosis.

Embodiment 88. A method of treating an autoimmune disease in a patient that is suspected of having the autoimmune disease or has been diagnosed with the autoimmune disease comprising administering a population of engineered T cells to the patient, wherein the engineered T cells comprise an exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 89. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

Embodiment 90. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 91. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 92. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 93. A method of treating an autoimmune disease in a patient that is suspected of having the autoimmune disease or has been diagnosed with the autoimmune disease comprising administering a population of engineered T cells to the patient, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 94. A method of treating an autoimmune disease in a patient that is suspected of having the autoimmune disease or has been diagnosed with the autoimmune disease comprising administering a population of engineered T cells to the patient, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 95. The method of any one of embodiments 87-94 further comprising evaluating the patient for and/or diagnosing the patient with the autoimmune disease prior to administering the population of engineered T cells to the patient.

Embodiment 96. The method of any one of embodiments 87-95, wherein the autoimmune disease is selected from the group consisting of lupus, systemic lupus erythematosus, multiple sclerosis, systemic sclerosis, vasculitis, Crohn's disease, Myasthenia Gravis, Stiff-Person syndrome and a pulmonary condition.

Embodiment 97. The method of any one of embodiments 87-96, wherein the patient is suspected of having an EBV infection or has been diagnosed as having an EBV infection.

Embodiment 98. The method of any one of embodiments 87-97, wherein the patient is suspected of having multiple sclerosis or has been diagnosed with multiple sclerosis.

Embodiment 99. The method of any one of embodiments 1-98, further comprising administering a second, third, fourth, fifth, or sixth dose of the engineered T cells to the patient.

Embodiment 100. The method of embodiment 99, wherein the CAR is the same in one or more of the first, second, third, fourth, fifth, and/or sixth dose of engineered T cells.

Embodiment 101. The method of embodiment 99, wherein the CAR is different in one or more of the first, second, third, fourth, fifth, and/or sixth dose of engineered T cells.

Embodiment 102. The method of any one of embodiments 1-101, wherein the CAR has an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 103. The method of any one of embodiments 1-102, wherein the CAR has a sequence of any one of SEQ ID NOs: 32, 34, 36, 117, 91, 92, 92, 128, 133, and 134.

Embodiment 104. The method of any one of embodiments 1-103, wherein the engineered T cells comprise a CD19-specific CAR and a CD20-specific CAR.

Embodiment 105. The method of embodiment 104, wherein the CD19-specific CAR has the CDR sequences of SEQ ID NO: 117 and the CD22 CAR has the CDR sequences of SEQ ID NO: 45.

Embodiment 106. The method of embodiment 105, wherein the CD19-specific CAR and the CD20-specific CAR are encoded by a single bicistronic polynucleotide.

Embodiment 107. The method of embodiment 105, wherein the CD19-specific CAR and the CD20-specific CAR are encoded by a single bispecific CAR.

Embodiment 108. The method of embodiment 105, wherein the CD19-specific CAR and the CD20-specific CAR are encoded by two separate polynucleotides.

Embodiment 109. The method of any one of embodiments 104-108, wherein the CD19 CAR T cells and CD20 CAR T cells are administered concomitantly.

Embodiment 110. The method of any one of embodiments 104-108, wherein the CD19 CAR+ T cells and CD20 CAR+ T cells are administered sequentially.

Embodiment 111. The method of embodiment 110, wherein the CD19 CAR+ T cells are administered prior to administration of the CD20 CAR+ T cells.

Embodiment 112. The method of embodiment 110, wherein the CD20 CAR+ T cells are administered prior to administration of the CD19 CAR+ T cells.

Embodiment 113. The method of any one of embodiments 104-112, wherein the number of cells administered as a therapeutically effective amount of the CD19 and/or CD20 CAR T cells is greater than the number of cells administered as a therapeutically effective amount of CD19 CAR T cells or CD20 CAR T cells alone.

Embodiment 114. The method of any one of embodiments 104-113, wherein the number of cells administered to as a therapeutically effective amount of the CD19 and/or CD20 CAR T cells is less than the number of cells administered as a therapeutically effective amount of CD19 CAR T cells or CD20 CAR T cells alone.

Embodiment 115. The method of any one of embodiments 1-114, wherein the engineered T cells comprise an EBV antigen-specific CAR and a CD20-specific CAR.

Embodiment 116. The method of embodiment 115, wherein the EBV antigen-specific CAR and the CD20-specific CAR are encoded by a single bicistronic polynucleotide.

Embodiment 117. The method of embodiment 115, wherein the EBV antigen-specific CAR and the CD20-specific CAR are encoded by a single bispecific CAR.

Embodiment 118. The method of embodiment 115, wherein the EBV antigen-specific CAR and the CD20-specific CAR are encoded by two separate polynucleotides.

Embodiment 119. The method of any one of embodiments 115-119, wherein the EBV antigen CAR T cells and CD20 CAR T cells are administered concomitantly.

Embodiment 120. The method of any one of embodiments 115-119, wherein the EBV antigen CAR+ T cells and CD20 CAR+ T cells are administered sequentially.

Embodiment 121. The method of embodiment 120, wherein the EBV antigen CAR+ T cells are administered prior to administration of the CD20 CAR+ T cells.

Embodiment 122. The method of embodiment 120, wherein the CD20 CAR+ T cells are administered prior to administration of the EBV antigen CAR+ T cells.

Embodiment 123. The method of any one of embodiments 115-122, wherein the number of cells administered as a therapeutically effective amount of the EBV antigen and/or CD20 CAR T cells is greater than the number of cells administered as a therapeutically effective amount of EBV antigen CAR T cells or CD20 CAR T cells alone.

Embodiment 124. The method of any one of embodiments 115-122, wherein the number of cells administered to as a therapeutically effective amount of the EBV antigen and/or CD20 CAR T cells is less than the number of cells administered as a therapeutically effective amount of EBV antigen CAR T cells or CD20 CAR T cells alone.

Embodiment 125. The method of any one of embodiments 1-124, wherein the engineered T cells comprise an EBV antigen-specific CAR and a CD19-specific CAR.

Embodiment 126. The method of embodiment 125, wherein the EBV antigen-specific CAR and the CD19-specific CAR are encoded by a single bicistronic polynucleotide.

Embodiment 127. The method of embodiment 126, wherein the EBV antigen-specific CAR and the CD19-specific CAR are encoded by a single bispecific CAR.

Embodiment 128. The method of embodiment 126, wherein the EBV antigen-specific CAR and the CD19-specific CAR are encoded by two separate polynucleotides.

Embodiment 129. The method of any one of embodiments 126-128, wherein the EBV antigen CAR T cells and CD19 CAR T cells are administered concomitantly.

Embodiment 130. The method of any one of embodiments 126-128, wherein the EBV antigen CAR+ T cells and CD19 CAR+ T cells are administered sequentially.

Embodiment 131. The method of embodiment 130, wherein the EBV antigen CAR+ T cells are administered prior to administration of the CD19 CAR+ T cells.

Embodiment 132. The method of embodiment 130, wherein the CD19 CAR+ T cells are administered prior to administration of the EBV antigen CAR+ T cells.

Embodiment 133. The method of any one of embodiments 126-132, wherein the number of cells administered as a therapeutically effective amount of the EBV antigen and/or CD19 CAR T cells is greater than the number of cells administered as a therapeutically effective amount of EBV antigen CAR T cells or CD19 CAR T cells alone.

Embodiment 134. The method of any one of embodiments 126-132, wherein the number of cells administered to as a therapeutically effective amount of the EBV antigen and/or CD19 CAR T cells is less than the number of cells administered as a therapeutically effective amount of EBV antigen CAR T cells or CD19 CAR T cells alone.

Embodiment 135. The method of any one of embodiments 1-134, wherein the engineered T cells comprise an EBV antigen-specific CAR and a CD22-specific CAR.

Embodiment 136. The method of embodiment 135, wherein the EBV antigen-specific CAR and the CD22-specific CAR are encoded by a single bicistronic polynucleotide.

Embodiment 137. The method of embodiment 135, wherein the EBV antigen-specific CAR and the CD22-specific CAR are encoded by a single bispecific CAR.

Embodiment 138. The method of embodiment 135, wherein the EBV antigen-specific CAR and the CD22-specific CAR are encoded by two separate polynucleotides.

Embodiment 139. The method of any one of embodiments 135-138, wherein the EBV antigen CAR T cells and CD22 CAR T cells are administered concomitantly.

Embodiment 140. The method of any one of embodiments 135-138, wherein the EBV antigen CAR+ T cells and CD22 CAR+ T cells are administered sequentially.

Embodiment 141. The method of embodiment 140, wherein the EBV antigen CAR+ T cells are administered prior to administration of the CD22 CAR+ T cells.

Embodiment 142. The method of embodiment 140, wherein the CD22 CAR+ T cells are administered prior to administration of the EBV antigen CAR+ T cells.

Embodiment 143. The method of any one of embodiments 135-142, wherein the number of cells administered as a therapeutically effective amount of the EBV antigen and/or CD22 CAR T cells is greater than the number of cells administered as a therapeutically effective amount of EBV antigen CAR T cells or CD22 CAR T cells alone.

Embodiment 144. The method of any one of embodiments 135-142, wherein the number of cells administered to as a therapeutically effective amount of the EBV antigen and/or CD22 CAR T cells is less than the number of cells administered as a therapeutically effective amount of EBV antigen CAR T cells or CD22 CAR T cells alone.

Embodiment 145. The method of any one of embodiments 1-144, wherein the engineered T cells are primary T cells, are propagated from a primary T cell or a progeny thereof, or are derived from a T cell differentiated from an iPSC or a progeny thereof.

Embodiment 146. The method of any one of embodiments 1-145, wherein the engineered T cells are differentiated cells derived from an induced pluripotent stem cell or a progeny thereof.

Embodiment 147. The method of embodiment 146, wherein the differentiated cells are a T cells or natural killer (NK) cells.

Embodiment 148. The method of any one of embodiments 1-147, wherein the engineered T cells are primary T cells or are progeny of primary immune cells, optionally wherein the progeny of primary immune cells are T cells or NK cells.

Embodiment 149. The method of any one of embodiments 1-148, wherein the engineered T cells comprise reduced expression of one or more MHC HLA class I molecules relative to an unaltered or unmodified wild-type or control cell.

Embodiment 150. The method of any one of embodiments 1-149, wherein the engineered T cells comprise reduced expression of one or more MHC HLA class II molecules relative to an unaltered or unmodified wild-type or control cell.

Embodiment 151. The method of any one of embodiments 1-150, wherein the engineered T cells comprise reduced expression of one or more MHC HLA class I molecules and of one or more MHC HLA class II molecules relative to an unaltered or unmodified wild-type or control cell.

Embodiment 152. The method of any one of embodiments 1-151, wherein the engineered T cells comprise reduced expression of B2M and/or CIITA relative to an unaltered or unmodified wild-type or control cell.

Embodiment 153. The method of embodiment 152, wherein the engineered T cells do not express B2M and/or CIITA.

Embodiment 154. The method of any one of embodiments 1-153, wherein the engineered T cells comprise reduced expression of TRAC and/or TRB.

Embodiment 155. The method of embodiment 154, wherein the engineered T cells do not express TRAC and/or TRB.

Embodiment 156. The method of any one of embodiments 1-155, wherein the engineered T cells comprise reduced expression of TRAC.

Embodiment 157. The method of embodiment 156, wherein the engineered T cells do not express TRAC.

Embodiment 158. The method of any one of embodiments 1-157, wherein the engineered T cells comprise reduced expression of TRB.

Embodiment 159. The method of embodiment 158, wherein the engineered T cells do not express TRB.

Embodiment 160. The method of any one of embodiments 1-159, wherein the engineered T cells comprise reduced expression of TRAC and TRB.

Embodiment 161. The method of any one of embodiments 1-160, wherein the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9, optionally wherein the one or more tolerogenic factors comprise CD47.

Embodiment 162. The method of any one of embodiments 1-161, wherein the CD19-specific CAR and the one or more tolerogenic factors are encoded by a single bicistronic polynucleotide.

Embodiment 163. The method of any one of embodiments 1-161, wherein the CD19-specific CAR and the CD47 are encoded by a single bicistronic polynucleotide.

Embodiment 164. The method of any one of embodiments 1-161, wherein the CD20-specific CAR and the one or more tolerogenic factors are encoded by a single bicistronic polynucleotide.

Embodiment 165. The method of any one of embodiments 1-161, wherein the CD20-specific CAR and the CD47 are encoded by a single bicistronic polynucleotide.

Embodiment 166. The method of any one of embodiments 1-165, wherein one or more of the first, second, and/or third exogenous polynucleotides or the bicistronic polynucleotide is inserted into a first, second, and/or third specific locus of at least one allele of the cell.

Embodiment 167. The method of embodiment 166, wherein the first, second and/or third specific loci are selected from the group consisting of a safe harbor locus, a target locus, an RHD locus, a B2M locus, a CIITA locus, a TRAC locus, and a TRB locus.

Embodiment 168. The method of embodiment 167, wherein the safe harbor locus is selected from the group consisting of a CCR5 locus, a PPP1R12C locus, a CLYBL locus, and a Rosa locus.

Embodiment 169. The method of embodiment 167, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

Embodiment 170. The method of any one of embodiments 1-169, wherein the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.

Embodiment 171. The method of embodiments 170, wherein the gene therapy vector is a retrovirus or a fusosome.

Embodiment 172. The method of any one of embodiments 1-171, wherein the first, second, and/or third exogenous polynucleotide or the bicistronic polynucleotide is introduced into the engineered T cells using CRISPR/Cas gene editing.

Embodiment 173. The method of embodiment 172, wherein the CRISPR/Cas system comprises a Cas effector protein selected from the group consisting of Cas9, Cas12a, and Cas12b.

Embodiment 174. The method or dosage regimen of embodiment 173, wherein the CRISPR/Cas system comprises a Cas effector protein selected from the group consisting of:
(a) optionally selected from the group consisting of Cas3, Cas8a, Cas5, Cas8b, Cas8c, Cas10d, Cse1, Cse2, Csy1, Csy2, Csy3, and GSU0054;
(b) optionally selected from the group consisting of Cas9, Csn2, and Cas4;
(c) optionally selected from the group consisting of Cas10, Csm2, Cmr5, Cas10, Csx11, and Csx10;
(d) optionally Csf1;
(e) optionally selected from the group consisting of Cas12a, Cas12b, Cas12c, C2c4, C2c8, C2c5, C2c10, C2c9, CasX (Cas12e), and CasY (Cas12d); and
(f) optionally selected from the group consisting of Cas13, Cas13a, C2c2, Cas13b, Cas13c, and Cas13d.

Embodiment 175. The method of any one of embodiments 172-174, wherein the CRISPR/Cas gene editing is carried out ex vivo from a donor subject.

Embodiment 176. The method of any one of embodiments 172-175, wherein the first, second, and/or third exogenous polynucleotide or the bicistronic polynucleotide is introduced into the engineered T cells using a lentiviral vector.

Embodiment 177. The method of any one of embodiments 1-176, wherein the engineered T cells evade NK cell mediated cytotoxicity upon administration to the patient.

Embodiment 178. The method of any one of embodiments 1-177, wherein the engineered T cells are protected from cell lysis by mature NK cells upon administration to the patient.

Embodiment 179. The method of any one of embodiments 1-178, wherein the engineered T cells evade macrophage-mediated cytotoxicity, optionally wherein the macrophage-mediated cytotoxicity involves phagocytosis and/or reactive oxygen species.

Embodiment 180. The method of any one of embodiments 1-179, wherein the engineered T cells do not induce an immune response to the cell upon administration to the patient.

Embodiment 181. The method of any one of embodiments 1-180, wherein the administration is selected from the group consisting of intravenous injection, intramuscular injection, intravascular injection, and transplantation.

Embodiment 182. The method of any one of embodiments 1-181, wherein the engineered T cells are administered before, during or after starting a different treatment regimen for the patient.

Embodiment 183. The method of embodiment 182, wherein the different treatment regimen is selected from the group consisting of re-dosing of the same or different cells, and pre-treatment, concurrent treatment, or subsequent treatment with an additional agent.

Embodiment 184. The method of embodiment 183, wherein the different cells are autologous T or NK cells or CAR-T cells expressing a first CAR that is different from a second CAR expressed by the engineered CAR-T cells.

Embodiment 185. The method of any one of embodiments 1-184, wherein the patient was treated with an immunodepleting therapy prior to administering the engineered T cells.

Embodiment 186. The method of embodiment 185, wherein the immunodepleting therapy comprises administration of fludarabine and/or cyclophosphamide.

Embodiment 187. The method of any one of embodiments 1-186, wherein the patient has undergone a prior antibody therapy.

Embodiment 188. The method of embodiment 187, wherein the antibody therapy is rituximab.

Embodiment 189. The method of embodiment 186, wherein the immunodepleting therapy comprises IV infusion of about 1-50 mg/m2 of fludarabine for about 1-7 days.

Embodiment 190. The method of embodiment 189, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.

Embodiment 191. The method of embodiment 189 or 190, wherein the immunodepleting therapy comprises IV infusion of about 30 mg/m2 of fludarabine for about 4 days.

Embodiment 192. The method of embodiment 186, wherein the immunodepleting therapy comprises IV infusion of about 100-1000 mg/m2 of cyclophosphamide for about 1-7 days.

Embodiment 193. The method of embodiment 192, wherein the immunodepleting therapy comprises IV infusion of about 100, about 200, about 300, about 400, about 500, about 600, about 700, about 800, about 900, about 1000 mg/m2 of cyclophosphamide for about 1, about 2, about 3, about 4, about 5, about 6, or about 7 days.

Embodiment 194. The method of embodiment 192 or 193, wherein the immunodepleting therapy comprises IV infusion of about 500 mg/m2 of cyclophosphamide for about 2 days.

Embodiment 195. The method of any one of embodiments 1-194, wherein at least about 40×104 engineered T cells are administered to the patient.

Embodiment 196. The method of any one of embodiments 1-195, wherein at least about 40×105 engineered T cells are administered to the patient.

Embodiment 197. The method of any one of embodiments 1-196, wherein the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 198. The method of any one of embodiments 1-197, wherein the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 199. The method of any one of embodiments 1-198, wherein the wild type cell or the control cell is a starting material.

Embodiment 200. Use of a population of engineered T cells for treating an EBV infection in a patient that is suspected of having an EBV infection or has been diagnosed with an EBV infection, wherein the engineered T cells comprise reduced expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs) wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 201. Use of a population of engineered T cells for treating an EBV infection in a patient that is suspected of having an EBV infection or has been diagnosed with an EBV infection, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs wherein the one or more CARs comprise an extracellular ligand-binding domain having specificity for CD19 or CD22, a hinge domain, a transmembrane domain, a co-stimulatory domain, and an intracellular signaling domain.

Embodiment 202. Use of a population of engineered T cells for treating an EBV infection in a patient that is suspected of having an EBV infection or has been diagnosed with an EBV infection, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 203. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 204. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 205. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 206. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 207. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 208. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TCR-alpha (TRAC) and/or TCR-beta (TRB) relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 209. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 210. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 211. Use of a population of engineered T cells for treating multiple sclerosis in a patient that is suspected of having multiple sclerosis or has been diagnosed with multiple sclerosis, wherein the engineered T cells comprise reduced expression of B2M relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 212. Use of a population of engineered T cells for treating multiple sclerosis in a patient that is suspected of having multiple sclerosis or has been diagnosed with multiple sclerosis, wherein the engineered T cells comprise reduced expression of B2M and CIITA relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 213. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, reduced expression of TRAC and/or TRB relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 214. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 215. Use of a population of engineered T cells for treating multiple sclerosis in a patient that is suspected of having multiple sclerosis or has been diagnosed with multiple sclerosis, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

Embodiment 216. The use according to any one of embodiments 200-215, wherein the one or more CARs comprise a CD8α hinge domain, a CD28 hinge domain, or an IgG4 hinge domain.

Embodiment 217. The use according to embodiment 216, wherein the one or more CARs comprise a CD8α hinge domain having the amino acid sequence of SEQ ID NO: 9.

Embodiment 218. The use according to embodiment 216, wherein the one or more CARs comprise a CD28 hinge domain having the amino acid sequence of SEQ ID NO: 10 or 113.

Embodiment 219. The use according to embodiment 216, wherein the one or more CARs comprise a IgG4 hinge domain having the amino acid sequence of SEQ ID NO: 11 or 12.

Embodiment 220. The use according to any one of embodiments 200-219, wherein the one or more CARs comprise a CD8α transmembrane domain or a CD28 transmembrane domain.

Embodiment 221. The use according to embodiment 220, wherein the one or more CARs comprise a CD8α transmembrane domain having the amino acid sequence of SEQ ID NO: 14.

Embodiment 222. The use according to embodiment 220, wherein the one or more CARs comprise a CD28 transmembrane domain having the amino acid sequence of SEQ ID NO: 15 or 114.

Embodiment 223. The use according to any one of embodiments 200-219, wherein the one or more CARs comprise a 4-1BB costimulatory domain, a CD28 costimulatory domain, or a CD37 signaling domain.

Embodiment 224. The use according to embodiment 223, wherein the one or more CARs comprise a 4-1BB costimulatory domain having the amino acid sequence of SEQ ID NO: 16.

Embodiment 225. The use according to embodiment 223, wherein the one or more CARs comprise a CD28 costimulatory domain having the amino acid sequence of SEQ ID NO: 17.

Embodiment 226. The use according to embodiment 223, wherein the one or more CARs comprise a CD3ζ signaling domain having the amino acid sequence of SEQ ID NO: 18 or 115.

Embodiment 227. The use according to any one of embodiments 200-226, wherein the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 228. The use according to any one of embodiments 200-227, wherein the one or more CARs have a sequence of any one of SEQ ID NOs: 32, 34, 36, 117, 91, 92, 92, 128, 133, or 134.

Embodiment 229. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD20 CAR having the CDR sequences of SEQ ID NO: 37.

Embodiment 230. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a EBV antigen CAR having the CDR sequences of SEQ ID NO: 133 or 134, and/or the CDR sequences from the V_{H}/V_{L} sequences of SEQ ID NOS: 129-132 or 135-172.

Embodiment 231. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117.

Embodiment 232. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD22 CAR having the CDR sequences of SEQ ID NO: 45.

Embodiment 233. Use of a population of engineered T cells for treating multiple sclerosis in a patient that is suspected of having multiple sclerosis or has been diagnosed with multiple sclerosis, wherein the engineered T cells comprise reduced expression of B2M, CIITA, and TRAC and/or TRB, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45.

Embodiment 234. Use of a population of engineered T cells for treating multiple sclerosis in a patient that is suspected of having multiple sclerosis or has been diagnosed with multiple sclerosis, wherein the engineered T cells comprise an exogenous polynucleotide encoding one or more CARs wherein the one or more CARs have a sequence of any one of SEQ ID NOs: 32, 34, 36, 117, 91, 92, 92, 128, 133, or 134, or wherein the one or more CARs have an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 235. The use of any one of embodiments 200-234 further comprising evaluating the patient for and/or diagnosing the patient with EBV infection and optionally multiple sclerosis prior to administering the population of engineered T cells to the patient.

Embodiment 236. The use of embodiment 235, wherein the diagnosis comprises evaluating the patient for EBV infection.

Embodiment 237. The use of embodiment 235 or 236, wherein the diagnosis comprises evaluating the patient for multiple sclerosis.

Embodiment 238. The use of any one of embodiments 200-237, wherein the treatment prevents multiple sclerosis.

Embodiment 239. The use of any one of embodiments 200-237, wherein the treatment treats multiple sclerosis.

Embodiment 240. The use of any one of embodiments 200-239, wherein the patient with the EBV infection has been diagnosed with multiple sclerosis.

Embodiment 241. The use of any one of embodiments 200-240, wherein the multiple sclerosis is relapsing-remitting multiple sclerosis, progressive relapsing multiple sclerosis, primary progressive multiple sclerosis, or secondary progressive multiple sclerosis.

Embodiment 242. The use of any one of embodiments 200-241, wherein the patient undergoes remission of multiple sclerosis following administration of the engineered T cells.

Embodiment 243. The use of any one of embodiments 200-242, wherein the patient with the EBV infection is undergoing treatment for the EBV infection.

Embodiment 244. The use of any one of embodiments 200-243, wherein the patient with the EBV infection has an active EBV infection.

Embodiment 245. The use of any one of embodiments 200-244, wherein the patient with the EBV infection has an inactive EBV infection.

Embodiment 246. The use of any one of embodiments 200-245, wherein the patient undergoes a reduced EBV infection following administration of the engineered T cells, optionally wherein the reduced EBV infection is characterized by reduced viral load.

Embodiment 247. The use of any one of embodiments 200-245, wherein the treatment prevents an EBV infection change from an inactive to an active EBV infection.

Embodiment 248. The use of any one of embodiments 200-247, wherein the use results in B cell depletion.

Embodiment 249. The use of any one of embodiments 200-248, wherein the engineered T cells comprise one or more of a CD19-specific CAR, a CD20-specific CAR, a CD22-specific CAR, a BCMA-specific CAR, a GPRC5D-specific CAR, a CD38-specific CAR, a CD70-specific CAR, a CD79b-specific CAR, and an EBV antigen-specific CAR.

Embodiment 250. Use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 251. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 252. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition. Embodiment 253. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding a tolerogenic factor, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 254. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 255. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 256. The use according to any one of embodiments 250-255, wherein the one or more CARs comprise a CD8α hinge domain, a CD28 hinge domain, or an IgG4 hinge domain.

Embodiment 257. The use according to embodiment 256, wherein the one or more CARs comprise a CD8α hinge domain having the amino acid sequence of SEQ ID NO: 9.

Embodiment 258. The use according to embodiment 256, wherein the one or more CARs comprise a CD28 hinge domain having the amino acid sequence of SEQ ID NO: 10 or 113.

Embodiment 259. The use according to embodiment 256, wherein the one or more CARs comprise a IgG4 hinge domain having the amino acid sequence of SEQ ID NO: 11 or 12.

Embodiment 260. The use according to any one of embodiments 250-259, wherein the one or more CARs comprise a CD8α transmembrane domain or a CD28 transmembrane domain.

Embodiment 261. The use according to embodiment 260, wherein the one or more CARs comprise a CD8α transmembrane domain having the amino acid sequence of SEQ ID NO: 14.

Embodiment 262. The use according to embodiment 260, wherein the one or more CARs comprise a CD28 transmembrane domain having the amino acid sequence of SEQ ID NO: 15 or 114.

Embodiment 263. The use according to any one of embodiments 250-262, wherein the one or more CARs comprise a 4-1BB costimulatory domain, a CD28 costimulatory domain, or a CD37 signaling domain.

Embodiment 264. The use according to embodiment 263, wherein the one or more CARs comprise a 4-1BB costimulatory domain having the amino acid sequence of SEQ ID NO: 16.

Embodiment 265. The use according to embodiment 263, wherein the one or more CARs comprise a CD28 costimulatory domain having the amino acid sequence of SEQ ID NO: 17.

Embodiment 266. The use according to embodiment 263, wherein the one or more CARs comprise a CD3ζ signaling domain having the amino acid sequence of SEQ ID NO: 18 or 115.

Embodiment 267. The use according to any one of embodiments 250-266, wherein the one or more CARs comprise an extracellular ligand-binding domain comprising an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 268. The use according to any one of embodiments 250-267, wherein the one or more CARs have a sequence of any one of SEQ ID NOs: 32, 34, 36, 117, 91, 92, 92, 128, 133, or 134.

Embodiment 269. Use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 270. The use according to embodiment 269, further comprising evaluating the patient for and/or diagnosing the patient with the autoimmune disease.

Embodiment 271. Use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a CD19 CAR having the CDR sequences of SEQ ID NO: 117 and a CD22 CAR having the CDR sequences of SEQ ID NO: 45, and wherein the autoimmune disease is selected from group consisting of lupus, systemic lupus erythematosus, multiple sclerosis, systemic sclerosis, vasculitis, Crohn's disease, Myasthenia Gravis, Stiff-Person syndrome, or a pulmonary condition.

Embodiment 272. The use of any one of embodiments 269-271, wherein the encoded CD19 CAR has a corresponding amino acid sequence set forth in SEQ ID NO:117 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 117, with the following components: CD8α signal peptide, FMC63 scFv (VL-Whitlow linker-VH), CD8α hinge domain, CD8α transmembrane domain, 4-1BB costimulatory domain, and CD37 signaling domain.

Embodiment 273. The use of any one of embodiments 269-272, wherein the encoded CD22 CAR has a corresponding amino acid sequence set forth in SEQ ID NO:45 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 45.

Embodiment 274. Use of a population of engineered T cells for treating an autoimmune disease in a patient that is suspected of having the autoimmune disease or has been diagnosed with the autoimmune disease, wherein the engineered T cells comprise reduced expression of B2M, CIITA, and TRAC, relative to an unaltered or unmodified wild-type or control cell, a first exogenous polynucleotide encoding CD47, and a second exogenous polynucleotide encoding one or more CARs, wherein the one or more CARs comprise a EBV antigen-specific CAR having the CDR sequences of SEQ ID NO: 133 or 134, and/or the CDR sequences from the VH/VL sequences of SEQ ID NOs: 129-132 or 135-172, and wherein the autoimmune disease is multiple sclerosis.

Embodiment 275. Use of a population of engineered T cells for treating an autoimmune disease in a patient that is suspected of having the autoimmune disease or has been diagnosed with the autoimmune disease, wherein the engineered T cells comprise an exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 276. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 277. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

Embodiment 278. Use of a population of engineered T cells for treating an autoimmune disease in a patient that is suspected of having the autoimmune disease or has been diagnosed with the autoimmune disease, wherein the engineered T cells comprise reduced expression of B2M and CITA relative to an unaltered or unmodified wild-type or control cell, a first exogenous polynucleotide encoding a tolerogenic factor, and a second exogenous polynucleotide encoding one or more CARs, wherein the one or more CARs have a sequence of any one of SEQ ID NOS: 32, 34, 36, and 117, or wherein the one or more CARs have an scFv sequence of any one of SEQ ID NOs: 19, 29, or 37.

Embodiment 279. Use of a population of engineered T cells for treating an autoimmune disease in a patient that is suspected of having the autoimmune disease or has been diagnosed with the autoimmune disease, wherein the engineered T cells comprise reduced expression of B2M relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 280. Use of a population of engineered T cells for treating an autoimmune disease in a patient that is suspected of having the autoimmune disease or has been diagnosed with the autoimmune disease, wherein the engineered T cells comprise reduced expression of B2M and CIITA relative to an unaltered or unmodified wild-type or control cell, a first exogenous polynucleotide encoding CD47, and a second exogenous polynucleotide encoding one or more CARs, wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 281. Use of a population of engineered T cells for treating an autoimmune disease in a patient that is suspected of having the autoimmune disease or has been diagnosed with the autoimmune disease, wherein the engineered T cells comprise reduced expression of one or more MHC class I and/or class II HLA molecules relative to an unaltered or unmodified wild-type or control cell, a first exogenous polynucleotide encoding CD47, and a second exogenous polynucleotide encoding one or more CARs wherein the one or more CARs have a sequence of any one of SEQ ID NOs: 32, 34, 36, and 117, or wherein the one or more CARs have an scFv sequence of any one of SEQ ID NOs: 19, 29, or 37.

Embodiment 282. The use of any one of embodiments 250-281 further comprising evaluating the patient for and/or diagnosing the patient with the autoimmune disease prior to administering the population of engineered T cells to the patient.

Embodiment 283. The use of any one of embodiments 250-282, wherein the autoimmune disease is selected from the group consisting of lupus, systemic lupus erythematosus, multiple sclerosis, systemic sclerosis, vasculitis, Crohn's disease, Myasthenia Gravis, Stiff-Person syndrome and a pulmonary condition.

Embodiment 284. The use of any one of embodiments 250-283, wherein the patient is suspected of having an EBV infection or has been diagnosed as having an EBV infection.

Embodiment 285. The use of any one of embodiments 250-284, wherein the patient is suspected of having multiple sclerosis or has been diagnosed with multiple sclerosis.

Embodiment 286. The use of any one of embodiments 250-285, further comprising administering a second, third, fourth, fifth, or sixth dose of the engineered T cells to the patient.

Embodiment 287. The use of embodiment 286, wherein the CAR is the same in one or more of the first, second, third, fourth, fifth, and/or sixth dose of engineered T cells.

Embodiment 288. The use of embodiment 288, wherein the CAR is different in one or more of the first, second, third, fourth, fifth, and/or sixth dose of engineered T cells.

Embodiment 289. The use of any one of embodiments 200-288, wherein the CAR has an scFv sequence of any one of SEQ ID NOs: 19, 29, 37, 45, 54, 85, 63, 72, or 118, or wherein the CARs have an scFv sequence comprising the heavy and light chain sequences of any one of SEQ ID NOs: 129-132 or 135-172.

Embodiment 290. The use of any one of embodiments 200-289, wherein the CAR has a sequence of any one of SEQ ID NOs: 32, 34, 36, 117, 91, 92, 92, 128, 133, and 134.

Embodiment 291. The use of any one of embodiments 200-290, wherein the engineered T cells comprise a CD19-specific CAR and a CD20-specific CAR.

Embodiment 292. The use of embodiment 291, wherein the CD19-specific CAR has the CDR sequences of SEQ ID NO: 117 and the CD22 CAR has the CDR sequences of SEQ ID NO: 45.

Embodiment 293. The use of embodiment 291, wherein the CD19-specific CAR and the CD20-specific CAR are encoded by a single bicistronic polynucleotide.

Embodiment 294. The use of embodiment 291, wherein the CD19-specific CAR and the CD20-specific CAR are encoded by a single bispecific CAR.

Embodiment 295. The use of embodiment 291, wherein the CD19-specific CAR and the CD20-specific CAR are encoded by two separate polynucleotides.

Embodiment 296. The use of any one of embodiments 291-295, wherein the CD19 CAR T cells and CD20 CAR T cells are administered concomitantly.

Embodiment 297. The use of any one of embodiments 291-295, wherein the CD19 CAR+ T cells and CD20 CAR+ T cells are administered sequentially.

Embodiment 298. The use of embodiment 297, wherein the CD19 CAR+ T cells are administered prior to administration of the CD20 CAR+ T cells.

Embodiment 299. The use of embodiment 297, wherein the CD20 CAR+ T cells are administered prior to administration of the CD19 CAR+ T cells.

Embodiment 300. The use of any one of embodiments 291-299, wherein the number of cells administered as a therapeutically effective amount of the CD19 and/or CD20 CAR T cells is greater than the number of cells administered as a therapeutically effective amount of CD19 CAR T cells or CD20 CAR T cells alone.

Embodiment 301. The use of any one of embodiments 291-299, wherein the number of cells administered to as a therapeutically effective amount of the CD19 and/or CD20 CAR T cells is less than the number of cells administered as a therapeutically effective amount of CD19 CAR T cells or CD20 CAR T cells alone.

Embodiment 302. The use of any one of embodiments 291-301, wherein the engineered T cells comprise an EBV antigen-specific CAR and a CD20-specific CAR.

Embodiment 303. The use of embodiment 302, wherein the EBV antigen-specific CAR and the CD20-specific CAR are encoded by a single bicistronic polynucleotide.

Embodiment 304. The use of embodiment 302, wherein the EBV antigen-specific CAR and the CD20-specific CAR are encoded by a single bispecific CAR.

Embodiment 305. The use of embodiment 302, wherein the EBV antigen-specific CAR and the CD20-specific CAR are encoded by two separate polynucleotides.

Embodiment 306. The use of any one of embodiments 302-305, wherein the EBV antigen CAR T cells and CD20 CAR T cells are administered concomitantly.

Embodiment 307. The use of any one of embodiments 302-305, wherein the EBV antigen CAR+ T cells and CD20 CAR+ T cells are administered sequentially.

Embodiment 308. The use of embodiment 307, wherein the EBV antigen CAR+ T cells are administered prior to administration of the CD20 CAR+ T cells.

Embodiment 309. The use of embodiment 307, wherein the CD20 CAR+ T cells are administered prior to administration of the EBV antigen CAR+ T cells.

Embodiment 310. The use of any one of embodiments 302-309, wherein the number of cells administered as a therapeutically effective amount of the EBV antigen and/or CD20 CAR T cells is greater than the number of cells administered as a therapeutically effective amount of EBV antigen CAR T cells or CD20 CAR T cells alone.

Embodiment 311. The use of any one of embodiments 302-309, wherein the number of cells administered to as a therapeutically effective amount of the EBV antigen and/or CD20 CAR T cells is less than the number of cells administered as a therapeutically effective amount of EBV antigen CAR T cells or CD20 CAR T cells alone.

Embodiment 312. The use of any one of embodiments 200-311, wherein the engineered T cells comprise an EBV antigen-specific CAR and a CD19-specific CAR.

Embodiment 313. The use of embodiment 312, wherein the EBV antigen-specific CAR and the CD19-specific CAR are encoded by a single bicistronic polynucleotide.

Embodiment 314. The use of embodiment 312, wherein the EBV antigen-specific CAR and the CD19-specific CAR are encoded by a single bispecific CAR.

Embodiment 315. The use of embodiment 312, wherein the EBV antigen-specific CAR and the CD19-specific CAR are encoded by two separate polynucleotides.

Embodiment 316. The use of any one of embodiments 312-315, wherein the EBV antigen CAR T cells and CD19 CAR T cells are administered concomitantly.

Embodiment 317. The use of any one of embodiments 312-315, wherein the EBV antigen CAR+ T cells and CD19 CAR+ T cells are administered sequentially.

Embodiment 318. The use of embodiment 317, wherein the EBV antigen CAR+ T cells are administered prior to administration of the CD19 CAR+ T cells.

Embodiment 319. The use of embodiment 317, wherein the CD19 CAR+ T cells are administered prior to administration of the EBV antigen CAR+ T cells.

Embodiment 320. The use of any one of embodiments 312-319, wherein the number of cells administered as a therapeutically effective amount of the EBV antigen and/or CD19 CAR T cells is greater than the number of cells administered as a therapeutically effective amount of EBV antigen CAR T cells or CD19 CAR T cells alone.

Embodiment 321. The use of any one of embodiments 312-319, wherein the number of cells administered to as a therapeutically effective amount of the EBV antigen and/or CD19 CAR T cells is less than the number of cells administered as a therapeutically effective amount of EBV antigen CAR T cells or CD19 CAR T cells alone.

Embodiment 322. The use of any one of embodiments 200-321, wherein the engineered T cells comprise an EBV antigen-specific CAR and a CD22-specific CAR.

Embodiment 323. The use of embodiment 322, wherein the EBV antigen-specific CAR and the CD22-specific CAR are encoded by a single bicistronic polynucleotide.

Embodiment 324. The use of embodiment 322, wherein the EBV antigen-specific CAR and the CD22-specific CAR are encoded by a single bispecific CAR.

Embodiment 325. The use of embodiment 322, wherein the EBV antigen-specific CAR and the CD22-specific CAR are encoded by two separate polynucleotides.

Embodiment 326. The use of any one of embodiments 322-325, wherein the EBV antigen CAR T cells and CD22 CAR T cells are administered concomitantly.

Embodiment 327. The use of any one of embodiments 322-325, wherein the EBV antigen CAR+ T cells and CD22 CAR+ T cells are administered sequentially.

Embodiment 328. The use of embodiment 327, wherein the EBV antigen CAR+ T cells are administered prior to administration of the CD22 CAR+ T cells.

Embodiment 329. The use of embodiment 327, wherein the CD22 CAR+ T cells are administered prior to administration of the EBV antigen CAR+ T cells.

Embodiment 330. The use of any one of embodiments 322-329, wherein the number of cells administered as a therapeutically effective amount of the EBV antigen and/or CD22 CAR T cells is greater than the number of cells administered as a therapeutically effective amount of EBV antigen CAR T cells or CD22 CAR T cells alone.

Embodiment 331. The use of any one of embodiments 322-329, wherein the number of cells administered to as a therapeutically effective amount of the EBV antigen and/or CD22 CAR T cells is less than the number of cells administered as a therapeutically effective amount of EBV antigen CAR T cells or CD22 CAR T cells alone.

Embodiment 332. The use of any one of embodiments 200-331, wherein the engineered T cells are primary T cells, are propagated from a primary T cell or a progeny thereof, or are derived from a T cell differentiated from an iPSC or a progeny thereof.

Embodiment 333. The use of any one of embodiments 200-331, wherein the engineered T cells are differentiated cells derived from an induced pluripotent stem cell or a progeny thereof.

Embodiment 334. The use of embodiment 333, wherein the differentiated cells are a T cells or natural killer (NK) cells.

Embodiment 335. The use of any one of embodiments 200-334, wherein the engineered T cells are primary T cells, are progeny of primary immune cells, optionally wherein the progeny of primary immune cells are T cells or NK cells.

Embodiment 336. The use of any one of embodiments 200-335, wherein the engineered T cells comprise reduced expression of one or more MHC HLA class I molecules relative to an unaltered or unmodified wild-type or control cell.

Embodiment 337. The use of any one of embodiments 200-336, wherein the engineered T cells comprise reduced expression of one or more MHC HLA class II molecules relative to an unaltered or unmodified wild-type or control cell.

Embodiment 338. The use of any one of embodiments 200-337, wherein the engineered T cells comprise reduced expression of one or more MHC HLA class I molecules and of one or more MHC HLA class II molecules relative to an unaltered or unmodified wild-type or control cell.

Embodiment 339. The use of any one of embodiments 200-338, wherein the engineered T cells comprise reduced expression of B2M and/or CIITA relative to an unaltered or unmodified wild-type or control cell.

Embodiment 340. The use of embodiment 339, wherein the engineered T cells do not express B2M and/or CIITA.

Embodiment 341. The use of any one of embodiments 200-340, wherein the engineered T cells comprise reduced expression of TRAC and/or TRB.

Embodiment 342. The use of embodiment 341, wherein the engineered T cells do not express TRAC and/or TRB.

Embodiment 343. The use of any one of embodiments 200-342, wherein the engineered T cells comprise reduced expression of TRAC.

Embodiment 344. The use of embodiment 343, wherein the engineered T cells do not express TRAC.

Embodiment 345. The use of any one of embodiments 200-344, wherein the engineered T cells comprise reduced expression of TRB.

Embodiment 346. The use of embodiment 345, wherein the engineered T cells do not express TRB.

Embodiment 347. The use of any one of embodiments 200-346, wherein the engineered T cells comprise reduced expression of TRAC and TRB.

Embodiment 348. The use of any one of embodiments 200-347, wherein the one or more tolerogenic factors are selected from the group consisting of CD47, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, FasL, CCL21, CCL22, Mfge8, A20/TNFAIP3, CD39, CR1, HLA-F, IL15-RF, MANF, and Serpinb9, optionally wherein the one or more tolerogenic factors comprise CD47.

Embodiment 349. The use of any one of embodiments 200-348, wherein the CD19-specific CAR and the one or more tolerogenic factors are encoded by a single bicistronic polynucleotide.

Embodiment 350. The use of any one of embodiments 200-348, wherein the CD19-specific CAR and the CD47 are encoded by a single bicistronic polynucleotide.

Embodiment 351. The use of any one of embodiments 200-348, wherein the CD20-specific CAR and the one or more tolerogenic factors are encoded by a single bicistronic polynucleotide.

Embodiment 352. The use of any one of embodiments 200-348, wherein the CD20-specific CAR and the CD47 are encoded by a single bicistronic polynucleotide.

Embodiment 353. The use of any one of embodiments 200-352, wherein one or more of the first, second, and/or third exogenous polynucleotides or the bicistronic polynucleotide is inserted into a first, second, and/or third specific locus of at least one allele of the cell.

Embodiment 354. The use of embodiment 353, wherein the first, second and/or third specific loci are selected from the group consisting of a safe harbor locus, a target locus, an RHD locus, a B2M locus, a CIITA locus, a TRAC locus, and a TRB locus.

Embodiment 355. The use of embodiment 354, wherein the safe harbor locus is selected from the group consisting of a CCR5 locus, a PPP1R12C locus, a CLYBL locus, and a Rosa locus.

Embodiment 356. The use of embodiment 354, wherein the target locus is selected from the group consisting of a CXCR4 locus, an ALB locus, a SHS231 locus, an F3 (CD142) locus, a MICA locus, a MICB locus, a LRP1 (CD91) locus, a HMGB1 locus, an ABO locus, a FUT1 locus, and a KDM5D locus.

Embodiment 357. The use of any one of embodiments 200-356, wherein the first, second, and/or third exogenous polynucleotide or the bicistronic polynucleotide is introduced into the engineered T cells using a gene therapy vector or a transposase system selected from the group consisting of transposases, PiggyBac transposons, Sleeping Beauty (SB11) transposons, Mos1 transposons, and Tol2 transposons.

Embodiment 358. The use of embodiments 357, wherein the gene therapy vector is a retrovirus or a fusosome.

Embodiment 359. The use of any one of embodiments 200-358, wherein the first, second, and/or third exogenous polynucleotide or the bicistronic polynucleotide is introduced into the engineered T cells using CRISPR/Cas gene editing.

Embodiment 360. The use of embodiment 259, wherein the CRISPR/Cas system comprises a Cas effector protein selected from the group consisting of Cas9, Cas12a, and Cas12b.

Embodiment 361. The use or dosage regimen of embodiment 360, wherein the CRISPR/Cas system comprises a Cas effector protein selected from the group consisting of:
(a) optionally selected from the group consisting of Cas3, Cas8a, Cas5, Cas8b, Cas8c, Cas10d, Cse1, Cse2, Csy1, Csy2, Csy3, and GSU0054;
(b) optionally selected from the group consisting of Cas9, Csn2, and Cas4;
(c) optionally selected from the group consisting of Cas10, Csm2, Cmr5, Cas10, Csx11, and Csx10;
(d) optionally Csf1;
(e) optionally selected from the group consisting of Cas12a, Cas12b, Cas12c, C2c4, C2c8, C2c5, C2c10, C2c9, CasX (Cas12e), and CasY (Cas12d); and
(f) optionally selected from the group consisting of Cas13, Cas13a, C2c2, Cas13b, Cas13c, and Cas13d.

Embodiment 362. The use of any one of embodiments 359-361, wherein the CRISPR/Cas gene editing is carried out ex vivo from a donor subject.

Embodiment 363. The use of any one of embodiments 359-362, wherein the first, second, and/or third exogenous polynucleotide or the bicistronic polynucleotide is introduced into the engineered T cells using a lentiviral vector.

Embodiment 364. The use of any one of embodiments 200-363, wherein the engineered T cells evade NK cell mediated cytotoxicity upon administration to the patient.

Embodiment 365. The use of any one of embodiments 200-364, wherein the engineered T cells are protected from cell lysis by mature NK cells upon administration to the patient.

Embodiment 366. The use of any one of embodiments 200-365, wherein the engineered T cells evade macrophage-mediated cytotoxicity, optionally wherein the macrophage-mediated cytotoxicity involves phagocytosis and/or reactive oxygen species.

Embodiment 367. The use of any one of embodiments 200-366, wherein the engineered T cells do not induce an immune response to the cell upon administration to the patient.

Embodiment 368. The use of any one of embodiments 200-367, wherein the administration is selected from the group consisting of intravenous injection, intramuscular injection, intravascular injection, and transplantation.

Embodiment 369. The use of any one of embodiments 200-368, wherein the engineered T cells are administered before, during or after starting a different treatment regimen for the patient.

Embodiment 370. The use of embodiment 369, wherein the different treatment regimen is selected from the group consisting of re-dosing of the same or different cells, and pre-treatment, concurrent treatment, or subsequent treatment with an additional agent.

Embodiment 371. The use of embodiment 370, wherein the different cells are autologous T or NK cells or CAR-T cells expressing a first CAR that is different from a second CAR expressed by the engineered CAR-T cells.

Embodiment 372. The use of any one of embodiments 200-371, wherein the patient was treated with an immunodepleting therapy prior to administering the engineered T cells.

Embodiment 373. The use of embodiment 372, wherein the immunodepleting therapy comprises administration of fludarabine and/or cyclophosphamide.

Embodiment 374. The use of any one of embodiments 200-373, wherein the patient has undergone a prior antibody therapy.

Embodiment 375. The use of embodiment 374, wherein the antibody therapy is rituximab.

Embodiment 376. The use of embodiment 373, wherein the immunodepleting therapy comprises IV infusion of about 1-50 mg/m2 of fludarabine for about 1-7 days.

Embodiment 377. The use of embodiment 376, wherein the immunodepleting therapy comprises IV infusion of about 1, about 5, about 10, about 20, about 30, about 40, or about 50 mg/m2 of fludarabine for about 1, about 2, about 3, about 4, about 5, about 6, or about 7 days.

Embodiment 378. The use of embodiment 376 or 377, wherein the immunodepleting therapy comprises IV infusion of about 30 mg/m2 of fludarabine for about 4 days.

Embodiment 379. The use of embodiment 373, wherein the immunodepleting therapy comprises IV infusion of about 100-1000 mg/m2 of cyclophosphamide for about 1-7 days.

Embodiment 380. The use of embodiment 379, wherein the immunodepleting therapy comprises IV infusion of about 100, about 200, about 300, about 400, about 500, about 600, about 700, about 800, about 900, about 1000 mg/m2 of cyclophosphamide for about 1, about 2, about 3, about 4, about 5, about 6, or about 7 days.

Embodiment 381. The use of embodiment 379 or 380, wherein the immunodepleting therapy comprises IV infusion of about 500 mg/m2 of cyclophosphamide for about 2 days.

Embodiment 382. The use of any one of embodiments 200-381, wherein at least about 40×104 engineered T cells are administered to the patient.

Embodiment 383. The use of any one of embodiments 200-382, wherein at least about 40×105 engineered T cells are administered to the patient.

Embodiment 384. The use of any one of embodiments 200-383, wherein the engineered T cells persist in the subject for at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 385. The use of any one of embodiments 200-384, wherein the therapeutic effect of the engineered T cells lasts for a duration of at least 4 weeks, at least 2 months, at least 3 months, at least 4 months, at least 5 months, at least 6 months, at least 7 months, at least 8 months, at least 9 months, at least 10 months, at least 11 months, at least 12 months, or longer.

Embodiment 386. The use of any one of embodiments 200-385, wherein the wild type cell or the control cell is a starting material.

Embodiment 387. The method of any one of embodiments 1-199 or the use of any one of embodiments 200-373, wherein the population of engineered T cells comprises an engineered T cell comprising one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a T cell that does not comprise the modifications.

Embodiment 388. An engineered cell comprising one or more modifications that (i) reduce expression of one or more MHC class I molecules and/or one or more MHC class II molecules, and/or (ii) increase expression of one or more tolerogenic factors, wherein the reduced expression of (i) and the increased expression of (ii) is relative to a cell of the same cell type that does not comprise the modifications.

Embodiment 389. The engineered cell of embodiment 388, wherein the one or more modifications in (i) reduce expression of:
a. one or more MHC class I molecules
b. one or more MHC class II molecules; or
c. one or more MHC class I molecules and one or more MHC class II molecules.

Embodiment 390. The engineered cell of embodiment 388 or embodiment 389, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

Embodiment 391. The engineered cell of embodiment 390, wherein the engineered cell does not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.

Embodiment 392. The engineered cell of any of embodiments 388-391, wherein the one or more modifications that increase expression comprise increased cell surface expression, and/or the one or more modifications that reduce expression comprise reduced cell surface expression.

Embodiment 393. The engineered cell of any of embodiments 388-392, wherein the one or more modifications in (i) reduce expression of one or more MHC class I molecules.

Embodiment 394. The engineered cell of any of embodiments 388-393, wherein the one or more modifications in (i) reduce expression of B2M.

Embodiment 395. The engineered cell of any of embodiments 388-394, wherein the one or more modifications in (i) reduce expression of HLA-A, HLA-B, and/or HLA-C.

Embodiment 396. The engineered cell of any of embodiments 388-395, wherein the one or more modifications in (i) reduce expression of one or more MHC class II molecules.

Embodiment 397. The engineered cell of any of embodiments 388-396, wherein the one or more modifications in (i) reduce expression of CIITA.

Embodiment 398. The engineered cell of any of embodiments 388-397, wherein the one or more modifications in (i) reduce expression of HLA-DM, HLA-DO, HLA-DP, HLA-DQ, HLA-DR, RFX5, RFXANK, and/or RFXAP.

Embodiment 399. The engineered cell of any of embodiments 388-398, wherein the one or more tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, A20/TNFAIP3, CD39, CR1, HLA-F, IL15-RF, MANF, and any combination thereof.

Embodiment 400. The engineered cell of any of embodiments 388-399, wherein the one or more tolerogenic factors comprise CD47.

Embodiment 401. The engineered cell of any of embodiments 388-400, wherein the one or more tolerogenic factors comprise CCL22.

Embodiment 402. The engineered cell of any of embodiments 388-401, wherein the one or more tolerogenic factors comprise CD16 or CD16 Fc receptor.

Embodiment 403. The engineered cell of any of embodiments 388-402, wherein the one or more tolerogenic factors comprise CD24.

Embodiment 404. The engineered cell of any of embodiments 388-403, wherein the one or more tolerogenic factors comprise CD39.

Embodiment 405. The engineered cell of any of embodiments 388-404, wherein the one or more tolerogenic factors comprise CR1.

Embodiment 406. The engineered cell of any of embodiments 388, wherein the one or more tolerogenic factors comprise CD52.

Embodiment 407. The engineered cell of any of embodiments 388-406, wherein the one or more tolerogenic factors comprise CD55.

Embodiment 408. The engineered cell of any of embodiments 388-407, wherein the one or more tolerogenic factors comprise CD200.

Embodiment 409. The engineered cell of any of embodiments 388-408, wherein the one or more tolerogenic factors comprise DUX4.

Embodiment 410. The engineered cell of any of embodiments 388-409, wherein the one or more tolerogenic factors comprise HLA-E.

Embodiment 411. The engineered cell of any of embodiments 388-410, wherein the one or more tolerogenic factors comprise HLA-G.

Embodiment 412. The engineered cell of any of embodiments 388-411, wherein the one or more tolerogenic factors comprise IDO1.

Embodiment 413. The engineered cell of any of embodiments 388-412, wherein the one or more tolerogenic factors comprise IL15-RF.

Embodiment 414. The engineered cell of any of embodiments 388-413, wherein the one or more tolerogenic factors comprise IL35.

Embodiment 415. The engineered cell of any of embodiments 388-414, wherein the one or more tolerogenic factors comprise PD-L1.

Embodiment 416. The engineered cell of any of embodiments 388-415, wherein the one or more tolerogenic factors comprise MANF.

Embodiment 417. The engineered cell of any of embodiments 388-416, wherein the one or more tolerogenic factors comprise A20/TNFAIP3.

Embodiment 418. The engineered cell of any of embodiments 388-417, wherein the one or more tolerogenic factors comprise HLA-E and CD47.

Embodiment 419. The engineered cell of any of embodiments 388-31, wherein the one or more tolerogenic factors comprise two or more tolerogenic factors selected from the group consisting of CD47, CD46, and CD59, optionally wherein the one or more tolerogenic factors comprise CD47, CD46, and CD59.

Embodiment 420. The engineered cell of any of embodiments 388-419, wherein the one or more tolerogenic factors comprise two or more tolerogenic factors selected from the group consisting of CD47 and CD39, optionally wherein the one or more tolerogenic factors comprise CD47 and CD39.

Embodiment 421. The engineered cell of any of embodiments 388-420, wherein the one or more tolerogenic factors comprise two or more tolerogenic factors selected from the group consisting of CD47 and CCL22, optionally wherein the one or more tolerogenic factors comprise CD47 and CCL22.

Embodiment 422. The engineered cell of any of embodiments 388-421, wherein the one or more tolerogenic factors comprise two or more tolerogenic factors selected from the group consisting of CD47, HLA-G and PD-L1, optionally wherein the one or more tolerogenic factors comprise CD47 and PD-L1, and optionally wherein the one or more tolerogenic factors comprise CD47, HLA-G and PD-L1.

Embodiment 423. The engineered cell of any of embodiments 388-422, wherein the one or more tolerogenic factors comprise two or more tolerogenic factors selected from the group consisting of CD24, CD47, and PD-L1, optionally wherein the one or more tolerogenic factors comprise CD24, CD47, and PD-L1.

Embodiment 424. The engineered cell of any of embodiments 388-423, wherein the one or more tolerogenic factors comprise two or more tolerogenic factors selected from the group consisting of HLA-E, CD24, CD47, and PD-L1, optionally wherein the one or more tolerogenic factors comprise HLA-E, CD24, CD47, and PD-L1.

Embodiment 425. The engineered cell of any of embodiments 388-424, wherein the one or more tolerogenic factors comprise two or more tolerogenic factors selected from the group consisting of CD46, CD55, CD59, and CR1, optionally wherein the one or more tolerogenic factors comprise CD46, CD55, CD59, and CR1.

Embodiment 426. The engineered cell of any of embodiments 388-425, wherein the one or more tolerogenic factors comprise two or more tolerogenic factors selected from the group consisting of HLA-E, CD46, CD55, CD59, and CR1, optionally wherein the one or more tolerogenic factors comprise HLA-E, CD46, CD55, CD59, and CR1.

Embodiment 427. The engineered cell of any of embodiments 388-426, wherein the one or more tolerogenic factors comprise two or more tolerogenic factors selected from the group consisting of HLA-E, CD24, CD47, PD-L1, CD46, CD55, CD59, and CR1, optionally wherein the one or more tolerogenic factors comprise HLA-E, CD24, CD47, PD-L1, CD46, CD55, CD59, and CR1.

Embodiment 428. The engineered cell of any of embodiments 388-427, wherein the one or more tolerogenic factors comprise HLA-E and PD-L1.

Embodiment 429. The engineered cell of any of embodiments 388-428, wherein the one or more tolerogenic factors comprise two or more tolerogenic factors selected from the group consisting of HLA-E, PD-L1, and A20/TNFAIP, optionally wherein the one or more tolerogenic factors comprise HLA-E, PD-L1, and A20/TNFAIP.

Embodiment 430. The engineered cell of any of embodiments 388-429, wherein the one or more tolerogenic factors comprise two or more tolerogenic factors selected from the group consisting of HLA-E, PD-L1, and MANF, optionally wherein the one or more tolerogenic factors comprise HLA-E, PD-L1, and MANF.

Embodiment 431. The engineered cell of any of embodiments 388-430, wherein the one or more tolerogenic factors comprise two or more tolerogenic factors selected from the group consisting of HLA-E, PD-L1, A20/TNFAIP, and MANF, optionally wherein the one or more tolerogenic factors comprise HLA-E, PD-L1, A20/TNFAIP, and MANF.

Embodiment 432. An engineered cell comprising one or more modifications that (i) reduce expression of one or more MHC class I molecules and one or more MHC class II molecules, and (ii) increase expression of CD47, wherein the reduced expression of (i) and the increased expression of (ii) is relative to a cell of the same cell type that does not comprise the modifications.

Embodiment 433. The engineered cell of embodiment 432, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

Embodiment 434. The engineered cell of embodiment 432 or embodiment 46, wherein the one or more modifications in (i) reduce expression of B2M.

Embodiment 435. The engineered cell of any of embodiments 432-434, wherein the one or more modifications in (i) reduce expression of HLA-A, HLA-B, and/or HLA-C.

Embodiment 436. The engineered cell of any of embodiments 432-435, wherein the one or more modifications in (i) reduce expression of CIITA.

Embodiment 437. The engineered cell of any of embodiments 432-435, wherein the one or more modifications in (i) reduce expression of HLA-DP, HLA-DR, and/or HLA-DQ.

Embodiment 438. The engineered cell of any of embodiments 388-437, wherein the engineered cell further comprises one or more modifications that increase expression of one or more additional tolerogenic factors.

Embodiment 439. The engineered cell embodiment 438, wherein the one or more additional tolerogenic factors comprise one or more tolerogenic factors selected from the group consisting of A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, A20/TNFAIP3, CD39, CR1, HLA-F, IL15-RF, MANF, and any combination thereof.

Embodiment 440. The engineered cell of embodiment 439, wherein the one or more additional tolerogenic factors comprise CD47.

Embodiment 441. The engineered cell of any one of embodiments 388-440, wherein the engineered cell further comprises one or more modifications that reduce expression of one or more additional molecules.

Embodiment 442. The engineered cell of embodiment 441, wherein the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

Embodiment 443. The engineered cell of embodiment 441 or 442, wherein the one or more additional molecules comprise one or more Y chromosome proteins, optionally Protocadherin-11 Y-linked (PCDH11Y) and/or Neuroligin-4 Y-linked (NLGN4Y).

Embodiment 444. The engineered cell of any of embodiments 441-443, wherein the one or more additional molecules comprise one or more NK cell ligands, optionally MIC-A and/or MIC-B.

Embodiment 445. The engineered cell of any of embodiments 441-444, wherein the one or more additional molecules comprise one or more proteins involved in oxidative or ER stress, optionally thioredoxin-interacting protein (TXNIP), PKR-like ER kinase (PERK), inositol-requiring enzyme 1α (IRE1α), and/or DJ-1 (PARK7).

Embodiment 446. The engineered cell of any of embodiments 441-446, wherein the one or more additional molecules comprise one or more blood antigen proteins, optionally ABO, FUT1 and/or RHD.

Embodiment 447. The engineered cell of any one of embodiments 388-446, wherein the engineered cell further comprises one or more modifications that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD3δ, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

Embodiment 448. The engineered cell of embodiment 447, wherein TRB is TRBC1, TRBC2, or TRBC1 and TRBC2.

Embodiment 449. The engineered cell of any of embodiments 388-448, wherein reduced expression comprises no cell surface expression or no detectable cell surface expression.

Embodiment 450. The engineered cell of any of embodiments 388-449, wherein reduced expression comprises reduced mRNA expression, optionally wherein reduced expression comprises no detectable mRNA expression.

Embodiment 451. The engineered cell of any of embodiments 388-460, wherein reduced expression comprises reduced protein expression or reduced protein activity, optionally wherein reduced expression comprises no detectable protein expression or protein activity.

Embodiment 452. The engineered cell of any of embodiments 388-451, wherein reduced expression comprises eliminating activity of a gene encoding or regulating the expression of i) the one or more MHC class I molecules and/or the one or more MHC class II molecules, or ii) the one or more additional molecules.

Embodiment 453. The engineered cell of any of embodiments 388-452, wherein reduced expression comprises inactivation or disruption of an allele of a gene encoding or regulating the expression of i) the one or more MHC class I molecules and/or the one or more MHC class II molecules, or ii) the one or more additional molecules.

Embodiment 454. The engineered cell of any of embodiments 388-453, wherein reduced expression comprises inactivation or disruption of both alleles of a gene encoding or regulating the expression of i) the one or more MHC class I molecules and/or the one or more MHC class II molecules, or ii) the one or more additional molecules.

Embodiment 455. The engineered cell of any of embodiments 388-454, wherein the one or more modifications to reduce expression comprises an indel in a gene encoding or regulating the expression of i) the one or more MHC class I molecules and/or the one or more MHC class II molecules, or ii) the one or more additional molecules.

Embodiment 456. The engineered cell of any of embodiments 388-455, wherein the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.

Embodiment 457. The engineered cell of any of embodiments 388-456, wherein the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.

Embodiment 458. The engineered cell of any of embodiments 388-456, wherein the one or more modifications to reduce expression comprises knocking out a gene encoding or regulating the expression of i) the one or more MHC class I molecules and/or the one or more MHC class II molecules, or ii) the one or more additional molecules.

Embodiment 459. The engineered cell of any of embodiments 388-458, wherein the engineered cell comprises one or more modifications that:
a. reduce expression of MHC class I and/or MHC class II molecules;
b. increase expression of CD47; and
c. increase expression of CCL22.

Embodiment 460. The engineered cell of any of embodiments 388-458, wherein the engineered cell comprises one or more modifications that:
a. reduce expression of MHC class I and/or MHC class II molecules;
b. increase expression of CD47; and
c. increase expression of CD39.

Embodiment 461. The engineered cell of any of embodiments 388-458, wherein the engineered cell comprises one or more modifications that:
a. reduce expression of MHC class I and/or MHC class II molecules;
b. increase expression of CD47; and
c. increase expression of CD46 and CD59.

Embodiment 462. The engineered cell of any of embodiments 388-458, wherein the engineered cell comprises one or more modifications that:
a. reduce expression of MHC class I and/or MHC class II molecules;
b. increase expression of CD47; and
c. increase expression of PD-L1.

Embodiment 463. The engineered cell of any of embodiments 388-458, wherein the engineered cell comprises one or more modifications that:
a. reduce expression of MHC class I and/or MHC class II molecules;
b. increase expression of CD47; and
c. increase expression of HLA-G and PD-L1.

Embodiment 464. The engineered cell of any of embodiments 388-458, wherein the engineered cell comprises one or more modifications that:
a. reduce expression of MHC class I and/or MHC class II molecules;
b. increase expression of CD47; and
c. reduced expression of CD142 (TF).

Embodiment 465. The engineered cell of any of embodiments 388-458, wherein the engineered cell comprises one or more modifications that:
a. reduce expression of MHC class I and/or MHC class II molecules;
b. increase expression of CD47; and
c. reduced expression of MIC-A and/or MIC-B.

Embodiment 466. The engineered cell of any of embodiments 388-458, wherein the engineered cell comprises one or more modifications that:
a. reduce expression of MHC class I and/or MHC class II molecules; and
b. increase expression of CD24.

Embodiment 467. The engineered cell of any of embodiments 388-458, wherein the engineered cell comprises one or more modifications that:
a. reduce expression of MHC class I and/or MHC class II molecules; and
b. increase expression of CD200.

Embodiment 468. The engineered cell of any of embodiments 388-458, wherein the engineered cell comprises one or more modifications that:
a. reduce expression of MHC class I and/or MHC class II molecules; and
b. increase expression of CD52.

Embodiment 469. The engineered cell of any of embodiments 388-458, wherein the engineered cell comprises one or more modifications that:
a. reduce expression of MHC class I and/or MHC class II molecules; and
b. increase expression of DUX4.

Embodiment 470. The engineered cell of any of embodiments 388-458, wherein the engineered cell comprises one or more modifications that:
a. reduce expression of MHC class I and/or MHC class II molecules; and
b. increase expression of IDO1.

Embodiment 471. The engineered cell of any of embodiments 388-458, wherein the engineered cell comprises one or more modifications that:
a. reduce expression of MHC class I and/or MHC class II molecules; and
b. increase expression of IL-35.

Embodiment 472. The engineered cell of any of embodiments 388-458, wherein the engineered cell comprises one or more modifications that:
a. reduce expression of MHC class I and/or MHC class II molecules; and
b. increase expression of PD-L1.

Embodiment 473. The engineered cell of any of embodiments 388-458, wherein the engineered cell comprises one or more modifications that:
a. reduce expression of MHC class I and/or MHC class II molecules; and
b. increase expression of HLA-E.

Embodiment 474. The engineered cell of any of embodiments 388-458, wherein the engineered cell comprises one or more modifications that:
a. reduce expression of MHC class I and/or MHC class II molecules; and
b. increase expression of HLA-G.

Embodiment 475. The engineered cell of any of embodiments 388-458, wherein the engineered cell comprises one or more modifications that:
a. reduce expression of MHC class I and/or MHC class II molecules;
b. reduce expression of CD155; and
c. increase expression of HLA-E.

Embodiment 476. The engineered cell of any of embodiments 388-458, wherein the engineered cell comprises one or more modifications that:
a. reduce expression of MHC class I molecules;
b. reduce expression of RFXANK;
c. increase expression of HLA-E.

Embodiment 477. The engineered cell of any of embodiments 388-458, wherein the engineered cell comprises one or more modifications that:
a. reduce expression of MHC class I and/or MHC class II molecules;
b. reduce expression of MIC-A and/or MIC-B;
c. increase expression of one or more of CD47, CD24 and PD-L1; and
d. increase expression of CD46, CD55, CD59 and CR1.

Embodiment 478. The engineered cell of any of embodiments 388-458, wherein the engineered cell comprises one or more modifications that:
a. reduce expression of MHC class I molecules;
b. reduce expression of MIC-A and/or MIC-B;
c. reduce expression of TXNIP; and
d. increase expression of PD-L1 and HLA-E.

Embodiment 479. The engineered cell of embodiment 477, wherein the modifications further increase expression of A20/TNFAIP3 and MANF.

Embodiment 480. The engineered of any one of embodiments 388-479, wherein the one or more modifications that reduce expression of MHC class I and/or MHC class II molecules consist of one or more modifications that reduce expression of MHC class I molecules.

Embodiment 481. The engineered of any one of embodiments 388-479, wherein the one or more modifications that reduce expression of MHC class I and/or MHC class II molecules consist of one or more modifications that reduce expression of MHC class II molecules.

Embodiment 482. The engineered of any one of embodiments 388-479, wherein the one or more modifications that reduce expression of MHC class I and/or MHC class II molecules consist of one or more modifications that reduce expression of MHC class I molecules and MHC class II molecules.

Embodiment 483. The engineered cell of embodiment 388-482, wherein increased expression comprises increased mRNA expression.

Embodiment 484. The engineered cell of embodiment 388-483, wherein increased expression comprises increased protein expression or protein activity.

Embodiment 485. The engineered cell of any one of embodiments 388-484, wherein increased expression comprises increasing activity of a gene encoding or regulating the expression of i) the one or more tolerogenic factors, or ii) the one or more additional tolerogenic factors.

Embodiment 486. The engineered cell of embodiment 485, wherein the gene is an endogenous gene and the one or more modifications comprise one or more modifications of an endogenous promoter.

Embodiment 487. The engineered cell of embodiment 485, wherein the gene is an endogenous gene and the one or more modifications comprise introduction of a heterologous promoter.

Embodiment 488. The engineered cell of embodiment 487, wherein the heterologous promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 489. The engineered cell of any of embodiments 388-481, wherein the engineered cell comprises one or more transgenes.

Embodiment 490. The engineered cell of embodiment 489, wherein the one or more transgenes encode at least one of the one or more tolerogenic factors or the one or more additional tolerogenic factors.

Embodiment 491. The engineered cell of embodiment 489 or 490, wherein the one or more transgenes encode at least one of the one or more additional tolerogenic factors.

Embodiment 492. The engineered cell of any one of embodiments 489-491, wherein the one or more transgenes encode one or more additional molecules.

Embodiment 493. The engineered cell of any of embodiments 489-492, wherein the one or more transgenes comprise one or more regulatory elements.

Embodiment 494. The engineered cell of any of embodiments 489-493, wherein the one or more transgenes are operably linked to the one or more regulatory elements.

Embodiment 495. The engineered cell of embodiment 493 or embodiment 107, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.

Embodiment 496. The engineered cell of embodiment 493, embodiment 495, or embodiment 107, wherein the one or more regulatory elements comprise a promoter.

Embodiment 497. The engineered cell of embodiment 495 or 496, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 498. The engineered cell of any of embodiments 489-497, wherein the engineered cell comprises one or more vectors encoding the one or more transgenes.

Embodiment 499. The engineered cell of embodiment 498, wherein at least one of the one or more vectors is a multicistronic vector.

Embodiment 500. The engineered cell of embodiment 499, wherein the multicistronic vector encodes at least one of the one or more tolerogenic factors or the one or more additional tolerogenic factors.

Embodiment 501. The engineered cell of embodiment 499 or embodiment 113, wherein the multicistronic vector further encodes at least one of the one or more tolerogenic factors or the one or more additional tolerogenic factors.

Embodiment 502. The engineered cell of embodiment of embodiment 500 or embodiment 501, wherein the multicistronic vector further encodes at least one of the one or more additional molecules.

Embodiment 503. The engineered cell of any one of embodiments 489-502, wherein the one or more transgenes are separated by one or more linker sequences.

Embodiment 504. The engineered cell of embodiment 503, wherein the one or more linker sequences comprise an IRES sequence or a cleavable peptide sequence.

Embodiment 505. The engineered cell of embodiment 504, wherein the cleavable peptide sequence comprises a self-cleavable peptide, optionally a 2A peptide.

Embodiment 506. The engineered cell of embodiment 505, wherein the 2A peptide is selected from the group consisting of a F2A sequence, an E2A sequence, a P2A sequence, and a T2A sequence.

Embodiment 507. The engineered cell of any of embodiments 504-506, wherein the cleavable peptide sequence comprises a protease cleavable sequence or a chemically cleavable sequence.

Embodiment 508. The engineered cell of any of embodiments 500-507, wherein the one or more tolerogenic factors, the one or more additional tolerogenic factors, and/or the one or more additional molecules are operably linked to the same promoter.

Embodiment 509. The engineered cell of any of embodiment 508, wherein the promoter is a constitutive promoter.

Embodiment 510. The engineered cell of embodiment 508 or 509, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

Embodiment 511. The engineered cell of any of embodiments 492-510, wherein the one or more additional molecules comprise a chimeric antigen receptor (CAR).

Embodiment 512. The engineered cell of embodiment 511, wherein the CAR comprises a signal peptide, an extracellular binding domain specific to CD19, a hinge domain, a transmembrane domain, an intracellular costimulatory domain, and/or an intracellular signaling domain.

Embodiment 513. The engineered cell of embodiment 511 or embodiment 125, wherein the CAR is specific for CD19, CD20, CD22, CD3δ, CD123, CD138, BCMA, or any combination thereof.

Embodiment 514. The engineered cell of embodiment 513, wherein the CAR is a CD19/CD22-bispecific CAR.

Embodiment 515. The engineered cell of any of embodiments 492-514, wherein the one or more additional molecules comprise one or more safety switches.

Embodiment 516. The engineered cell of embodiment 515, wherein the one or more safety switches are capable of controlled killing of the engineered cell.

Embodiment 517. The engineered cell of embodiment 515 or 516, wherein the one or more safety switches induce controlled cell death in the presence of a drug or prodrug, or upon activation by a selective exogenous compound.

Embodiment 518. The engineered cell of any of embodiments 515-517, wherein the one or more safety switches comprise is an inducible protein capable of inducing apoptosis of the engineered cell.

Embodiment 519. The engineered cell of embodiment 518, wherein the inducible protein capable of inducing apoptosis of the engineered cell is a caspase protein.

Embodiment 520. The engineered cell of embodiment 519, wherein the caspase protein is caspase 9.

Embodiment 521. The engineered cell of any of embodiments 515-520, wherein the one or more safety switches comprise one or more suicide genes.

Embodiment 522. 135. The engineered cell of embodiment 521, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 523. The engineered cell of any of embodiments 489-522, wherein at least one of the one or more transgenes are integrated into the genome of the engineered cell.

Embodiment 524. The engineered cell of embodiment 523, wherein integration is by non-targeted insertion into the genome of the engineered cell.

Embodiment 525. The engineered cell of embodiment 524, wherein integration is by non-targeted insertion into the genome of the engineered cell using a lentiviral vector.

Embodiment 526. The engineered cell of embodiment 523, wherein integration is by targeted insertion into a target genomic locus of the engineered cell.

Embodiment 527. The engineered cell of embodiment 526, wherein targeted insertion is by nuclease-mediated gene editing with homology-directed repair.

Embodiment 528. The engineered cell of embodiment 526 or 527, wherein the target genomic locus is selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

Embodiment 529. The engineered cell of any of embodiments 388-528, wherein the genome of the engineered cell comprises on or more gene edits in one or more genes encoding the one or more molecules of any of embodiments 388-141 having reduced expression.

Embodiment 530. The engineered cell of any of embodiments 388-529, wherein the engineered cell comprises a genome editing complex.

Embodiment 531. The engineered cell of embodiment 530, wherein the genome editing complex comprises a genome targeting entity and a genome modifying entity.

Embodiment 532. The engineered cell of embodiment 531, wherein the genome targeting entity localizes the genome editing complex to the target locus, optionally wherein the genome targeting entity is a nucleic acid-guided targeting entity.

Embodiment 533. The engineered cell of embodiment 531 or embodiment 532, wherein the genome targeting entity comprises a transcription activator-like effector (TALE) binding protein, a zinc finger (ZF) binding protein, a Meganuclease, a Cas protein, a TnpB protein, a homing endonuclease, an endonuclease-deficient-Cas protein, an enzymatically inactive Cas protein, a nucleic acid programmable DNA binding protein, or a functional portion thereof.

Embodiment 534. The engineered cell of any of embodiments 531-533, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, or a functional portion thereof.

Embodiment 535. The engineered cell of embodiment 531, wherein the genome modifying entity cleaves, deaminates, nicks, polymerizes, interrogates, integrates, cuts, unwinds, breaks, alters, methylates, demethylates, or otherwise destabilizes the target locus.

Embodiment 536. The engineered cell of embodiment 531 or embodiment 535, wherein the genome modifying entity comprises a recombinase, integrase, transposase, endonuclease, exonuclease, nickase, helicase, polymerase, reverse transcriptase, deaminase, flippase, methylase, demethylase, acetylase, a nucleic acid modifying protein, an RNA modifying protein, a DNA modifying protein, Argonaute protein, an epigenetic modifying protein, a histone modifying protein, or a functional portion thereof.

Embodiment 537. The engineered cell of embodiment 536, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, APOBEC1, cytidine deaminase, adenosine deaminase, uracil glycosylase inhibitor (UGI), adenine base editors (ABE), cytosine base editors (CBE), reverse transcriptase, serine integrase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

Embodiment 538. The engineered cell of any of embodiments 531-537, wherein the genome targeting entity and the genome modifying entity are different domains of a single polypeptide.

Embodiment 539. The engineered cell of any of embodiments 531-538, wherein the genome editing entity and genome modifying entity are two different polypeptides that are operably linked together.

Embodiment 540. The engineered cell of any of embodiments 531-538, wherein the genome editing entity and genome modifying entity are two different polypeptides that are not linked together.

Embodiment 541. The engineered cell of any of embodiments 462-469, wherein the modification is by a genome-modifying protein.

Embodiment 542. The engineered cell of any of embodiments 470, wherein the modification by a genome-modifying protein is modification by a CRISPR-associated transposase, prime editing, or Programmable Addition via Site-specific Targeting Elements (PASTE).

Embodiment 543. The engineered cell of any of embodiments 470-471, wherein the modification by the genome-modifying protein is nuclease-mediated gene editing.

Embodiment 544. The engineered cell of embodiment 472, wherein the nuclease-mediated gene editing is by a zinc finger nuclease (ZFN), a TAL-effector nuclease (TALEN), or a CRISPR-Cas combination that targets the B2M gene, optionally wherein the Cas is selected from a Cas9 or a Cas12.

Embodiment 545. The engineered cell of any of embodiments 470-472, wherein the modification by the genome-modifying protein is performed by one or more proteins selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

Embodiment 546. 158. The engineered cell of embodiment 473, wherein the nuclease-mediated gene editing is by a CRISPR-Cas combination and the CRISPR-Cas combination comprises a guide RNA (gRNA) having a targeting domain that is complementary to at least one target site within the B2M gene.

Embodiment 547. The engineered cell of embodiment 475, wherein the CRISPR-Cas combination is a ribonucleoprotein (RNP) complex comprising the gRNA and a Cas protein.

Embodiment 548. The engineered cell of any of embodiments 1-547, wherein the engineered cell is a human cell or an animal cell.

Embodiment 549. The engineered cell of embodiment 548, wherein the animal cell is a porcine cell, a bovine cell, or an ovine cell.

Embodiment 550. The engineered cell of embodiment 548, wherein the engineered cell is a human cell.

Embodiment 551. The engineered cell of any of embodiments 388-550, wherein the engineered cell is a stem cell or progenitor cell.

Embodiment 552. The engineered cell of embodiment 551, wherein the engineered cell is a differentiated cell derived from the stem cell or progenitor cell.

Embodiment 553. The engineered cell of embodiment 551 or 552, wherein the stem cell or progenitor cell is selected from the group consisting of an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell.

Embodiment 554. The engineered cell of any of embodiments 388-550, wherein the engineered cell is a differentiated cell derived from a pluripotent stem cell or a progeny thereof.

Embodiment 555. The engineered cell of embodiment 554, wherein the pluripotent stem cell is an induced pluripotent stem cell.

Embodiment 556. The engineered cell of any of embodiments 388-550, wherein the engineered cell is a primary cell isolated from a donor subject.

Embodiment 557. The engineered cell of embodiment 556, wherein the donor subject is healthy or is not suspected of having a disease or condition at the time the donor sample is obtained from the individual donor.

Embodiment 558. The engineered cell of any of embodiments 388-557, wherein the engineered cell is selected from the group consisting of an islet cell, a beta islet cell, a pancreatic islet cell, an immune cell, a B cell, a T cell, a natural killer (NK) cell, a natural killer T (NKT) cell, a macrophage cell, an endothelial cell, a muscle cell, a cardiac muscle cell, a smooth muscle cell, a skeletal muscle cell, a dopaminergic neuron, a retinal pigmented epithelium cell, an optic cell, a hepatocyte, a thyroid cell, a skin cell, a glial progenitor cell, a neural cell, a cardiac cell, a stem cell, a hematopoietic stem cell, an induced pluripotent stem cell (iPSC), a mesenchymal stem cell (MSC), an embryonic stem cell (ESC), a pluripotent stem cell (PSC), and a blood cell.

Embodiment 559. The engineered cell of any of embodiments 388-558, wherein the cell is ABO blood group type O.

Embodiment 560. The engineered cell of any of embodiments 388-559, wherein the cell comprises a functional ABO A allele and/or a functional ABO B allele.

Embodiment 561. The engineered cell of any of embodiments 388-560, wherein the cell is Rhesus factor negative (Rh−).

Embodiment 562. The engineered cell of any of embodiments 388-560, wherein the cell is Rhesus factor positive (Rh+).

Embodiment 563. A method of generating the engineered cell of any of embodiments 388-562 comprising

Embodiment 564. a. obtaining a cell; and

Embodiment 565. b. introducing the one or more modifications of any of embodiments 388-562 into the cell.

Embodiment 566. The method of embodiment 563, wherein the method further comprises selecting the engineered cell from a population of cells based on the presence and/or level of one or more of the modifications.

Embodiment 567. The method of embodiment 563 or 564, wherein the cell is a stem cell or a progenitor cell and the method further comprises differentiating the stem cell or the progenitor cell.

Embodiment 568. The method of embodiment 563 or 564, wherein the cell is a pluripotent stem cell or a progeny thereof and the method comprises differentiating the pluripotent stem cell or progeny thereof.

Embodiment 569. The method of embodiment 563 or 564, wherein the cell is a primary cell.

Embodiment 570. The method of any of embodiments 563-567, wherein the method comprises introducing one or more gene edits into the genome of the cell.

Embodiment 571. The method of embodiment 568, wherein the one or more gene edits are introduced into the genome of the cell by non-targeted insertion.

Embodiment 572. The method of embodiment 568, wherein the one or more gene edits are introduced into the genome of the cell by targeted insertion.

Embodiment 573. The method of embodiment 568 or 570, wherein the one or more gene edits are introduced into one or more genes encoding the one or more molecules of any of embodiments 388-561.

Embodiment 574. The method of embodiment 571, wherein the engineered cell has increased expression of the one or more molecules encoded by the one or more edited genes.

Embodiment 575. The method of embodiment 571 or 572, wherein the engineered cell has reduced expression of the one or more molecules encoded by the one or more edited genes.

Embodiment 576. The method of any of embodiments 568-185, wherein the one or more gene edits are introduced into the genome of cell using at least one of the genome editing complexes of any of embodiments 530-547.

Embodiment 577. The method of any of embodiments 568-574, wherein the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

Embodiment 578. An engineered cell produced according to the method of any of embodiments 563-575.

Embodiment 579. The engineered cell of any of embodiments 388-562 and 576, wherein the engineered cell, or progeny or differentiated cells have increased capability to evade NK cell mediated cytotoxicity upon administration to a subject as compared to a cell of the same type that does not comprise the one or more modifications.

Embodiment 580. The engineered cell of any of embodiments 388-562, 576 and 577, wherein the engineered cell, or progeny or differentiated cells derived from the engineered cell undergo reduced cell lysis by mature NK cells upon administration to a subject as compared to a cell of the same type that does not comprise the one or more modifications.

Embodiment 581. The engineered cell of any of embodiments 388-562 and 576-578, wherein the engineered cell, or progeny or differentiated cells derived from the engineered cell induce a reduced immune response upon administration to a subject as compared to a cell of the same type that does not comprise the one or more modifications.

Embodiment 582. The engineered cell of any of embodiments 388-562 and 576-579, wherein the engineered cell, or progeny or differentiated cells derived from the engineered cell induce a reduced systemic inflammatory response upon administration to a subject as compared to a cell of the same type that does not comprise the one or more modifications.

Embodiment 583. The engineered cell of any of embodiments 388-562 and 576-580, wherein the engineered cell, or progeny or differentiated cells derived from the engineered cell induce a reduced local inflammatory response upon administration to a subject as compared to a cell of the same type that does not comprise the one or more modifications.

Embodiment 584. The engineered cell of any of embodiments 388-562 and 576-581, wherein the engineered cell, or progeny or differentiated cells derived from the engineered cell induce reduced complement pathway activation upon administration to a subject as compared to a cell [of the same type] that does not comprise the one or more modifications.

Embodiment 585. The engineered cell of any of embodiments 388-562 and 576-582, wherein the engineered cell, or progeny or differentiated cells derived from the engineered cell retain the ability to engraft and function upon administration to a subject.

Embodiment 586. The engineered cell of any of embodiments 388-562 and 576-583, wherein the engineered cell, or progeny or differentiated cells derived from the engineered cell has increased ability to engraft and function upon administration to a subject as compared to a cell [of the same type] that does not comprise the one or more modifications.

Embodiment 587. A population of engineered cells comprising a plurality of the engineered cells of any of embodiments 388-562 and 576-584.

Embodiment 588. The population of engineered cells of embodiment 585, wherein at least about 30% of cells in the population comprise the plurality of the engineered cells.

Embodiment 589. The population of engineered cells of embodiment 585 or embodiment 586, wherein the plurality of the engineered cells are primary cells isolated from more than one donor subject.

Embodiment 590. The population of engineered cells of embodiment 587, wherein each donor subject is healthy or is not suspected of having a disease or condition at the time the donor sample is obtained from the individual donor.

Embodiment 591. A method of producing a composition comprising the engineered cell of any of embodiments 1-562 and 576-196 or the population of engineered cells of any of embodiments 585-588 comprising
a. obtaining the cell of any of embodiments 548-562;
b. introducing the one or more modifications of any of embodiments 388-562 into the cell;
c. selecting the engineered cell or selecting the population of engineered cells from a population of cells based on a level of the one or more of the modifications; and
d. formulating the composition comprising the selected engineered cell or the selected population of engineered cells.

Embodiment 592. The method of embodiment 589, wherein method comprises selecting the engineered cell or the population of engineered cells based on the level of cell surface expression of the one or more modified molecules in any of embodiments 388-561.

Embodiment 593. The method of embodiment 589 or embodiment 590, wherein the engineered cell or the population of engineered cells are selected based on a level of the one or more modified molecules having reduced expression in the engineered cell or the population of engineered cells.

Embodiment 594. The method of any of embodiments 589-591, wherein the engineered cell or the population of engineered cells are selected based on a level of the one or more modified molecules having increased expression in the engineered cell or the population of engineered cells.

Embodiment 595. The method of any of embodiments 589-592, wherein the method comprises formulating the composition in a pharmaceutically acceptable additive, carrier, diluent, or excipient.

Embodiment 596. The method of embodiment 593, wherein the pharmaceutically acceptable additive, carrier, diluent, or excipient comprises a pharmaceutically acceptable buffer.

Embodiment 597. The method of embodiment 594, wherein the pharmaceutically acceptable buffer comprises neutral buffer saline or phosphate buffered saline.

Embodiment 598. The method of any of embodiments 589-595, wherein the method comprises formulating the composition with Plasma-Lyte A®, dextrose, dextran, sodium chloride, human serum albumin (HSA), dimethylsulfoxide (DMSO), or a combination thereof.

Embodiment 599. The method of any of embodiments 589-596, wherein the method comprises formulating the composition with a cryoprotectant.

Embodiment 600. The method of any of embodiments 589-597, wherein the method comprises formulating the composition in a serum-free cryopreservation medium comprising a cryoprotectant.

Embodiment 601. The method of embodiment 597 or embodiment 598, wherein the cryoprotectant comprises DMSO.

Embodiment 602. The method of embodiment 598 or embodiment 599, wherein the serum-free cryopreservation medium comprises about 5% to about 10% DMSO (v/v).

Embodiment 603. The method of any of embodiments 598-600, wherein the serum-free cryopreservation medium comprises about 10% DMSO (v/v).

Embodiment 604. The method of any of embodiments 589-601, wherein the method further comprises storing the composition in a container.

Embodiment 605. The method of any of embodiments 589-602, wherein the method further comprises thawing the cell before step (b).

Embodiment 606. 216. The method of any of embodiments 589-603, wherein the method further comprises freezing the engineered cell, the population of engineered cells, or the composition.

Embodiment 607. The method of embodiment 604, wherein the engineered cell or the population of engineered cells are frozen after step (b).

Embodiment 608. The method of embodiment 605, wherein the engineered cell or the population of engineered cells are thawed before step (c).

Embodiment 609. The method of embodiment 604, wherein the engineered cell or the population of engineered cells are frozen after step (c).

Embodiment 610. The method of embodiment 607, wherein the engineered cell or the population of engineered cells are thawed before step (d).

Embodiment 611. The method of embodiment 604, wherein the engineered cell or the population of engineered cells are frozen after step (c).

Embodiment 612. The method of any of embodiments 589-609, wherein the composition is frozen after step (d).

Embodiment 613. A composition comprising the engineered cell of any of embodiments 1-562 and 576-196 or the population of engineered cells of any of embodiments 585-588.

Embodiment 614. A composition produced by the method of any one of embodiments 589-610.

Embodiment 615. The composition of embodiment 611 or embodiment 612, wherein the composition comprises a pharmaceutically acceptable additive, carrier, diluent, or excipient.

Embodiment 616. The composition of any of embodiments 611-613, wherein the composition is sterile.

Embodiment 617. A container comprising the composition of any of embodiments 612-614.

Embodiment 618. The container of embodiment 615, wherein the container is a sterile bag.

Embodiment 619. The container of embodiment 616, wherein the sterile bag is a cryopreservation-compatible bag.

Embodiment 620. A kit comprising the composition of any of embodiments 612-614 or the container of any of embodiments 615-617.

Embodiment 621. The kit of embodiment 618, wherein the kit further comprises instructions for using the engineered cells or the population of engineered cells.

Embodiment 622. A method of treating a condition or disease in a subject in need thereof comprising administering to the subject an effective amount of the engineered cell of any of embodiments 1-562 and 576-196, the population of engineered cells of any of embodiments 585-588, or the composition of any of embodiments 611-613, optionally wherein the disease or condition is a cellular deficiency.

Embodiment 623. The method of embodiment 620, wherein the condition or disease is selected from the group consisting of diabetes, cancer, vascularization disorders, ocular disease, thyroid disease, skin diseases, and liver diseases.

Embodiment 624. The method of embodiment 620 or 621, wherein the condition or disease is associated with diabetes or is diabetes, optionally wherein the diabetes is Type I diabetes.

Embodiment 625. The method of embodiment 622, wherein the population of engineered cells is a population of islet cells, including beta islet cells.

Embodiment 626. The method of embodiment 623, wherein the islet cells are selected from the group consisting of an islet progenitor cell, an immature islet cell, and a mature islet cell.

Embodiment 627. The method of embodiment 620, wherein the condition or disease is associated with a vascular condition or disease or is a vascular condition or disease.

Embodiment 628. The method of embodiment 625, wherein the engineered cell or the population of engineered cells comprises an endothelial cell.

Embodiment 629. The method of embodiment 620, wherein the condition or disease is associated with autoimmune thyroiditis or is autoimmune thyroiditis.

Embodiment 630. The method of embodiment 627, wherein the engineered cell or the population of engineered cells comprise a thyroid progenitor cell.

Embodiment 631. The method of embodiment 620, wherein the condition or disease is associated with a liver disease or is liver disease.

Embodiment 632. The method of embodiment 629, wherein the liver disease comprises cirrhosis of the liver.

Embodiment 633. The method of embodiment 629 or 630, wherein the engineered cell or the population of engineered cells comprise a hepatocyte or a hepatic progenitor cell.

Embodiment 634. The method of embodiment 620, wherein the condition or disease is associated with a corneal disease or is corneal disease.

Embodiment 635. The method of embodiment 632, wherein the corneal disease is Fuchs dystrophy or congenital hereditary endothelial dystrophy.

Embodiment 636. The method of embodiment 632 or 633, wherein engineered cell or the population of engineered cells comprise a corneal endothelial progenitor cell or a corneal endothelial cells.

Embodiment 637. The method of embodiment 620, wherein the condition or disease is associated with a kidney disease or is kidney disease.

Embodiment 638. The method of embodiment 635, wherein the engineered cell or the population of engineered cells comprise a renal precursor cell or a renal cell.

Embodiment 639. The method of embodiment 620, wherein the condition or disease is associated with a cancer or is cancer.

Embodiment 640. The method of embodiment 637, wherein the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

Embodiment 641. The method of embodiment 637 or 638, wherein the engineered cell or the population of engineered cells comprise a T cell, an NK cell, or an NKT cell.

Embodiment 642. The method of embodiment 620, wherein the condition or disease is associated with a hematopoietic disease or disorder or is a hematopoietic disease or disorder.

Embodiment 643. The method of embodiment 640, wherein the hematopoietic disease or disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.

Embodiment 644. The method of embodiment 620, wherein the condition or disease is associated with leukemia or myeloma or is leukemia or myeloma.

Embodiment 645. The method of embodiment 620, wherein the condition or disease is associated with an autoimmune disease or condition or is an autoimmune disease or condition.

Embodiment 646. The method of embodiment 643, wherein the autoimmune disease or condition is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopeniarpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

Embodiment 647. The method of any of embodiments 640-644, wherein engineered cell or the population of engineered cells comprises a hematopoietic stem cell (HSC) or a derivative thereof.

Embodiment 648. The method of embodiment 620, wherein the condition or disease is associated with Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS), or wherein the disease or condition is Parkinson's disease, Huntington disease, multiple sclerosis, a neurodegenerative disease or condition, attention deficit hyperactivity disorder (ADHD), Tourette Syndrome (TS), schizophrenia, psychosis, depression, a neuropsychiatric disorder stroke, or amyotrophic lateral sclerosis (ALS).

Embodiment 649. The method of embodiment 646, wherein the engineered cell or the population of engineered cells comprise a neural cell or a glial cell.

Embodiment 650. The method of any of embodiments 620-647, wherein the engineered cell or the population of engineered cells are expanded and cryopreserved prior to administration.

Embodiment 651. The method of any of embodiments 620-648, wherein the method comprises intravenous injection, intramuscular injection, intravascular injection, or transplantation of the engineered cell, the population of engineered cells, or the composition.

Embodiment 652. The method of embodiment 649, wherein transplantation comprises intravascular injection or intramuscular injection.

Embodiment 653. The method of any of embodiments 620-650, wherein the method further comprises administering one or more immunosuppressive agents to the subject.

Embodiment 654. The method of any of embodiments 620-651, wherein the subject has been administered one or more immunosuppressive agents.

Embodiment 655. The method of embodiment 651 or embodiment 652, wherein the one or more immunosuppressive agents are a small molecule or an antibody.

Embodiment 656. The method of any of embodiments 651-653, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), an immunomodulatory agent, and an immunosuppressive antibody.

Embodiment 657. The method of any of embodiments 651-654, wherein the one or more immunosuppressive agents comprise cyclosporine.

Embodiment 658. The method of any of embodiments 651-654, wherein the one or more immunosuppressive agents comprise mycophenolate mofetil.

Embodiment 659. The method of any of embodiments 651-654, wherein the one or more immunosuppressive agents comprise a corticosteroid.

Embodiment 660. The method of any of embodiments 651-654, wherein the one or more immunosuppressive agents comprise cyclophosphamide.

Embodiment 661. The method of any of embodiments 651-654, wherein the one or more immunosuppressive agents comprise rapamycin.

Embodiment 662. The method of any of embodiments 651-654, wherein the one or more immunosuppressive agents comprise tacrolimus (FK-506).

Embodiment 663. The method of any of embodiments 651-654, wherein the one or more immunosuppressive agents comprise anti-thymocyte globulin.

Embodiment 664. The method of any of embodiments 651-654, wherein the one or more immunosuppressive agents are one or more immunomodulatory agents.

Embodiment 665. The method of embodiment 662, wherein the one or more immunomodulatory agents are a small molecule or an antibody.

Embodiment 666. The method of embodiment 662 or embodiment 663, wherein the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

Embodiment 667. The method of any of embodiments 651-664, wherein the one or more immunosuppressive agents are or have been administered to the subject prior to administration of the engineered cell, the population of engineered cells, or the composition.

Embodiment 668. The method of any of embodiments 651-665, wherein the one or more immunosuppressive agents are or have been administered to the subject at least 1, 2, 3, 4, 5, 6, 7, 8, 9, 10, 11, 12, 13, or 14 days prior to administration of the engineered cell, the population of engineered cells, or the composition.

Embodiment 669. The method of any of embodiments 651-666, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the engineered cell, the population of engineered cells, or the composition.

Embodiment 670. The method of any of embodiments 651-664, wherein the one or more immunosuppressive agents are or have been administered to the subject after administration of the engineered cell, the population of engineered cells, or the composition.

Embodiment 671. The method of any of embodiments 651-664 and 668, wherein the one or more immunosuppressive agents are or have been administered to the subject at least 1, 2, 3, 4, 5, 6, 7, 8, 9, 10, 11, 12, 13, or 14 days after administration of the engineered cell, the population of engineered cells, or the composition.

Embodiment 672. The method of any of embodiments 651-664, 668 and 669, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the engineered cell, the population of engineered cells, or the composition.

Embodiment 673. The method of any of embodiments 651-664, wherein the one or more immunosuppressive agents are or have been administered to the subject on the same day as the first administration of the engineered cell, the population of engineered cells, or the composition.

Embodiment 674. The method of any of embodiments 651-664, wherein the one or more immunosuppressive agents are or have been administered to the subject after administration of a first and/or second administration of the engineered cell, the population of engineered cells, or the composition.

Embodiment 675. The method of any of embodiments 651-664, wherein the one or more immunosuppressive agents are or have been administered to the subject prior to administration of a first and/or second administration of the engineered cell, the population of engineered cells, or the composition.

Embodiment 676. The method of any of embodiments 651-664, wherein the one or more immunosuppressive agents are or have been administered to the subject at least 1, 2, 3, 4, 5, 6, 7, 8, 9, 10, 11, 12, 13, or 14 days prior to administration of a first and/or second administration of the engineered cell, the population of engineered cells, or the composition.

Embodiment 677. The method of any of embodiments 651-664, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the engineered cell, the population of engineered cells, or the composition.

Embodiment 678. The method of any of embodiments 651-664, wherein the one or more immunosuppressive agents are or have been administered to the subject at least 1, 2, 3, 4, 5, 6, 7, 8, 9, 10, 11, 12, 13, or 14 days after administration of a first and/or second administration of the engineered cell, the population of engineered cells, or the composition.

Embodiment 679. The method of any of embodiments 651-664, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the engineered cell, the population of engineered cells, or the composition.

Embodiment 680. The method of any of embodiments 651-677, wherein the one or more immunosuppressive agents are administered at a lower dosage as compared to the dosage administered to reduce immune rejection of a cell that does not comprise the one or more modifications of the engineered cell or the population of engineered cells.

Embodiment 681. The method of any of embodiments 620-678, wherein the method further comprises activating the safety switch to induce controlled cell death after the administration of the engineered cell, the population of engineered cells, or the composition to the subject.

Embodiment 682. The method of any of embodiments 620-679, wherein the suicide gene or the suicide switch is activated to induce controlled cell death after the administration of the one or more immunosuppressive agents to the subject.

Embodiment 683. The method of any of embodiments 620-679, wherein the suicide gene or the suicide switch is activated to induce controlled cell death prior to the administration of the one or more immunosuppressive agents to the subject.

Embodiment 684. The method of any of embodiments 620-681, wherein the safety switch is activated to induce controlled cell death in the event of cytotoxicity or other negative consequences to the subject.

Embodiment 685. The method of any of embodiments 620-682, wherein the method comprises administering an agent that allows for depletion of the engineered cell, the population of engineered cells, or the composition.

Embodiment 686. The method of embodiment 683, wherein the agent that allows for depletion of the engineered cell is an antibody that recognizes a protein expressed on the cell surface.

Embodiment 687. The method of embodiment 684, wherein the antibody is selected from the group consisting of an antibody that recognizes CCR4, CD16, CD19, CD20, CD30, EGFR, GD2, HER1, HER2, MUC1, PSMA, and RQR8.

Embodiment 688. The method of embodiment 684 or embodiment 685, wherein the antibody is selected from the group consisting of mogamulizumab, AFM13, MOR208, obinutuzumab, ublituximab, ocaratuzumab, rituximab, rituximab-RIIb, tomuzotuximab, RO5083945 (GA201), cetuximab, Hul4.18K322A, Hul4.18-IL2, Hu3F8, dinituximab, c.60C3-RIIc, and biosimilars thereof.

Embodiment 689. The method of any of embodiments 620-684, wherein the method comprises administering an agent that recognizes the one or more tolerogenic factors or the one or more additional tolerogenic factors on the cell surface.

Embodiment 690. The method of any of embodiments 620-687, wherein the method further comprises administering one or more additional therapeutic agents to the subject.

Embodiment 691. The method of any of embodiments 620-687, wherein the subject has been administered one or more additional therapeutic agents.

Embodiment 692. The method of any of embodiments 620-689, wherein the method further comprises monitoring the therapeutic efficacy of the method.

Embodiment 693. The method of any of embodiments 620-690, further comprising monitoring the prophylactic efficacy of the method.

Embodiment 694. The method of embodiment 690 or embodiment 691, wherein the method is repeated until a desired suppression of one or more disease symptoms occurs.

1. A method of treating a patient with an Epstein Barr Virus (EBV) infection comprising administering a population of engineered T cells to the patient, wherein the engineered T cells comprise reduced cell surface expression of one or more major histocompatibility complex (MHC) class I and/or class II human leukocyte antigen (HLA) molecules relative to an unaltered or unmodified wild-type or control cell, a first exogenous polynucleotide encoding a tolerogenic factor, and a second exogenous polynucleotide encoding one or more chimeric antigen receptors (CARs) wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain.

2-50. (canceled)

51. A method of treating an autoimmune disease in a patient that is suspected of having the autoimmune disease or has been diagnosed with the autoimmune disease comprising administering a population of engineered T cells to the patient, wherein the engineered T cells comprise reduced cell surface expression of one or more MHC class I and/or class II HLA molecules relative to an unaltered or unmodified wild-type or control cell, and comprise one or more exogenous polynucleotides, wherein the one or more exogenous polynucleotides encode:
a tolerogenic factor chosen from A20/TNFAIP3, C1-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof, and
one or more CARs, wherein the one or more CARs comprise an extracellular ligand-binding domain having specificity for CD19, CD20, CD22, BCMA, or an EBV antigen, a hinge domain, a transmembrane domain, a co-stimulatory domain, and an intracellular signaling domain,
and wherein the autoimmune disease is lupus, systemic lupus erythematosus, multiple sclerosis, systemic sclerosis, vasculitis, Crohn's disease, Myasthenia Gravis, Stiff-Person syndrome, or a pulmonary condition.

52-56. (canceled)

57. The method according to claim 51, wherein the one or more CARs comprise;
a hinge domain chosen from a CD8α hinge domain having the amino acid sequence of SEQ ID NO: 9, a CD28 hinge domain having the amino acid sequence of SEQ ID NO: 10 or 113, and an IgG4 hinge domain having the amino acid sequence of SEQ ID NO: 11 or 12;
a transmembrane domain chosen from a CD8α transmembrane domain having the amino acid sequence of SEQ ID NO: 14 and a CD28 transmembrane domain having the amino acid sequence of SEQ ID NO: 15 or 114;
a costimulatory domain chosen from a CD28 costimulatory domain having the amino acid sequence of SEQ ID NO: 17 and a 4-1BB costimulatory domain having the amino acid sequence of SEQ ID NO: 16; and/or
a CD3ζ signaling domain having the amino acid sequence of SEQ ID NO: 18 or 115.

58-67. (canceled)

68. The method according to claim 51, wherein the one or more CARs have a sequence of any one of SEQ ID NOs: 32, 34, 36, 45, 54, 117, 91, 92, 92, 128, 133, or 134.

69-70. (canceled)

71. The method of claim 51, wherein the engineered T cells comprise reduced expression of B2M, CIITA, TRAC, and/or TRB relative to an unaltered or unmodified wild-type or control cell, the tolerogenic factor is CD47, and the one or more CARs comprise a CD19-specific CAR.

72. The method of claim 71, wherein the CD19-specific CAR has an amino acid sequence chosen from: SEQ ID NOs: 32, 34, 36, and 117.

73. The method of claim 71, wherein the one or more CARs further comprise a CD22-specific CAR.

74. (canceled)

75. The method of claim 72, wherein the CD19-specific CAR has a sequence of SEQ ID NO: 117.

76. The method of claim 73, wherein the CD22-specific CAR has a sequence chosen from SEQ ID NOs: 45 and 54.

77-85. (canceled)

86. The method of claim 51, further comprising administering a second, third, fourth, fifth, or sixth dose of the engineered T cells to the patient.

87-92. (canceled)

93. The method of claim 73, wherein the CD19-specific CAR and the CD22-specific CAR are encoded by a single bicistronic polynucleotide.

94-95. (canceled)

96. The method of claim 73, wherein the CD19-specific CAR T cells and CD22-specific CAR T cells are administered concomitantly.

97-131. (canceled)

132. The method of claim 51, wherein the engineered T cells are primary T cells, are propagated from a primary T cell or a progeny thereof, or are derived from a T cell differentiated from an iPSC or a progeny thereof.

133-149. (canceled)

150. The method of claim 93, wherein the CD19-specific CAR, the CD22-specific CAR, and the CD47 are encoded by a single bicistronic polynucleotide.

151-153. (canceled)

154. The method of claim 51, wherein the one or more exogenous polynucleotides are inserted into one or more loci chosen from a safe harbor locus, a target locus, an RHD locus, a B2M locus, a CIITA locus, a TRAC locus, and a TRB locus.

155-158. (canceled)

158. The method of claim 51, wherein the one or more exogenous polynucleotides are introduced into the engineered T cells using a retrovirus or a fusosome.

159-166. (canceled)

167. The method of claim 51, wherein the engineered T cells do not induce an immune response to the cell upon administration to the patient.

168. The method of claim 51, wherein the administration is selected from the group consisting of intravenous injection, intramuscular injection, intravascular injection, and transplantation.

169-171. (canceled)

172. The method of claim 51, wherein the patient was treated with an immunodepleting therapy prior to administering the engineered T cells.

173-181. (canceled)

182. The method of claim 51, wherein at least about 40×10⁴ engineered T cells are administered to the patient.

183. (canceled)

184. The method of claim 51, wherein the engineered T cells persist in the subject for at least 4 weeks.

185-374. (canceled)

375. An engineered T cell comprising one or more modifications that (i) reduce cell surface expression of or disrupt one or more MHC class I molecules and/or one or more MHC class II molecules, and/or (ii) increase expression of one or more tolerogenic factors, wherein the reduced expression of (i) and the increased expression of (ii) is relative to a comparable T cell that does not comprise the modifications.

376-406. (canceled)
